(19) United States
(12) Patent Application Publication
(10) Pub. No.: US 2023/0332195 A1
(43) Pub. Date: Oct. 19, 2023

(54) GENETICALLY MODIFIED HOST CELLS PRODUCING BENZYLISOQUINOLINE ALKALOIDS

(71) Applicant: RIVER STONE BIOTECH APS, Copenhagen (DK)

(72) Inventors: Angela De CARVALHO, Copenhagen (DK); Swee Chuang Lim HALLWYL, Copenhagen (DK); Esben Halkjaer HANSEN, Copenhagen (DK); Jens HOUGHTON-LARSEN, Copenhagen (DK)

(73) Assignee: RIVER STONE BIOTECH APS, Copenhagen (DK)

(21) Appl. No.: 17/768,834

(22) PCT Filed: Oct. 9, 2020
(86) PCT No.: PCT/EP2020/078496

§ 371 Date: Apr. 13, 2022

(30) Foreign Application Priority Data
Oct. 10, 2019 (EP) 19202337.2
Apr. 15, 2020 (EP) 20169590.5

(51) Int. Cl. C12P 17/18; C12N 15/52; C12N 9/02; C12N 9/88; C12N 9/06; C12N 15/79

(52) U.S. Cl. CPC C12P 17/18; C12N 15/52; C12N 9/0071; C12N 9/88; C12N 9/0028; C12N 15/79; C12Y 114/16002; C12Y 402/01078; C12Y 114/19; C12Y 105/01027

(57) ABSTRACT

The invention relate to genetically modified hosts cell comprising a pathway having enhanced production of one or more benzylisoquinoline alkaloids wherein the cell expresses heterologous insect genes encoding insect demethylases converting thebaine into northebaine, thebaine into oripavine, thebaine into nororipavine and/or oripavine into nororipavine.

### FIELD OF THE INVENTION

The present disclosure relates to methods of producing benzylisoquinoline alkaloids by use of genetically modified host cells expressing one or more genes in an operative metabolic pathway producing the benzylisoquinoline alkaloids or their precursors as well as optionally subjecting the benzylisoquinoline alkaloids for chemical conversion to produce additional useful benzylisoquinoline alkaloid derivatives.

### BACKGROUND OF THE INVENTION

Effective production of pharmaceutical opioids by biotransformation, such as wholly or partly by fermentation of genetically engineered strains and/or by bioconversion, requires complex engineering and optimization of metabolic pathways producing the opioids or their precursors and optionally further chemical modifications. Some pharmaceutical opioids such as buprenorphine, naltrexone, naloxone and nalbuphine require demethylation of benzylisoquinoline alkaloids such as thebaine and/or oripavine and an N-alkylation of the demethylated benzylisoquinoline alkaloid. In the art this demethylation step and the subsequent N-alkylation step is achieved chemically and the chemical N-demethylation of benzylisoquinoline alkaloids preceding the N-alkylation is one of the most critical steps in the chemical synthesis of pharmaceutical opioids, as it has low efficiency and produces highly toxic waste.

Benzylisoquinoline alkaloids for the demethylation step can be provided by production using genetically modified cull cultures comprising the right pathway and/or extraction from plant material. Moreover, genetically modified yeasts comprising certain heterologous fungal Mucorales P450 enzymes capable of converting e.g. thebaine into northebaine and/or demethylated reticulin derivatives are known in the art eg. from WO2018229306.

However, for efficiently producing pharmaceutical opioids there is a continuous desire and need for improving and optimising both pathways in genetically modified microbial strains producing benzylisoquinoline alkaloids as well as critical steps of demethylating benzylisoquinoline alkaloids and improving further chemical modification of benzylisoquinoline alkaloids to produce compounds of particular desirable pharmacological properties with higher efficiency and less waste problems.

### SUMMARY OF THE INVENTION

Over this background art several improved pathway enzymes as well improvements in auxiliary cellular mechanisms have been identified to be surprisingly efficient at producing highly pure benzylisoquinoline alkaloids as well at demethylating benzylisoquinoline alkaloids thebaine and/or oripavine in host cells into the corresponding northebaine and/or nororipavine-auxiliary cellular mechanisms including transportation of precursors and products, limitation of precursor loss to competing cellular reactions and/or formation of by-products by unspecific enzymes.

Accordingly, the present invention provides in a first aspect a genetically modified host cell comprising a pathway having enhanced production of one or more benzylisoquinoline alkaloids wherein the cell comprises one or more features selected from:
a) expression of one or more heterologous genes encoding a demethylase capable of converting thebaine into northebaine, thebaine into oripavine, thebaine into nororipavine and/or oripavine into nororipavine;
b) expression of one or more heterologous genes encoding a tyrosine hydroxylase (TH) converting L-tyrosine into L-dopa, wherein the TH has at least 70% identity to the TH comprised in 7, 9, 11, 13, 15, 17, 19, 21, 23, 25, 27, 29, 31, 33, 35, 37, 39, 41, 43, 45, 47, 49, 51, 53, 55, 57, 59, 61, 63 or 65;
c) reduction or elimination of activity of one or more dehydrogenases native to the host cell comprised in SEQ ID NO: 663, 665, 667, 669, 671, 673, 675, 677, 679, 681, 683, 685, 687, 689, 691, 693, 695, 697, 699, 701, 703 or 705;
d) reduction or elimination of activity of one or more reductases native to the host cell comprised in SEQ ID NO: 707, 709, 711, 713, 715, 717, 719, 721, 723, 725, 727, 729 or 731;
e) expression of one or more heterologous genes encoding a norcoclaurine synthase (NCS) converting Dopamine and 4-HPAA into (S)-norcoclaurine, wherein the NCS has at least 70% identity to the NCS comprised in SEQ ID NO: 73 OR 76;
f) expression of one or more heterologous genes encoding
   i) a fused 1,2-dehydroreticuline synthase-1,2-dehydroreticuline reductase (DRS-DRR) converting (S)-Reticuline into (R)-reticuline, wherein
      ia) the DRS-DDR has at least 70% identity to the DRS-DRR comprised in SEQ ID NO: 92, 94, 96; or
      ib) the DRS moiety has at least 70%, identity to the DRS comprised in SEQ ID NO: 98, 100, 102, 104 or 106; and the DRR moiety has at least 70% identity to the DRR comprised in SEQ ID NO: 108 or 110; or
   ii) a DRS having at least 70% identity to the DRS comprised in SEQ ID NO: 98, 100, 102, 104 or 106; and a DRR having at least 70% identity to the DRR comprised in SEQ ID NO: 108 or 110;
   iii) a fused 1,2-dehydroreticuline synthase-1,2-dehydroreticuline reductase (DRS-DRR) converting (S)-Reticuline into (R)-reticuline selected from DRS-DDR's having at least 70% identity to the DRS-DRR comprised in SEQ ID NO: 92, 94, 96; and/or
   iv) a 1,2-dehydroreticuline synthase (DRS) selected from DRSs having at least 70% identity to the DRS comprised in SEQ ID NO: 98, 100, 102, 104 or 106; and a 1,2-dehydroreticuline reductases (DDR) selected from DDR's having at least 70% identity to the DRR comprised in SEQ ID NO: 108 or 110;
g) expression of one or more heterologous genes encoding a thebaine synthase (THS) converting 7-O-acetylsalutaridinol into thebaine, wherein the THS has at least 70% identity to the THS comprised in SEQ ID NO: 126, 127, 128, 129, 131, 133, 134, 136 or 138; and
h) expression of one or more heterologous genes encoding a transporter protein capable of increasing uptake or export in the host cell of a reticuline derivative selected from transporter proteins having at least 70% identity to the transporter protein comprised in SEQ ID NO: 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407,409, 411, 413, 415, 417, 419, 421,423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 733, 735, 795, 797, 799, 801, 803, 805, 807, 809, 811, 813, 815, 817, 819, 821, 823 or 825.

In a further aspect the invention provides a polynucleotide construct comprising a polynucleotide sequence encoding a heterologous enzymes or transporter protein of the invention operably linked to one or more control sequences.

In a further aspect the invention provides a cell culture, comprising the host cell of the invention and a growth medium.

In a further aspect the invention provides a method for producing a benzylisoquinoline alkaloid comprising:
a) culturing the cell culture of the invention at conditions allowing the cell to produce the benzylisoquinoline alkaloid; and
b) optionally recovering and/or isolating the benzylisoquinoline alkaloid.

In a further aspect the invention provides a fermentation composition comprising the cell culture of the invention and the benzylisoquinoline alkaloid comprised therein.

In a further aspect the invention provides a composition comprising the fermentation composition of the invention and one or more carriers, agents, additives and/or excipients.

In a further aspect the invention provides a pharmaceutical composition comprising the fermentation composition of the invention and one or more pharmaceutical grade excipient, additives and/or adjuvants.

In a further aspect the invention provides a method for preparing the pharmaceutical composition of the invention comprising mixing the fermentation composition of the invention with one or more pharmaceutical grade excipient, additives and/or adjuvants.

In a further aspect the invention provides a method for preventing, treating and/or relieving a disease comprising administering a therapeutically effective amount of the pharmaceutical composition of the invention to a mammal.

### DESCRIPTION OF DRAWINGS AND FIGURES

FIG. 1 shows the pathway for making the benzylisoquinoline alkaloid precursor tyrosine via the Shikimate pathway and additional steps for producing (s)-norcoclaurine.

FIG. 2 depicts a range of benzylisoquinoline alkaloid compounds having pharmaceutical properties which are derivatives of (S)-norcoclaurine.

FIG. 3 shows a schematic representation of the biosynthetic pathway from glucose to thebaine in genetically modified S. cerevisiae strains. Enzymes from NCS to SAT/THS as well as Tyrosine hydroxylase (TH) and DOPA decarboxylase (DODC) are enzymes expressed from heterologous genes.

FIG. 4 shows a stacked bar-diagram made from 3 culture samples analysed by LC-MS. The diagram shows production of reticuline and thebaine in mg/l as described in example 22.

FIG. 5 shows a bar-diagram made from culture samples analysed by LC-MS. Cultures done and shown in triplicates. Production of thebaine in mg/l in yeast strains as described in example 22. The S-to-R-Reticuline (STORR) enzyme activities were expressed in the yeast strain as native (fused) Papaver somniferum DRS-DRR enzyme SEQ ID NO: 96 (called PsSTORR in figure), as separate Papaver somniferum DRS (SEQ ID NO: 98) and DRR (SEQ ID NO: 108) domains called PsCYP82Y2+PsAKR in figure), as separate Papaver somniferum DRS and Streptomyces tsukubaensis Imine reductase (SEQ ID NO: 94) enzymes (called PsCYP82Y2+StIRED in figure), as separate Papaver rhoeas DRS (SEQ ID NO: 101) and Papaver somniferum DRR enzymes (called PrCYP82_AKO60176+PsAKR in figure), or as separate Papaver somniferum DRS and Papaver rhoeas DRR enzymes ((SEQ ID NO: 110) (called PsCYP82Y2+PrAKR_AKO60177 in figure).

FIG. 6 shows a bar-diagram made from culture samples analysed by LC-MS. Cultures done and shown in triplicates. Production of thebaine in mg/l in yeast strains as described in example 22. The bar diagram shows that the three different artificial DRS variants ProID60 (SEQ ID NO: 102), ProID66 (SEQ ID NO: 104) and ProID79 (SEQ ID NO: 106) all significantly improve production of thebaine as compare to the PsAKR (DRS) (SEQ ID NO: 98) when expressed together with the PsAKR (DRR) in the strain described in example 22. ProID79 (SEQ ID NO: 106) appears to be the best.

FIG. 7 shows a bar-diagram made from culture samples analysed by LC-MS. Cultures done in triplicates and shown as average of triplicates including standard deviation error bars. Production of thebaine in mg/l in yeast strains as described in example 23. The bar diagram shows that expression of the three different artificial Thebaine synthases called PROths2_138 (SEQ ID NO: 134), PROths2_143 (SEQ ID NO: 136) and PROths2_116 (SEQ ID NO: 13138) improve or show similar production levels of thebaine as compared to the native P. somniferum THS2 enzyme. PROths2_138 show a significant improvement in activity as compare to the native P. somniferum THS2 enzyme.

FIG. 8 shows a bar-diagram made from culture samples analysed by LC-MS. Cultures done and shown in triplicates. Bar diagram showing the production of Northebaine in S. cerevisiae by expression of two different N-demethylases in a Thebaine producing strain as described in example 24. The CYP450 demethylase and CPR of fungal origin are called CYPDN_91 (SEQ ID NO: 251) and CPR gene ceICPR (SEQ ID NO: 306). The figure legend MothCYP_CPR means expression of insect (moth) CYP450 demethylase HaCYP6AE15v2 (SEQ ID NO: 141) and CPR gene HaCPR_E7E2N6 (SEQ ID NO: 304).

FIG. 9 shows samples from different timepoints (X-axis, hours) during a fed-batch fermentation with strain expressing the n-demethylase CYPDN_91 (SEQ ID NO: 251) and CPR gene ceICPR (SEQ ID NO: 306) in a thebaine producing S. cerevisiae strain as described in example 24. Samples analysed by LC-MS and thebaine and Northebaine production in mg/l is shown as stacked bar-diagram.

FIG. 10 shows thebaine and oripavine production in mg/l demonstrated in a thebaine producer strain (sOD310) as described in example 25. Samples from three cultures were analysed on LC-MS and shown as bars.

FIG. 11 shows the activity of N-terminal variants of HaCYP6AE15v2 expressed in S. cerevisiae and its bioconversion of oripavine to nororipavine in strains expressing N-terminal variants and N-terminal variants combined with single mutations of HaCYP6AE15v2 cytochrome P450 enzyme, grown in DELFT minimal medium at pH 4.5 with 500 μM of oripavine. HaCYP6AE15v2 was truncated between amino acids 2 and 21 to generate truncated HaCYP6AE15v2_t. In FIG. 11 HaCYP6AE15v2 is also referred to as HaCYP6AE15v or HaCYP6AE15.

FIG. 12 shows the activity of N-terminal variants of Hv_CYP_A0A2A4JAM9 expressed in S. cerevisiae and its bioconversion of oripavine to nororipavine in strains expressing N-terminal of Hv_CYP_A0A2A4JAM9 cytochrome P450 enzyme, grown in DELFT minimal medium at pH 4.5 with 500 μM of oripavine. Hv_CYP_A0A2A4JAM9 was truncated between amino acids 2 and 21 to generate truncated Hv_CYP_A0A2A4JAM9_t. In FIG. 12 Hv_CYP_A0A2A4JAM9 is also referred to as Hv_A0A2A4JAM9 or HvA0A2A4JAM9.

FIG. 13 shows sequence alignment of data set >70% ID to Hv_CYP_A0A2A4JAM9 including HaCYP6AE15v2. The amino acids shaded in grey, represents the different residues compared with the consensus sequence. The residues in the black box correspond to the active site residues, according to modeling predictions. In this alignment the most active sequences Hv_CYP_A0A2A4JAM9 and HaCYP6AE15v2 are provided as the top sequences in the alignment for reference. This multiple sequence alignment was performed locally with Clustal Omega program and alignment visualization with CLC workbench 8.0. In FIG. 13 Hv_CYP_A0A2A4JAM9 is also be referred to as Hv_CYP_A0A2A4JAM, while HaCYP6AE15v2 is referred to as 15v2.

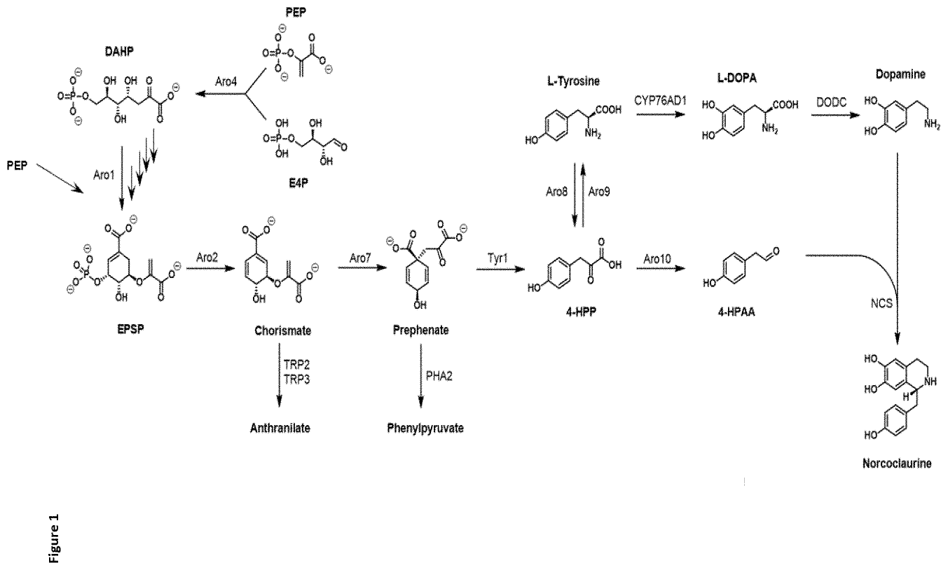

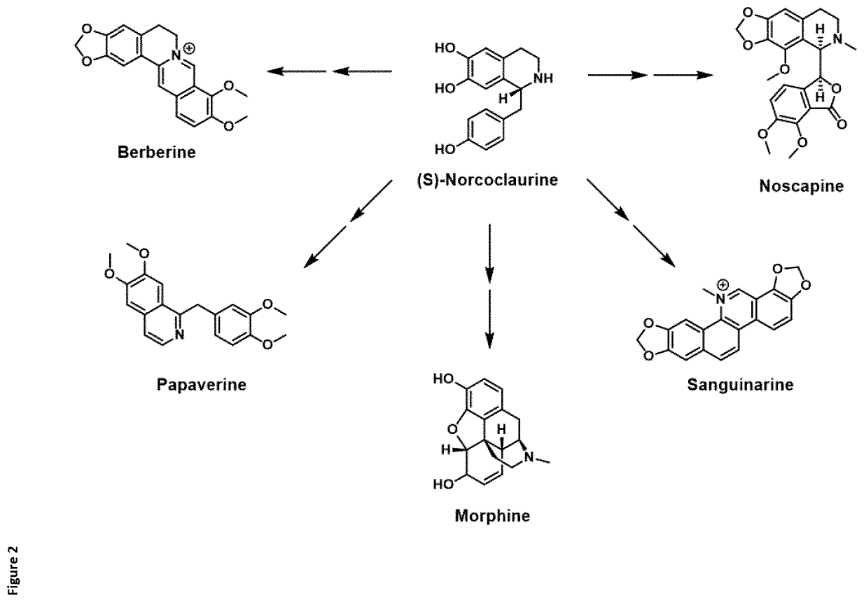

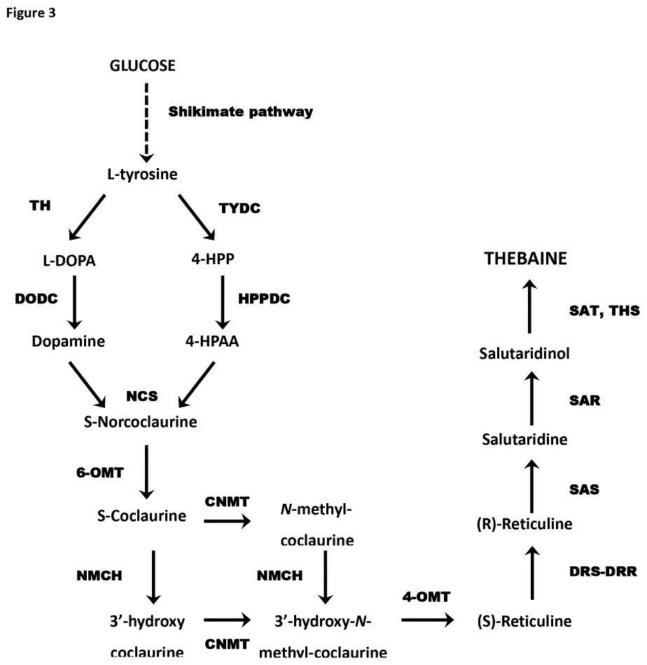

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION OF THE INVENTION

### Definitions

Any EC numbers used herein refers to Enzyme Nomenclature 1992 from NC-IUBMB, Academic Press, San Diego, California, including 30 supplements 1-5 published in Eur. J. Bio-chem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see e.g. http://enzyme.expasy.org/. The term “PEP” as used herein refers to phosphoenol pyruvate.

The term “E4P” as used herein refers to erythrose-4-phosphate

The term “Aro4” as used herein refers to DAHP synthase catalyzing the reaction of PEP and E4P into DAHP.

The term “DAHP” as used herein refers to 3-deoxy-D-arabino-2-heptulosonic acid 7-phosphate.

The term “Aro1” as used herein refers to EPSP synthase catalyzing conversion of DAHP into EPSP.

The term “EPSP” as used herein refers to 5-enolpyruvylshikimate-3-phosphate. The term “Aro2” as used herein refers to chorismate synthase catalyzing conversion of EPSP into chorismate.

The term “Tyr1” as used herein refers to prephenate dehydrogenase catalyzing conversion of prephenate into 4-HPP

The term “4-HPP” as used herein refers to 4-hydroxyphenylpyruvate

The term “Aro8” and “Aro9” as used herein refers to aromatic aminotransferase reversibly catalyzing conversion of 4-HPP into L-tyrosine

The term “ARO10” or HPPDC as used herein refers to hydroxyphenylpyruvate decarboxylase catalyzing 4-HPP into 4-HPAA.

The term “4-HPAA” as used herein refers to 4-Hydroxyphenylacetaldehyde.

The term “TH” as used herein refers to a cytochrome P450 enzyme having tyrosine hydroxylase activity and converting L-tyrosine into L-DOPA.

The term “demethylase” as used herein refers to a P450 enzyme, capable of demethylating thebaine into northebaine, thebaine into oripavine, thebaine into nororipavine and/or oripavine into nororipavine.

The term “DRS” as used herein refers to 1,2-dehydroreticuline synthase, a cytochrome P450 enzyme which catalyze conversion of (S)-Reticuline into 1,2-dehydroreticuline.

The term “DRR” as used herein refers to 1,2-dehydroreticuline reductase which catalyzes conversion of 1,2-dehydroreticuline to (R)-Reticuline.

The term “DRS-DRR” as used herein refers to 1,2-dehydroreticuline synthase-1,2-dehydroreticuline reductase fused complex catalyzing conversion of (S)-Reticuline into (R)-reticuline. This complex may also be referred to as STORR or REPI. DRS-DRR or DRS together with DRR are also categorised as epimerases or isomerases.

The term “CPR” as used herein refers to a cytochrome P450 reductase catalyzing the electron transfer (from NADPH) to a cytochrome P450 enzyme of the pathway, typically in the endoplasmic reticulum of a eukaryotic cell. For distinction and as disclosed herein CPR's are divided into demethylase-CPR used for CPR's capable of reducing demethylases; DRS-CPR used for CPR's capable of reducing DRS and TH-CPR used for CPR's capable of reducing TH. Demethylase-CPR, DRS-CPR and TH-CPR may be identical or different, depending on the P450 to be reduced.

The term “Cytochrome P450 enzyme” or “P450 enzymes” or “P450” as used herein interchangeably refers to a family of monooxygenases enzymes containing heme as a cofactor. P450s are also known as “CYPs”. For distinction and as disclosed herein P450 enzymes are divided into demethylase P450s; DRS P450s, and TH P450s.

The term “family CYP6” as used herein about some demethylases refers to demethylases having >40% amino acid sequence identity to any known demethylase belonging to CYP6 family as defined by Nelson 2006, Cytochrome P450 Nomenclature, included herein by reference.

The term “family CYP76” as used herein about some THs refers to THs having tyrosine hydroxylase activity and capable of catalyzing L-tyrosine into L-DOPA.

The term “DODC” and TYDC” as used herein refers to L-dopa decarboxylase and tyrosine decarboxylase respectively catalyzing conversion of L-DOPA into dopamine and tyrosine into 4-HPP.

The term “MAO” as used herein refers to monoamine oxidase catalyzing conversion of dopamine to 3,4 DHPAA

The term “DHPAA” as used herein refers to 3,4-dihydroxyphenylacetaldehyde.

The term “NCS” as used herein refers to Norcoclaurine synthase catalyzing conversion of dopamine and 4-HPAA into Norcoclaurine.

The term “6-OMT” as used herein refers to 6-O-methyltransferase catalyzing conversion of (S)-norcoclaurine to (S)-Coclaurine

The term “CNMT” as used herein refers to Coclaurine-N-methyltransferase catalyzing conversion of (S)-Coclaurine to (S)—N-Methylcoclaurine and/or (S)-3′-hydroxycoclaurine to (S)-3′-hydroxy-N-methyl-coclaurine.

The term “NMCH” as used herein refers to N-methylcoclaurine 3′-monooxygenase catalyzing conversion of (S)-Coclaurine to (S)-3′-hydroxycoclaurine and/or (S)—N-Methylcoclaurine to (S)-3′-Hydroxy-N-Methylcoclaurine

The term “4′-OMT” as used herein refers to 3′-hydroxy-N-methyl-(S)-coclaurine 4′-O-methyltransferase catalyzing conversion of (S)-3′-Hydroxy-N-Methylcoclaurine to (S)-reticuline.

The term “SAS” as used herein refers to salutaridine synthase catalyzing conversion of (R)-reticuline to Salutaridine.

The term “SAR” as used herein refers to salutaridine reductase catalyzing conversion of Salutaridine to Salutaridinol.

The term “SAT” as used herein refers to salutaridinol 7-O-acetyltransferase catalyzing conversion of Salutaridinol to 7-O-acetylsalutaridinol.

The term “THS” as used herein refers to thebaine synthase catalyzing conversion of 7-O-acetylsalutaridinol into thebaine.

The term “BIA” or “benzylisoquinoline alkaloid” as used herein refers to a compound of the general formula A:

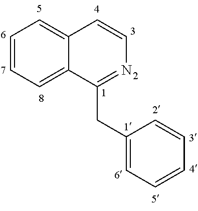

which is the structural backbone of many alkaloids with a wide variety of structures, or to alkaloid products deriving from formula A of the general formula B also known as morphinans:

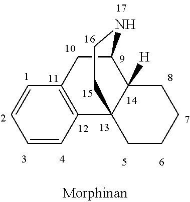

The terms “heterologous” or “recombinant” or “genetically modified” and their grammatical equivalents as used herein interchangeably refers to entities “derived from a different species or cell”. For example, a heterologous or recombinant polynucleotide gene is a gene in a host cell not naturally containing that gene, i.e. the gene is from a different species or cell type than the host cell. The terms as used herein about host cells refers to host cells comprising and expressing heterologous or recombinant polynucleotide genes.

The term “pathway” or “metabolic pathway” as used herein is intended to mean an enzyme acting in a live cell to convert a chemical substrate into a chemical product. A pathway may include one enzyme or multiple enzymes acting in sequence. A pathway including only one enzyme may also herein be referred to as “bioconversion” in particular relevant for embodiments where the cell of the invention is fed with a precursor or substrate to be converted by the enzyme into a desired benzylisoquinoline alkaloid. Enzymes are characterized by having catalytic activity, which can change the chemical structure of the substrate(s). An enzyme may have more than one substrate and produce more than one product. The enzyme may also depend on cofactors, which can be inorganic chemical compounds or organic compounds (co-factor and/or co-enzymes). The NADPH-dependent cytochrome P450 reductase (CPR) is an electron donor to cytochromes P450 (CYPs). CPR shuttles electrons from NADPH through the Flavin Adenine Dinucleotide (FAD) and Flavin Mononucleotide (FMN) coenzymes into the iron of the prosthetic heme-group of the CYP. The term “operative biosynthetic metabolic pathway” refers to a metabolic pathway that occurs in a live recombinant host, as described herein.

The term “in vivo”, as used herein refers to within a living cell or organism, including, for example animal, a plant or a microorganism.

The term “in vitro”, as used herein refers to outside a living cell or organism, including, without limitation, for example, in a microwell plate, a tube, a flask, a beaker, a tank, a reactor and the like.

The term “in planta”, as used herein refers to within a plant or plant cell.

The term “substrate” or “precursor”, as used herein refers to any compound that can be converted into a different compound. For example, thebaine can be a substrate for P450 and can be converted by demethuylation into Northebaine. For clarity, substrates and/or precursors include both compounds generated in situ by a enzymatic reaction in a cell or exogenously provided compounds, such as exogenously provided organic molecules which the host cell can metabolize into a desired compound.

Term “endogenous” or “native” as used herein refers to a gene or a polypeptide in a host cell which originates from the same host cell.

The term “deletion” as used herein refers to manipulation of a gene so that it is no longer expressed in a host cell.

The term “disruption” as used herein refers to manipulation of a gene or any of the machinery participating in the expression the gene, so that it is no longer expressed in a host cell.

The term “attenuation” as used herein refers to manipulation of a gene or any of the machinery participating in the expression the gene, so that it the expression of the gene is reduced as compared to expression without the manipulation.

The terms “substantially” or “approximately” or “about”, as used herein refers to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value the terms of degree can include a range of values plus or minus 10% from that value. For example, deviation from a value can include a specified value plus or minus a certain percentage from that value, such as plus or minus 9%, 8%, 7%, 6%, 5%, 4%, 3%, 2%, or 1% from the specified value.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The term “isolated” as used herein about a compound, refers to any compound, which by means of human intervention, has been put in a form or environment that differs from the form or environment in which it is found in nature. Isolated compounds include but is no limited to compounds of the invention for which the ratio of the compounds relative to other constituents with which they are associated in nature is increased or decreased. In an important embodiment the amount of compound is increased relative to other constituents with which the compound is associated in nature. In an embodiment the compound of the invention may be isolated into a pure or substantially pure form. In this context a substantially pure compound means that the compound is separated from other extraneous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other extraneous or unwanted material usually associated with the compound when expressed natively or recombinantly. In an embodiment the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “non-naturally occurring” as used herein about a substance, refers to any substance that is not normally found in nature or natural biological systems. In this context the term “found in nature or in natural biological systems” does not include the finding of a substance in nature resulting from releasing the substance to nature by deliberate or accidental human intervention. Non-naturally occurring substances may include substances completely or partially synthetized by human intervention and/or substances prepared by human modification of a natural substance.

The term “% identity” is used herein about the relatedness between two amino acid sequences or between two nucleotide sequences.

The term “% identity” as used herein about amino acid or nucleotide sequences refers to the degree of identity in percent between two amino acid sequences obtained when using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

$$\frac{identical amino acid residues}{L ength of alignment - total number of gaps in alignment} \times 100$$

The term “% identity” as used herein about nucleotide sequences refers to the degree of identity in percent between two nucleotide sequences obtained when using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

$$\frac{identical deoxyribonucleotides}{L ength of alignment - total number of gaps in alignment} \times 100$$

The protein sequences of the present invention can further be used as a “query sequence” to perform a search against sequence databases, for example to identify other family members or related sequences. Such searches can be performed using the BLAST programs. Software for performing BLAST analyses is publicly available through the National Center for Biotechnology Information (http://www.ncbi.nlm.nih.gov). BLASTP is used for amino acid sequences and BLASTN for nucleotide sequences. The BLAST program uses as defaults:
Cost to open gap: default=5 for nucleotides/11 for proteins
Cost to extend gap: default=2 for nucleotides/1 for proteins
Penalty for nucleotide mismatch: default=−3
Reward for nucleotide match: default=1
Expect value: default=10
Wordsize: default=11 for nucleotides/28 for megablast/3 for proteins.

Furthermore, the degree of local identity between the amino acid sequence query or nucleic acid sequence query and the retrieved homologous sequences is determined by the BLAST program. However only those sequence segments are compared that give a match above a certain threshold. Accordingly, the program calculates the identity only for these matching segments. Therefore, the identity calculated in this way is referred to as local identity. Alternatively, % identity for any candidate nucleic acid or amino acid sequence relative to a reference sequence can be determined as follows. A reference sequence (e.g., a nucleic acid sequence or an amino acid sequence described herein) is aligned to one or more candidate sequences using the computer program Clustal Omega (version 1.2.1, default parameters), which allows alignments of nucleic acid or polypeptide sequences to be carried out across their entire length (global alignment). Chenna et al., 2003, Nucleic Acids Res. 31(13):3497-500.

Clustal Omega calculates the best match between a reference and one or more candidate sequences, and aligns them so that identities, similarities and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: % age; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method:% age; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gin, Glu, Arg, and Lys; residue-specific gap penalties: on. The Clustal Omega output is a sequence alignment that reflects the relationship between sequences. Clustal Omega can be run, for example, at the Baylor College of Medicine Search Launcher site on the World Wide Web (searchlauncher.bcm.tmc.edu/multi-align/multi-align.html) and at the European Bioinformatics Institute site at http://www.ebi.ac.uk/Tools/msa/clustalo/. To determine a % identity of a candidate nucleic acid or amino acid sequence to a reference sequence, the sequences are aligned using Clustal Omega, the number of identical matches in the alignment is divided by the length of the reference sequence, and the result is multiplied by 100. It is noted that the % identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The term “mature polypeptide” or “mature enzyme” as used herein refers to a polypeptide in its final active form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide.

The term “cDNA” refers to a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The term “coding sequence” refers to a nucleotide sequence, which directly specifies the amino acid sequence of a polypeptide. The boundaries of the coding sequence are generally determined by an open reading frame, which begins with a start codon such as ATG, GTG, orTTG and ends with a stop codon such as TAA, TAG, or TGA. The coding sequence may be a genomic DNA, cDNA, synthetic DNA, or a combination thereof.

The term “control sequence” as used herein refers to a nucleotide sequence necessary for expression of a polynucleotide encoding a polypeptide. A control sequence may be native (i.e., from the same gene) or heterologous or foreign (i.e., from a different gene) to the polynucleotide encoding the polypeptide. Control sequences include, but are not limited to leader sequences, polyadenylation sequence, pro-peptide coding sequence, promoter sequences, signal peptide coding sequence, translation terminator (stop) sequences and transcription terminator (stop) sequences. To be operational control sequences usually must include promoter sequences, transcriptional and translational stop signals. Control sequences may be provided with linkers for the purpose of introducing specific restriction sites facilitating ligation of the control sequences with a coding region of a polynucleotide encoding a polypeptide.

The term “expression” includes any step involved in the production of a polypeptide including, but not limited to, transcription, post-transcriptional modification, translation, post-translational modification, and secretion.

The term “expression vector” refers to a DNA molecule, either single- or double stranded, either linear or circular, which comprises a polynucleotide encoding a polypeptide and is operably linked to control sequences that provide for its expression. Expression vectors include expression cassettes for the integration of genes into a host cell as well as plasmids and/or chromosomes comprising such genes.

The term “host cell” refers to any cell type that is susceptible to transformation, transfection, transduction, or the like with a nucleic acid construct or expression vector comprising a polynucleotide of the present invention. Host cell encompasses any progeny of a parent cell that is not identical to the parent cell due to mutations that occur during replication.

The term “polynucleotide construct” refers to a polynucleotide, either single- or double stranded, which is isolated from a naturally occurring gene or is modified to contain segments of nucleic acids in a manner that would not otherwise exist in nature or which is synthetic, and which comprises a polynucleotide encoding a polypeptide and one or more control sequences.

The term “operably linked” refers to a configuration in which a control sequence is placed at an appropriate position relative to the coding polynucleotide such that the control sequence directs expression of the coding polynucleotide.

The terms “nucleotide sequence and “polynucleotide” are used herein interchangeably.

The term “comprise” and “include” as used throughout the specification and the accompanying items as well as variations such as “comprises”, “comprising”, “includes” and “including” are to be interpreted inclusively. These words are intended to convey the possible inclusion of other elements or integers not specifically recited, where the context allows.

The articles “a” and “an” are used herein refers to one or to more than one (i.e. to one or at least one) of the grammatical object of the article. By way of example, “an element” may mean one element or more than one element.

Terms like “preferably”, “commonly”, “particularly”, and “typically” are not utilized herein to limit the scope of the itemed invention or to imply that certain features are critical, essential, or even important to the structure or function of the itemed invention. Rather, these terms are merely intended to highlight alternative or additional features that can or cannot be utilized in a particular embodiment of the present invention.

The term “cell culture” as used herein refers to a culture medium comprising a plurality of host cells of the invention. A cell culture may comprise a single strain of host cells or may comprise two or more distinct host cell strains. The culture medium may be any medium that may comprise a recombinant host, e.g., a liquid medium (i.e., a culture broth) or a semi-solid medium, and may comprise additional components, e.g., a carbon source such as dextrose, sucrose, glycerol, or acetate; a nitrogen source such as ammonium sulfate, urea, or amino acids; a phosphate source; vitamins; trace elements; salts; amino acids; nucleobases; yeast extract; aminoglycoside antibiotics such as G418 and hygromycin B.

All methods described herein can be performed in any suitable order of steps unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

All percentages, ratios and proportions herein are by weight, unless otherwise specified. A weight percent (weight %, also as wt. %) of a component, unless specifically stated to the contrary, is based on the total weight of the composition in which the component is included (e.g., on the total amount of the reaction mixture).

Terms used herein may be preceded and/or followed by a single dash, “ ”, or a double dash, “=”, to indicate the bond order of the bond between the named substituent and its parent moiety; a single dash indicates a single bond and a double dash indicates a double bond or a pair of single bonds in the case of a spiro-substituent. In the absence of a single or double dash it is understood that a single bond is formed between the substituent and its parent moiety; further, substituents are intended to be read “left to right” with reference to the chemical structure referred to unless a dash indicates otherwise. For example, arylalkyl, arylalkyl-, and alkylaryl indicate the same functionality.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety can refer to a monovalent radical (e.g. CH3-CH2-), in some circumstances a bivalent linking moiety can be “alkyl,” in which case those skilled in the art will understand the alkyl to be a divalent radical (e.g., —CH2-CH2-), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for 0, and 2, 4, or 6 for S, depending on the oxidation state of the S). Nitrogens in the presently disclosed compounds can be hypervalent, e.g., an N-oxide or tetrasubstituted ammonium salt. On occasion a moiety may be defined, for example, as —B-(A)a, wherein a is 0 or 1. In such instances, when a is 0 the moiety is —B and when a is 1 the moiety is —B-A.

As used herein, the term “alkyl” or “alkane” includes a saturated hydrocarbon having a designed number of carbon atoms, such as 1 to 40 carbons (i.e., inclusive of 1 and 40), 1 to 35 carbons, 1 to 25 carbons, 1 to 20 carbons, or 1, 2, 3, 4, 5, 6, 7, 8, 9, 10, 11, 12, 13, 14, 15, 16, 17, or 18. Alkyl groups or alkanes may be straight or branched and depending on context, may be a monovalent radical or a divalent radical (i.e., an alkylene group). For example, the moiety “—(C1 C6 alkyl)O—” signifies connection of an oxygen through an alkylene bridge having from 1 to 6 carbons and C1-C3 alkyl represents methyl, ethyl, and propyl moieties. Examples of “alkyl” include, for example, methyl, ethyl, propyl, isopropyl, butyl, iso, sec and tert butyl, pentyl, and hexyl. Examples of “alkane” include, for example, methane, ethane, propane, isopropane, butane, isobutane, sec-butane, tert-butane, pentane, hexane, heptane, and octane.

The term “alkoxy” represents an alkyl group of indicated number of carbon atoms attached to the parent molecular moiety through an oxygen bridge. Examples of “alkoxy” include, for example, methoxy, ethoxy, propoxy, and isopropoxy.

The term “alkenyl” as used herein, unsaturated hydrocarbon containing from 2 to 10 carbons (i.e., inclusive of 2 and 10), 2 to 8 carbons, 2 to 6 carbons, or 2, 3, 4, 5 or 6, unless otherwise specified, and containing at least one carbon-carbon double bond. Alkenyl group may be straight or branched and depending on context, may be a monovalent radical or a divalent radical (i.e., an alkenylene group). For example, the moiety “—(C2 C6 alkenyl)O—” signifies connection of an oxygen through an alkenylene bridge having from 2 to 6 carbons. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkynyl” as used herein, unsaturated hydrocarbon containing from 2 to 10 carbons (i.e., inclusive of 2 and 10), 2 to 8 carbons, 2 to 6 carbons, or 2, 3, 4, 5 or 6 unless otherwise specified, and containing at least one carbon-carbon triple bond. Alkynyl group may be straight or branched and depending on context, may be a monovalent radical or a divalent radical (i.e., an alkynylene group). For example, the moiety “—(C2 C6 alkynyl)O—” signifies connection of an oxygen through an alkynylene bridge having from 2 to 6 carbons. Representative examples of alkynyl include, but are not limited to, acetylenyl, 1-propynyl, 2-propynyl, 3-butynyl, 2-pentynyl, and 1-butynyl.

The term “aryl” represents an aromatic ring system having a single ring (e.g., phenyl) which is optionally fused to other aromatic hydrocarbon rings or non-aromatic hydrocarbon or heterocyclic rings. “Aryl” includes ring systems having multiple condensed rings and in which at least one is carbocyclic and aromatic, (e.g., 1,2,3,4 tetrahydronaphthyl, naphthyl). Examples of aryl groups include phenyl, 1 naphthyl, 2 naphthyl, indanyl, indenyl, dihydronaphthyl, fluorenyl, tetralinyl, and 6,7,8,9-tetrahydro-5H-benzo[a]cycloheptenyl. “Aryl” also includes ring systems having a first carbocyclic, aromatic ring fused to a nonaromatic heterocycle, for example, 1H-2,3 dihydrobenzofuranyl and tetrahydroisoquinolinyl. The aryl groups herein are unsubstituted or, when specified as “optionally substituted”, can unless stated otherwise be substituted in one or more substitutable positions with various groups as indicated.

The term “heteroaryl” refers to an aromatic ring system containing at least one aromatic heteroatom selected from nitrogen, oxygen and sulfur in an aromatic ring. Most commonly, the heteroaryl groups will have 1, 2, 3, or 4 heteroatoms. The heteroaryl may be fused to one or more non-aromatic rings, for example, cycloalkyl or heterocycloalkyl rings, wherein the cycloalkyl and heterocycloalkyl rings are described herein. In one embodiment of the present compounds the heteroaryl group is bonded to the remainder of the structure through an atom in a heteroaryl group aromatic ring. In another embodiment, the heteroaryl group is bonded to the remainder of the structure through a non-aromatic ring atom. Examples of heteroaryl groups include, for example, pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, benzo[1,4]oxazinyl, triazolyl, tetrazolyl, isothiazolyl, naphthyridinyl, isochromanyl, chromanyl, isoindolinyl, isobenzothienyl, benzoxazolyl, pyridopyridinyl, purinyl, benzodioxolyl, triazinyl, pteridinyl, benzothiazolyl, imidazopyridinyl, imidazothiazolyl, benzisoxazinyl, benzoxazinyl, benzopyranyl, benzothiopyranyl, chromonyl, chromanonyl, pyridinyl N-oxide, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S oxide, benzothiopyranyl S,S dioxide. Preferred heteroaryl groups include pyridyl, pyrimidyl, quinolinyl, indolyl, pyrrolyl, furanyl, thienyl and imidazolyl, pyrazolyl, indazolyl, thiazolyl and benzothiazolyl. In certain embodiments, each heteroaryl is selected from pyridyl, pyrimidinyl, pyridazinyl, pyrazinyl, imidazolyl, isoxazolyl, pyrazolyl, oxazolyl, thiazolyl, furanyl, thienyl, pyrrolyl, oxadiazolyl, thiadiazolyl, triazolyl, tetrazolyl, isothiazolyl, pyridinyl N-oxide, pyrrolyl N-oxide, pyrimidinyl N-oxide, pyridazinyl N-oxide, pyrazinyl N-oxide, imidazolyl N-oxide, isoxazolyl N-oxide, oxazolyl N-oxide, thiazolyl N-oxide, pyrrolyl N-oxide, oxadiazolyl N-oxide, thiadiazolyl N-oxide, triazolyl N-oxide, and tetrazolyl N-oxide. Preferred heteroaryl groups include pyridyl, pyrimidyl, quinolinyl, indolyl, pyrrolyl, furanyl, thienyl, imidazolyl, pyrazolyl, indazolyl, thiazolyl and benzothiazolyl. The heteroaryl groups herein are unsubstituted or, when specified as “optionally substituted”, can unless stated otherwise be substituted in one or more substitutable positions with various groups, as indicated.

The term “heterocycloalkyl” refers to a non-aromatic ring or ring system containing at least one heteroatom that is preferably selected from nitrogen, oxygen and sulfur, wherein said heteroatom is in a non aromatic ring. The heterocycloalkyl may have 1, 2, 3 or 4 heteroatoms. The heterocycloalkyl may be saturated (i.e., a heterocycloalkyl) or partially unsaturated (i.e., a heterocycloalkenyl). Heterocycloalkyl includes monocyclic groups of three to eight annular atoms as well as bicyclic and polycyclic ring systems, including bridged and fused systems, wherein each ring includes three to eight annular atoms. The heterocycloalkyl ring is optionally fused to other heterocycloalkyl rings and/or non-aromatic hydrocarbon rings. In certain embodiments, the heterocycloalkyl groups have from 3 to 7 members in a single ring. In other embodiments, heterocycloalkyl groups have 5 or 6 members in a single ring. In some embodiments, the heterocycloalkyl groups have 3, 4, 5, 6 or 7 members in a single ring. Examples of heterocycloalkyl groups include, for example, azabicyclo[2.2.2]octyl (in each case also “quinuclidinyl” or a quinuclidine derivative), azabicyclo[3.2.1]octyl, 2,5-diazabicyclo[2.2.1]heptyl, morpholinyl, thiomorpholinyl, thiomorpholinyl S oxide, thiomorpholinyl S,S dioxide, 2 oxazolidonyl, piperazinyl, homopiperazinyl, piperazinonyl, pyrrolidinyl, azepanyl, azetidinyl, pyrrolinyl, tetrahydropyranyl, piperidinyl, tetrahydrofuranyl, tetrahydrothienyl, 3,4-dihydroisoquinolin-2(1H)-yl, isoindolindionyl, homopiperidinyl, homomorpholinyl, homothiomorpholinyl, homothiomorpholinyl S,S dioxide, oxazolidinonyl, dihydropyrazolyl, dihydropyrrolyl, dihydropyrazinyl, dihydropyridinyl, dihydropyrimidinyl, dihydrofuryl, dihydropyranyl, imidazolidonyl, tetrahydrothienyl S oxide, tetrahydrothienyl S,S dioxide and homothiomorpholinyl S oxide. Especially desirable heterocycloalkyl groups include morpholinyl, 3,4-dihydroisoquinolin-2(1H)-yl, tetrahydropyranyl, piperidinyl, aza bicyclo[2.2.2]octyl, γ butyrolactonyl (i.e., an oxo substituted tetrahydrofuranyl), γ butryolactamyl (i.e., an oxo substituted pyrrolidine), pyrrolidinyl, piperazinyl, azepanyl, azetidinyl, thiomorpholinyl, thiomorpholinyl S,S dioxide, 2 oxazolidonyl, imidazolidonyl, isoindolindionyl, piperazinonyl. The heterocycloalkyl groups herein are unsubstituted or, when specified as “optionally substituted”, can unless stated otherwise be substituted in one or more substitutable positions with various groups, as indicated.

The term “cycloalkyl” or “cycloalkane” refers to a non-aromatic carbocyclic ring or ring system, which may be saturated (i.e., a cycloalkyl, a cycloalkane) or partially unsaturated (i.e., a cycloalkenyl). The cycloalkyl ring can be optionally fused to or otherwise attached (e.g., bridged systems) to other cycloalkyl rings. Certain examples of cycloalkyl groups or cycloalkanes present in the disclosed compounds have from 3 to 7 members in a single ring, such as having 5 or 6 members in a single ring. In some embodiments, the cycloalkyl groups have 3, 4, 5, 6 or 7 members in a single ring. Examples of cycloalkyl groups include, for example, cyclohexyl, cyclopentyl, cyclobutyl, cyclopropyl, tetrahydronaphthyl and bicyclo[2.2.1]heptane. Examples of cycloalkanes include, for example, cyclohexane, methylcyclohexane, cyclohexanone, cyclohexanol, cyclopentane, cycloheptane, and cycloctane. The cycloalkyl groups herein are unsubstituted or, when specified as “optionally substituted”, may be substituted in one or more substitutable positions with various groups, as indicated.

The term “ring system” encompasses monocycles, as well as fused and/or bridged polycycles.

The terms “halogen” or “halo” indicate fluorine, chlorine, bromine, and iodine. In certain embodiments of each and every embodiment described herein, the term “halogen” or “halo” refers to fluorine or chlorine. In certain embodiments of each and every embodiment described herein, the term “halogen” or “halo” refers to fluorine.

The term “halide” indicates fluoride, chloride, bromide, and iodide. In certain embodiments of each and every embodiment described herein, the term “halide” refers to bromide or chloride.

The term “substituted,” when used to modify a specified group or radical, means that one or more hydrogen atoms of the specified group or radical are each, independently of one another, replaced with the same or different substituent groups as defined below, unless specified otherwise.

Specific protecting groups may be used to protect reactive functionalities of a starting material or intermediate to prepare a desired product. In general, the need for such protecting groups as well as the conditions necessary to attach and remove such groups will be apparent to those skilled in the art of organic synthesis. An authoritative account describing the many alternatives to the trained practitioner are J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4.sup.th edition, Vol. 15/I, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

As used herein, the term “benzyl” (“Bn”) includes unsubstituted (i.e., (C6H5)-CH2-) and substituted benzyl (i.e., benzyl substituted at the 2-, 3-, and/or 4-position with C1-C8 alkyl or halide). The person of ordinary skill in the art will appreciate that oxygen protecting groups include alkoxycarbonyl, acyl, acetal, ether, ester, silyl ether, alkylsulfonyl, and arylsulfonyl. Exemplary oxygen protecting groups include allyl, triphenylmethyl (trityl or Tr), benzyl, methanesulfonyl, p-toluenesulfonyl, p-methoxybenzyl (PMB), p-methoxyphenyl (PMP), methoxymethyl (MOM), p-methoxyethoxymethyl (MEM), tetrahydropyranyl (THP), ethoxyethyl (EE), methylthiomethyl (MTM), 2-methoxy-2-propyl (MOP), 2-trimethylsilylethoxymethyl (SEM), benzoate (BZ), allyl carbonate, 2.2.2-trichloroethyl carbonate (Troc), 2-trimethylsilylethyl carbonate, trimethylsilyl (TMS), triethylsilyl (TES), triisopropylsilyl (TIPS), triphenylsilyl (TPS), t-butyldimethylsilyl (TBDMS), and t-butyldiphenylsilyl (TBDPS). A variety of protecting groups for the oxygen and the synthesis thereof may be found in “Protective Groups in Organic Synthesis” by T. W. Greene and P. G. M. Wuts, John Wiley & Sons, 1999. In certain embodiments, an appropriate oxygen protecting goup may be used in place of benzyl.

### Genetically Modified Host Cells

Microorganisms optimized to produce benzylisoquinoline alkaloids are in great need and even more so host cells optimized to demethylate benzylisoquinoline alkaloids such as thebaine and/or oripavine into the corresponding northebaine and/or nororipavine, which are in high demand for chemical conversion into other pharmaceutically relevant benzylisoquinoline alkaloids.

The invention provides in a first aspect such genetically modified host cell comprising a pathway having enhanced production of one or more benzylisoquinoline alkaloids wherein the cell comprises one or more features selected from:
a) expression of one or more heterologous genes encoding a demethylase capable of converting thebaine into northebaine, thebaine into oripavine, thebaine into nororipavine and/or oripavine into nororipavine;
b) expression of one or more heterologous genes encoding a tyrosine hydroxylases (TH) converting L-tyrosine into L-dopa selected from TH's having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the TH comprised in 7, 9, 11, 13, 15, 17, 19, 21, 23, 25, 27, 29, 31, 33, 35, 37, 39, 41, 43, 45, 47, 49, 51, 53, 55, 57, 59, 61, 63 or 65;
c) reduction or elimination of activity of one or more dehydrogenases native to the host cell selected from the dehydrogenases comprised in SEQ ID NO: 663, 665, 667, 669, 671, 673, 675, 677, 679, 681, 683, 685, 687, 689, 691, 693, 695, 697, 699, 701, 703 or 705;
d) reduction or elimination of activity of one or more reductases native to the host cell selected from the reductases comprised in SEQ ID NO: 707, 709, 711, 713, 715, 717, 719, 721, 723, 725, 727, 729 or 731;
e) expression of one or more heterologous genes encoding a norcoclaurine synthases (NCS) converting Dopamine and 4-HPAA into (S)-norcoclaurine selected from NCS's having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the NCS comprised in SEQ ID NO: 73 OR 76;
f) expression of one or more heterologous genes encoding
   i) a fused 1,2-dehydroreticuline synthase-1,2-dehydroreticuline reductases (DRS-DRR) converting (S)-Reticuline into (R)-reticuline, wherein
      ia) the DRS-DDRs has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRS-DRR comprised in SEQ ID NO: 92, 94, 96; or
      ib) the DRS moiety has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRS comprised in SEQ ID NO: 98, 100, 102, 104 or 106; and the DRR moiety has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRR comprised in SEQ ID NO: 108 or 110; or
   ii) a DRS having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRS comprised in SEQ ID NO: 98, 100, 102, 104 or 106; and a DRR having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRR comprised in SEQ ID NO: 108 or 110;
g) expression of one or more heterologous genes encoding a thebaine synthase (THS) converting 7-O-acetylsalutaridinol into thebaine selected from THS's having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the THS comprised in SEQ ID NO: 126, 127, 128, 129, 131, 133, 134, 136, 138; and
h) expression of one or more heterologous genes encoding a transporter protein capable of increasing uptake in the host cell of a reticuline derivative selected from transporter proteins having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the transporter protein comprised in SEQ ID NO: 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 733, 735, 795, 797, 799, 801, 803, 805, 807, 809, 811, 813, 815, 817, 819, 821, 823 or 825.

### Heterologous Demethylase

In a further aspect the genetically modified host cells of the invention expresses, alone or in combination with other heterologous genes of the invention, one or more heterologous genes encoding one or more demethylases capable of converting thebaine into northebaine, thebaine into oripavine, thebaine into nororipavine and/or oripavine into nororipavine. The demethylase of the invention can be any suitable demethylase capable of converting thebaine into northebaine, thebaine into oripavine, thebaine into nororipavine and/or oripavine into nororipavine, which is heterologous to the host cell and which cooperates well with the other enzymes of the benzylisoquinoline alkaloid pathway and/or the auxiliary cellular mechanisms.

In a particular embodiment the demethylase have specificity towards producing the nor-compounds and produces less by-products. It has been identified that in particular insect demethylase, when expressed in a genetically modified host cell possess a hitherto unprecedented high product specificity producing a high product:by-product ratio, where the product:by-product is either (Northebaine):(thebaine N-oxide), (Northebaine):(northebaine oxaziridine), (Nororipavine):(oripavine N-oxide) and/or (Nororipavine):(nororipavine oxaziridine). Aside from more effectively converting more thebaine and/or oripaving into the desired corresponding nor-compounds, for in vivo conversion the insect demethylase of the invention also produces less N-oxide or oxaziridine by-products and this property provide advantage over the art, since such by-products may impact negatively of the cell function as well as they may interfere with efficiency of any subsequent chemical conversion steps and lower the efficiency of production. Accordingly, in one embodiment the demethylase of the invention have a product:by-product molar ratio of at least 2.0, such as at least 2.25, such as at least 2.5, such as at least 2.75, such as at least 3.0, such as at least 3.25, such as at least 3.5, such as at least 3.75, such as at least 4.0, such as at least 4.5, such as at least 5.0, such as at least 10.0, such as at least 25, such as at least 50, such as at least 75, such as at least 100 and wherein when the product is northebaine then the by-product is thebaine N-oxide and/or northebaine oxaziridine and when the product is nororipavine then the by-product is oripavine N-oxide and/or nororipavine oxaziridine.

The insect demethylase of the invention remarkably displays N-demethylation activity and/or O-activity, whereby it is capable of converting thebaine of the formula I into northebaine of the formula II:

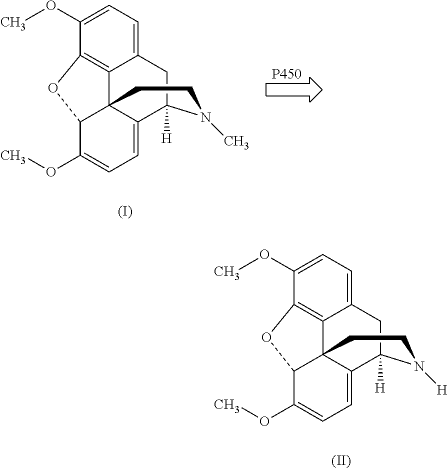

converting thebaine of the formula I into oripavine of the formula (III)

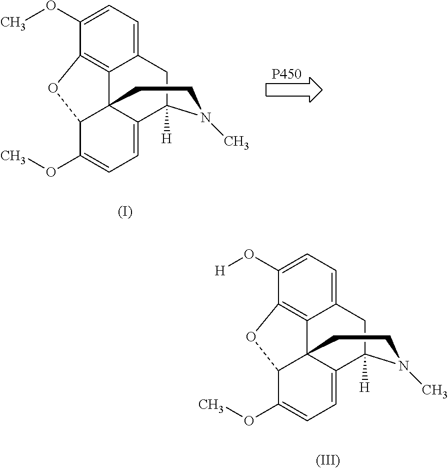

and/or converting oripavine of the formula (III) into nororipavine of formula IV

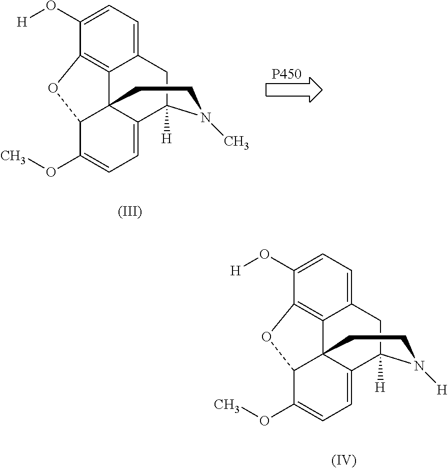

Further, the present inventors have found that demethylases derived from insects and in particular demethylases of family CYP6, are remarkably effective in converting thebaine and/or oripavine into the corresponding nor-compounds producing less by-products. Therefore, in one embodiment the demethylase of the invention is derived from an insect and in another embodiment the demethylase of the invention is of family CYP6. Relevant insects include those which feeds on plants with high contents of thebaine and/or oripavine such as poppy and include moths of the order Lepidoptera, such as moths of the genus Helicoverpa, Spodoptera, Cnaphalocrocis, Bombyx and Heliothis. Demethylases from the species Helicoverpa armigera, Spodoptera exigua, Cnaphalocrocis medinalis, Bombyx mandarina and Heliothis virescens, are particularly useful. Without being bound to the theory the present inventors contemplate that insects feeding from plants containing a high level of thebaine and/or oripavine, as a protection mechanism, during evolution have developed enzymes converting these potentially harmful substrates.

Examples of insect demethylases which works remarkably well in converting thebaine and/or oripavine with low formation of by-products in a heterologous host cell includes the demethylases selected from of SEQ ID NO: 140, 142, 144, 146, 148, 150, 152, 154, 156, 158, 160, 162, 164, 166, 168, 170, 172,174, 176, 178, 180, 182, 184, 186, 188, 190, 192,194, 196, 827, 829, 831, 833, 835, 837, 839, 841, 843, 845, 847, 849, 851, 853, 855, 857, 859, 861, 863, 865, 867 and 869. No such demethylase nor anyone with any close homology has previously been reported useful in a host and let alone with the remarkably high efficiency. Accordingly, in a further embodiment the demethylase of the invention comprises a polypeptide selected from the group consisting of:
a) a demethylase which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase comprised in any one of SEQ ID NO: 140, 142, 144, 146, 148, 150, 152, 154, 156, 158, 160, 162, 164, 166, 168, 170, 172, 174, 176, 178, 180, 182, 184, 186, 188, 190, 192, 194, 196, 827, 829, 831, 833, 835, 837, 839, 841, 843, 845, 847, 849, 851, 853, 855, 857, 859, 861, 863, 865, 867 and 869;
b) a demethylase encoded by a polynucleotide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to a polynucleotide comprised in any one of SEQ ID NO: 141, 143, 145, 147, 149, 151, 153, 155, 157, 159, 161, 163, 165, 167, 169, 171, 173, 175, 177, 179, 181, 183, 185, 187, 189, 191, 193, 195, 197, 828, 830, 832, 834, 836, 838, 840, 842, 844, 846, 848, 850, 852, 854, 856, 858, 860, 862, 864, 866, 868 and 870 or genomic DNA thereof; and
c) a functional variant of the demethylase of (a) or (b) capable of converting thebaine into northebaine, thebaine into oripavine, thebaine into nororipavine and/or oripavine into nororipavine.

In particular the insect demethylase is
a) a demethylase comprised in any one of SEQ ID NO: 140, 142, 144, 146, 148, 150, 152, 154, 156, 158, 160, 162, 164, 166, 168, 170, 172, 174, 176, 178, 180, 182, 184, 186, 188, 190, 192, 194, 196, 827, 829, 831, 833, 835, 837, 839, 841, 843, 845, 847, 849, 851, 853, 855, 857, 859, 861, 863, 865, 867 and 869; or
b) a demethylase encoded by a polynucleotide comprised in any one of SEQ ID NO: or genomic DNA thereof encoding the P450 comprised in any one of SEQ ID NO: 141, 143, 145, 147, 149, 151, 153, 155, 157, 159, 161, 163, 165, 167, 169, 171, 173, 175, 177, 179, 181, 183, 185, 187, 189, 191, 193, 195, 197, 828, 830, 832, 834, 836, 838, 840, 842, 844, 846, 848, 850, 852, 854, 856, 858, 860, 862, 864, 866, 868 and 870.

Alternatively, the demethylase of the invention can be derived from a fungus, in particular fungi of a genus selected from Rhizopus, Lichtheimia, Syncephalastrum, Cunninghamella, Mucor, Parasitella, Absidia, Choanephora, Bifiguratus and Choanephora. In a more specific embodiment the P450 may be derived from a fungal species selected from Rhizopus microspores, Rhizopus azygosporus, Rhizopus stolonifera, Rhizopus oryzae, Rhizopus delemar, Lichtheimia corymbifera, Lichtheimia ramose, Syncephalastrum racemosum, Cunninghamella echinulate, Mucor circinelloides, Mucor ambiguous, Parasitella parasitica, Absidia repens, Absidia glauca, Choanephora cucurbitarum, Bifiguratus adelaidae and Choanephora cucurbitarum.

Examples of fungal demethylases which works well in converting thebaine and/or oripavine with low formation of by-products in a heterologous host cell includes the demethylase selected from SEQ ID NO: 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288 or 290. No such demethylase has previously been reported so effective and useful in a host cell and let alone with the remarkable high efficiency. Accordingly, in a further embodiment the demethylase of the invention comprises a polypeptide selected from the group consisting of:
a) a demethylase which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase comprised in any one of SEQ ID NO: 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288 and 290;
b) a demethylase encoded by a polynucleotide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the polynucleotide comprised in any one of SEQ ID NO: 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289 and 291 or genomic DNA thereof; and
c) a functional variant of the demethylase of (a) or (b) capable of converting thebaine into northebaine, thebaine into oripavine, thebaine into nororipavine and/or oripavine into nororipavine.

In particular the fungal demethylase is:
a) the demethylase comprised in any one of SEQ ID NO: 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288 and 290; or
b) the demethylase encoded by a polynucleotide comprised in any one of SEQ ID NO: 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289 and 291 or genomic DNA thereof.

A particular demethylase of the invention is one which does not comprise one or more of the amino acids selected from:
a) Valine at a position corresponding to V75 of SEQ ID NO: 290;
b) Isoleucine at a position corresponding to I79 of SEQ ID NO: 290;
c) Isoleucine at a position corresponding to V83 of SEQ ID NO: 290;
d) Asparagine at a position corresponding to N84 of SEQ ID NO: 290;
e) Arginine at a position corresponding to R86 of SEQ ID NO: 290;
f) Aspartic acid at a position corresponding to D87 of SEQ ID NO: 290;
g) Glutamic acid at a position corresponding to E126 of SEQ ID NO: 290;
h) Threonine at a position corresponding to T145 of SEQ ID NO: 290;
i) Asparagine at a position corresponding to N172 of SEQ ID NO: 290;
j) Threonine at a position corresponding to T193 of SEQ ID NO: 290;
k) Glycine at a position corresponding to G218 of SEQ ID NO: 290;
l) Isoleucine at a position corresponding to I236 of SEQ ID NO: 290;
m) Alanine at a position corresponding to A258 of SEQ ID NO: 290;
n) Methionine at a position corresponding to M259 of SEQ ID NO: 290;
o) Aspartic acid at a position corresponding to D298 of SEQ ID NO: 290;
p) Leucine at a position corresponding to L430 of SEQ ID NO: 290;
q) Histidine at a position corresponding to H448 of SEQ ID NO: 290;
r) Asparagine at a position corresponding to N503 of SEQ ID NO: 290;
s) Proline at a position corresponding to P506 of SEQ ID NO: 290;
t) Phenylalanine at a position corresponding to F507 of SEQ ID NO: 290;
u) Asparagine at a position corresponding to N508 of SEQ ID NO: 290; and
v) Valine at a position corresponding to V509 of SEQ ID NO: 290;

Further to this embodiment the demethylase may not comprise histidine at a position corresponding to H448 of SEQ ID NO: 290, asparagine at a position corresponding to H508 of SEQ ID NO: 290 and/or valine at a position corresponding to H509 of SEQ ID NO: 290. Still further to this embodiment the demethylase may comprise tyrosine at the position corresponding to position 448 of SEQ ID NO: 290, threonine at the position corresponding to position corresponding to H508 of SEQ ID NO: 290 and/or glycine at the position corresponding to position corresponding to H509 of SEQ ID NO: 290. Within this embodiment the demethylase may specifically be the P450 of SEQ ID NO: 250 or SEQ ID NO: 252.

The demethylase of SEQ ID NO: 218, 220, 222, 224, 226, 228, 236, 240, 250, 252, 254 and 268 have in addition to N-demethylase activity also 0-demethylase activity (ODM) and are capable of demethylating thebaine of the formula I into oripavine of the formula III as described supra.

In a separate embodiment the cell of the invention further comprises a demethylase-CPR capable of reducing and/or regenerating the demethylase enzyme. The demethylase-CPR may also be heterologous to the cell.

Some demethylases may work better together with a demethylase-CPR from a related source so in a particular embodiment where the demethylase is an insect demethylase, the demethylase-CPR may also advantageously be an insect demethylase-CPR, such as a demethylase-CPR derived from an insect of the order Lepidoptera, such as the insect demethylase-CPR derived from an insect of the genus Helicoverpa, Heliothis or Spodoptera such as demethylase-CPR derived from an insect of the species Helicoverpa armigera, Heliothis virescens or Spodoptera exigua.

In particular, the insect demethylase-CPR may comprise a polypeptide selected from the group consisting of:
a) a demethylase-CPR which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase-CPR comprised in SEQ ID NO: 292, 294, 296, 298, 300 or 302;
b) a demethylase-CPR encoded by a polynucleotide which is at least 20% identical to the polynucleotide comprised in SEQ ID NO: 293, 295, 297, 299, 301, 303 or 305 or genomic DNA thereof; and
c) a functional variant of the demethylase-CPR of (a) or (b) capable of reducing/regenerating the demethylase of the invention.

In another embodiment where the demethylase is a fungal demethylase the demethylase-CPR may advantageously be a fungal demethylase-CPR. In particular, the fungal demethylase-CPR may comprise a polypeptide selected from the group consisting of:
a) a demethylase-CPR which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase-CPR comprised in SEQ ID NO: 305;
b) a demethylase-CPR encoded by a polynucleotide which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the polynucleotide comprised in SEQ ID NO: 306 or genomic DNA thereof; and
c) a functional variant of the demethylase-CPR of (a) or (b) capable of reducing/regenerating the demethylase.

Further suitable Demethylases are disclosed in WO2018/229306 or WO2018/075670, which is hereby incorporated by reference in their entirety.

In one embodiment the heterologous demethylase is an artificial mutant. In one type of mutations the naturally occurring leader/signal sequence has been mutated to improve the performance eg. by wholly or partially replacing the leader/signal sequence with a leader/signal sequence from another enzyme. Examples of such mutations are SEQ ID NOS: 845, 847, 851, 853, 857, 859, 863, 865, 867 and 869.

In another embodiment the demethylase is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase comprised in SEQ ID NO 152 (Hv_CYP_A0A2A4JAM9) and has one or more mutations corresponding to A110X, H242X, and/or V224X, such as A110N, 242P and/or V224I, preferably all three mutations A110N+H242P+V224I.

In another embodiment the demethylase is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase comprised in SEQ ID NO 140 (HaCYP6AE15v2) and has one or more mutations corresponding to A316X and/or D392X, such as A316G and/or D392E preferably both.

Further the invention provides mutant insect demethylases comprising one or more mutations in the signal sequence of the naturally occurring insect demethylase. In these insect demethylases the signal sequence may have been wholly or partially replaced by a signal sequence from another enzyme. Suitably such mutant demethylases have least 70%, such as at least 80%, such as at least 90%, such as at least 95%, such as at least 96%, such as at least 97%, such as at least 98%, such as at least 99%, such as 100% identity to the demethylase comprised in SEQ ID NO: 845, 847, 851, 853, 857, 859, 863, 865, 867 or 869. Also mutant insect demethylases are provided having least 70%, such as at least 80%, such as at least 90%, such as at least 95%, such as at least 96%, such as at least 97%, such as at least 98%, such as at least 99%, such as 100% identity to the demethylase comprised in SEQ ID NO: 152 and comprising one or more mutations corresponding to A110X, H242X, and/or V224X, optionally A110N, H242P and/or V224I. Still further, mutant insect demethylases are provided having at least 70%, such as at least 80%, such as at least 90%, such as at least 95%, such as at least 96%, such as at least 97%, such as at least 98%, such as at least 99%, such as 100% identity to the demethylase comprised in SEQ ID NO: 140 and comprising one or more mutations corresponding to A316X and/or D392X, optionally A316G and/or D392E.

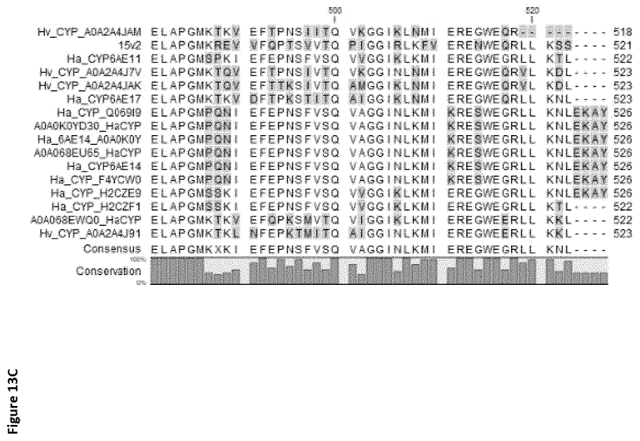

Analysis comparing the best performing insect demethylases (see example 41) was shown share structural sequence features in the form of amino acid positions conserved within the active and high preforming insect demethylases. Accordingly, the insect demethylase of the invention comprise one or more conserved amino acids corresponding to amino acids selected from positions G103, H111, K167, E198, R219, L223, I256, A259, L273, V284, I309, L314, Q517, L160, N216 and/or R443 of SEQ ID NO: 152 (Hv_CYP_A0A2A4JAM9) or any conservative substitutions thereof. In a special embodiment the selected one or more conserved amino acid is/are in or near the active site of the demethylase corresponding to G103, H111 and L314 of SEQ ID NO: 152 or any conservative substitutions thereof. Conservative substitutions which may be considered includes but are not limited to i) aliphatic substitutions, such as between G, A, V, L and I; ii) Hydroxyl or sulfur/selenium-containing substitutions such as between S, C, T and M; iii) aromatic substitutions such as between F, Y, and W, iv) basic substitutions, such as between H, K and R; and v) acidic and amidic substitutions, such as between D, E, N and Q. For example, L160 SEQ ID NO: 152 may also V160 and is considered a conservative substitution (see table 43-3 and FIG. 13). For clarity as regards positions “corresponding” to conserved positions in SEQ ID NO: 152, such positions also include positions which has a different number in a candidate sequence, but which still corresponds and compares to the conserved position in SEQ ID NO: 152 upon alignment with the candidate sequence. Such shifts in numbers occurs e.g. when making amino acid additions or extensions to a candidate sequence. Additional exemplary conservative substitutions are defined in sequence alignment software tools, for example Clustal W, based on additional structural considerations. The software output uses one dot or two dots in the output to indicate the degree of conservation. Examples of tolerated conservative substitutions are those corresponding to I256V, L160M, N216S, R443K of SEQ ID NO: 152. In a particular embodiment the demethylase comprises comprise one or more conserved amino acids corresponding to amino acids selected from positions G103, H111, K167, E198, R219, L223, I256, A259, L273, V284, I309, L314, Q517, L160, N216 and/or R443 of SEQ ID NO: 152 (Hv_CYP_A0A2A4JAM9) or any conservative substitutions thereof and comprises a polypeptide which is at least 60% identical to the insect demethylase comprised in SEQ ID NO: 152.

Heterologous TH—Tyrosine hydroxylase

In another aspect the host cell of the invention expresses alone or in combination with other heterologous genes of the invention one or more heterologous genes encoding a tyrosine hydroxylases. The TH of the invention may suitably be any natural or mutant TH capable of catalyzing L-tyrosine into L-DOPA. Particularly, the TH is of the CYP76 family. In a special embodiment the TH has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the TH comprised in SEQ ID NO: 7, 9, 11, 13, 15, 17, 19, 21, 23, 25, 27, 29, 31, 33, 35, 37, 39, 41, 43, 45, 47, 49, 51, 53, 55, 57, 59, 61, 63 or 65. In a separate embodiment the TH has at least 90%, such as at least 95%, such as at least 96%, such as at least 97%, such as at least 98%, such as at least 99%, such as 100% identity to the TH comprised in SEQ ID NO: 7, 9, 11, 13, 15, 17, 19, 21, 23 or 25. Further suitable THs are disclosed in PCT/EP2020/050610 (unpublished) and WO2016/049364, which are hereby incorporated by reference in its entirety.

### Reducing or Eliminating Enzymes Lowering Performance of the Benzylisoquinoline Alkaloid Pathway

In another aspect the host cell of the invention is genetically modified to reduce or eliminate (knockout) activity of one or more native enzymes, which negatively impacts on the production of benzylisoquinoline alkaloid. Such manipulation may be achieved in several ways, all applicable to the host cell of the invention. Reduction or elimination of enzyme activity may be accomplished by disrupting, deleting and/or attenuating expression of the gene encoding the enzyme and/or the translation of the RNA into the protein, eg. by deleting or mutating the gene. Alternatively, and/or in addition, the enzyme may also be mutated to a less active or non-active variant. In reducing or eliminating activity of enzymes native to the host care should be taken to balance the positive impact on production of benzylisoquinoline alkaloid and the potential negative impact on cellular viability and growth for maintain an acceptable level of vital cellular functions.

Reduction or elimination of activity of enzymes native to the host cell, particularly includes reduction or elimination enzymes shunting precursors or products away from the benzylisoquinoline alkaloid pathway, so that they become unavailable for producing benzylisoquinoline alkaloids. One such group of such enzymes is dehydrogenases native to the host cell and in particular dehydrogenases comprised in SEQ ID NO: 663, 665, 667, 669, 671, 673, 675, 677, 679, 681, 683, 685, 687, 689, 691, 693, 695, 697, 699, 701, 703 or 705. Another group of such enzymes are reductases native to the host cell and in particular reductases comprised in SEQ ID NO: 707, 709, 711, 713, 715, 717, 719, 721, 723, 725, 727, 729 or 731. Preferred targets of reduction or elimination are one or more enzymes comprised in SEQ ID NO: 665 (ADH6), 669 (YPR1), 671 (AAD3), 675 (ADH3), 679 (ALD6), 705 (HFD1), 709 (ALD4), 713 (GRE2), 717 (YDR541C), 721 (ARI1), 729 (PHA2) or 731 (TRP3). Reduction or elimination of one or more the enzymes comprised in 705 (HFD1), 713 (GRE2) or 721 (ARI1), is particularly useful.

Further useful knockouts include:

| Gene name | Systematic name* | Function (ref) |
|---|---|---|
| ALD2 | YMR170C | Aldehyde dehydrogenase ^{(1,2)} |
| ALD3 | YMR169C | Aldehyde dehydrogenase ^{(1,2)} |
| ALD5 | YER073W | Aldehyde dehydrogenase ⁽¹⁾ |
| ADH1 | YOL086C | Alcohol dehydrogenase ⁽¹⁾ |
| ADH2 | YMR303C | Alcohol dehydrogenase ⁽¹⁾ |
| ADH4 | YGL256W | Alcohol dehydrogenase ⁽¹⁾ |
| ADH5 | YBR145W | Alcohol dehydrogenase ⁽¹⁾ |
| ADH7 | YCR105W | Alcohol dehydrogenase ^{(1,2)} |
| SFA1 | YDL168W | Formaldehyde dehydrogenase (^{1,2)} |
| YGL039W | YGL039W | Similar to ARI1 ^{(1,2)} |
| GCY1 | YOR120W | Similar to ARI1 ^{(1,2)} |
| AAD14 | YNL331C | Similar to ARI1 ^{(1,2)} |
| AAD4 | YDL243C | Similar to ARI1 ^{(1,2)} |
| TRP2 | YER090W | Production of tryptophan ⁽¹⁾ |

WO2019/243624 ⁽¹⁾ and Pyne et al, BioRxiv preprint 2019 ⁽²⁾; all hereby incorporated by reference in their entirety. Sequences of the table can also be found in Saccharomyces genome database (https://www.yeastgenome.org), incorporated herein by reference. Further suitable knockouts are disclosed in WO2018/029282, WO2019/157383

WO2019/243624⁽¹⁾ and Pyne et al, BioRxiv preprint 2019⁽²⁾; all hereby incorporated by reference in their entirety. Sequences of the table can also be found in Saccharomyces genome database (https://www.yeastgenome.org), incorporated herein by reference. Further suitable knockouts are disclosed in WO2018/029282, WO2019/157383

### Heterologous Norcoclaurine Synthase (NCS)

In a further aspect the host cell of the invention expresses alone or in combination with other heterologous genes of the invention one or more heterologous gene encoding a norcoclaurine synthase (NCS). The NCS of the invention may suitably be any natural or mutant NCS capable of converting Dopamine and 4-HPAA into (S)-norcoclaurine. In a special embodiment the NCS has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the NCS comprised in SEQ ID NO: 73 OR 76. In a separate embodiment the NCS has at least 90%, such as at least 95%, such as at least 96%, such as at least 97%, such as at least 98%, such as at least 99%, such as 100% identity to the NCS comprised in SEQ ID NO: 73 OR 76. Further suitable NCSs are disclosed in WO2018/229305, WO2014/143744, WO2019/165551 and US2015267233, which is hereby incorporated by reference in its entirety.

### Heterologous STORR

In a further aspect the host cell of the invention expresses alone or in combination with other heterologous genes of the invention one or more heterologous genes encoding enzymes capable of epimerizing/isomerizing one benzylisoquinoline alkaloid to a benzylisoquinoline alkaloid isomer, such as for example (S)-Reticuline into (R)-reticuline. In a special embodiment the epimerase is:
i) a fused 1,2-dehydroreticuline synthase-1,2-dehydroreticuline reductases (DRS-DRR) converting (S)-Reticuline into (R)-reticuline, wherein
   ia) the DRS-DDRs has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRS-DRR comprised in SEQ ID NO: 92, 94, 96; or
   ib) the DRS moiety has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRS comprised in SEQ ID NO: 98, 100, 102, 104 or 106; and the DRR moiety has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRR comprised in SEQ ID NO: 108 or 110; or
ii) a DRS having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRS comprised in SEQ ID NO: 98, 100, 102, 104 or 106; and a DRR having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRR comprised in SEQ ID NO: 108 or 110.

In a particular embodiment the DRR moiety of the epimerase, whether fused to the DRS or separate an Imine reductase, preferably a StIRED such as the reductases comprised in SEQ ID NO. 108 or 110.

Further suitable epimerases/isomerases are disclosed in WO2015/081437, WO2016/183023, WO2015/173590, WO2018/000089, WO2019/028390 and WO2019/165551 which are hereby incorporated by reference in their entirety.

### Heterologous THS

In another aspect the host cell of the invention expresses alone or in combination with other heterologous genes of the invention one or more heterologous genes encoding a thebaine synthase (THS). The THS of the invention may suitably be any natural or mutant THS capable of converting 7-O-acetylsalutaridinol into thebaine. In a special embodiment the THS has is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the THS comprised in SEQ ID NO: 126, 127, 128, 129, 131, 133, 134, 136 or 138. In particular SEQ ID NO: 134 and 136 are very efficient thebaine synthases.

Further suitable THSs are disclosed in WO2018/005553, WO2014/143744 and WO2019/165551, which are hereby incorporated by reference in their entirety.

### Heterologous Transporters

In another aspect the host cell of the invention expresses alone or in combination with other heterologous genes of the invention one or more heterologous genes encoding transporter protein. The transporter protein of the invention may suitably be any natural or mutant transporter protein capable of uptake or export in the host cell of a reticuline derivative, such as thebaine, northebaine, oripavine and/or nororipavine.

In a special embodiment the transporter protein has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the transporter protein comprised in SEQ ID NO: 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401,403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485,487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 733, 735, 795, 797, 799, 801, 803, 805, 807, 809, 811, 813, 815, 817, 819, 821, 823 or 825.

Selecting the optimal transporter for given pathway setup may dependent on choice of other enzymes such as the demethylase. So, in a particular embodiment when incorporating a demethylase, especially an insect demethylases, converting oripavine into nor-oripavine, insect transporters are preferred. In particular transporters T180_McoPUP3_46 (SEQ ID NO: 595), T193_AanPUP3_55 (SEQ ID NO: 613), T149_AcoPUP3_59 (SEQ ID NO: 537) and/or T165_AcoPUP3_13 (SEQ ID NO: 567) have shown particularly effective. In another particular embodiment when in incorporating a demethylase, especially an insect demethylases, converting thebaine into northebaine, insect transporters are preferred. In particular transporters T193_AanPUP3_55 (SEQ ID NO: 613), T198_AcoT97_GA (SEQ ID NO: 623), T149_AcoPUP3_59 (SEQ ID NO: 537) and/or T122_PsoPUP3_17 (SEQ ID NO: 487) have shown particularly effective. Further suitable transporter proteins are disclosed in WO2020/078837, which is hereby incorporated by reference in its entirety.

In a further separate embodiment, the transporter may be an Equilibrative Nucleoside Transporter (ENT) as described in Boswell-Casteel and Hays, 2017. Equilibrative Nucleoside Transporters including those belonging to the SLC29A/ENT transporter (TC 2.A.57) family (https://www.uniprot.org) have been shown herein to be capable of demethylase-mediated bioconversion of methylated benzylisoquinoline alkaloids to the corresponding nor-benzylisoquinoline alkaloids—in particular oripavine to nororipavine—in a highly efficient manner. Such improvements in yield are particularly remarkable and represent a significant step forward towards a sustainable, secure, and scalable biosynthetic means of producing these compounds.

The Equilibrative Nucleoside Transporter may particularly be an insect Equilibrative Nucleoside Transporter, including the transporters having at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the transporter protein comprised in SEQ ID NOS: 795, 797, 799, 801, 803, 805, 807, 809, 811, 813, 815, 817, 819, 821, 823 or 825, especially SEQ ID NOS: 795, 797, 799, 801.

The useful insect transporters disclosed herein have not hitherto been demonstrated to benefit production of benzylisoquinoline alkaloids when incorporated heterologously in genetically modified microorganisms comprising pathways producing benzylisoquinoline alkaloids. Accordingly, in a separate aspect the invention provides a genetically modified host cell comprising a pathway having enhanced production of one or more benzylisoquinoline alkaloids wherein the cell expresses one or more heterologous genes encoding an insect derived transporter protein increasing the cellular uptake or secretion of a benzylisoquinoline alkaloid precursor, said precursor preferably being a benzylisoquinoline alkaloid itself. Particular insect transporters include transporter proteins from the insect genera of Helicoverpa, Heliothis or Pectinophora, in particular from species of Pectinophora gossypiella, Helicoverpa armigera or Heliothis virescens. In particular the transporter proteins have at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the transporter protein comprised in SEQ ID NO: 631, 633, 637, 649, 651, 653, 655, 657 or 659. Moreover, the genetically modified cell of the invention may comprise one or more copies of genes encoding one or more insect transporter proteins such as genes/polynucleotides which is at least 70% identical to the transporter encoding polynucleotide comprised in SEQ ID NO: 632, 634, 638, 652, 654, 656, 658 or 660 or genomic DNA thereof.

### Further Enzymes of the Benzylisoquinoline Alkaloid Pathway

In another aspect the host cell of the invention expresses in combination with other heterologous genes of the invention one or more further heterologous or native enzymes of the benzylisoquinoline alkaloid pathway. In a particular embodiment the host cell of the invention expresses one or more genes encoding polypeptides selected from:
a) a 3-deoxy-D-arabino-2-heptulosonic acid 7-phosphate synthase (DAHP synthase) converting PEP and E4P into DAHP;
b) a 5-enolpyruvylshikimate-3-phosphate (EPSP) synthase (aro1) converting 3-phosphoshikimate and PEP into EPSP;
c) an aro1 polypeptide converting DHAP and PEP into EPSP;
d) a chorismate synthase converting EPSP into Chorismate;
e) a chorismate mutase converting Chorismate into prephenate;
f) a prephenate dehydrogenase (Tyr1) converting prephenate into 4-HPP;
g) an aromatic aminotransferase converting 4-HPP into L-Tyrosine;
h) a TH-CPR capable of reducing TH;
i) a L-dopa decarboxylase (DODC) converting L-dopa into dopamine;
j) a Tyrosine decarboxylase (TYDC) converting L-dopa into dopamine;
k) a hydroxyphenylpyruvate decarboxylase (HPPDC) converting 4-HPP into 4-HPPA;
l) a monoamine oxidase converting dopamine into 3,4-DHPAA;
m) a 6-O-methyltransferase (6-OMT) converting (S)-norcoclaurine into (S)-Coclaurine and/or norlaudanosoline into (S)-3′-Hydroxy-coclaurine;
n) a Coclaurine-N-methyltransferase (CNMT) converting (S)-Coclaurine into (S)—N-Methylcoclaurine and/or (S)-3′-hydroxycoclaurine into (S)-3′-hydroxy-N-methyl-coclaurine;
o) a N-methylcoclaurine hydroxylase (NMCH) converting (S)-Coclaurine into (S)-3′-hydroxycoclaurine and/or (S)—N-Methylcoclaurine into (S)-3′-Hydroxy-N-Methylcoclaurine;
p) a 3′-hydroxy-N-methyl-(S)-coclaurine 4′-O-methyltransferase (4′-OMT) converting (S)-3′-Hydroxy-N-Methylcoclaurine into (S)-Reticuline;
q) a DRS-CPR capable of reducing DRS-DRR;
r) a salutaridine synthase (SAS) converting (R)-reticuline into Salutaridine;
s) a salutaridine reductase (SAR) converting Salutaridine to Salutaridinol; and
t) a salutaridinol 7-O-acetyltransferase (SAT) converting Salutaridinol into 7-O-acetylsalutaridinol.

In a special embodiment the corresponding:
a) DAHP synthase has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DAHP synthase comprised in SEQ ID NO: 1
b) chorismate mutase has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the chorismate synthase comprised in SEQ ID NO: 3;
c) TH-CPR has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the TH-CPR comprised in SEQ ID NO: 67;
d) DODC has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DODC comprised in SEQ ID NO: 69 or 71;
e) 6-OMT has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the 6-OMT comprised in SEQ ID NO: 79 or 81;
f) CNMT has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the CNMT comprised in SEQ ID NO: 82 or 84;
g) NMCH has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the NMCH comprised in EQ ID NO: 85 OR 87;
h) 4′-OMT has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the 4′-OMT comprised in SEQ ID NO: 89 or 91;
i) DRS-CPR has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the DRS-CPR comprised in SEQ ID NO: 112 or 114;
j) SAS has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the SAS comprised in SEQ ID NO: 116 or 118;
k) SAR has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the SAR comprised in SEQ ID NO: 120 or 122;
l) SAT has at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identity to the SAT comprised in SEQ ID NO: 123 or 125; and

Further suitable enzymes of the benzylisoquinoline alkaloid pathway are disclosed in US2019100781 and WO2019/165551, which is hereby incorporated by reference in their entirety.

### Additional Cell Modifications Improving Production of Benzylisoquinoline Alkaloids

During the efforts of improving cellular production of recombinant cellular production of benzylisoquinoline alkaloids, several additional useful modifications to cells improving the cellular performance was discovered. In a first aspect it was found that cytosolic heme levels in a production host cell is a significant limiting factor in production of demethylated nor-benzylisoquinoline alkaloids such as nororipavine and/or northebaine and that modifications to the cell increasing the cytosolic heme levels strongly benefits production of such demethylated nor-benzylisoquinoline alkaloids. Accordingly, in one embodiment the host cell is further modified to increase availability of heme in the cell, in particular by modifying expression of one or more heme expression co-factors in the cell.

In one embodiment the heme availability can be increased by overexpressing and/or co-expressing one or more rate-limiting enzymes from the heme pathway, including but not limited to HEM2, HEM3 and/or HEM12. Overexpression of such genes can be accomplished for example by increasing the number of copies of integrated genes and/or by using stronger promoters of other factors increase translation or transcription of the gene. Preferably both an increase in copy number and use of an appropriate combination of stronger and weaker promoters are used to increase availability of heme. Useful promoters for these gene include pPYK1, pSED1, pKEX2, pTEF1, pTDH3 and pPGK1, where pTEF1, pTDH3 and pPGK1 are the stronger ones. In another embodiment heme variability is increased by disrupting, deleting and/or attenuating any heme-down regulating genes, such as HMX1. In another embodiment heme availability is increased by adding a heme production booster agent such as hemin (Protchenko et al., 2003 and Krainer et al., 2015, respectively).

In a further aspect it was found that overexpressing and/or co-expressing P450 helper genes in a production host cell significantly benefits production of demethylated nor-benzylisoquinoline alkaloids. Such P450 helper genes includes, but is not limited to:
a) DAP1, which encodes a heme-binding protein involved in the regulation the function of cytochrome P450 (Hughes et al., 2007);
b) HAC1, a transcription factor that modulates the unfolded protein response (Kawahara T, et al., 1997);
c) KAR2, HSP82, CNE1, SSA1, CPR6, FES1, HSP104 and STI1 involved in protein processing as well as heat shock response (Yu et al., 2017).
   In a still further aspect, it was found that increasing cytosolic levels of NADPH by overexpressing and/or co-expressing genes in the pentose metabolic pathway significantly benefits production of demethylated nor-benzylisoquinoline alkaloids. Such genes include but is not limited to ZWF1 and GND1 genes from the pentose phosphate pathway (Stincone et al., 2015).

In a further aspect it was found that detoxifying the genetically modified cell from formaldehyde, a toxic by-product released during cytochrome P450 N-demethylation reaction (Wehner E P et al., 1993 and Kalász H et al., 1998), significantly benefits production of demethylated nor-benzylisoquinoline alkaloids. Lowering formation of cytosolic formaldehyde in the cell can be achieved modifying genes encoding factors regulating formaldehyde levels and/or toxicity. Such genes/factors include but is not limited to SFA1, which when overexpressed and/or co-expressed reduce formaldehyde levels and/or toxicity and thereby increase production of demethylated nor-benzylisoquinoline alkaloids.

### Functional Homologues

Functional homologs (also referred herein to as functional variants) of the enzymes/polypeptides described herein are also suitable for use in producing benzylisoquinoline alkaloid in the genetically modified host cell. A functional homolog is a polypeptide that has sequence similarity to a reference polypeptide, and that carries out one or more of the biochemical or physiological function(s) of the reference polypeptide. A functional homolog and the reference polypeptide can be a natural occurring polypeptide, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered functional homologs. The term “functional homolog” is sometimes applied to the nucleic acid that encodes a functionally homologous polypeptide. Functional homologs can be identified by analysis of nucleotide and polypeptide sequence alignments. For example, performing a query on a database of nucleotide or polypeptide sequences can identify homologs of benzylisoquinoline alkaloid biosynthesis polypeptides. Sequence analysis can involve BLAST, Reciprocal BLAST, or PSI-BLAST analysis of non-redundant databases using a UGT amino acid sequence as the reference sequence. Amino acid sequence is, in some instances, deduced from the nucleotide sequence. Those polypeptides in the database that have greater than 40% sequence identity are candidates for further evaluation for suitability as a benzylisoquinoline alkaloid biosynthesis polypeptide. Amino acid sequence similarity allows for conservative amino acid substitutions, such as substitution of one hydrophobic residue for another or substitution of one polar residue for another. If desired, manual inspection of such candidates can be carried out to narrow the number of candidates to be further evaluated. Manual inspection can be performed by selecting those candidates that appear to have domains present in benzylisoquinoline alkaloid biosynthesis polypeptides, e.g., conserved functional domains. In some embodiments, nucleic acids and polypeptides are identified from transcriptome data based on expression levels rather than by using BLAST analysis. Methods for conservative substitution is known to the skilled person, see for example https://www.ncbi.nlm.nih.gov/pmc/articles/PMC1449787/ or https://link/pringer.com/article/10.1007/BF02300754.

Conserved regions can be identified by locating a region within the primary amino acid sequence of a benzylisoquinoline alkaloid biosynthesis polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on for example the World Wide Web at sanger.ac.uk/Software/Pfam/ and pfam.janelia.org/. The information included at the Pfam database is described in Sonnhammer et al. (1998); Sonnhammer et al. (1997); and Bateman et al. (1999). Conserved regions also can be determined by aligning sequences of the same or related polypeptides from closely related species. Closely related species preferably are from the same family. In some embodiments, alignment of sequences from two different species is adequate to identify such homologs.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

For example, polypeptides suitable for producing benzylisoquinoline alkaloids in a genetically modified host cell include functional homologs of TH's, NCS's, 6-OMT's, CNMT's, NMCH's, 4′-OMT's, DRS-DRR's, SAS's, SAR's, SAT's, THS's, CPR's and demethylating P450's.

Methods to modify the substrate specificity of benzylisoquinoline alkaloids pathway enzymes are known to those skilled in the art, and include without limitation site-directed/rational mutagenesis approaches, random directed evolution approaches and combinations in which random mutagenesis/saturation techniques are performed near the active site of the enzyme. For example see Osmani et al. (2009).

A candidate sequence typically has a length that is from 80% to 200% of the length of the reference sequence, e.g., 82, 85, 87, 89, 90, 93, 95, 97, 99, 100, 105, 110, 115, 120, 130, 140, 150, 160, 170, 180, 190, or 200% of the length of the reference sequence. A functional homolog polypeptide typically has a length that is from 95% to 105% of the length of the reference sequence, e.g., 90, 93, 95, 97, 99, 100, 105, 110, 115, or 120% of the length of the reference sequence, or any range between.

It will be appreciated that functional benzylisoquinoline alkaloids pathway enzymes/polypeptides can include additional amino acids that are not involved in the enzymatic activities carried out by the enzymes. In some embodiments, such enzymes are fusion proteins. The terms “chimera,” “fusion polypeptide,” “fusion protein,” “fusion enzyme,” “fusion construct,” “chimeric protein,” “chimeric polypeptide,” “chimeric construct,” and “chimeric enzyme” can be used interchangeably herein to refer to proteins engineered through the joining of two or more genes that code for different proteins. In some embodiments, a nucleic acid sequence encoding a benzylisoquinoline alkaloids pathway enzyme/polypeptide can include a tag sequence that encodes a “tag” designed to facilitate subsequent manipulation (e.g., to facilitate purification or detection), secretion, or localization of the encoded enzyme. Tag sequences can be inserted in the nucleic acid sequence encoding the polypeptide such that the encoded tag is located at either the carboxyl or amino terminus of the polypeptide. Non-limiting examples of encoded tags include green fluorescent protein (GFP), human influenza hemagglutinin (HA), glutathione S transferase (GST), polyhistidine-tag (HIS tag), and Flag™ tag (Kodak, New Haven, CT). Other examples of tags include a chloroplast transit peptide, a mitochondrial transit peptide, an amyloplast peptide, signal peptide, or a secretion tag.

In some embodiments, a fusion protein is a protein altered by domain swapping. As used herein, the term “domain swapping” is used to describe the process of replacing a domain of a first protein with a domain of a second protein. In some embodiments, the domain of the first protein and the domain of the second protein are functionally identical or functionally similar. In some embodiments, the structure and/or sequence of the domain of the second protein differs from the structure and/or sequence of the domain of the first protein. In some embodiments, a benzylisoquinoline alkaloids pathway enzyme/polypeptide is altered by domain swapping.

### Nucleotides Expressed by the Host Cell

In another aspect the host cell of the invention expresses one or more polynucleotides or genes selected from:
a) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the DAHP synthase encoding polynucleotide comprised in SEQ ID NO: 2 or genomic DNA thereof;
b) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the chorismate mutase encoding polynucleotide comprised in SEQ ID NO: 4 or genomic DNA thereof;
c) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the TH encoding polynucleotide comprised in SEQ ID NO: 8, 10, 12, 14, 16, 18, 20, 22, 24, 26, 28, 30, 32, 34, 36, 38, 40, 42, 44, 46, 48, 50, 52, 54, 56, 58, 60, 62, 64 or 66 or genomic DNA thereof;
d) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the TH-CPR encoding polynucleotide comprised in SEQ ID NO: 68 or genomic DNA thereof;
e) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the DODC encoding polynucleotide comprised in SEQ ID NO: 70 or 72 or genomic DNA thereof;
f) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the NCS encoding polynucleotide comprised in SEQ ID NO: 74 or 77 or genomic DNA thereof;
g) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the 6-OMT encoding polynucleotide comprised in SEQ ID NO: 80 or genomic DNA thereof;
h) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the CNMT encoding polynucleotide comprised in SEQ ID NO: 83 or genomic DNA thereof;
i) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the NMCH encoding polynucleotide comprised in SEQ ID NO: 86 or 88 or genomic DNA thereof;
j) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the 4′-OMT encoding polynucleotide comprised in SEQ ID NO: 90 or genomic DNA thereof;
k) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the DRS-DRR encoding polynucleotide comprised in SEQ ID NO: 93, 95 or 97 or genomic DNA thereof;
l) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the DRS encoding polynucleotide comprised in SEQ ID NO: 99, 101, 103, 105 or 107 or genomic DNA thereof;
m) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the DRR encoding polynucleotide comprised in SEQ ID NO: 109 or 111 or genomic DNA thereof;
n) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the DRS-CPR encoding polynucleotide comprised in SEQ ID NO: 113 or 115 or genomic DNA thereof;
o) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the SAS encoding polynucleotide comprised in SEQ ID NO: 117 or 119 or genomic DNA thereof;
p) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the SAR encoding polynucleotide comprised in SEQ ID NO: 121 or genomic DNA thereof;
q) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the SAT encoding polynucleotide comprised in SEQ ID NO: 124 or genomic DNA thereof;
r) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the THS encoding polynucleotide comprised in SEQ ID NO: 130, 132, 135, 137 or 139 or genomic DNA thereof;
s) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the ODM encoding polynucleotide comprised in SEQ ID NO: 219, 221, 223, 225, 227, 229, 237, 241, 251, 253, 255 and 267 or genomic DNA thereof;
t) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase encoding polynucleotide comprised in any one of SEQ ID NO: 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 828, 830, 832, 834, 836, 838, 840, 842, 844, 846, 848, 850, 852, 854, 856, 858, 860, 862, 864, 866, 868 and 870 or genomic DNA thereof;
u) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the demethylase-CPR encoding polynucleotide comprised in any one of SEQ ID NO: 293, 295, 297, 299, 301, 303, 304 or 306 or genomic DNA thereof; and
v) one or more polynucleotides which is at least 20%, such as at least 40%, such as at least 50%, such as at least 60%, such as at least 70%, such as at least 80%, such as at least 90%, such as at least 95%, such as at least 96%, such as at least 97%, such as at least 98%, such as at least 99%, such as 100% identical to the transporter encoding polynucleotide comprised in SEQ ID NO: 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 734 or 736 or genomic DNA thereof.

Any nucleotides disclosed herein may be codon optimized for expression in a particular selected host using methods available to the skilled person or commercially available from technology providers-see for example Gene Reports Volume 9, December 2017, Pages 46-53: Strategies of codon optimization for high-level heterologous protein expression in microbial expression systems, incorporated herein by reference. Examples of codon optimized genes are those of SEQ ID NOS: 771, 773, 775, 777, 779, 781, 783, 785, 787, 789, 791 and 793.

### Host Cells.

The cell of the invention may be any host cell suitable for hosting and expressing the P450s of the invention and converting thebaine and/or oripavine into the corresponding nor-compounds.

In particular the cell of the invention may be an eukaryote cell selected from the group consisting of mammalian, insect, plant, or fungal cellsin another embodiment the cell is a fungal cell selected from the phylas consisting of Ascomycota, Basidiomycota, Neocallimastigomycota, Glomeromycota, Blastocladiomycota, Chytridiomycota, Zygomycota, Oomycota and Microsporidia. A particularly useful fungal cell is a yeast cell selected from the group consisting of ascosporogenous yeast (Endomycetales), basidiosporogenous yeast, and Fungi Imperfecti yeast (Blastomycetes). Such yeast cells may further be selected from the genera consisting of Saccharomyces, Kluveromyces, Candida, Pichia, Debaromyces, Hansenula, Yarrowia, Zygosaccharomyces, and Schizosaccharomyces. More specifically the yeast cell may be selected from the species consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica.

An alternative fungal host cell of the invention is a filamentous fungal cell. Such filamentous fungal cell may be selected from the phylas consisting of Ascomycota, Eumycota and Oomycota, more specifically selected from the genera consisting of Acremonium, Aspergillus, Aureobasidium, Bjerkandera, Ceriporiopsis, Chrysosporium, Coprinus, Corio/us, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma. In important embodiments the filamentous fungal cell may be selected from the species consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.

In one embodiment the cell is a plant cell for example of the genus Physcomitrella or Papaver, in particular Papaver somniferum. Other plant cells can be of the family Solanaceae, such genuses of Nicotiana, such as Nicotiana benthamiana. In addition to plant cells the invention also provides an isolated plant, e.g., a transgenic plant, plant part comprising the benzylisoquinoline alkaloid pathway polypeptides of the invention and producing the benzylisoquinoline alkaloids of the invention in useful quantities. The compound may be recovered from the plant or plant part. The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn). Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana. Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats. Also included within the scope of the present invention is any the progeny of such plants, plant parts, and plant cells. The transgenic plant or plant cells comprising the operative pathway of the invention and produce the compound of the invention may be constructed in accordance with methods known in the art. In short, the plant or plant cell is constructed by incorporating one or more expression vectors of the invention into the plant host genome or chloroplast genome and propagating the resulting modified plant or plant cell into a transgenic plant or plant cell. The expression vector conveniently comprises the polynucleotide construct of the invention. The choice of regulatory sequences, such as promoter and terminator sequences and optionally signal or transit sequences, is determined, for example, on the basis of when, where, and how the pathway polypeptides is desired to be expressed. For instance, the expression of a gene encoding a pathway enzyme polypeptide may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506. For constitutive expression, the 358-CaMV, the maize ubiquitin 1, or the rice actin 1 promoter may be used (Franck et al., 1980, Cell 21: 285-294; Christensen et al., 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards and Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant Cell Physiol. 39: 885-889), a Vicia faba promoter from the legumin B4 and the unknown seed protein gene from Vicia faba (Conrad et al., 1998, J. Plant Physiol. 152: 708-711), a promoter from a seed oil body protein (Chen et al., 1998, Plant Cell Physiol. 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiol. 102: 991-1000), the chlorella virus adenine methyltransferase gene promoter (Mitra and Higgins, 1994, Plant Mol. Biol. 26: 85-93), the aldP gene promoter from rice (Kagaya et al., 1995, Mol. Gen. Genet. 248: 668-674), or a wound inducible promoter such as the potato pin2 promoter (Xu et al., 1993, Plant Mol. Biol. 22: 573-588). Likewise, the promoter may be induced by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals. A promoter enhancer element may also be used to achieve higher expression in the plant. For instance, the promoter enhancer element may be an intron that is placed between the promoter and the polynucleotide encoding a polypeptide or domain. For instance, Xu et al., 1993, supra, disclose the use of the first intron of the rice actin 1 gene to enhance expression. The selectable marker gene and any other parts of the expression construct may be chosen from those available in the art. The polynucleotide construct or expression vector is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274). Agrobacterium tumefaciens-mediated gene transfer is a method for generating transgenic dicots (for a review, see Hooykas and Schilperoort, 1992, Plant Mol. Biol. 19: 15-38) and for transforming monocots, although other transformation methods may be used for these plants. A method for generating transgenic monocots is particle bombardment (microscopic gold or tungsten particles coated with the transforming DNA) of embryonic calli or developing embryos (Christou, 1992, Plant J. 2: 275-281; Shimamoto, 1994, Curr. Opin. Biotechnol. 5: 158-162; Vasil et al., 1992, Bio/Technology 10: 667-674). An alternative method for transformation of monocots is based on protoplast transformation as described by Omirulleh et al., 1993, Plant Mo/. Biol. 21: 415-428. Additional transformation methods include those described in U.S. Pat. Nos. 6,395,966 and 7,151,204 (both incorporated herein by reference in their entirety). Following transformation, the transformants having incorporated the expression vector or polynucleotide construct of the invention are selected and regenerated into whole plants according to methods well known in the art. Often the transformation procedure is designed for the selective elimination of selection genes either during regeneration or in the following generations by using, for example, co-transformation with two separate T-DNA constructs or site specific excision of the selection gene by a specific recombinase. In addition to direct transformation of a particular plant genotype with a polynucleotide construct of the invention, transgenic plants may be made by crossing a plant comprising the construct to a second plant lacking the construct. For example, a polynucleotide construct encoding a glycosyl transferase of the invention can be introduced into a particular plant variety by crossing, without the need for ever directly transforming a plant of that given variety. Therefore, the invention encompasses not only a plant directly regenerated from cells which have been transformed in accordance with the invention, but also the progeny of such plants. As used herein, progeny may refer to the offspring of any generation of a parent plant prepared in accordance with the present invention. Such progeny may include a polynucleotide construct of the invention. Crossing results in the introduction of a transgene into a plant line by cross pollinating a starting line with a donor plant line. Non-limiting examples of such steps are described in U.S. Pat. No. 7,151,204. Plants may be generated through a process of backcross conversion. For example, plants include plants referred to as a backcross converted genotype, line, inbred, or hybrid. Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

The cell of the invention may be even further modified by one or more of
a) attenuating, disrupting and/or deleting one or more native or endogenous genes of the cell;
b) inserting two or more copies of polynucleotides encoding the P450s, the demethylase-CPR's and/or one or more of the polypeptides comprised in the operative metabolic pathway;
c) increasing the amount of a substrate for at least one polypeptide of the operative metabolic pathway; and/or
d) increasing tolerance towards one or more substrates, intermediates, or product molecules from the operative metabolic pathway.

### Polynucleotide Constructs and Expression Vectors

In a separate aspect the invention also provides a polynucleotide construct comprising a polynucleotide sequence encoding a heterologous enzymes or transporter protein of the invention operably linked to one or more control sequences, which direct expression of the heterologous enzyme or transporter protein in the host cell harbouring the polynucleotide construct. Conditions for the expression should be compatible with the control sequences. In particular, the control sequence is heterologous to the polynucleotide encoding the heterologous enzyme or transporter protein and in one embodiment the polynucleotide sequence encoding the heterologous enzyme or transporter protein and the control sequence are both heterologous to the host cell comprising the construct. In one embodiment the polynucleotide construct is an expression vector, comprising the polynucleotide sequence encoding the heterologous enzyme or transporter protein of the invention operably linked to the one or more control sequences.

Polynucleotides may be manipulated in a variety of ways allow expression of the heterologous enzyme or transporter protein. Manipulation of the polynucleotide prior to its insertion into an expression vector may be desirable or necessary depending on the expression vector. The techniques for modifying polynucleotides utilizing recombinant DNA methods are well known in the art.

The control sequence may be a promoter, which is a polynucleotide that is recognized by a host cell for expression of a polynucleotide. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell. The promoter may also be an inducible promoter.

Examples of suitable promoters for directing transcription of the nucleic acid construct of the invention in fungal host cell are promoters obtained from the genes for Aspergillus nidulans acetamidase, Aspergillus niger neutral α-amylase, Aspergillus niger acid stable α-amylase, Aspergillus niger or Aspergillus awamori glucoamylase (glaA), Aspergillus gpdA promoter, Aspergillus oryzae TAKA amylase, Aspergillus oryzae alkaline protease, Aspergillus oryzae triose phosphate isomerase, A. niger or A. awamori endoxylanase (xlnA) or β-xylosidase (xlnD), Fusarium oxysporum trypsin-like protease (WO 96/00787), Fusarium venenatum amyloglucosidase (WO2000/56900), Fusarium venenatum Dania (WO 00/56900), Fusarium venenatum Quinn (WO 00/56900), Rhizomucor miehei lipase, Rhizomucor miehei aspartic proteinase, Trichoderma reesei 3-glucosidase, Trichoderma reesei cellobiohydrolase I, Trichoderma reesei cellobiohydrolase 1l, Trichoderma reesei endoglucanase 1, Trichoderma reesei endoglucanase II, Trichoderma reesei endoglucanase III, Trichoderma reesei endoglucanase IV, Trichoderma reesei endoglucanase V, Trichoderma reesei xylanase i, Trichoderma reesei xylanase II, Trichoderma reesei D3-xylosidase, as well as the NA2-tpi promoter and mutant, truncated, and hybrid promoters thereof. NA2-tpi promoter is a modified promoter from an Aspergillus neutral α-amylase gene in which the untranslated leader has been replaced by an untranslated leader from an Aspergillus triose phosphate isomerase gene. Examples of such promoters include modified promoters from an Aspergillus niger neutral α-amylase gene in which the untranslated leader has been replaced by an untranslated leader from an Aspergillus nidulans or Aspergillus oryzae triose phosphate isomerase gene. Other examples of promoters are the promoters described in WO2006/092396, WO2005/100573 and WO2008/098933, incorporated herein by reference.

Examples of suitable promoters for directing transcription of the nucleic acid construct of the invention in a yeast host include the glyceraldehyde-3-phosphate dehydrogenase promoter, PgpdA or promoters obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488. Selecting a suitable promoter for expression in yeast is well know and is well understood by persons skilled in the art.

The control sequence may also be a transcription terminator, which is recognized by a host cell to terminate transcription. The terminator is operably linked to the 3′-terminus of the polynucleotide encoding the polypeptide. Any terminator that is functional in the host cell may be used.

Useful terminators for fungal host cells can be obtained from the genes encoding Aspergillus nidulans anthranilate synthase, Aspergillus niger glucoamylase, Aspergillus niger α-glucosidase, Aspergillus oryzae TAKA amylase, and Fusarium oxysporum trypsin-like protease; while useful terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Useful leaders for fungal host cells can be obtained from the genes for Aspergillus oryzae TAKA amylase and Aspergillus nidulans triose phosphate isomerase, while useful leaders for yeast host cells can be obtained from the genes for Saccharomyces cerevisiae enolase (ENO-1), Saccharomyces cerevisiae 3-phosphoglycerate kinase, Saccharomyces cerevisiae α-factor, and Saccharomyces cerevisiae alcohol dehydrogenase/glyceraldehyde-3-phosphate dehydrogenase (ADH2/GAP).

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell may be used. Useful polyadenylation sequences for fungal host cells can be obtained from the genes for Aspergillus nidulans anthranilate synthase, Aspergillus niger glucoamylase, Aspergillus niger α-glucosidase Aspergillus oryzae TAKA amylase, and Fusarium oxysporum trypsin-like protease; while useful polyadenylation sequences for yeast host cells are described by Guo and Sherman, 1995, Mol. Cellular Biol. 15: 5983-5990.

It may also be desirable to add regulatory sequences that regulate expression of the polypeptide relative to the growth of the host cell. Examples of regulatory systems are those that cause expression of the gene to be turned on or off in response to a chemical or physical stimulus, including the presence of a regulatory compound. In fungi, the Aspergillus niger glucoamylase promoter, Aspergillus oryzae TAKA α-amylase promoter, and Aspergillus oryzae glucoamylase promoter may be used; while in yeast, the ADH2 system or GAL 1 system may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals.

Various nucleotide sequences in addition to the polynucleotide construct of the invention may be joined together to produce a recombinant expression vector, which may include one or more convenient restriction sites to allow for insertion or substitution of the polynucleotide sequence encoding the P450 of the invention at such sites. The recombinant expression vector may be any vector (e.g., a plasmid or virus or chromosomal) that can be conveniently subjected to recombinant DNA procedures and can bring about expression of the P450 encoding polynucleotide. The choice of the vector will typically depend on the compatibility of the vector with the host cell into which the vector is to be introduced. The vector may be an autonomously replicating vector, i.e., a vector that exists as an extrachromosomal entity, the replication of which is independent of chromosomal replication, e.g., a plasmid (linear or closed circular plasmid), an extrachromosomal element, a minichromosome, or an artificial chromosome. The vector may contain any means for assuring self-replication. Alternatively, the vector may, when introduced into the host cell, integrate into the genome and replicate together with the chromosome(s) into which it has been integrated. Furthermore, a single vector or plasmid or two or more vectors or plasmids that together contain the total DNA to be introduced into the genome of the host cell, or a transposon, may be used.

The vector may contain one or more selectable markers that permit easy selection of transformed, transfected, transduced, or the like cells. A selectable marker is a gene from which the product provides for biocide or viral resistance, resistance to heavy metals, prototrophy to auxotrophs, and the like. Useful selectable markers for fungal host cells include amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Useful selectable markers for yeast host cells include, but are not limited to, ADE2, HIS3, LEU2, LYS2, MET3, TRP1, and URA3.

The vector may further contain element(s) that permits integration of the vector into genome of the host cell or permits autonomous replication of the vector in the cell independent of the genome. For integration into the host cell genome, the vector may rely on the polynucleotide encoding the P450 or any other element of the vector for integration into the genome by homologous or non-homologous recombination. Alternatively, the vector may contain additional polynucleotides for directing integration by homologous recombination into the genome of the host cell at precise location(s) in the chromosome(s). To increase the likelihood of integration at a precise location, the integrational elements should contain a sufficient number of nucleic acids, such as 100 to 10,000 base pairs, such as 400 to 10,000 base pairs, and such as 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” refers to a polynucleotide that enables a plasmid or vector to replicate in vivo. Useful origins of replication for fungal cells include AMA 1 and ANSI (Gems et al., 1991, Gene 98: 61-67; Cullen et al., 1987, Nucleic Acids Res. 15: 9163-9175; WO 00/24883). Isolation of the AMA 1 sequence and construction of plasmids or vectors comprising the gene can be accomplished using the methods disclosed in WO2000/24883. Useful origins of replication for yeast host cells are the 2-micron origin of replication, ARS1, ARS4, the combination of ARS1 and CEN3, and the combination of ARS4 and CEN6.

As mentioned, supra, more than one copy of a polynucleotide encoding the P450 of the invention may be inserted into a host cell to increase production of the P450. An increase in the copy number can be obtained by integrating one or more additional copies of the enzyme coding sequence into the host cell genome or by including an amplifiable selectable marker gene with the polynucleotide, so that cells containing amplified copies of the selectable marker gene—and thereby additional copies of the polynucleotide—can be selected by cultivating the cells in the presence of the appropriate selectable agent. The procedures used to ligate the elements described above to construct the recombinant expression vectors of the present disclosure are well known to one skilled in the art (see, e.g., Sambrook et al., 1989, supra).

In alignment with the above the vehicles of this disclose also include those comprising a microbial host cell comprising the polynucleotide construct as described, supra.

### Cultures

The invention also provides a cell culture, comprising any host cell of the invention and a growth medium. Suitable growth mediums for host cells such as mammalian, insect, plant, fungal and/or yeast cells are known in the art.

### Methods of Producing Compounds of the Invention.

The invention also provides a method for producing a benzylisoquinoline alkaloid in particular thebaine, northebaine, oripavine and/or nororipavine and/or a derivative thereof comprising
a) culturing the cell culture of the invention at conditions allowing the cell to produce the benzylisoquinoline alkaloid; and
b) optionally recovering and/or isolating the benzylisoquinoline alkaloid.

The cell culture can be cultivated in a nutrient medium and at conditions suitable for production of the northebaine and/or nororipavine of the invention and/or propagating cell count using methods known in the art. For example, the culture may be cultivated by shake flask cultivation, or small-scale or large-scale fermentation (including continuous, batch, fed-batch, or solid-state fermentations) in laboratory or industrial fermenters in a suitable medium and under conditions allowing the host cells to grow and/or propagate, optionally to be recovered and/or isolated.

The cultivation can take place in a suitable nutrient medium comprising carbon and nitrogen sources and inorganic salts, using procedures known in the art. Suitable media are available from commercial suppliers or may be prepared according to published recipes (e.g. from catalogues of the American Type Culture Collection). The selection of the appropriate medium may be based on the choice of host cell and/or based on the regulatory requirements for the host cell. Such media are available in the art. The medium may, if desired, contain additional components favoring the transformed expression hosts over other potentially contaminating microorganisms. Accordingly, in an embodiment a suitable nutrient medium comprises a carbon source (e.g. glucose, maltose, molasses, starch, cellulose, xylan, pectin, lignocellolytic biomass hydrolysate, etc.), a nitrogen source (e. g. ammonium sulphate, ammonium nitrate, ammonium chloride, etc.), an organic nitrogen source (e.g. yeast extract, malt extract, peptone, etc.) and inorganic nutrient sources (e.g. phosphate, magnesium, potassium, zinc, iron, etc.).

The cultivation of the host cell may be performed over a period of from about 0.5 to about 30 days. The cultivation process may be a batch process, continuous or fed-batch process, suitably performed at a temperature in the range of 0-100° C. or 0-80° C., for example, from about 0° C. to about 50° C. and/or at a pH, for example, from about 2 to about 10. Preferred fermentation conditions for yeast and filamentous fungi are a temperature in the range of from about 25° C. to about 55° C. and at a pH of from about 3 to about 9. The appropriate conditions are usually selected based on the choice of host cell. Accordingly, in an embodiment the method of the invention further comprises one or more elements selected from:
a) culturing the cell culture in a nutrient medium;
b) culturing the cell culture under aerobic or anaerobic conditions
c) culturing the cell culture under agitation;
d) culturing the cell culture at a temperature of between 25 to 50° C.;
e) culturing the cell culture at a pH of between 3-9; and
f) culturing the cell culture for between 10 hours to 30 days.

In a special embodiment wherein the host cell of the invention express a demethylase converting thebaine to northebaine in the cell, a demethylase-CPR and a transporter, it has been found that for optimal production of northebaines a pH from 6 to 8, such as from 6.5 to 7.5, such as about 7.0 should be maintained for the culturation/fermentation. In another special embodiment wherein the host cell of the invention express a demethylase converting oripavine to nororipavine in the cell, a demethylase-CPR and a transporter, it has been found that for optimal production of nororipavine at a pH from 3.5 to 5.5, such as from 3.0 to 5.0, such as about 4.5 should be maintained for the culturation/fermentation.

The cell culture of the invention may be recovered and or isolated using methods known in the art. For example, the compound(s) may be recovered from the nutrient medium by conventional procedures including, but not limited to, centrifugation, filtration, spray-drying, or lyophilization. In a particular embodiment the method includes a recovery and/or isolation step comprising separating a liquid phase of the cell or cell culture from a solid phase of the cell or cell culture to obtain a supernatant comprising the benzylisoquinoline alkaloid, eg. thebaine, northebaine, oripavine and/or nororipavine and subjecting the supernatant to one or more steps selected from:
a) contacting the supernatant with one or more adsorbent resins in order to obtain at least a portion of the produced benzylisoquinoline alkaloid, then optionally recovering the benzylisoquinoline alkaloid from the resin in a concentrated solution prior to precipitation or crystallisation of the benzylisoquinoline alkaloid;
b) contacting the supernatant with one or more ion exchange or reversed-phase chromatography columns in order to obtain at least a portion of the benzylisoquinoline alkaloid, then optionally recovering the benzylisoquinoline alkaloid from the resin in a concentrated solution prior to precipitation or crystallisation of the benzylisoquinoline alkaloid;
c) extracting the benzylisoquinoline alkaloid from the supernatant, such as by liquid-liquid extraction into an immisible solvent, then optionally evaporating the solvent to concentrate and precipitate the benzylisoquinoline alkaloid or performing further liquid-liquid extraction to recover and concentrate benzylisoquinoline alkaloid prior to crystallisation or precipitation or in order to directly perform a further chemical reaction on benzylisoquinoline alkaloid; and
d) evaporating the solvent of the supernatant to concentrate or precipitate the benzylisoquinoline alkaloid;
   thereby recovering and/or isolating the benzylisoquinoline alkaloid benzylisoquinoline alkaloid benzylisoquinoline alkaloid.

The method of the invention may comprise one or more in vitro steps in the process of producing the benzylisoquinoline alkaloid. It may also comprise one or more in vivo steps performed in another cell, such as a plant cell, for example a cell of Papaver somniferum For example, thebaine and/or oripavine or precursors thereof may be produced in a plant, such as poppy (Papaver somniferum) and isolated therefrom and then fed to a cell culture of the invention for conversions ion into northebaine and/or nororipavine. Accordingly, in one embodiment the method of the invention further comprises feeding the cell culture with exogenous thebaine, oripavine and/or a precursor thereof, and even further where the exogenous thebaine, oripavine and/or precursor thereof is a plant extract.

In one embodiment of the invention the benzylisoquinoline alkaloid is in particular, the benzylisoquinoline alkaloid is selected from one or more of thebaine, northebaine, oripavine and nororipavine.

Where thebaine, oripavine, northebaine and/or nororipavine and/or any upstream benzylisoquinoline alkaloid precursors is not the desired end-product further steps may be added to the method of the invention either chemically or biologically modifying the thebaine, northebaine, oripavine and/or nororipavine. Desired end products may be for example buprenorphine, naltrexone, naloxone or nalbuphine. Buprenorphine and other semisynthetic opioids are, or can be, made from thebaine (Hudlicky, Can. J. Chem. 93(5):492-501 (2015)). One route to buprenorphine is made up of 6 major steps, starting from thebaine. (Machara et al., Adv. Synth. Catal. 354(4):613-26 (2012); Werner et al., J. Org. Chem. 76(11):4628-34 (2011)). There, the first 3 steps are a Diels-Alder reaction of thebaine with methyl vinyl ketone to form a 4+2 product, hydrogenation of the carbon-carbon double bond of the resultant product, and addition of a tertiary butyl group via a Grignard reaction. The final steps are N- and O-demethylation and cyclopropyl alkylation. The number of steps can increase to 8, if the N- and O-demethylation and N-alkylation steps are performed in 2 stages, rather than 1. The order of the hydrogenation and Grignard steps may be reversed but most, if not all, economically viable preparations include the 3 above-mentioned steps prior to the N-demethylation step.

The N-demethylation of this known method can involve highly toxic reagents such as cyanogen bromide (von Braun, J. Chem. Ber., 33:1438-1452 (1900)) and chloroformate reagents (Cooley et al., Synthesis, 1:1-7 (1989); Olofson et al., J. Org. Chem., 49:2081-2082 (1984)) or may proceed in low yield, for example, by producing N-oxide intermediates (Polonovski reaction: Kok et al., Adv Synth. Catal., 351:283-286 (2009); Dong et al., J. Org. Chem., 72:9881-9885 (2007)). These methods generate significant amounts of toxic waste. The harsh conditions used for demethylation (e.g., strong bases and high temperatures) generate a significant amount of impurities, requiring additional purification and lowering yields. Attempts to reduce impurities and improve yields have been made by avoiding the O-demethylation step, by using oripavine as starting material, but a principal obstacle to an efficient synthesis remains the N-demethylation step.

Accordingly, for chemically converting thebaine, oripavine, northebaine and/or nororipavine into buprenophine or other opiate alkaloid derivatives there remains a need for an improved route of synthesis, such as a route that is shorter, more efficient (due to, e.g., improved total yield, decreased impurities), and/or produces less toxic waste. One challenge of known methods for preparation of buprenorphine is the exchange of the N-methyl group for an N-cyclopropyl group. N-demethylation methods can involve highly toxic reagents such as cyanogen bromide (von Braun, J. Chem. Ber., 33:1438-1452 (1900)) and chloroformate reagents (Cooley et al., Synthesis, 1:1-7 (1989); Olofson et al., J. Org. Chem., 49:2081-2082 (1984)) or may proceed in low yield, for example, by producing N-oxide intermediates (Polonovski reaction: Kok et al., Adv Synth. Catal., 351:283-286 (2009); Dong et al., J. Org. Chem., 72:9881-9885 (2007)). These methods generate significant amounts of toxic waste. The harsh conditions used for demethylation (e.g., strong bases and high temperatures) generate a significant amount of impurities, requiring additional purification and lowering yields. Attempts to reduce impurities and improve yields have been made by avoiding the O-demethylation step, by using oripavine as starting material, but a principal obstacle to an efficient synthesis remains the N-demethylation step.

As disclosed herein the present invention offers 1-2 fewer chemical demethylation steps reducing use or production of environmentally unfriendly chemicals, and it offers yield improvement and time by omitting those steps.

In the present invention to increase yields, decrease costs, and stabilize intermediates before further processing, the thebaine, northebaine, oripavine and/or nororipavine produced by fermentation/biotransformation can be subjected to minimal purification steps. For example, fermentation broth can be subjected to a cell removal step (centrifugation or filtration) and a concentration step. Further, if starting from nororipavine, the nororipavine can be add a protecting group in a first bisbenzylation step using benzyl bromide, to form 3,17-bisbenzylnororipavine using the semipurified nororipavine. Subsequent Diels Alder reaction with methyl vinyl ketone, Grignard reaction using t-butylmagnesium halide, a hydrogenation step using Pd/C, and N-alkylation reaction with cyclopropylmethylbromide as described below can be done to yield buprenorphine.

Accordingly, in an embodiment the method of the invention includes converting thebaine, oripavine, northebaine and/or nororipavine or alternatively a benzylisoquinoline alkaloid of the general formula R1-V-H (V):

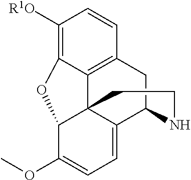

into for example buprenorphine:

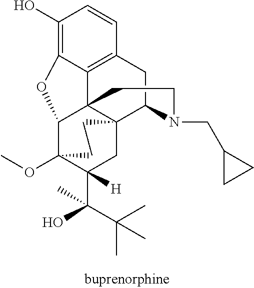 [SMILES: CO[C@]12CC[C@@]3(C[C@@H]1[C@](C)(O)C(C)(C)C)[C@H]1Cc4ccc(O)c5c4[C@@]3(CCN1CC1CC1)[C@H]2O5]

by applying, in sequence, a bis-benzylation step, a Diels-Alder step and a Grignard step.

In particular for converting nororipavine, HO—V—H (VI), of the general formula:

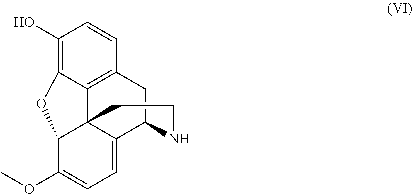

into buprenophine the method of the invention comprises steps of:
a) in a first solvent system S-1 comprising a polar protic solvent, reacting the compound HO—VI-H (VI), with benzyl halide, benzyl sulfonate, or activated benzyl alcohol (e.g. activated with a sulfonate group such as a p-toluene sulfonyl group or a methyl sulfonyl group, or with triphenylphosphine) to provide a compound BnO—VI-Bn (VI) of the general formula:

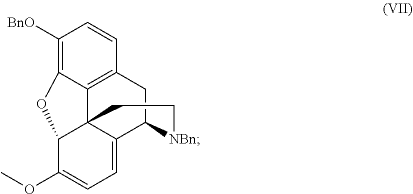

a preparation of Compound BnO-I-Bn, as an intermediate towards noroxymorphone and ultimately towards naltrexone and naloxone, was described in Helv. Chim. Acta 92:1359-65 (2009);
b) in a second solvent system S-2 comprising a polar protic solvent, reacting compound BnO—VI-Bn (VII) with methyl vinyl ketone to provide a compound BnO—VI-Bn (VIII) of the general formula:

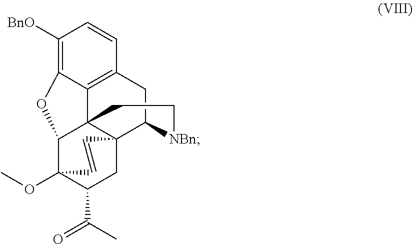

c) in a third solvent system S-3 comprising a nonpolar solvent, reacting Compound BnO—VII-Bn (VIII) with a tert-butylmagnesium compound to provide a compound BnO-VIIIA-Bn (IX) of the general formula:

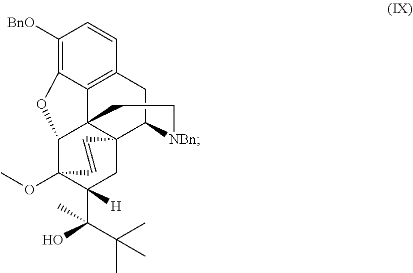

d) reacting Compound BnO-VIIIA-Bn (IX) with H2 in the presence of a hydrogenation catalyst to provide a compound HO—IX—H (X) of the general formula:

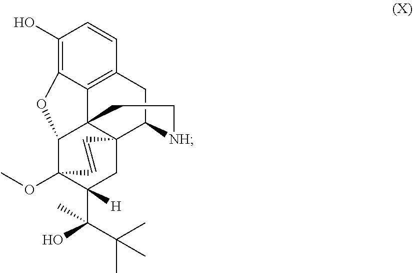

e) reacting Compound HO—IX—H (X) with
   i) cyclopropane carboxaldehyde followed by a hydride source; or:
   ii) cyclopropanecarboxylic acid halide followed by a reducing agent; or
   iii) cyclopropylmethyl halide or activated cyclopropane methanol;
   to provide buprenorphine.
      Step a) in the Method for Converting HO—V-H (V) into Buprenophine

In some embodiments, the benzyl halide of step a) above is benzyl chloride or benzyl bromide. In some embodiments, the reaction of step a) above is performed in the presence of a strong base, e.g., an alkali metal hydride.

S-1 may comprise at least one protic solvent having a dielectric constant of at least at least about 12, or at least about 13, or at least about 14, or at least about 15, or at least about 16, or at least about 18, or at least about 20.

In certain embodiments as otherwise described herein, S-1 comprises at least about 50 vol. % of at least one protic solvent having a dielectric constant of at least about 12. In various other embodiments, the at least one protic solvent is present in an amount of at least 60 vol. %, or at least 70 vol. %, or at least 75 vol. %, or at least 80 vol. %, or at least 90 vol. %, or at least 95 vol. %, such as at least about 50 vol. %, or at least about 75 vol. %, or at least about 90 vol. % of the said protic solvent.

In some embodiments, S-1 comprises at least one protic solvent having a polarity index of at least about 3, or at least about 3.5, or at least about 3.75, or at least about 4. As used herein, the solvent polarity index of a solvent can be determined according to Snyder, e.g. as reported in Snyder, L. R. “Classification of the Solvent Properties of Common Liquids.” J. Chromatogr. (1978) 16:6, 223-234. In various embodiments, S-1 comprises at least about 50 vol. %, or at least about 75 vol. %, or at least about 90 vol. % of at least one protic solvent having a polarity index of at least about 3, or at least 3.5, or at least 3.75, or at least 4.

In some embodiments as otherwise described herein, S-1 comprises a C1-C4 alcohol (e.g., methanol, ethanol, n-propanol, isopropanol, n-butanol, or sec-butanol) and optionally water. In various embodiments, S-1 comprises about 50-100 vol. % isopropanol and 0-50 vol. % water.

In some embodiments, the benzyl halide, benzyl sulfonate, or activated benzyl alcohol is reacted at a temperature within the range of about −20° C. to about 40° C., e.g., about −20° C. to about 35° C., or about −20° C. to about 30° C., or about −20° C. to about 25° C., or about −20° C. to about 20° C., or about −20° C. to about 15° C., or about −20° C. to about 10° C., or about −20° C. to about 5° C., or about −20° C. to about 0° C., or about −15° C. to about 40° C., or about −10° C. to about 40° C., or about −5° C. to about 40° C., or about 0° C. to about 40° C., or about 5° C. to about 20° C., or about 10° C. to about 40° C., or about 15° C. to about 40° C., or about 20° C. to about 40° C., or about −15° C. to about 35° C., or about −10° C. to about 30° C., or about −5° C. to about 25° C., or about 0° C. to about 20° C., or about 5° C. to about 15° C. In some embodiments, the benzyl halide, benzyl sulfonate, or activated benzyl alcohol is reacted for a period of time within the range of about 1 h to about 2 days, e.g., 2 h to about 2 days, 3 h to about 2 days, 6 hours to about 2 days, about 12 hours to about 2 days, or about 18 hours to about 2 days, or about 1 day to about 2 days, or about 1.25 days to about 2 days, or about 1.5 days to about 2 days, or about 6 hours to about 1.75 days, or about 6 hours to about 1.5 days, or about 6 hours to about 1.25 days, or about 6 hours to about 1 day, or about 6 hours to about 18 hours, or about 12 hours to about 1.75 days, or about 18 hours to about 1.5 days, or about 1 h to about 1 day, or about 1 h to about 12 h, or about 1 h to about 6 h, or about 1 h to about 4 h.

Step b) in the Method for Converting HO—V-H (VI) into Buprenophine

In some embodiments, the methyl vinyl ketone is reacted at a temperature within the range of about 40° C. to about 120° C., e.g., about 45° C. to about 120° C., or about 50° C. to about 120° C., or about 55° C. to about 120° C., or about 60° C. to about 120° C., or about 65° C. to about 120° C., or about 70° C. to about 120° C., or about 75° C. to about 120° C., or about 80° C. to about 120° C., or about 85° C. to 120° C., or about 90° C. to about 120° C., or about 40° C. to about 115° C., or about 40° C. to about 110° C., or about 40° C. to about 105° C., or about 40° C. to about 100° C., or about 40° C. to about 95° C., or about 40° C. to about 90° C., or about 40° C. to about 85° C., or about 40° C. to about 80° C., or about 40° C. to about 75° C., or about 40° C. to about 70° C., or about 45° C. to about 115° C., or about 50° C. to about 110° C., or about 55° C. to about 105° C., or about 60° C. to about 100° C., or about 65° C. to about 95° C., or about 70° C. to about 90° C. In some embodiments, the methyl vinyl ketone is reacted for a period of time within the range of about 2 hours to about 2 days, e.g., about 4 hours to about 2 days, or about 6 hours to about 2 days, or about 12 hours to about 2 days, or about 18 hours to about 2 days, or about 1 days to about 2 days, or about 1.25 days to about 2 days, or about 1.5 days to about 2 days, or about 2 hours to about 1.75 days, or about 2 hours to about 1.5 days, or about 2 hours to about 1.25 days, or about 2 hours to about 1 day, or about 2 hours to about 18 hours, or about 2 hours to about 12 hours, or about 4 hours to about 1.75 days, or about 6 hours to about 1.5 days, or about 12 hours to about 1.25 days, or about 18 hours to about 1 day.

In certain embodiments as otherwise described herein, the reaction of step b) is carried out under oxygen, e.g., a mixture of inert gas and oxygen having a different composition than that of air. In certain embodiments, the reaction is carried out in an atmosphere wherein inert gas (e.g., argon) is present as greater than 5 vol. % (e.g., greater than 20 vol. %, or greater than 50 vol. %), and oxygen is present as less than 25 vol. % (e.g., less than 21 vol. %, or less than 20 vol. %, or less than 10 vol. %, or less than 5 vol. %). In certain embodiments, the reaction is carried out in an atmosphere wherein oxygen is present at between 1 vol. % and about 21 vol. %, or between 3 vol. % and 20 vol. %, or between 5 vol. % and 20 vol. %, or between 10 vol. % and 20 vol. %, or between 1 vol. % and 20 vol. %, or between 1 vol. % and 15 vol. %, or between 1 vol. % and 10 vol. %, or between 1 vol. % and 7 vol. %, or between 1 vol. % and 5 vol. %. In certain other embodiments, the reaction is performed in substantially inert atmosphere (e.g., oxygen is present at less than 0.1 vol. %, or less than 0.01 vol. %, or less than 0.001 vol. %).

In certain other embodiments, the reaction of step b) is carried out under a mixture of gases approximately the same as air (e.g., dry air). In other embodiments, the reaction is carried out wherein the ratio of inert gas to gaseous oxygen is approximately 79 vol. % to 21 vol. %.

It has been found that the use of some amount of oxygen in the atmosphere of the reaction in step b) serves to increase the yield of the reaction. Without being bound by theory, it is presently believed that trace oxygen prevents methyl vinyl ketone polymerization, allowing for additional methyl vinyl ketone monomers to be present as reactants. Beyond enhancing the yield, providing a reaction atmosphere containing at least some oxygen generally requires less rigorous reaction condition and equipment, especially at scale, as rigorous oxygen exclusion is no longer required. Together, this development allows for a more efficient synthetic protocol and enhanced reaction yields with lower capital expenditures.

In some embodiments, the second solvent system S-2 has a dielectric constant of at least about 12, or at least about 13, or at least about 14. In various other embodiments as described herein, the dielectric constant of S-2 is at least about 15, or at least about 16, or at least about 18, or at least about 20.

In certain embodiments as otherwise described herein, S-2 comprises at least about 50 vol. % of at least one protic solvent having a dielectric constant of at least about 12. In various other embodiments, the at least one protic solvent is present in an amount of at least 60 vol. %, or at least 70 vol. %, or at least 75 vol. %, or at least 80 vol. %, or at least 90 vol. %, or at least 95 vol. %. In certain embodiments, the at least one protic solvent has a dielectric constant of at least 13, or at least 14, or at least 15, or at least 16, or at least 18, or at least 20.

In some embodiments, S-2 comprises at least one protic solvent having a polarity index of at least about 3, or at least about 3.5, or at least about 3.75, or at least about 4. In various embodiments, S-2 comprises at least about 50 vol. %, or at least about 75 vol. %, or at least about 90 vol. % of at least one protic solvent having a polarity index of at least about 3, or at least 3.5, or at least 3.75, or at least 4.

In some embodiments as otherwise described herein, S-2 comprises a C1-C4 alcohol (e.g., methanol, ethanol, n-propanol, isopropanol, n-butanol, or sec-butanol) and optionally water. In various embodiments, S-2 comprises about 50-100 vol. % isopropanol and 0-50 vol. % water. In certain desirable embodiments, S-2 has substantially the same composition as S-1, described above.

In certain desirable embodiments, reactions in steps a) and b) can be performed sequentially, advantageously without intervening purification and without substantial removal of solvent system S-1. In certain embodiments as otherwise described herein, S-2 has substantially the same composition as S-1. In such embodiments, the reactants and purification steps of step b) comprise the crude reaction product of step a). Accordingly, in certain embodiments as otherwise described herein, the methyl vinyl ketone of step b) is added to a crude reaction product of step a), the crude reaction product comprising solvent S-1 and BnO—VII-Bn (VIII).

As noted above, the reaction of b) can be carried out without substantial removal of solvent or purification (e.g. chromatography). However, the person of ordinary skill in the art will appreciate that it may be necessary to adjust the pH of the crude reaction product of step a) before performing step b). The pH may be adjusted with a wide variety of acids known in the art. For example, in certain embodiments, the pH is adjusted with the addition of acetic acid or hydrochloric acid. For example, the pH may be adjusted with a water-diluted acid such as 10% acetic acid, or 10% hydrochloric acid. In various embodiments as otherwise described herein, the pH is adjusted to be about neutral, e.g., between 6 and 8.

Step c) in the Method for Converting HO—V-H (VI) into Buprenophine

In some embodiments, the tert-butylmagnesium compound is a tert-butylmagnesium halide. For example, the tert-butylmagnesium compound is tert-butylmagnesium chloride or tert-butylmagnesium bromide. In some embodiments, the reaction is performed in a solvent comprising a nonpolar solvent, e.g., tert-butylmethyl ether, 2-methyl-tetrahydrofuran, diethyl ether, dimethoxymethane, benzene, toluene, or a mixture of thereof.

In some embodiments, the tert-butylmagnesium compound is reacted at a temperature within the range of about 15° C. to about 100° C., e.g., about 20° C. to about 100° C., or about 25° C. to about 100° C., or about 30° C. to about 100° C., or about 15° C. to about 95° C., or about 15° C. to about 90° C., or about 15° C. to about 85° C., or about 20° C. to about 95° C., or about 25° C. to about 90° C. In some embodiments, the tert-butylmagnesium halide is reacted for a period of time within the range of about 30 minutes to about 8 hours, e.g., about 1 hours to about 8 hours, or about 1.5 hours to about 8 hours, or about 2 hours to about 8 hours, or about 2.5 hours to about 8 hours, or about 3 hours to about 8 hours, or about 3.5 hours to about 8 hours, or about 4 hours to about 8 hours, or about 4.5 hours to about 8 hours, or about 5 hours to about 8 hours, or about 30 minutes to about 7.5 hours, or about 30 minutes to about 7 hours, or about 30 minutes to about 6.5 hours, or about 30 minutes to about 6 hours, or about 30 minutes to about 5.5 hours, or about 30 minutes to about 5 hours, or about 30 minutes to about 4.5 hours, or about 30 minutes to about 4 hours, or about 30 minutes to about 3.5 hours, or about 1 hour to about 7.5 hours, or about 1.5 hours to about 7 hours, or about 2 hours to about 6.5 hours, or about 2.5 hours to about 6 hours, or about 3 hours to about 5.5 hours.

As described above, the third solvent system S-3 comprises a nonpolar solvent. In certain embodiments as otherwise described herein, the third solvent system 5-3 comprises at least one at least one nonpolar solvent having a dielectric constant of at most about 8, or at most about 7, or at most about 6, or at most about 5, or at most about 4, or at most about 3. For example, in various embodiments, S-3 comprises at least 60 vol. %, or at least 70 vol. %, or at least 75 vol. %, or at least 80 vol. %, or at least 90 vol. %, or at least 95 vol. % of the at least one nonpolar solvent having a dielectric constant of at most 8, or at most 7, or at most 6, or at most 5, or at most 4, or at most 3. In further embodiments, the nonpolar solvent that comprises S-3 has a polarity index of less than 4, or less than 3, or less than 2, or less than 1. For example, in various embodiments, S-3 comprises at least 60 vol. %, or at least 70 vol. %, or at least 75 vol. %, or at least 80 vol. %, or at least 90 vol. %, or at least 95 vol. % of the at least one nonpolar solvent has a polarity index of less than 4, or less than 3, or less than 2, or less than 1.

In certain desirable embodiments, polar solvents or solvents with large dielectric constants are not substantially present in S-3, or are present in S-3 in a relatively small amount. For example, in certain embodiments, S-3 comprises less than about 20 vol. %, or less than about 10 vol. %, or less than about 5 vol. %, or less than about 1 vol. % of a total amount of solvents having a dielectric constant of greater than 4, or greater than 6, or greater than 8. In various embodiments as otherwise described herein, S-3 comprises less than about 20 vol. %, or less than about 10 vol. %, or less than about 5 vol. %, or less than about 1 vol. % of a total amount of solvents having a polarity index of 2 or greater, or 3 or greater, or 4 or greater.

In some embodiments, S-3 comprises 30-90 vol. % of one or more C5-C10 alkanes and/or C5-C10 cycloalkanes. In certain embodiments, the alkanes and/or cycloalkanes are substituted (e.g., perfluorocyclohexane, perfluorohexane, etc.). For example, in certain embodiments the one or more alkanes and/or cycloalkanes include cyclohexane. In other embodiments, the one or more alkanes and/or cycloalkanes is cyclohexane. For example, S-3 may comprise 10-50 vol. % toluene (e.g., 20-50 vol. % toluene, or 30-50 vol. % toluene), 30-90 vol. % cyclohexane (e.g., 40-90 vol. % cyclohexane, or 40-70 vol. % cyclohexane), and up to 30 vol % tetrahydrofuran (e.g., up to 20 vol. % tetrahydrofuran, or up to 10 vol. % tetrahydrofuran, or up to 5 vol. % tetrahydrofuran).

It is known in the art that certain Grignard reagents (e.g., tert-butylmagnesium halide) disproportionate to the bis-alkyl and bis-halide species, with the disproportionation favored under certain reaction and solvent conditions (e.g. a more polar solvent). Without wishing to be bound by theory, the present inventors believe that the reaction conditions of step c) described herein can increase the concentration of the bis adducts present in solution, advantageously improving the yield of BnO-VIIIA-Bn (IX). Accordingly, in certain embodiments, the tert-butylmagnesium compound comprises one or both of a tert-butylmagnesium halide and di-tert-butylmagnesium. For example, in some embodiments, the tert-butylmagnesium compound comprises a tert-butylmagnesium halide and di-tert-butylmagnesium. In certain embodiments, a proportion of the magnesium dihalide (e.g., magnesium dichloride) precipitates from solution. For example, substantially all of the magnesium dihalide may precipitate from solution. Alternatively, in certain other embodiments, there is essentially no precipitate formed from the Grignard reagent.

Step d) in the Method for Converting HO—V-H (VI) into Buprenophine

In some embodiments, the hydrogenation catalyst comprises nickel, palladium, platinum, rhodium, or ruthenium. In some embodiments, the hydrogenation catalyst comprises platinum or palladium, supported on carbon. In some embodiments, the reaction is performed in a solvent comprising a polar protic or aprotic solvent, e.g., n-butanol, isopropanol, ethanol, methanol, N-methylpyrrolidone, tetrahydrofuran, ethyl acetate, acetone, dimethylformamide, acetonitrile, dimethylsulfoxide, propylene carbonate, or a mixture thereof.

In some embodiments, the hydrogen is reacted at a temperature within the range of about 15° C. to about 120° C., e.g., about 20° C. to about 120° C., or about 30° C. to about 120° C., or about 40° C. to about 120° C., or about 15° C. to about 115° C., or about 20° C. to about 110° C., or about 30° C. to about 105° C., or about 40° C. to about 115° C., or about 50° C. to about 110° C. In some embodiments, the hydrogen is reacted for a period of time within the range of about 6 hours to about 3 days, e.g., about 12 hours to about 3 days, or about 18 hours to about 3 days, or about 1 day to about 3 days, or about 1.25 days to about 3 days, or about 1.5 days to about 3 days, or about 6 hours to about 2.75 days, or about 6 hours to about 2.5 days, or about 6 hours to about 2.25 days, or about 6 hours to about 2 day, or about 6 hours to about 36 hours, or about 12 hours to about 2.5 days, or about 24 hours to about 2 days. In some embodiments, the hydrogen is reacted at a pressure within the range of about 1 atm to about 3 atm, e.g., about 1.25 atm to about 3 atm, or about 1.5 atm to about 3 atm, or about 1.75 atm to about 3 atm, or about 2 atm to about 3 atm, or about 1 atm to about 2.75 atm, or about 1 atm to about 2.5 atm, or about 1 atm to about 2.25 atm, or about 1 atm to about 2 atm, or about 1.25 atm to about 2.75 atm, or about 1.5 atm to about 2.5 atm, or about 1.75 atm to about 2.25 atm.

Step e.i) in the Method for Converting HO—V-H (VI) into Buprenophine

In some embodiments, the hydride source is formic acid, hydrogen, sodium cyanoborohydride, sodium borohydride, or sodium triacetoxy borohydride. In some embodiments, the hydride source is formic acid. In some embodiments, the reaction is catalyzed by a ruthenium(I) complex or a ruthenium(II) complex, e.g., a dichloro(p-cymene)ruthenium(II) dimer. In some embodiments, the reaction is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof. In some embodiments, the reaction is performed in the presence of a trialkylamine, e.g., triethylamine, diisopropylethylamine, 4-methyl-morpholine, or N-methyl-piperidine.

In some embodiments, the cyclopropane carboxaldehyde is reacted at a temperature within the range of about 30° C. to about 90° C., e.g., about 35° C. to about 90° C., or about 40° C. to about 90° C., or about 45° C. to about 90° C., or about 50° C. to about 90° C., or about 55° C. to about 90° C., or about 60° C. to about 90° C., or about 65° C. to about 90° C., or about 70° C. to about 90° C., or about 30° C. to about 85° C., or about 30° C. to about 80° C., or about 30° C. to about 75° C., or about 30° C. to about 70° C., or about 30° C. to about 65° C., or about 30° C. to about 60° C., or about 30° C. to about 55° C., or about 30° C. to about 50° C., or about 35° C. to about 85° C., or about 40° C. to about 80° C., or about 45° C. to about 75° C., or about 50° C. to about 70° C., or about 55° C. to about 65° C. In some embodiments, the cyclopropane carboxaldehyde is reacted for a period of time within the range of about 30 minutes to about 5 hours, e.g., about 1 hour to about 5 hours, or about 1.5 hours to about 5 hours, or about 2 hours to about 5 hours, or about 2.5 hours to about 5 hours, or about 3 hours to about 5 hours, or about 3.5 hours to about 5 hours, or about 4 hours to about 5 hours, or about 30 minutes to about 4.5 hours, or about 30 minutes to about 4 hours, or about 30 minutes to about 3.5 hours, or about 30 minutes to about 3 hours, or about 30 minutes to about 2.5 hours, or about 30 minutes to about 2 hours, or about 30 minutes to about 1.5 hours.

Step e.ii) in the Method for Converting HO—V-H (VI) into Buprenophine

In some embodiments, the cyclopropanecarboxylic acid halide is cyclopropanecarboxylic acid chloride, cyclopropanecarboxylic acid anhydride, cyclopropanecarboxylic acid bromide, or an activated cyclopropanecarboxylic acid (e.g., an activated cyclopropanecarboxylic acid formed by reaction with an alcohol such as pentafluorophenol, 4-nitrophenol, N-hydroxysuccinimide, N-hydroxymaleimide, 1-Hydroxybenzotriazole, or 1-hydroxy-7-azabenzotriazole). In some embodiments, the reducing agent is LiAlH4 or NaBH4. In some embodiments, the reaction with cyclopropanecarboxylic acid halide is performed in a solvent comprising a nonpolar solvent, e.g., dichloromethane, chloroform, toluene, 1,4-dioxane, diethyl ether, benzene, or a mixture thereof. In some embodiments, the reaction with a reducing agent is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof.

In some embodiments, the cyclopropanecarboxylic acid halide is reacted at a temperature within the range of about −20° C. to about 40° C., e.g., about −20° C. to about 35° C., or about −20° C. to about 30° C., or about −20° C. to about 25° C., or about −20° C. to about 20° C., or about −20° C. to about 15° C., or about −20° C. to about 10° C., or about −20° C. to about 5° C., or about −20° C. to about 0° C., or about −15° C. to about 40° C., or about −10° C. to about 40° C., or about −5° C. to about 40° C., or about 0° C. to about 40° C., or about 5° C. to about 20° C., or about 10° C. to about 40° C., or about 15° C. to about 40° C., or about 20° C. to about 40° C., or about −15° C. to about 35° C., or about −10° C. to about 30° C., or about −5° C. to about 25° C., or about 0° C. to about 20° C., or about 5° C. to about 15° C. In some embodiments, the cyclopropanecarboxylic acid halide is reacted for a period of time within the range of about 6 hours to about 2 days, e.g., about 12 hours to about 2 days, or about 18 hours to about 2 days, or about 1 day to about 2 days, or about 1.25 days to about 2 days, or about 1.5 days to about 2 days, or about 6 hours to about 1.75 days, or about 6 hours to about 1.5 days, or about 6 hours to about 1.25 days, or about 6 hours to about 1 day, or about 6 hours to about 18 hours, or about 12 hours to about 1.75 days, or about 18 hours to about 1.5 days. In some embodiments, the reducing agent is reacted at a temperature within the range of about 35° C. to about 85° C., e.g., about 40° C. to about 85° C., or about 45° C. to about 85° C., or about 50° C. to about 85° C., or about 55° C. to about 85° C., or about 60° C. to about 85° C., or about 65° C. to about 85° C., or about 35° C. to about 80° C., or about 35° C. to about 75° C., or about 35° C. to about 70° C., or about 35° C. to about 65° C., or about 35° C. to about 60° C., or about 35° C. to about 55° C., or about 40° C. to about 80° C., or about 45° C. to about 75° C., or about 50° C. to about 70° C., or about 55° C. to about 65° C. In some embodiments, the reducing agent is reacted for a period of time within the range of about 5 minutes to about 3 hours, e.g., or about 10 minutes to about 3 hours, or about 15 minutes to about 3 hours, or about 30 minutes to about 3 hours, or about 45 minutes to about 3 hours, or about 1 hour to about 3 hours, or about 1.25 hours to about 3 hours, or about 1.5 hours to about 3 hours, or about 1.75 hours to about 3 hours, or about 2 hours to about 3 hours, or about 5 minutes to about 2.75 hours, or about 5 minutes to about 2.5 hours, or about 5 minutes to about 2.25 hours, or about 5 minutes to about 2 hours, or about 5 minutes to about 1.75 hours, or about 5 minutes to about 1.5 hours, or about 5 minutes to about 1.25 hours, or about 5 minutes to about 1 hour, or about 10 minutes to about 2.75 hours, or about 15 minutes to about 2.5 hours, or about 30 minutes to about 2.25 hours, or about 45 minutes to about 2 hours, or about 1 hour to about 1.75 hours.

Step e.iii) in the Method for Converting HO—V-H (VI) into Buprenophine

In some embodiments, the cyclopropylmethyl halide is cyclopropylmethyl chloride or cyclopropylmethyl bromide. In some embodiments, the reaction is performed in the presence of a trialkylamine, e.g., triethylamine, diisopropylethylamine, 4-methyl-morpholine, or N-methyl-piperidine. In some embodiments, the reaction is performed in a solvent comprising a polar protic solvent, e.g., n-butanol, isopropanol, ethanol, methanol, water, or a mixture thereof.

In some embodiments, the cyclopropylmethyl halide or activated cyclopropane methanol is reacted at a temperature within the range of about 40° C. to about 120° C., e.g., about 45° C. to about 120° C., or about 50° C. to about 120° C., or about 55° C. to about 120° C., or about 60° C. to about 120° C., or about 65° C. to about 120° C., or about 70° C. to about 120° C., or about 75° C. to about 120° C., or about 80° C. to about 120° C., or about 85° C. to 120° C., or about 90° C. to about 120° C., or about 40° C. to about 115° C., or about 40° C. to about 110° C., or about 40° C. to about 105° C., or about 40° C. to about 100° C., or about 40° C. to about 95° C., or about 40° C. to about 90° C., or about 40° C. to about 85° C., or about 40° C. to about 80° C., or about 40° C. to about 75° C., or about 40° C. to about 70° C., or about 45° C. to about 115° C., or about 50° C. to about 110° C., or about 55° C. to about 105° C., or about 60° C. to about 100° C., or about 65° C. to about 95° C., or about 70° C. to about 90° C. In some embodiments, the cyclopropylmethyl halide or activated cyclopropane methanol is reacted for a period of time within the range of about 30 minutes to about 6 hours, e.g., about 1 hours to about 6 hours, or about 1.5 hours to about 6 hours, or about 2 hours to about 6 hours, or about 2.5 hours to about 6 hours, or about 3 hours to about 6 hours, or about 3.5 hours to about 6 hours, or about 4 hours to about 6 hours, or about 30 minutes to about 5.5 hours, or about 30 minutes to about 5 hours, or about 30 minutes to about 4.5 hours, or about 30 minutes to about 4 hours, or about 30 minutes to about 3.5 hours, or about 30 minutes to about 3 hours, or about 30 minutes to about 2.5 hours, or about 1 hours to about 5.5 hours, or about 1.5 hours to about 5 hours, or about 2 hours to about 4.5 hours, or about 2.5 hours to about 4 hours.

A person of ordinary skill in the art will appreciate that additional steps such as, for example, purification (e.g., crystallization) or formation of an addition salt (e.g., formation of buprenorphine-HCl) may be included in the methods of the disclosure as otherwise described herein. Further examples of suitable modifications are disclosed in U.S. provisional application 62/962,335 and WO2018229306 and WO2018211331 included herein by reference in their entirety.

In addition to buprenorphine, other industrially useful products can be synthesized from the thebaine, oripavine, northebaine and/or nororipavine of the invention optionally post fermentation/bioconversion added a protecting group by bisbenzylation as described above for nororipavine. Such useful products include “Nals” compounds, such as naloxone, nalmefene, nalbuphine, naltrexone and methylnaltrexone, which can also be synthesized through a common intermediate noroxymorphone. Thebaine and oripavine of the invention can also be used to produce a range of therapeutically important molecules, including oxycodone, hydrocodone, hydromorphone and oxymorphone, the latter also used as an intermediate for the manufacture of “Nals”.

As shown by A. Sipos, S. Berenyi and S. Antus (Helvetica Chimica Acta Vol 92 (2009) pp 1359-1365) nororipavine (also known as oripavidine) may be converted to noroxymorphone using a benzylation reaction, an oxidation reaction using peracid, and catalytic hydrogenation. Subsequent N-Alkylation with 3-Bromo-1-propene will yield Naloxone, further alkylation with cyclopropylmethylbromide will yield naltrexone. B. Gutmann et al describe the utility of the intermediate noroxymorphone for the “Nal” compounds, synthesis commencing with thebaine or oripavine rather than nororipavine (European Journal of Organic Chemistry 2017, 914-927). T. Hudlicky (Can. J. Chem 93: 492-501 (2015)) similarly describes methods for production of buprenorphine, naltrexone, naloxone, and nalbuphine from thebaine and oripavine. Numerous patent applications describe similar methods for synthesis of these useful pharmaceutical compounds such as WO 2009/003270, WO 2009/079013, WO 2010/063291, WO 2010/136039, WO 2012/059103, WO 2012/151669, WO 2012/149633, WO 2013/08365, WO 2013/113120, WO 2013/164383, WO 2013/119886, WO 2005/028483 A1, WO 2005/084412, WO 2007/137782, WO 2009/003272, WO 2009/152571A1, WO 2009/152577A1, WO 2011/032214, and WO 2012/018872. It is clear that a scalable and stable supply chain of thebaine, oripavine, northebaine or nororipavine made by the fermentation/biotransformation of the invention is useful as starting material for the chemical conversion of the invention as disclosed herein.

Currently known methods for producing semisynthetic opioids (including oxycodone, hydrocodone, hydromorphone, oxymorphone, naloxone, naltrexone, nalmefene, methylnaltrexone, noroxymorphone, buprenorphine) include production via chemical synthesis from thebaine, oripavine, morphine and codeine, mostly commonly from thebaine or oripavine, all four compounds produced by extraction from the opium poppy (Papaver somniferum). The lack of a commercial supply of nororipavine is in part due to the inability of the opium poppy to produce commercially viable concentrations of nororipavine, which is believed to be due to the lack of a naturally occurring N-demethylase enzyme in the opium poppy. High yielding industrially applicable methods of synthesis of nororipavine have not previously been disclosed and production of commercially relevant quantities of nororipavine have not hitherto been available. Thebaine, oripavine, northebaine and/or in particular nororipavine are attractive for use as a starting material due to their chemical structure and functionality allowing efficient installation of the hydroxy group at C-14 position and/or for performing the Diels-Alder reaction on the methoxydiene moiety to produce the backbone of buprenorphine. Nororipavine produced by fermentation/bioconversion has the additional advantage over thebaine and oripavine that the difficult chemical N-demethylation is already completed further enhancing the utility as a starting material for buprenorphine or “Nals” synthesis. (Machara et. al. Georg Thieme Verlag Stuttgart—New York—Synthesis 2016, 48, 1803-1813).

Separation methods for opiates and other alkaloids are well-known in the art. See, for example Tolkachev et al. (1983), Janicot et al. (1988), Barbier (1950), Ramanathan and Chandra (1980), Hosztafi (2014), Hamerslag (1950), Thorton (1992), Heumann (1957), Swiss patent 457433 (1935), GB713689 (1952), GB1586626 (1977), U.S. Pat. No. 6,723,894B2 (2002), and U.S. Pat. No. 6,054,584A (1996)—all incorporate by reference. In further specific embodiments when producing nororipavine by fermentation, either de-novo or by bioconversion of a fed precursor, isolation of the Nororipavine contained in the fermentation broth can be achieved by several routes encompassing the typical unit operations of solid-liquid separation (e.g. ultra and nanofiltration membrane filtration, centrifugal separation, pressure or vacuum filtration through filter membranes or filter aids) residual biomass washing to maximise recovery (with various medium including water, acids, bases and solvents), and the concentration and selective removal of nororipavine from related fermentation products and any residual starting material by direct crystallisation as the Nororipavine base or a selective salt, by adsorption and de-adsorption on solid supports (e.g. ion-exchange resins, molecular imprinted polymers), by liquid-liquid extraction, supercritical fluids extraction, or by reaction with other chemicals to directly form a desired derivative of nororipavine. These downstream reactions could include the addition of new functional groups to add functionality to the secondary nitrogen position to form a tertiary nitrogen (e.g. alkylation) or to the phenolic hydroxide position (e.g. benzylation), to oxidise or reduce to form new products (e.g. introduction of 14-hydroxy-) or reactions directly on diene bond to form new products (e.g. Diels Alder reaction). Incorporating the formation of new products within the processing of the fermentation broth may provide more selective separation from related impurities, improve isolation characteristics such as filtration speed, incorporate a required downstream process step eliminating the need for isolation of Nororipavine as a process intermediate (known as process telescoping), provide a less reactive more stable isolated product and improve overall process yield.

An exemplary embodiment consists of solid-liquid separation by ultrafiltration of the fermentation broth in order to remove cellular matter and higher molecular weight components, resulting in further concentration of the broth containing Nororipavine. A wash of the clarified solids can then be performed with a dilute acid and can be combined with the clarified broth. A clarified broth can be treated with compounds that form insoluble complexes with divalent cations and clarified by separation, such as filtration or centrifugal separation. Alternatively nanofiltration can be used for partial deionization as well.

The pH of the combined clarified broth and water wash may in an embodiment be adjusted, preferably just prior to, or after, being contacted with an immiscible solvent such as toluene, xylene, amyl alcohol, isobutanol, benzyl alcohol or a mixture of similar solvents in order to maximise selective extraction of the Nororipavine into a Nororipavine rich solvent. Contact with the solvent phase may be carried out in batch or continuous mode, optimally as a multi-stage counter current system.

In an embodiment the Nororipavine rich organic phase can be extracted in another liquid-liquid extraction step using either an alkaline or acid aqueous solution to produce a concentrated Nororipavine aqueous solution. Contact with the solvent phase may be carried out in batch or continuous mode optimally as a multi-stage counter current system. The aqueous solution of Nororipavine can optionally be isolated by direct addition of acid or base to precipitate the Nororipavine which is then filtered, washed and dried. In another embodiment the solution can be mixed with solvent prior to reaction with and excess of Benzyl bromide (or similar blocking reactant) to form and precipitate 3,17, bisbenzylnororipavine bisbenzyl. The resultant slurry can be cooled, filtered and washed with water and dried.

### Fermentation Composition

The invention further provides a fermentation composition comprising the cell culture of the invention and the benzylisoquionoline alkaloid comprised therein.

In one embodiment at least 10%, 25%, 50%, such as at least 75%, such as at least 95%, such as at least 99% of the cells of the fermentation composition of the invention are lysed. Further in the fermentation composition of the invention at least 10%, 25%, 50%, such as at least 75%, such as at least 95%, such as at least 99% of solid cellular material may have been removed and separated from a liquid phase. Moreover, in addition to benzylisoquionoline alkaloid the fermentation composition of the invention may comprise one or more compounds selected from trace metals, vitamins, salts, yeast nitrogen base, carbon source, YNB, and/or amino acids of the fermentation. In particular the fermentation composition of the invention comprise a concentration of benzylisoquionoline alkaloid is at least 1 mg/kg composition, such as at least 5 mg/kg, such as at least 10 mg/kg, such as at least 20 mg/kg, such as at least 50 mg/kg, such as at least 100 mg/kg, such as at least 500 mg/kg, such as at least 1000 mg/kg, such as at least 5000 mg/kg, such as at least 10000 mg/kg, such as at least 50000 mg/kg.

### Compositions and Use

In a further aspect the invention provides a composition comprising the fermentation composition of the invention and one or more carriers, agents, additives and/or excipients. Carriers, agents, additives and/or excipients includes formulation additives, stabilising agent, fillers and the like. The composition may be formulated into a dry solid form by using methods known in the art, such as spray drying, spray cooling, lyophilization, flash freezing, granulation, microgranulation, encapsulation or microencapsulation. The composition may also be formulated into liquid stabilized form using methods known in the art, such as formulation into a stabilized liquid comprising one or more stabilizers such as sugars and/or polyols (e.g. sugar alcohols) and/or organic acids (e.g. lactic acid).

Still further the invention provides a pharmaceutical composition comprising the fermentation composition of the invention preceding item and one or more pharmaceutical grade excipient, additives and/or adjuvants. The pharmaceutical composition can be in form of a powder, tablet or capsule, or it can be liquid in the form of a pharmaceutical solution, suspension, lotion or ointment. The pharmaceutical composition can also be incorporated into suitable delivery systems such as for buccal administration or as a patch for transdermal administration.

The invention further provides a method for preparing the pharmaceutical composition of the invention comprising mixing the fermentation composition of the invention with one or more pharmaceutical grade excipient, additives and/or adjuvants.

The pharmaceutical composition is suitably used as a medicament in a method for treating and/or relieving a disease and/or medical condition, in particular in a mammal. Accordingly, the invention further provides a method for preventing, treating and/or relieving a disease and/or medical condition comprising administering a therapeutically effective amount of the pharmaceutical composition of the invention to a mammal in need of treatment and/or relief. Diseases and/or medical conditions treatable or relievable by the pharmaceutical composition includes but is not limited to pain, infections, tussive conditions, parasitic conditions, cytotoxic conditions, opiate poisoning conditions and/or cancerous conditions. Appropriate and effective dosages of benzylisoquionoline alkaloids are known in the art. The pharmaceutical preparation can be administered parenterally, such as topically, epicutaneously, sublingually, buccally, nasally, intradermally, intralesionally, (intra)ocularly, intravenously, intramuscular, intrapulmonary and/or intravaginally. The pharmaceutical composition can also be administered enterally to the gastrointestinal tract.

### Sequences

The present application contains a Sequence Listing prepared in Patentin ver 3.5 submitted electronically in ST25 format which is hereby incorporated by reference in its entirety. The following sequences are included:

TABLE A

| | | | | | |
|---|---|---|---|---|---|
| SEQ ID | Amino acid | DAHP | Aro4fbr | From | Artificial |
| NO: 1 | sequence | | | | |
| | of | | | | |
| SEQ ID | DNA coding | DAHP | Aro4fbr | From | Artificial |
| NO: 2 | sequence | | | | |
| | of | | | | |
| SEQ ID | Amino acid | chorismate mutase | ARO7fbr | From | Artificial |
| NO: 3 | sequence | | | | |
| | of | | | | |
| SEQ ID | DNA coding | chorismate mutase | ARO7fbr | From | Artificial |
| NO: 4 | sequence | | | | |
| | of | | | | |
| SEQ ID | Amino acid | Tyr1 | Tyr1 | From | S. cerevisiae |
| NO: 5 | sequence | | | | |
| | of | | | | |
| SEQ ID | DNA coding | Tyr1 | Tyr1 | From | S. cerevisiae |
| NO: 6 | sequence | | | | |
| | of | | | | |
| SEQ ID | Amino acid | TH | SoCYP76ADr9 | From | Spinacia oleracea |
| NO: 7 | sequence | | | | |
| | of | | | | |
| SEQ ID | DNA coding | TH | SoCYP76ADr9 | From | Spinacia oleracea |
| NO: 8 | sequence | | | | |
| | of | | | | |
| SEQ ID | Amino acid | TH | OfCYP76ADr12 | From | Opuntia ficus-indica |
| NO: 9 | sequence | | | | |
| | of | | | | |
| SEQ ID | DNA coding | TH | OfCYP76ADr12 | From | Opuntia ficus-indica |
| NO: 10 | sequence | | | | |
| | of | | | | |
| SEQ ID | Amino acid | TH | FlCYP76ADr11 | From | Froelichia latifolia |
| NO: 11 | sequence | | | | |
| | of | | | | |
| SEQ ID | DNA coding | TH | FlCYP76ADr11 | From | Froelichia latifolia |
| NO: 12 | sequence | | | | |
| | of | | | | |
| SEQ ID | Amino acid | TH | BvCYP76ADr10 | From | Beta vulgaris |
| NO: 13 | sequence | | | | |
| | of | | | | |
| SEQ ID | DNA coding | TH | BvCYP76ADr10 | From | Beta vulgaris |
| NO: 14 | sequence | | | | |
| | of | | | | |
| SEQ ID | Amino acid | TH | AnCYP76ADr17 | From | Abronia nealleyi |
| NO: 15 | sequence | | | | |
| | of | | | | |
| SEQ ID | DNA coding | TH | AnCYP76ADr17 | From | Abronia nealleyi |
| NO: 16 | sequence | | | | |
| | of | | | | |
| SEQ ID | Amino acid | TH | BvCYP76ADr8 | From | Beta vulgaris |
| NO: 17 | sequence | | | | |
| | of | | | | |
| SEQ ID | DNA coding | TH | BvCYP76ADr8 | From | Beta vulgaris |
| NO: 18 | sequence | | | | |
| | of | | | | |
| SEQ ID | Amino acid | TH | BvCYP76Ar7 | From | Beta vulgaris |
| NO: 19 | sequence | | | | |
| | of | | | | |
| SEQ ID | DNA coding | TH | BvCYP76Ar7 | From | Beta vulgaris |
| NO: 20 | sequence | | | | |
| | of | | | | |
| SEQ ID | Amino acid | TH | BvCYP76ADr6 | From | Beta vulgaris |
| NO: 21 | sequence | | | | |
| | of | | | | |
| SEQ ID | DNA coding | TH | BvCYP76ADr6 | From | Beta vulgaris |
| NO: 22 | sequence | | | | |
| | of | | | | |
| SEQ ID | Amino acid | TH | CbCYP76ADr28 | From | Cleretum |
| NO: 23 | sequence | | | | bellidiforme |
| | of | | | | |
| SEQ ID | DNA coding | TH | CbCYP76ADr28 | From | Cleretum |
| NO: 24 | sequence | | | | bellidiforme |
| | of | | | | |
| SEQ ID | Amino acid | TH | EvCYP76ADr20 | From | Ercilla volubilis |
| NO: 25 | sequence | | | | |
| | of | | | | |
| SEQ ID | DNA coding | TH | EvCYP76ADr20 | From | Ercilla volubilis |
| NO: 26 | sequence | | | | |
| | of | | | | |
| SEQ ID | Amino acid | TH | PdCYP76ADr21 | From | Phytolacca dioica |
| NO: 27 | sequence | | | | |
| | of | | | | |
| SEQ ID | DNA coding | TH | PdCYP76ADr21 | From | Phytolacca dioica |
| NO: 28 | sequence | | | | |
| | of | | | | |
| SEQ ID | Amino acid | TH | AoCYP76ADr16 | From | Acleisanthes obtuse |
| NO: 29 | sequence | | | | |
| | of | | | | |
| SEQ ID | DNA coding | TH | AoCYP76ADr16 | From | Acleisanthes obtuse |
| NO: 30 | sequence | | | | |
| | of | | | | |
| SEQ ID | Amino acid | TH | MmCYP76ADr18 | From | Mirabilis multiflora |
| NO: 31 | sequence | | | | |
| | of | | | | |
| SEQ ID | DNA coding | TH | MmCYP76ADr18 | From | Mirabilis multiflora |
| NO: 32 | sequence | | | | |
| | of | | | | |
| SEQ ID | Amino acid | TH | AoCYP76ADr24 | From | Acleisanthes obtuse |
| NO: 33 | sequence | | | | |
| | of | | | | |
| SEQ ID | DNA coding | TH | AoCYP76ADr24 | From | Acleisanthes obtuse |
| NO: 34 | sequence | | | | |
| | of | | | | |
| SEQ ID | Amino acid | TH | AnCYP76ADr27 | From | Abronia nealleyi |
| NO: 35 | sequence | | | | |
| | of | | | | |
| SEQ ID | DNA coding | TH | AnCYP76ADr27 | From | Abronia nealleyi |
| NO: 36 | sequence | | | | |
| | of | | | | |
| SEQ ID | Amino acid | TH | PaCYP76ADr19 | From | Phytolacca |
| NO: 37 | sequence | | | | americana |
| | of | | | | |
| SEQ ID | DNA coding | TH | PaCYP76ADr19 | From | Phytolacca |
| NO: 38 | sequence | | | | americana |
| | of | | | | |
| SEQ ID | Amino acid | TH | CqCYP76ADr5 | From | Chenopodium |
| NO: 39 | sequence | | | | quinoa |
| | of | | | | |
| SEQ ID | DNA coding | TH | CqCYP76ADr5 | From | Chenopodium |
| NO: 40 | sequence | | | | quinoa |
| | of | | | | |
| SEQ ID | Amino acid | TH | MmCYP76ADr22 | From | Mirabilis multiflora |
| NO: 41 | sequence | | | | |
| | of | | | | |
| SEQ ID | DNA coding | TH | MmCYP76ADr22 | From | Mirabilis multiflora |
| NO: 42 | sequence | | | | |
| | of | | | | |
| SEQ ID | Amino acid | TH | CqCYP76ADr4 | From | Chenopodium |
| NO: 43 | sequence | | | | quinoa |
| | of | | | | |
| SEQ ID | DNA coding | TH | CqCYP76ADr4 | From | Chenopodium |
| NO: 44 | sequence | | | | quinoa |
| | of | | | | |
| SEQ ID | Amino acid | TH | PaCYP76ADr14 | From | Phytolacca |
| NO: 45 | sequence | | | | americana |
| | of | | | | |
| SEQ ID | DNA coding | TH | PaCYP76ADr14 | From | Phytolacca |
| NO: 46 | sequence | | | | americana |
| | of | | | | |
| SEQ ID | Amino acid | TH | AnCYP76ADr23 | From | Abronia nealleyi |
| NO: 47 | sequence | | | | |
| | of | | | | |
| SEQ ID | DNA coding | TH | AnCYP76ADr23 | From | Abronia nealleyi |
| NO: 48 | sequence | | | | |
| | of | | | | |
| SEQ ID | Amino acid | TH | SoCYP76ADr2 | From | Spinacia oleracea |
| NO: 49 | sequence | | | | |
| | of | | | | |
| SEQ ID | DNA coding | TH | SoCYP76ADr2 | From | Spinacia oleracea |
| NO: 50 | sequence | | | | |
| | of | | | | |
| SEQ ID | Amino acid | TH | SoCYP76ADr3 | From | Spinacia oleracea |
| NO: 51 | sequence | | | | |
| | of | | | | |
| SEQ ID | DNA coding | TH | SoCYP76ADr3 | From | Spinacia oleracea |
| NO: 52 | sequence | | | | |
| | of | | | | |
| SEQ ID | Amino acid | TH | SoCYP76ADr1 | From | Spinacia oleracea |
| NO: 53 | sequence | | | | |
| | of | | | | |
| SEQ ID | DNA coding | TH | SoCYP76ADr1 | From | Spinacia oleracea |
| NO: 54 | sequence | | | | |
| | of | | | | |
| SEQ ID | Amino acid | TH | CqCYP76ADr13 | From | Chenopodium |
| NO: 55 | sequence | | | | quinoa |
| | of | | | | |
| SEQ ID | DNA coding | TH | CqCYP76ADr13 | From | Chenopodium |
| NO: 56 | sequence | | | | quinoa |
| | of | | | | |
| SEQ ID | Amino acid | TH | SoCYP76ADr15 | From | Spinacia oleracea |
| NO: 57 | sequence | | | | |
| | of | | | | |
| SEQ ID | DNA coding | TH | SoCYP76ADr15 | From | Spinacia oleracea |
| NO: 58 | sequence | | | | |
| | of | | | | |
| SEQ ID | Amino acid | TH | MjCYP76ADr26 | From | Mirabilis jalapa |
| NO: 59 | sequence | | | | |
| | of | | | | |
| SEQ ID | DNA coding | TH | MjCYP76ADr26 | From | Mirabilis jalapa |
| NO: 60 | sequence | | | | |
| | of | | | | |
| SEQ ID | Amino acid | TH | MmCYP76ADr25 | From | Mirabilis multiflora |
| NO: 61 | sequence | | | | |
| | of | | | | |
| SEQ ID | DNA coding | TH | MmCYP76ADr25 | From | Mirabilis multiflora |
| NO: 62 | sequence | | | | |
| | of | | | | |
| SEQ ID | Amino acid | TH | BvCYP76AD1VM | From | Beta vulgaris |
| NO: 63 | sequence | | | | |
| | of | | | | |
| SEQ ID | DNA coding | TH | BvCYP76AD1VM | From | Beta vulgaris |
| NO: 64 | sequence | | | | |
| | of | | | | |
| SEQ ID | Amino acid | TH | CYP76AD1_2mut | From | Artificial |
| NO: 65 | sequence | | | | |
| | of | | | | |
| SEQ ID | DNA coding | TH | CYP76AD1_2mut | From | Artificial |
| NO: 66 | sequence | | | | |
| | of | | | | |
| SEQ ID | Amino acid | CPR′″ | BvCPR1 | From | Beta vulgaris |
| NO: 67 | sequence | | | | |
| | of | | | | |
| SEQ ID | DNA coding | CPR′″ | BvCPR1 | From | Beta vulgaris |
| NO: 68 | sequence | | | | |
| | of | | | | |
| SEQ ID | Amino acid | DoDC | PpDoDC | From | Pseudomonas |
| NO: 69 | sequence | | | | putida |
| | of | | | | |
| SEQ ID | DNA coding | DoDC | PpDoDC | From | Pseudomonas |
| NO: 70 | sequence | | | | putida |
| | of | | | | |
| SEQ ID | Amino acid | DoDC | PpDoDC | From | Pseudomonas |
| NO: 71 | sequence | | | | putida |
| | of | | | | |
| SEQ ID | DNA coding | DoDC | PpDoDC | From | Pseudomonas |
| NO: 72 | sequence | | | | putida |
| | of | | | | |
| SEQ ID | Amino acid | NCS | d19CjNCS | From | Coptis japonica |
| NO: 73 | sequence | | | | |
| | of | | | | |
| SEQ ID | DNA coding | NCS | d19CjNCS | From | Coptis japonica |
| NO: 74 | sequence | | | | |
| | of | | | | |
| SEQ ID | DNA coding | NCS | d19CjNCS | From | Coptis japonica |
| NO: 75 | sequence | | | | |
| | of | | | | |
| SEQ ID | Amino acid | NCS | HDEL_CjNCS_V152 | From | Artificial |
| NO: 76 | sequence | | | | |
| | of | | | | |
| SEQ ID | DNA coding | NCS | HDEL_CjNCS_V152 | From | Artificial |
| NO: 77 | sequence | | | | |
| | of | | | | |
| SEQ ID | DNA coding | Integration plasmid | pRIV40 | From | Artificial |
| NO: 78 | sequence | | | | |
| | of | | | | |
| SEQ ID | Amino acid | 6-OMT | Ps6OMT_Q6WUC1 | From | Papaver |
| NO: 79 | sequence | | | | somniferum |
| | of | | | | |
| SEQ ID | DNA coding | 6-OMT | Ps6OMT_Q6WUC1 | From | Papaver |
| NO: 80 | sequence | | | | somniferum |
| | of | | | | |
| SEQ ID | Amino acid | 6-OMT | | From | Papaver |
| NO: 81 | sequence | | | | somniferum |
| | of | | | | |
| SEQ ID | Amino acid | CNMT | CjCNMT | From | Coptis japonica |
| NO: 82 | sequence | | | | |
| | of | | | | |
| SEQ ID | DNA coding | CNMT | CjCNMT | From | Coptis japonica |
| NO: 83 | sequence | | | | |
| | of | | | | |
| SEQ ID | Amino acid | CNMT | | From | Papaver |
| NO: 84 | sequence | | | | somniferum |
| | of | | | | |
| SEQ ID | Amino acid | NMCH | EcNMCH | From | Eschscholzia |
| NO: 85 | sequence | | | | californica |
| | of | | | | |
| SEQ ID | DNA coding | NMCH | EcNMCH | From | Eschscholzia |
| NO: 86 | sequence | | | | californica |
| | of | | | | |
| SEQ ID | Amino acid | NMCH | | From | Eschscholzia |
| NO: 87 | sequence | | | | californica |
| | of | | | | |
| SEQ ID | DNA coding | NMCH | | From | Eschscholzia |
| NO: 88 | sequence | | | | californica |
| | of | | | | |
| SEQ ID | Amino acid | 4′-OMT | Cj4OMT | From | Coptis japonica |
| NO: 89 | sequence | | | | |
| | of | | | | |
| SEQ ID | DNA coding | 4′-OMT | Cj4OMT | From | Coptis japonica |
| NO: 90 | sequence | | | | |
| | of | | | | |
| SEQ ID | Amino acid | 4′-OMT | | From | Papaver |
| NO: 91 | sequence | | | | somniferum |
| | of | | | | |
| SEQ ID | Amino acid | STORR | DRS-DRR | From | Papaver bracteatum |
| NO: 92 | sequence | | | | |
| | of | | | | |
| SEQ ID | DNA coding | STORR | DRS-DRR | From | Papaver bracteatum |
| NO: 93 | sequence | | | | |
| | of | | | | |
| SEQ ID | Amino acid | STORR | StIRED | From | Streptomyces |
| NO: 94 | sequence | | | | tsukubaensis |
| | of | | | | |
| SEQ ID | DNA coding | STORR | StIRED | From | Streptomyces |
| NO: 95 | sequence | | | | tsukubaensis |
| | of | | | | |
| SEQ ID | Amino acid | STORR | PsSTORR | From | Papaver |
| NO: 96 | sequence | | | | somniferum |
| | of | | | | |
| SEQ ID | DNA coding | STORR | PsSTORR | From | Papaver |
| NO: 97 | sequence | | | | somniferum |
| | of | | | | |
| SEQ ID | Amino acid | STORR P450 | PsCYP82Y2 | From | Papaver |
| NO: 98 | sequence | | | | somniferum |
| | of | | | | |
| SEQ ID | DNA coding | STORR P450 | PsCYP82Y2 | From | Papaver |
| NO: 99 | sequence | | | | somniferum |
| | of | | | | |
| SEQ ID | Amino acid | STORR P450 | PrCYP82Y2-like | From | Papaver rhoeas |
| NO: 100 | sequence | | | | |
| | of | | | | |
| SEQ ID | DNA coding | STORR P450 | PrCYP82Y2-like | From | Papaver rhoeas |
| NO: 101 | sequence | | | | |
| | of | | | | |
| SEQ ID | Amino acid | STORR P450 | proID60 | From | Artificial |
| NO: 102 | sequence | | | | |
| | of | | | | |
| SEQ ID | DNA coding | STORR P450 | proID60 | From | Artificial |
| NO: 103 | sequence | | | | |
| | of | | | | |
| SEQ ID | Amino acid | STORR P450 | proID66 | From | Artificial |
| NO: 104 | sequence | | | | |
| | of | | | | |
| SEQ ID | DNA coding | STORR P450 | proID66 | From | Artificial |
| NO: 105 | sequence | | | | |
| | of | | | | |
| SEQ ID | Amino acid | STORR P450 | proID79 | From | Artificial |
| NO: 106 | sequence | | | | |
| | of | | | | |
| SEQ ID | DNA coding | STORR P450 | proID79 | From | Artificial |
| NO: 107 | sequence | | | | |
| | of | | | | |
| SEQ ID | Amino acid | STORR Reductase | PsAKR | From | Papaver |
| NO: 108 | sequence | | | | somniferum |
| | of | | | | |
| SEQ ID | DNA coding | STORR Reductase | PsAKR | From | Papaver |
| NO: 109 | sequence | | | | somniferum |
| | of | | | | |
| SEQ ID | Amino acid | STORR Reductase | PrAKR | From | Papaver rhoeas |
| NO: 110 | sequence | | | | |
| | of | | | | |
| SEQ ID | DNA coding | STORR Reductase | PrAKR | From | Papaver rhoeas |
| NO: 111 | sequence | | | | |
| | of | | | | |
| SEQ ID | Amino acid | CPR″ | PsCPR | From | Papaver |
| NO: 112 | sequence | | | | somniferum |
| | of | | | | |
| SEQ ID | DNA coding | CPR″ | PsCPR | From | Papaver |
| NO: 113 | sequence | | | | somniferum |
| | of | | | | |
| SEQ ID | Amino acid | CPR″ | AtATR1 | From | Arabidopsis thaliana |
| NO: 114 | sequence | | | | |
| | of | | | | |
| SEQ ID | DNA coding | CPR″ | AtATR1 | From | Arabidopsis thaliana |
| NO: 115 | sequence | | | | |
| | of | | | | |
| SEQ ID | Amino acid | SAS | PbSAS | From | Papaver bracteatum |
| NO: 116 | sequence | | | | |
| | of | | | | |
| SEQ ID | DNA coding | SAS | PbSAS | From | Papaver bracteatum |
| NO: 117 | sequence | | | | |
| | of | | | | |
| SEQ ID | Amino acid | SAS | — | From | Papaver bracteatum |
| NO: 118 | sequence | | | | |
| | of | | | | |
| SEQ ID | DNA coding | SAS | — | From | Papaver bracteatum |
| NO: 119 | sequence | | | | |
| | of | | | | |
| SEQ ID | Amino acid | SAR | pbSalR | From | Papaver bracteatum |
| NO: 120 | sequence | | | | |
| | of | | | | |
| SEQ ID | DNA coding | SAR | pbSalR | From | Papaver bracteatum |
| NO: 121 | sequence | | | | |
| | of | | | | |
| SEQ ID | Amino acid | SAR | — | From | Papaver bracteatum |
| NO: 122 | sequence | | | | |
| | of | | | | |
| SEQ ID | Amino acid | SAT | PsSAT | From | Papaver |
| NO: 123 | sequence | | | | somniferum |
| | of | | | | |
| SEQ ID | DNA coding | SAT | PsSAT | From | Papaver |
| NO: 124 | sequence | | | | somniferum |
| | of | | | | |
| SEQ ID | Amino acid | SAT | — | From | Papaver |
| NO: 125 | sequence | | | | somniferum |
| | of | | | | |
| SEQ ID | Amino acid | THS | HA BetV1M | From | Papaver |
| NO: 126 | sequence | | | | somniferum |
| | of | | | | |
| SEQ ID | Amino acid | THS | BETV1L HA | From | Papaver |
| NO: 127 | sequence | | | | somniferum |
| | of | | | | |
| SEQ ID | Amino acid | THS | — | From | Papaver |
| NO: 128 | sequence | | | | somniferum |
| | of | | | | |
| SEQ ID | Amino acid | THS | PsTHS1 | From | Papaver |
| NO: 129 | sequence | | | | somniferum |
| | of | | | | |
| SEQ ID | DNA coding | THS | PsTHS1 | From | Papaver |
| NO: 130 | sequence | | | | somniferum |
| | of | | | | |
| SEQ ID | Amino acid | THS | PsTHS2 | From | Papaver |
| NO: 131 | sequence | | | | somniferum |
| | of | | | | |
| SEQ ID | DNA coding | THS | PsTHS2 | From | Papaver |
| NO: 132 | sequence | | | | somniferum |
| | of | | | | |
| SEQ ID | Amino acid | THS | — | From | Papaver |
| NO: 133 | sequence | | | | somniferum |
| | of | | | | |
| SEQ ID | Amino acid | THS | PROths2_138 | From | Artificial |
| NO: 134 | sequence | | | | |
| | of | | | | |
| SEQ ID | DNA coding | THS | PROths2_138 | From | Artificial |
| NO: 135 | sequence | | | | |
| | of | | | | |
| SEQ ID | Amino acid | THS | PROths2_143 | From | Artificial |
| NO: 136 | sequence | | | | |
| | of | | | | |
| SEQ ID | DNA coding | THS | PROths2_143 | From | Artificial |
| NO: 137 | sequence | | | | |
| | of | | | | |
| SEQ ID | Amino acid | THS | PROths2_116 | From | Artificial |
| NO: 138 | sequence | | | | |
| | of | | | | |
| SEQ ID | DNA coding | THS | PROths2_116 | From | Artificial |
| NO: 139 | sequence | | | | |
| | of | | | | |
| SEQ ID | Amino acid | P450 | HaCYP6AE15v2 protein | From | Helicoverpa |
| NO: 140 | sequence | | | | armigera |
| | of | | | | |
| SEQ ID | DNA coding | P450 | HaCYP6AE15v2 *DNA | From | Helicoverpa |
| NO: 141 | sequence | | | | armigera |
| | of | | | | |
| SEQ ID | Amino acid | P450 | HaCYP6AE19 protein | From | Helicoverpa |
| NO: 142 | sequence | | | | armigera |
| | of | | | | |
| SEQ ID | DNA coding | P450 | HaCYP6AE19 *DNA | From | Helicoverpa |
| NO: 143 | sequence | | | | armigera |
| | of | | | | |
| SEQ ID | Amino acid | P450 | HaCYP6AE11 protein | From | Helicoverpa |
| NO: 144 | sequence | | | | armigera |
| | of | | | | |
| SEQ ID | DNA coding | P450 | HaCYP6AE11 *DNA | From | Helicoverpa |
| NO: 145 | sequence | | | | armigera |
| | of | | | | |
| SEQ ID | Amino acid | P450 | HaCYP6AE17 protein | From | Helicoverpa |
| NO: 146 | sequence | | | | armigera |
| | of | | | | |
| SEQ ID | DNA coding | P450 | HaCYP6AE17 *DNA | From | Helicoverpa |
| NO: 147 | sequence | | | | armigera |
| | of | | | | |
| SEQ ID | Amino acid | P450 | HaCYP6AE24 protein | From | Helicoverpa |
| NO: 148 | sequence | | | | armigera |
| | of | | | | |
| SEQ ID | DNA coding | P450 | HaCYP6AE24 *DNA | From | Helicoverpa |
| NO: 149 | sequence | | | | armigera |
| | of | | | | |
| SEQ ID | Amino acid | P450 | HaCYP6AE20v2 protein | From | Helicoverpa |
| NO: 150 | sequence | | | | armigera |
| | of | | | | |
| SEQ ID | DNA coding | P450 | HaCYP6AE20v2 *DNA | From | Helicoverpa |
| NO: 151 | sequence | | | | armigera |
| | of | | | | |
| SEQ ID | Amino acid | P450 | Hv_CYP_A0A2A4JAM9 protein | From | Heliothis virescens |
| NO: 152 | sequence | | | | |
| | of | | | | |
| SEQ ID | DNA coding | P450 | Hv_CYP_A0A2A4JAM9 *DNA | From | Heliothis virescens |
| NO: 153 | sequence | | | | |
| | of | | | | |
| SEQ ID | Amino acid | P450 | Hv_CYP_A0A2A4JAK3 protein | From | Heliothis virescens |
| NO: 154 | sequence | | | | |
| | of | | | | |
| SEQ ID | DNA coding | P450 | Hv_CYP_A0A2A4JAK3 *DNA | From | Heliothis virescens |
| NO: 155 | sequence | | | | |
| | of | | | | |
| SEQ ID | Amino acid | P450 | Se_CYP6AE68 protein | From | Spodoptera exigua |
| NO: 156 | sequence | | | | |
| | of | | | | |
| SEQ ID | DNA coding | P450 | Se_CYP6AE68 *DNA | From | Spodoptera exigua |
| NO: 157 | sequence | | | | |
| | of | | | | |
| SEQ ID | Amino acid | P450 | Hv_CYP_A0A2A4J7V4 protein | From | Heliothis virescens |
| NO: 158 | sequence | | | | |
| | of | | | | |
| SEQ ID | DNA coding | P450 | Hv_CYP_A0A2A4J7V4 *DNA | From | Heliothis virescens |
| NO: 159 | sequence | | | | |
| | of | | | | |
| SEQ ID | Amino acid | P450 | CmCYP6_A0A0C5CGV6 protein | From | Cnaphalocrocis |
| NO: 160 | sequence | | | | medinalis |
| | of | | | | |
| SEQ ID | DNA coding | P450 | CmCYP6_A0A0C5CGV6 *DNA | From | Cnaphalocrocis |
| NO: 161 | sequence | | | | medinalis |
| | of | | | | |
| SEQ ID | Amino acid | P450 | BmCYP6AE9_A9QW15 protein | From | Bombyx mandarina |
| NO: 162 | sequence | | | | |
| | of | | | | |
| SEQ ID | DNA coding | P450 | BmCYP6AE9_A9QW15 *DNA | From | Bombyx mandarina |
| NO: 163 | sequence | | | | |
| | of | | | | |
| SEQ ID | Amino acid | P450 | Bm_CYP6AE9 protein | From | Bombyx mori |
| NO: 164 | sequence | | | | |
| | of | | | | |
| SEQ ID | DNA coding | P450 | Bm_CYP6AE9 *DNA | From | Bombyx mori |
| NO: 165 | sequence | | | | |
| | of | | | | |
| SEQ ID | Amino acid | P450 | Se_CYP6AE10 protein | From | Spodoptera exigua |
| NO: 166 | sequence | | | | |
| | of | | | | |
| SEQ ID | DNA coding | P450 | Se_CYP6AE10 *DNA | From | Spodoptera exigua |
| NO: 167 | sequence | | | | |
| | of | | | | |
| SEQ ID | Amino acid | P450 | Sf_CYP_A0A2H1WID4 protein | From | Spodoptera |
| NO: 168 | sequence | | | | frugiperda |
| | of | | | | |
| SEQ ID | DNA coding | P450 | Sf_CYP_A0A2H1WID4 *DNA | From | Spodoptera |
| NO: 169 | sequence | | | | frugiperda |
| | of | | | | |
| SEQ ID | Amino acid | P450 | Sf_CYP_A0A2H1V0E7 protein | From | Spodoptera |
| NO: 170 | sequence | | | | frugiperda |
| | of | | | | |
| SEQ ID | DNA coding | P450 | Sf_CYP_A0A2H1V0E7 *DNA | From | Spodoptera |
| NO: 171 | sequence | | | | frugiperda |
| | of | | | | |
| SEQ ID | Amino acid | P450 | Ha_CYP6AE12 protein | From | Helicoverpa |
| NO: 172 | sequence | | | | armigera |
| | of | | | | |
| SEQ ID | DNA coding | P450 | Ha_CYP6AE12 *DNA | From | Helicoverpa |
| NO: 173 | sequence | | | | armigera |
| | of | | | | |
| SEQ ID | Amino acid | P450 | Sf_CYP6AE44 protein | From | Spodoptera |
| NO: 174 | sequence | | | | frugiperda |
| | of | | | | |
| SEQ ID | DNA coding | P450 | Sf_CYP6AE44 *DNA | From | Spodoptera |
| NO: 175 | sequence | | | | frugiperda |
| | of | | | | |
| SEQ ID | Amino acid | P450 | HaCYP6AE_A0A068F0X7 protein | From | Helicoverpa |
| NO: 176 | sequence | | | | armigera |
| | of | | | | |
| SEQ ID | DNA coding | P450 | HaCYP6AE_A0A068F0X7 *DNA | From | Helicoverpa |
| NO: 177 | sequence | | | | armigera |
| | of | | | | |
| SEQ ID | Amino acid | P450 | DpCYP_Q7YZS2 protein | From | Depressaria |
| NO: 178 | sequence | | | | pastinacella |
| | of | | | | |
| SEQ ID | DNA coding | P450 | DpCYP_Q7YZS2 *DNA | From | Depressaria |
| NO: 179 | sequence | | | | pastinacella |
| | of | | | | |
| SEQ ID | Amino acid | P450 | Sf_CYP_A0A2H1V0E7 protein | From | Spodoptera |
| NO: 180 | sequence | | | | frugiperda |
| | of | | | | |
| SEQ ID | DNA coding | P450 | Sf_CYP_A0A2H1V0E7 *DNA | From | Spodoptera |
| NO: 181 | sequence | | | | frugiperda |
| | of | | | | |
| SEQ ID | Amino acid | P450 | BmCYP6AE2_L0N7C5 protein | From | Bombyx mori |
| NO: 182 | sequence | | | | |
| | of | | | | |
| SEQ ID | DNA coding | P450 | BmCYP6AE2_L0N7C5 *DNA | From | Bombyx mori |
| NO: 183 | sequence | | | | |
| | of | | | | |
| SEQ ID | Amino acid | P450 | BmCYP_C1KJL7 protein | From | Bombyx mandarina |
| NO: 184 | sequence | | | | |
| | of | | | | |
| SEQ ID | DNA coding | P450 | BmCYP_C1KJL7 *DNA | From | Bombyx mandarina |
| NO: 185 | sequence | | | | |
| | of | | | | |
| SEQ ID | Amino acid | P450 | ZfCYP6AE27_D2JLK6 protein | From | Zygaena filipendulae |
| NO: 186 | sequence | | | | |
| | of | | | | |
| SEQ ID | DNA coding | P450 | ZfCYP6AE27_D2JLK6 *DNA | From | Zygaena filipendulae |
| NO: 187 | sequence | | | | |
| | of | | | | |
| SEQ ID | Amino acid | P450 | BmCyp6AE21_B6VFR9 protein | From | Bombyx mori |
| NO: 188 | sequence | | | | |
| | of | | | | |
| SEQ ID | DNA coding | P450 | BmCyp6AE21_B6VFR9 *DNA | From | Bombyx mori |
| NO: 189 | sequence | | | | |
| | of | | | | |
| SEQ ID | Amino acid | P450 | BmCYP6AE7_A4GUB8 protein | From | Bombyx mori |
| NO: 190 | sequence | | | | |
| | of | | | | |
| SEQ ID | DNA coding | P450 | BmCYP6AE7_A4GUB8 *DNA | From | Bombyx mori |
| NO: 191 | sequence | | | | |
| | of | | | | |
| SEQ ID | Amino acid | P450 | CmCYP6_A0A0C5C1I6 protein | From | Cnaphalocrocis |
| NO: 192 | sequence | | | | medinalis |
| | of | | | | |
| SEQ ID | DNA coding | P450 | CmCYP6_A0A0C5C1I6 *DNA | From | Cnaphalocrocis |
| NO: 193 | sequence | | | | medinalis |
| | of | | | | |
| SEQ ID | Amino acid | P450 | SeCYP6_A0A248QEH8 protein | From | Spodoptera exigua |
| NO: 194 | sequence | | | | |
| | of | | | | |
| SEQ ID | DNA coding | P450 | SeCYP6_A0A248QEH8 *DNA | From | Spodoptera exigua |
| NO: 195 | sequence | | | | |
| | of | | | | |
| SEQ ID | Amino acid | P450 | BmCYP6AE9_A5HKM1 protein | From | Bombyx mori |
| NO: 196 | sequence | | | | |
| | of | | | | |
| SEQ ID | DNA coding | P450 | BmCYP6AE9_A5HKM1 *DNA | From | Bombyx mori |
| NO: 197 | sequence | | | | |
| | of | | | | |
| SEQ ID | Amino acid | P450 | CYPDN_39 protein | From | Rhizopus |
| NO: 198 | sequence | | | | microsporus |
| | of | | | | |
| SEQ ID | DNA coding | P450 | CYPDN_39 gene | From | Rhizopus |
| NO: 199 | sequence | | | | microsporus |
| | of | | | | |
| SEQ ID | Amino acid | P450 | CYPDN_41 protein | From | Rhizopus |
| NO: 200 | sequence | | | | microsporus |
| | of | | | | |
| SEQ ID | DNA coding | P450 | CYPDN_41 gene | From | Rhizopus |
| NO: 201 | sequence | | | | microsporus |
| | of | | | | |
| SEQ ID | Amino acid | P450 | CYPDN_43 protein | From | Lichtheimia |
| NO: 202 | sequence | | | | corymbifera |
| | of | | | | |
| SEQ ID | DNA coding | P450 | CYPDN_43 gene | From | Lichtheimia |
| NO: 203 | sequence | | | | corymbifera |
| | of | | | | |
| SEQ ID | Amino acid | P450 | CYPDN_44 protein | From | Lichtheimia ramosa |
| NO: 204 | sequence | | | | |
| | of | | | | |
| SEQ ID | DNA coding | P450 | CYPDN_44 gene | From | Lichtheimia ramosa |
| NO: 205 | sequence | | | | |
| | of | | | | |
| SEQ ID | Amino acid | P450 | CYPDN_45 protein | From | Rhizopus |
| NO: 206 | sequence | | | | microsporus |
| | of | | | | |
| SEQ ID | DNA coding | P450 | CYPDN_45 gene | From | Rhizopus |
| NO: 207 | sequence | | | | microsporus |
| | of | | | | |
| SEQ ID | Amino acid | P450 | CYPDN_50 protein | From | Lichtheimia ramosa |
| NO: 208 | sequence | | | | |
| | of | | | | |
| SEQ ID | DNA coding | P450 | CYPDN_50 gene | From | Lichtheimia ramosa |
| NO: 209 | sequence | | | | |
| | of | | | | |
| SEQ ID | Amino acid | P450 | CYPDN_51 protein | From | Lichtheimia ramosa |
| NO: 210 | sequence | | | | |
| | of | | | | |
| SEQ ID | DNA coding | P450 | CYPDN_51 gene | From | Lichtheimia ramosa |
| NO: 211 | sequence | | | | |
| | of | | | | |
| SEQ ID | Amino acid | P450 | CYPDN_57 protein | From | Syncephalastrum |
| NO: 212 | sequence | | | | racemosum |
| | of | | | | |
| SEQ ID | DNA coding | P450 | CYPDN_57 gene | From | Syncephalastrum |
| NO: 213 | sequence | | | | racemosum |
| | of | | | | |
| SEQ ID | Amino acid | P450 | CYPDN_59 protein | From | Cunninghamella |
| NO: 214 | sequence | | | | echinulata |
| | of | | | | |
| SEQ ID | DNA coding | P450 | CYPDN_59 gene | From | Cunninghamella |
| NO: 215 | sequence | | | | echinulata |
| | of | | | | |
| SEQ ID | Amino acid | P450 | CYPDN_61 protein | From | Rhizopus |
| NO: 216 | sequence | | | | azygosporus |
| | of | | | | |
| SEQ ID | DNA coding | P450 | CYPDN_61 gene | From | Rhizopus |
| NO: 217 | sequence | | | | azygosporus |
| | of | | | | |
| SEQ ID | Amino acid | P450 | CYPDN_62 protein | From | Rhizopus |
| NO: 218 | sequence | | | | azygosporus |
| | of | | | | |
| SEQ ID | DNA coding | P450 | CYPDN_62 gene | From | Rhizopus |
| NO: 219 | sequence | | | | azygosporus |
| | of | | | | |
| SEQ ID | Amino acid | P450 | CYPDN_63 protein | From | Rhizopus |
| NO: 220 | sequence | | | | microsporus |
| | of | | | | |
| SEQ ID | DNA coding | P450 | CYPDN_63 gene | From | Rhizopus |
| NO: 221 | sequence | | | | microsporus |
| | of | | | | |
| SEQ ID | Amino acid | P450 | CYPDN_64 protein | From | Mucor circinelloides |
| NO: 222 | sequence | | | | f. circinelloides |
| | of | | | | |
| SEQ ID | DNA coding | P450 | CYPDN_64 gene | From | Mucor circinelloides |
| NO: 223 | sequence | | | | f. circinelloides |
| | of | | | | |
| SEQ ID | Amino acid | P450 | CYPDN_65 protein | From | Mucor ambiguus |
| NO: 224 | sequence | | | | |
| | of | | | | |
| SEQ ID | DNA coding | P450 | CYPDN_65 gene | From | Mucor ambiguus |
| NO: 225 | sequence | | | | |
| | of | | | | |
| SEQ ID | Amino acid | P450 | CYPDN_67 protein | From | Syncephalastrum |
| NO: 226 | sequence | | | | racemosum |
| | of | | | | |
| SEQ ID | DNA coding | P450 | CYPDN_67 gene | From | Syncephalastrum |
| NO: 227 | sequence | | | | racemosum |
| | of | | | | |
| SEQ ID | Amino acid | P450 | CYPDN_68 protein | From | Parasitella parasitica |
| NO: 228 | sequence | | | | |
| | of | | | | |
| SEQ ID | DNA coding | P450 | CYPDN_68 gene | From | Parasitella parasitica |
| NO: 229 | sequence | | | | |
| | of | | | | |
| SEQ ID | Amino acid | P450 | CYPDN_69 protein | From | Syncephalastrum |
| NO: 230 | sequence | | | | racemosum |
| | of | | | | |
| SEQ ID | DNA coding | P450 | CYPDN_69 gene | From | Syncephalastrum |
| NO: 231 | sequence | | | | racemosum |
| | of | | | | |
| SEQ ID | Amino acid | P450 | CYPDN_70 protein | From | Lichtheimia ramosa |
| NO: 232 | sequence | | | | |
| | of | | | | |
| SEQ ID | DNA coding | P450 | CYPDN_70 gene | From | Lichtheimia ramosa |
| NO: 233 | sequence | | | | |
| | of | | | | |
| SEQ ID | Amino acid | P450 | CYPDN_74 protein | From | Lichtheimia |
| NO: 234 | sequence | | | | corymbifera |
| | of | | | | |
| SEQ ID | DNA coding | P450 | CYPDN_74 gene | From | Lichtheimia |
| NO: 235 | sequence | | | | corymbifera |
| | of | | | | |
| SEQ ID | Amino acid | P450 | CYPDN_75 protein | From | Absidia repens |
| NO: 236 | sequence | | | | |
| | of | | | | |
| SEQ ID | DNA coding | P450 | CYPDN_75 gene | From | Absidia repens |
| NO: 237 | sequence | | | | |
| | of | | | | |
| SEQ ID | Amino acid | P450 | CYPDN_77 protein | From | Lichtheimia |
| NO: 238 | sequence | | | | corymbifera |
| | of | | | | |
| SEQ ID | DNA coding | P450 | CYPDN_77 gene | From | Lichtheimia |
| NO: 239 | sequence | | | | corymbifera |
| | of | | | | |
| SEQ ID | Amino acid | P450 | CYPDN_80 protein | From | Absidia glauca |
| NO: 240 | sequence | | | | |
| | of | | | | |
| SEQ ID | DNA coding | P450 | CYPDN_80 gene | From | Absidia glauca |
| NO: 241 | sequence | | | | |
| | of | | | | |
| SEQ ID | Amino acid | P450 | CYPDN_82 protein | From | Choanephora |
| NO: 242 | sequence | | | | cucurbitarum |
| | of | | | | |
| SEQ ID | DNA coding | P450 | CYPDN_82 gene | From | Choanephora |
| NO: 243 | sequence | | | | cucurbitarum |
| | of | | | | |
| SEQ ID | Amino acid | P450 | CYPDN_84 protein | From | Absidia glauca |
| NO: 244 | sequence | | | | |
| | of | | | | |
| SEQ ID | DNA coding | P450 | CYPDN_84 gene | From | Absidia glauca |
| NO: 245 | sequence | | | | |
| | of | | | | |
| SEQ ID | Amino acid | P450 | CYPDN_85 protein | From | Absidia repens |
| NO: 246 | sequence | | | | |
| | of | | | | |
| SEQ ID | DNA coding | P450 | CYPDN_85 gene | From | Absidia repens |
| NO: 247 | sequence | | | | |
| | of | | | | |
| SEQ ID | Amino acid | P450 | CYPDN_86 protein | From | Absidia repens |
| NO: 248 | sequence | | | | |
| | of | | | | |
| SEQ ID | DNA coding | P450 | CYPDN_86 gene | From | Absidia repens |
| NO: 249 | sequence | | | | |
| | of | | | | |
| SEQ ID | Amino acid | P450 | CYPDN_91 protein | From | Rhizopus |
| NO: 250 | sequence | | | | microsporus |
| | of | | | | |
| SEQ ID | DNA coding | P450 | CYPDN_91 gene | From | Rhizopus |
| NO: 251 | sequence | | | | microsporus |
| | of | | | | |
| SEQ ID | Amino acid | P450 | CYPDN_92 protein | From | Rhizopus |
| NO: 252 | sequence | | | | azygosporus |
| | of | | | | |
| SEQ ID | DNA coding | P450 | CYPDN_92 gene | From | Rhizopus |
| NO: 253 | sequence | | | | azygosporus |
| | of | | | | |
| SEQ ID | Amino acid | P450 | CYPDN_93 protein | From | Rhizopus |
| NO: 254 | sequence | | | | azygosporus |
| | of | | | | |
| SEQ ID | DNA coding | P450 | CYPDN_93 gene | From | Rhizopus |
| NO: 255 | sequence | | | | azygosporus |
| | of | | | | |
| SEQ ID | Amino acid | P450 | CYPDN_95 protein | From | Bifiguratus |
| NO: 256 | sequence | | | | adelaidae |
| | of | | | | |
| SEQ ID | DNA coding | P450 | CYPDN_95 gene | From | Bifiguratus |
| NO: 257 | sequence | | | | adelaidae |
| | of | | | | |
| SEQ ID | Amino acid | P450 | CYPDN_98 protein | From | Rhizopus stolonifer |
| NO: 258 | sequence | | | | |
| | of | | | | |
| SEQ ID | DNA coding | P450 | CYPDN_98 gene | From | Rhizopus stolonifer |
| NO: 259 | sequence | | | | |
| | of | | | | |
| SEQ ID | Amino acid | P450 | CYPDN_100 protein | From | Rhizopus oryzae |
| NO: 260 | sequence | | | | |
| | of | | | | |
| SEQ ID | DNA coding | P450 | CYPDN_100 gene | From | Rhizopus oryzae |
| NO: 261 | sequence | | | | |
| | of | | | | |
| SEQ ID | Amino acid | P450 | CYPDN_101 protein | From | Rhizopus |
| NO: 262 | sequence | | | | microsporus |
| | of | | | | |
| SEQ ID | DNA coding | P450 | CYPDN_101 gene | From | Rhizopus |
| NO: 263 | sequence | | | | microsporus |
| | of | | | | |
| SEQ ID | Amino acid | P450 | CYPDN_103 protein | From | Rhizopus delemar |
| NO: 264 | sequence | | | | RA 99-880 |
| | of | | | | |
| SEQ ID | DNA coding | P450 | CYPDN_103 gene | From | Rhizopus delemar |
| NO: 265 | sequence | | | | RA 99-880 |
| | of | | | | |
| SEQ ID | Amino acid | P450 | CYPDN_104 protein | From | Rhizopus stolonifer |
| NO: 266 | sequence | | | | |
| | of | | | | |
| SEQ ID | DNA coding | P450 | CYPDN_104 gene | From | Rhizopus stolonifer |
| NO: 267 | sequence | | | | |
| | of | | | | |
| SEQ ID | Amino acid | P450 | CYPDN_105 protein | From | Rhizopus |
| NO: 268 | sequence | | | | azygosporus |
| | of | | | | |
| SEQ ID | DNA coding | P450 | CYPDN_105 gene | From | Rhizopus |
| NO: 269 | sequence | | | | azygosporus |
| | of | | | | |
| SEQ ID | Amino acid | P450 | CYPDN_108 protein | From | Mucor circinelloides |
| NO: 270 | sequence | | | | f. circinelloides |
| | of | | | | |
| SEQ ID | DNA coding | P450 | CYPDN_108 gene | From | Mucor circinelloides |
| NO: 271 | sequence | | | | f. circinelloides |
| | of | | | | |
| SEQ ID | Amino acid | P450 | CYPDN_109 protein | From | Mucor circinelloides |
| NO: 272 | sequence | | | | f. circinelloides |
| | of | | | | |
| SEQ ID | DNA coding | P450 | CYPDN_109 gene | From | Mucor circinelloides |
| NO: 273 | sequence | | | | f. circinelloides |
| | of | | | | |
| SEQ ID | Amino acid | P450 | CYPDN_110 protein | From | Mucor circinelloides |
| NO: 274 | sequence | | | | f. lusitanicus |
| | of | | | | |
| SEQ ID | DNA coding | P450 | CYPDN_110 gene | From | Mucor circinelloides |
| NO: 275 | sequence | | | | f. lusitanicus |
| | of | | | | |
| SEQ ID | Amino acid | P450 | CYPDN_112 protein | From | Choanephora |
| NO: 276 | sequence | | | | cucurbitarum |
| | of | | | | |
| SEQ ID | DNA coding | P450 | CYPDN_112 gene | From | Choanephora |
| NO: 277 | sequence | | | | cucurbitarum |
| | of | | | | |
| SEQ ID | Amino acid | P450 | CYPDN_115 protein | From | Lichtheimia |
| NO: 278 | sequence | | | | corymbifera |
| | of | | | | |
| SEQ ID | DNA coding | P450 | CYPDN_115 gene | From | Lichtheimia |
| NO: 279 | sequence | | | | corymbifera |
| | of | | | | |
| SEQ ID | Amino acid | P450 | CYPDN_117 protein | From | Lichtheimia |
| NO: 280 | sequence | | | | corymbifera |
| | of | | | | |
| SEQ ID | DNA coding | P450 | CYPDN_117 gene | From | Lichtheimia |
| NO: 281 | sequence | | | | corymbifera |
| | of | | | | |
| SEQ ID | Amino acid | P450 | CYPDN_118 protein | From | Lichtheimia ramosa |
| NO: 282 | sequence | | | | |
| | of | | | | |
| SEQ ID | DNA coding | P450 | CYPDN_118 gene | From | Lichtheimia ramosa |
| NO: 283 | sequence | | | | |
| | of | | | | |
| SEQ ID | Amino acid | P450 | CYPDN_119 protein | From | Lichtheimia |
| NO: 284 | sequence | | | | corymbifera |
| | of | | | | |
| SEQ ID | DNA coding | P450 | CYPDN_119 gene | From | Lichtheimia |
| NO: 285 | sequence | | | | corymbifera |
| | of | | | | |
| SEQ ID | Amino acid | P450 | CYPDN_120 protein | From | Lichtheimia ramosa |
| NO: 286 | sequence | | | | |
| | of | | | | |
| SEQ ID | DNA coding | P450 | CYPDN_120 gene | From | Lichtheimia ramosa |
| NO: 287 | sequence | | | | |
| | of | | | | |
| SEQ ID | Amino acid | P450 | CYPDN_123 protein | From | Lichtheimia ramosa |
| NO: 288 | sequence | | | | |
| | of | | | | |
| SEQ ID | DNA coding | P450 | CYPDN_123 gene | From | Lichtheimia ramosa |
| NO: 289 | sequence | | | | |
| | of | | | | |
| SEQ ID | Amino acid | P450 | CYPDN_8 protein | From | Rhizopus |
| NO: 290 | sequence | | | | microsporus |
| | of | | | | |
| SEQ ID | DNA coding | P450 | CYPDN_8 *DNA | From | Rhizopus |
| NO: 291 | sequence | | | | microsporus |
| | of | | | | |
| SEQ ID | Amino acid | CPR′ | HaCPR_E0A3A7 protein | From | Helicoverpa |
| NO: 292 | sequence | | | | armigera |
| | of | | | | |
| SEQ ID | DNA coding | CPR′ | HaCPR_E0A3A7 *DNA | From | Helicoverpa |
| NO: 293 | sequence | | | | armigera |
| | of | | | | |
| SEQ ID | Amino acid | CPR′ | Se_CPR_F1DI27 | From | Spodoptera exigua |
| NO: 294 | sequence | | | | |
| | of | | | | |
| SEQ ID | DNA coding | CPR′ | Se_CPR_F1DI27 | From | Spodoptera exigua |
| NO: 295 | sequence | | | | |
| | of | | | | |
| SEQ ID | Amino acid | CPR′ | Bm_CPR_Q9NKV3 | From | Bombyx mori |
| NO: 296 | sequence | | | | |
| | of | | | | |
| SEQ ID | DNA coding | CPR′ | Bm_CPR_Q9NKV3 | From | Bombyx mori |
| NO: 297 | sequence | | | | |
| | of | | | | |
| SEQ ID | Amino acid | CPR′ | BmCPR_A0FGR6 | From | Bombyx mandarina |
| NO: 298 | sequence | | | | |
| | of | | | | |
| SEQ ID | DNA coding | CPR′ | BmCPR_A0FGR6 | From | Bombyx mandarina |
| NO: 299 | sequence | | | | |
| | of | | | | |
| SEQ ID | Amino acid | CPR′ | ZfCPR_A0A346M705 | From | Zygaena filipendulae |
| NO: 300 | sequence | | | | |
| | of | | | | |
| SEQ ID | DNA coding | CPR′ | ZfCPR_A0A346M705 | From | Zygaena filipendulae |
| NO: 301 | sequence | | | | |
| | of | | | | |
| SEQ ID | Amino acid | CPR′ | CmCPR_A0A1S5ZY34 | From | Cnaphalocrocis |
| NO: 302 | sequence | | | | medinalis |
| | of | | | | |
| SEQ ID | DNA coding | CPR′ | CmCPR_A0A1S5ZY34 | From | Cnaphalocrocis |
| NO: 303 | sequence | | | | medinalis |
| | of | | | | |
| SEQ ID | DNA coding | CPR′ | HaCPR_E7E2N6 | From | Helicoverpa |
| NO: 304 | sequence | | | | armigera |
| | of | | | | |
| SEQ ID | Amino acid | CPR′ | CeCPR protein | From | Cunninghamella |
| NO: 305 | sequence | | | | elegans |
| | of | | | | |
| SEQ ID | DNA coding | CPR′ | CeCPR gene | From | Cunninghamella |
| NO: 306 | sequence | | | | elegans |
| | of | | | | |
| SEQ ID | Amino acid | Transporter | T1_CjaMDR1_GA | From | Camellia japonica |
| NO: 307 | sequence | | | | |
| | of | | | | |
| SEQ ID | DNA coding | Transporter | T1_CjaMDR1_GA | From | Camellia japonica |
| NO: 308 | sequence | | | | |
| | of | | | | |
| SEQ ID | Amino acid | Transporter | T3_NcaNPF_GA | From | Noccaea |
| NO: 309 | sequence | | | | caerulescens |
| | of | | | | |
| SEQ ID | DNA coding | Transporter | T3_NcaNPF_GA | From | Noccaea |
| NO: 310 | sequence | | | | caerulescens |
| | of | | | | |
| SEQ ID | Amino acid | Transporter | T4_EsaGTR_GA | From | Eutrema |
| NO: 311 | sequence | | | | salsugineum |
| | of | | | | |
| SEQ ID | DNA coding | Transporter | T4_EsaGTR_GA | From | Eutrema |
| NO: 312 | sequence | | | | salsugineum |
| | of | | | | |
| SEQ ID | Amino acid | Transporter | T5_AlyPOT_GA | From | Arabidopsis lyrata |
| NO: 313 | sequence | | | | subsp. lyrata |
| | of | | | | |
| SEQ ID | DNA coding | Transporter | T5_AlyPOT_GA | From | Arabidopsis lyrata |
| NO: 314 | sequence | | | | subsp. lyrata |
| | of | | | | |
| SEQ ID | Amino acid | Transporter | T6_CruGTR_GA | From | Capsella rubella |
| NO: 315 | sequence | | | | |
| | of | | | | |
| SEQ ID | DNA coding | Transporter | T6_CruGTR_GA | From | Capsella rubella |
| NO: 316 | sequence | | | | |
| | of | | | | |
| SEQ ID | Amino acid | Transporter | T7_PtrPOT_GA | From | Populus trichocarpa |
| NO: 317 | sequence | | | | |
| | of | | | | |
| SEQ ID | DNA coding | Transporter | T7_PtrPOT_GA | From | Populus trichocarpa |
| NO: 318 | sequence | | | | |
| | of | | | | |
| SEQ ID | Amino acid | Transporter | T8_BnaMFS_GA | From | Brassica napus |
| NO: 319 | sequence | | | | |
| | of | | | | |
| SEQ ID | DNA coding | Transporter | T8_BnaMFS_GA | From | Brassica napus |
| NO: 320 | sequence | | | | |
| | of | | | | |
| SEQ ID | Amino acid | Transporter | T10_BolGTR_GA | From | Brassica oleracea |
| NO: 321 | sequence | | | | var. oleracea |
| | of | | | | |
| SEQ ID | DNA coding | Transporter | T10_BolGTR_GA | From | Brassica oleracea |
| NO: 322 | sequence | | | | var. oleracea |
| | of | | | | |
| SEQ ID | Amino acid | Transporter | T11_AthGTR1_GA | From | Arabidopsis thaliana |
| NO: 323 | sequence | | | | |
| | of | | | | |
| SEQ ID | DNA coding | Transporter | T11_AthGTR1_GA | From | Arabidopsis thaliana |
| NO: 324 | sequence | | | | |
| | of | | | | |
| SEQ ID | Amino acid | Transporter | T12_PsoNPF1_GA | From | Papaver |
| NO: 325 | sequence | | | | somniferum |
| | of | | | | |
| SEQ ID | DNA coding | Transporter | T12_PsoNPF1_GA | From | Papaver |
| NO: 326 | sequence | | | | somniferum |
| | of | | | | |
| SEQ ID | Amino acid | Transporter | T14_PsoNPF3_GA | From | Papaver |
| NO: 327 | sequence | | | | somniferum |
| | of | | | | |
| SEQ ID | DNA coding | Transporter | T14_PsoNPF3_GA | From | Papaver |
| NO: 328 | sequence | | | | somniferum |
| | of | | | | |
| SEQ ID | Amino acid | Transporter | T15_PsoNPF4_GA | From | Papaver |
| NO: 329 | sequence | | | | somniferum |
| | of | | | | |
| SEQ ID | DNA coding | Transporter | T15_PsoNPF4_GA | From | Papaver |
| NO: 330 | sequence | | | | somniferum |
| | of | | | | |
| SEQ ID | Amino acid | Transporter | T17_PsoNPF6_GA | From | Papaver |
| NO: 331 | sequence | | | | somniferum |
| | of | | | | |
| SEQ ID | DNA coding | Transporter | T17_PsoNPF6_GA | From | Papaver |
| NO: 332 | sequence | | | | somniferum |
| | of | | | | |
| SEQ ID | Amino acid | Transporter | T18_PsoNPF7_GA | From | Papaver |
| NO: 333 | sequence | | | | somniferum |
| | of | | | | |
| SEQ ID | DNA coding | Transporter | T18_PsoNPF7_GA | From | Papaver |
| NO: 334 | sequence | | | | somniferum |
| | of | | | | |
| SEQ ID | Amino acid | Transporter | T19_RmiPTR2_GA | From | Rhizopus |
| NO: 335 | sequence | | | | microsporus |
| | of | | | | |
| SEQ ID | DNA coding | Transporter | T19_RmiPTR2_GA | From | Rhizopus |
| NO: 336 | sequence | | | | microsporus |
| | of | | | | |
| SEQ ID | Amino acid | Transporter | T20_RmiPTR2_v2_GA | From | Rhizopus |
| NO: 337 | sequence | | | | microsporus |
| | of | | | | |
| SEQ ID | DNA coding | Transporter | T20_RmiPTR2_v2_GA | From | Rhizopus |
| NO: 338 | sequence | | | | microsporus |
| | of | | | | |
| SEQ ID | Amino acid | Transporter | T21_RalPTR2_GA | From | Rozella allomycis |
| NO: 339 | sequence | | | | |
| | of | | | | |
| SEQ ID | DNA coding | Transporter | T21_RalPTR2_GA | From | Rozella allomycis |
| NO: 340 | sequence | | | | |
| | of | | | | |
| SEQ ID | Amino acid | Transporter | T22_CanPOT_GA | From | Catenaria |
| NO: 341 | sequence | | | | anguillulae |
| | of | | | | |
| SEQ ID | DNA coding | Transporter | T22_CanPOT_GA | From | Catenaria |
| NO: 342 | sequence | | | | anguillulae |
| | of | | | | |
| SEQ ID | Amino acid | Transporter | T23_ArePOT_GA | From | Absidia repens |
| NO: 343 | sequence | | | | |
| | of | | | | |
| SEQ ID | DNA coding | Transporter | T23_ArePOT_GA | From | Absidia repens |
| NO: 344 | sequence | | | | |
| | of | | | | |
| SEQ ID | Amino acid | Transporter | T24_SlyPTR2_GA | From | Stemphylium |
| NO: 345 | sequence | | | | lycopersici |
| | of | | | | |
| SEQ ID | DNA coding | Transporter | T24_SlyPTR2_GA | From | Stemphylium |
| NO: 346 | sequence | | | | lycopersici |
| | of | | | | |
| SEQ ID | Amino acid | Transporter | T25_AorPOT_GA | From | Aspergillus oryzae |
| NO: 347 | sequence | | | | |
| | of | | | | |
| SEQ ID | DNA coding | Transporter | T25_AorPOT_GA | From | Aspergillus oryzae |
| NO: 348 | sequence | | | | |
| | of | | | | |
| SEQ ID | Amino acid | Transporter | T26_NfuPOT_GA | From | Neosartorya |
| NO: 349 | sequence | | | | fumigata |
| | of | | | | |
| SEQ ID | DNA coding | Transporter | T26_NfuPOT_GA | From | Neosartorya |
| NO: 350 | sequence | | | | fumigata |
| | of | | | | |
| SEQ ID | Amino acid | Transporter | T27_FoxPOT_GA | From | Fusarium |
| NO: 351 | sequence | | | | oxysporum |
| | of | | | | |
| SEQ ID | DNA coding | Transporter | T27_FoxPOT_GA | From | Fusarium |
| NO: 352 | sequence | | | | oxysporum |
| | of | | | | |
| SEQ ID | Amino acid | Transporter | T28_MciPOT_GA | From | Mucor circinelloides |
| NO: 353 | sequence | | | | f. circinelloides |
| | of | | | | |
| SEQ ID | DNA coding | Transporter | T28_MciPOT_GA | From | Mucor circinelloides |
| NO: 354 | sequence | | | | f. circinelloides |
| | of | | | | |
| SEQ ID | Amino acid | Transporter | T29_AcaPOT_GA | From | Aspergillus |
| NO: 355 | sequence | | | | calidoustus |
| | of | | | | |
| SEQ ID | DNA coding | Transporter | T29_AcaPOT_GA | From | Aspergillus |
| NO: 356 | sequence | | | | calidoustus |
| | of | | | | |
| SEQ ID | Amino acid | Transporter | T30_MlyPOT_GA | From | Microbotryum |
| NO: 357 | sequence | | | | lychnidis-dioicae |
| | of | | | | |
| SEQ ID | DNA coding | Transporter | T30_MlyPOT_GA | From | Microbotryum |
| NO: 358 | sequence | | | | lychnidis-dioicae |
| | of | | | | |
| SEQ ID | Amino acid | Transporter | T31_TgaPOT_GA | From | Trichoderma gamsii |
| NO: 359 | sequence | | | | |
| | of | | | | |
| SEQ ID | DNA coding | Transporter | T31_TgaPOT_GA | From | Trichoderma gamsii |
| NO: 360 | sequence | | | | |
| | of | | | | |
| SEQ ID | Amino acid | Transporter | T32_AarPOT_GA | From | Aspergillus |
| NO: 361 | sequence | | | | arachidicola |
| | of | | | | |
| SEQ ID | DNA coding | Transporter | T32_AarPOT_GA | From | Aspergillus |
| NO: 362 | sequence | | | | arachidicola |
| | of | | | | |
| SEQ ID | Amino acid | Transporter | T33_CcuPTR2_GA | From | Choanephora |
| NO: 363 | sequence | | | | cucurbitarum |
| | of | | | | |
| SEQ ID | DNA coding | Transporter | T33_CcuPTR2_GA | From | Choanephora |
| NO: 364 | sequence | | | | cucurbitarum |
| | of | | | | |
| SEQ ID | Amino acid | Transporter | T34_HvePOT_GA | From | Hesseltinella |
| NO: 365 | sequence | | | | vesiculosa |
| | of | | | | |
| SEQ ID | DNA coding | Transporter | T34_HvePOT_GA | From | Hesseltinella |
| NO: 366 | sequence | | | | vesiculosa |
| | of | | | | |
| SEQ ID | Amino acid | Transporter | T35_EcuPOT_GA | From | Encephalitozoon |
| NO: 367 | sequence | | | | cuniculi |
| | of | | | | |
| SEQ ID | DNA coding | Transporter | T35_EcuPOT_GA | From | Encephalitozoon |
| NO: 368 | sequence | | | | cuniculi |
| | of | | | | |
| SEQ ID | Amino acid | Transporter | T36_RnePOT_GA | From | Rosellinia necatrix |
| NO: 369 | sequence | | | | |
| | of | | | | |
| SEQ ID | DNA coding | Transporter | T36_RnePOT_GA | From | Rosellinia necatrix |
| NO: 370 | sequence | | | | |
| | of | | | | |
| SEQ ID | Amino acid | Transporter | T37_OcoPOT_GA | From | Ordospora colligata |
| NO: 371 | sequence | | | | |
| | of | | | | |
| SEQ ID | DNA coding | Transporter | T37_OcoPOT_GA | From | Ordospora colligata |
| NO: 372 | sequence | | | | |
| | of | | | | |
| SEQ ID | Amino acid | Transporter | T38_ScuPTR2_GA | From | Smittium culicis |
| NO: 373 | sequence | | | | |
| | of | | | | |
| SEQ ID | DNA coding | Transporter | T38_ScuPTR2_GA | From | Smittium culicis |
| NO: 374 | sequence | | | | |
| | of | | | | |
| SEQ ID | Amino acid | Transporter | T39_CgrPOT_GA | From | Colletotrichum |
| NO: 375 | sequence | | | | graminicola |
| | of | | | | |
| SEQ ID | DNA coding | Transporter | T39_CgrPOT_GA | From | Colletotrichum |
| NO: 376 | sequence | | | | graminicola |
| | of | | | | |
| SEQ ID | Amino acid | Transporter | T40_EdePOT_GA | From | Exophiala |
| NO: 377 | sequence | | | | dermatitidis |
| | of | | | | |
| SEQ ID | DNA coding | Transporter | T40_EdePOT_GA | From | Exophiala |
| NO: 378 | sequence | | | | dermatitidis |
| | of | | | | |
| SEQ ID | Amino acid | Transporter | T41_CalPTR2_GA | From | Candida albicans |
| NO: 379 | sequence | | | | |
| | of | | | | |
| SEQ ID | DNA coding | Transporter | T41_CalPTR2_GA | From | Candida albicans |
| NO: 380 | sequence | | | | |
| | of | | | | |
| SEQ ID | Amino acid | Transporter | T44_CcaMFS_GA | From | Cajanus cajan |
| NO: 381 | sequence | | | | |
| | of | | | | |
| SEQ ID | DNA coding | Transporter | T44_CcaMFS_GA | From | Cajanus cajan |
| NO: 382 | sequence | | | | |
| | of | | | | |
| SEQ ID | Amino acid | Transporter | T45_PanPOT_GA | From | Parasponia |
| NO: 383 | sequence | | | | andersonii |
| | of | | | | |
| SEQ ID | DNA coding | Transporter | T45_PanPOT_GA | From | Parasponia |
| NO: 384 | sequence | | | | andersonii |
| | of | | | | |
| SEQ ID | Amino acid | Transporter | T46_RchPOT_GA | From | Rosa chinensis |
| NO: 385 | sequence | | | | |
| | of | | | | |
| SEQ ID | DNA coding | Transporter | T46_RchPOT_GA | From | Rosa chinensis |
| NO: 386 | sequence | | | | |
| | of | | | | |
| SEQ ID | Amino acid | Transporter | T47_PbeNPF_GA | From | Pyrus betulifolia |
| NO: 387 | sequence | | | | |
| | of | | | | |
| SEQ ID | DNA coding | Transporter | T47_PbeNPF_GA | From | Pyrus betulifolia |
| NO: 388 | sequence | | | | |
| | of | | | | |
| SEQ ID | Amino acid | Transporter | T48_CcaPOT_GA | From | Corchorus capsularis |
| NO: 389 | sequence | | | | |
| | of | | | | |
| SEQ ID | DNA coding | Transporter | T48_CcaPOT_GA | From | Corchorus capsularis |
| NO: 390 | sequence | | | | |
| | of | | | | |
| SEQ ID | Amino acid | Transporter | T49_HanPOT_GA | From | Helianthus annuus |
| NO: 391 | sequence | | | | |
| | of | | | | |
| SEQ ID | DNA coding | Transporter | T49_HanPOT_GA | From | Helianthus annuus |
| NO: 392 | sequence | | | | |
| | of | | | | |
| SEQ ID | Amino acid | Transporter | T50_HimPOT_GA | From | Handroanthus |
| NO: 393 | sequence | | | | impetiginosus |
| | of | | | | |
| SEQ ID | DNA coding | Transporter | T50_HimPOT_GA | From | Handroanthus |
| NO: 394 | sequence | | | | impetiginosus |
| | of | | | | |
| SEQ ID | Amino acid | Transporter | T51_TorPOT_GA | From | Trema orientalis |
| NO: 395 | sequence | | | | |
| | of | | | | |
| SEQ ID | DNA coding | Transporter | T51_TorPOT_GA | From | Trema orientalis |
| NO: 396 | sequence | | | | |
| | of | | | | |
| SEQ ID | Amino acid | Transporter | T52_BmePTR2_GA | From | Basidiobolus |
| NO: 397 | sequence | | | | meristosporus |
| | of | | | | |
| SEQ ID | DNA coding | Transporter | T52_BmePTR2_GA | From | Basidiobolus |
| NO: 398 | sequence | | | | meristosporus |
| | of | | | | |
| SEQ ID | Amino acid | Transporter | T53_EhePOT_GA | From | Encephalitozoon |
| NO: 399 | sequence | | | | hellem |
| | of | | | | |
| SEQ ID | DNA coding | Transporter | T53_EhePOT_GA | From | Encephalitozoon |
| NO: 400 | sequence | | | | hellem |
| | of | | | | |
| SEQ ID | Amino acid | Transporter | T54_MelPOT_GA | From | Mortierella elongata |
| NO: 401 | sequence | | | | |
| | of | | | | |
| SEQ ID | DNA coding | Transporter | T54_MelPOT_GA | From | Mortierella elongata |
| NO: 402 | sequence | | | | |
| | of | | | | |
| SEQ ID | Amino acid | Transporter | T55_NsyNPF_GA | From | Nicotiana sylvestris |
| NO: 403 | sequence | | | | |
| | of | | | | |
| SEQ ID | DNA coding | Transporter | T55_NsyNPF_GA | From | Nicotiana sylvestris |
| NO: 404 | sequence | | | | |
| | of | | | | |
| SEQ ID | Amino acid | Transporter | T56_CanNPF_GA | From | Capsicum annuum |
| NO: 405 | sequence | | | | |
| | of | | | | |
| SEQ ID | DNA coding | Transporter | T56_CanNPF_GA | From | Capsicum annuum |
| NO: 406 | sequence | | | | |
| | of | | | | |
| SEQ ID | Amino acid | Transporter | T57_AcoNPF_GA | From | Aquilegia coerulea |
| NO: 407 | sequence | | | | |
| | of | | | | |
| SEQ ID | DNA coding | Transporter | T57_AcoNPF_GA | From | Aquilegia coerulea |
| NO: 408 | sequence | | | | |
| | of | | | | |
| SEQ ID | Amino acid | Transporter | T59_AmeNPF1_GA | From | Argemone mexican |
| NO: 409 | sequence | | | | |
| | of | | | | |
| SEQ ID | DNA coding | Transporter | T59_AmeNPF1_GA | From | Argemone mexican |
| NO: 410 | sequence | | | | |
| | of | | | | |
| SEQ ID | Amino acid | Transporter | T60_AmeNPF2_GA | From | Argemone mexican |
| NO: 411 | sequence | | | | |
| | of | | | | |
| SEQ ID | DNA coding | Transporter | T60_AmeNPF2_GA | From | Argemone mexican |
| NO: 412 | sequence | | | | |
| | of | | | | |
| SEQ ID | Amino acid | Transporter | T61_TwiNPF_GA | From | Tripterygium |
| NO: 413 | sequence | | | | wilfordii |
| | of | | | | |
| SEQ ID | DNA coding | Transporter | T61_TwiNPF_GA | From | Tripterygium |
| NO: 414 | sequence | | | | wilfordii |
| | of | | | | |
| SEQ ID | Amino acid | Transporter | T62_SmaNPF_GA | From | Swietenia mahagoni |
| NO: 415 | sequence | | | | |
| | of | | | | |
| SEQ ID | DNA coding | Transporter | T62_SmaNPF_GA | From | Swietenia mahagoni |
| NO: 416 | sequence | | | | |
| | of | | | | |
| SEQ ID | Amino acid | Transporter | T63_CfoNPF_GA | From | Coleus forskohlii |
| NO: 417 | sequence | | | | |
| | of | | | | |
| SEQ ID | DNA coding | Transporter | T63_CfoNPF_GA | From | Coleus forskohlii |
| NO: 418 | sequence | | | | |
| | of | | | | |
| SEQ ID | Amino acid | Transporter | T64_XsiNPF_GA | From | Xanthorhiza |
| NO: 419 | sequence | | | | simplicissima |
| | of | | | | |
| SEQ ID | DNA coding | Transporter | T64_XsiNPF_GA | From | Xanthorhiza |
| NO: 420 | sequence | | | | simplicissima |
| | of | | | | |
| SEQ ID | Amino acid | Transporter | T66_TelNPF_GA | From | Tabernaemontana |
| NO: 421 | sequence | | | | elegans |
| | of | | | | |
| SEQ ID | DNA coding | Transporter | T66_TelNPF_GA | From | Tabernaemontana |
| NO: 422 | sequence | | | | elegans |
| | of | | | | |
| SEQ ID | Amino acid | Transporter | T67_SdiNPF_GA | From | Stylophorum |
| NO: 423 | sequence | | | | diphyllum |
| | of | | | | |
| SEQ ID | DNA coding | Transporter | T67_SdiNPF_GA | From | Stylophorum |
| NO: 424 | sequence | | | | diphyllum |
| | of | | | | |
| SEQ ID | Amino acid | Transporter | T68_RseNPF_GA | From | Rauwolfia |
| NO: 425 | sequence | | | | serpentina |
| | of | | | | |
| SEQ ID | DNA coding | Transporter | T68_RseNPF_GA | From | Rauwolfia |
| NO: 426 | sequence | | | | serpentina |
| | of | | | | |
| SEQ ID | Amino acid | Transporter | T69_PhoNPF_GA | From | pelargonium × |
| NO: 427 | sequence | | | | hortorum |
| | of | | | | |
| SEQ ID | DNA coding | Transporter | T69_PhoNPF_GA | From | pelargonium × |
| NO: 428 | sequence | | | | hortorum |
| | of | | | | |
| SEQ ID | Amino acid | Transporter | T70_CmaNPF_GA | From | Chelidonium majus |
| NO: 429 | sequence | | | | |
| | of | | | | |
| SEQ ID | DNA coding | Transporter | T70_CmaNPF_GA | From | Chelidonium majus |
| NO: 430 | sequence | | | | |
| | of | | | | |
| SEQ ID | Amino acid | Transporter | T71_CchNPF_GA | From | Corydalis |
| NO: 431 | sequence | | | | chelanthifolia |
| | of | | | | |
| SEQ ID | DNA coding | Transporter | T71_CchNPF_GA | From | Corydalis |
| NO: 432 | sequence | | | | chelanthifolia |
| | of | | | | |
| SEQ ID | Amino acid | Transporter | T72_TcoNPF_GA | From | Tinospora_cordifolia |
| NO: 433 | sequence | | | | |
| | of | | | | |
| SEQ ID | DNA coding | Transporter | T72_TcoNPF_GA | From | Tinospora_cordifolia |
| NO: 434 | sequence | | | | |
| | of | | | | |
| SEQ ID | Amino acid | Transporter | T73_PbrNPF1_GA | From | Papaver bracteatum |
| NO: 435 | sequence | | | | |
| | of | | | | |
| SEQ ID | DNA coding | Transporter | T73_PbrNPF1_GA | From | Papaver bracteatum |
| NO: 436 | sequence | | | | |
| | of | | | | |
| SEQ ID | Amino acid | Transporter | T74_PbrNPF2_GA | From | Papaver bracteatum |
| NO: 437 | sequence | | | | |
| | of | | | | |
| SEQ ID | DNA coding | Transporter | T74_PbrNPF2_GA | From | Papaver bracteatum |
| NO: 438 | sequence | | | | |
| | of | | | | |
| SEQ ID | Amino acid | Transporter | T75_PbrNPF3_GA | From | Papaver bracteatum |
| NO: 439 | sequence | | | | |
| | of | | | | |
| SEQ ID | DNA coding | Transporter | T75_PbrNPF3_GA | From | Papaver bracteatum |
| NO: 440 | sequence | | | | |
| | of | | | | |
| SEQ ID | Amino acid | Transporter | T76_AhuNPF_GA | From | Amsonia hubrichtii |
| NO: 441 | sequence | | | | |
| | of | | | | |
| SEQ ID | DNA coding | Transporter | T76_AhuNPF_GA | From | Amsonia hubrichtii |
| NO: 442 | sequence | | | | |
| | of | | | | |
| SEQ ID | Amino acid | Transporter | T77_POCNPF_GA | From | Platanus |
| NO: 443 | sequence | | | | occidentalis |
| | of | | | | |
| SEQ ID | DNA coding | Transporter | T77_PocNPF_GA | From | Platanus |
| NO: 444 | sequence | | | | occidentalis |
| | of | | | | |
| SEQ ID | Amino acid | Transporter | T78_VofNPF_GA | From | Valeriana officinalis |
| NO: 445 | sequence | | | | |
| | of | | | | |
| SEQ ID | DNA coding | Transporter | T78_VofNPF_GA | From | Valeriana officinalis |
| NO: 446 | sequence | | | | |
| | of | | | | |
| SEQ ID | Amino acid | Transporter | T79_EcaNPF_GA | From | Eschscholzia |
| NO: 447 | sequence | | | | californica |
| | of | | | | |
| SEQ ID | DNA coding | Transporter | T79_EcaNPF_GA | From | Eschscholzia |
| NO: 448 | sequence | | | | californica |
| | of | | | | |
| SEQ ID | Amino acid | Transporter | T80_CroNPF_GA | From | Catharanthus roseus |
| NO: 449 | sequence | | | | |
| | of | | | | |
| SEQ ID | DNA coding | Transporter | T80_CroNPF_GA | From | Catharanthus roseus |
| NO: 450 | sequence | | | | |
| | of | | | | |
| SEQ ID | Amino acid | Transporter | T81_HcaNPF_GA | From | Hypericum |
| NO: 451 | sequence | | | | perforatum |
| | of | | | | |
| SEQ ID | DNA coding | Transporter | T81_HcaNPF_GA | From | Hypericum |
| NO: 452 | sequence | | | | perforatum |
| | of | | | | |
| SEQ ID | Amino acid | Transporter | T82_NsaNPF_GA | From | Nigella sativa |
| NO: 453 | sequence | | | | |
| | of | | | | |
| SEQ ID | DNA coding | Transporter | T82_NsaNPF_GA | From | Nigella sativa |
| NO: 454 | sequence | | | | |
| | of | | | | |
| SEQ ID | Amino acid | Transporter | T83_ScaNPF_GA | From | Sanguinaria |
| NO: 455 | sequence | | | | canadensis |
| | of | | | | |
| SEQ ID | DNA coding | Transporter | T83_ScaNPF_GA | From | Sanguinaria |
| NO: 456 | sequence | | | | canadensis |
| | of | | | | |
| SEQ ID | Amino acid | Transporter | T84_TflNPF_GA | From | Thalictrum flavum |
| NO: 457 | sequence | | | | |
| | of | | | | |
| SEQ ID | DNA coding | Transporter | T84_TflNPF_GA | From | Thalictrum flavum |
| NO: 458 | sequence | | | | |
| | of | | | | |
| SEQ ID | Amino acid | Transporter | T85_GflNPF_GA | From | Glaucium Flavum |
| NO: 459 | sequence | | | | |
| | of | | | | |
| SEQ ID | DNA coding | Transporter | T85_GflNPF_GA | From | Glaucium Flavum |
| NO: 460 | sequence | | | | |
| | of | | | | |
| SEQ ID | Amino acid | Transporter | T97_ScaT14_GA | From | Sanguinaria |
| NO: 461 | sequence | | | | canadensis |
| | of | | | | |
| SEQ ID | DNA coding | Transporter | T97_ScaT14_GA | From | Sanguinaria |
| NO: 462 | sequence | | | | canadensis |
| | of | | | | |
| SEQ ID | Amino acid | Transporter | T101_McoPUP3_1 | From | Macleaya cordata |
| NO: 463 | sequence | | | | |
| | of | | | | |
| SEQ ID | DNA coding | Transporter | T101_McoPUP3_1 | From | Macleaya cordata |
| NO: 464 | sequence | | | | |
| | of | | | | |
| SEQ ID | Amino acid | Transporter | T102_PsoPUP3_1 | From | Papaver |
| NO: 465 | sequence | | | | somniferum |
| | of | | | | |
| SEQ ID | DNA coding | Transporter | T102_PsoPUP3_1 | From | Papaver |
| NO: 466 | sequence | | | | somniferum |
| | of | | | | |
| SEQ ID | Amino acid | Transporter | T103_PsoPUP3_2 | From | Papaver |
| NO: 467 | sequence | | | | somniferum |
| | of | | | | |
| SEQ ID | DNA coding | Transporter | T103_PsoPUP3_2 | From | Papaver |
| NO: 468 | sequence | | | | somniferum |
| | of | | | | |
| SEQ ID | Amino acid | Transporter | T104_PsoPUP3_3 | From | Papaver |
| NO: 469 | sequence | | | | somniferum |
| | of | | | | |
| SEQ ID | DNA coding | Transporter | T104_PsoPUP3_3 | From | Papaver |
| NO: 470 | sequence | | | | somniferum |
| | of | | | | |
| SEQ ID | Amino acid | Transporter | T105_PsoPUP-L | From | Papaver |
| NO: 471 | sequence | | | | somniferum |
| | of | | | | |
| SEQ ID | DNA coding | Transporter | T105_PsoPUP-L | From | Papaver |
| NO: 472 | sequence | | | | somniferum |
| | of | | | | |
| SEQ ID | Amino acid | Transporter | T109_GflPUP3_83 | From | Glaucium Flavum |
| NO: 473 | sequence | | | | |
| | of | | | | |
| SEQ ID | DNA coding | Transporter | T109_GflPUP3_83 | From | Glaucium Flavum |
| NO: 474 | sequence | | | | |
| | of | | | | |
| SEQ ID | Amino acid | Transporter | T113_PsoPUP3_32 | From | Papaver |
| NO: 475 | sequence | | | | somniferum |
| | of | | | | |
| SEQ ID | DNA coding | Transporter | T113_PsoPUP3_32 | From | Papaver |
| NO: 476 | sequence | | | | somniferum |
| | of | | | | |
| SEQ ID | Amino acid | Transporter | T114_TorPUP3_40 | From | Trema orientale |
| NO: 477 | sequence | | | | |
| | of | | | | |
| SEQ ID | DNA coding | Transporter | T114_TorPUP3_40 | From | Trema orientale |
| NO: 478 | sequence | | | | |
| | of | | | | |
| SEQ ID | Amino acid | Transporter | T115_CsaPUP3_48 | From | Cucumis sativus |
| NO: 479 | sequence | | | | |
| | of | | | | |
| SEQ ID | DNA coding | Transporter | T115_CsaPUP3_48 | From | Cucumis sativus |
| NO: 480 | sequence | | | | |
| | of | | | | |
| SEQ ID | Amino acid | Transporter | T116_HanPUP3_56 | From | Helianthus annuus |
| NO: 481 | sequence | | | | |
| | of | | | | |
| SEQ ID | DNA coding | Transporter | T116_HanPUP3_56 | From | Helianthus annuus |
| NO: 482 | sequence | | | | |
| | of | | | | |
| SEQ ID | Amino acid | Transporter | T117_MacPUP3_64 | From | Musa acuminata |
| NO: 483 | sequence | | | | |
| | of | | | | |
| SEQ ID | DNA coding | Transporter | T117_MacPUP3_64 | From | Musa acuminata |
| NO: 484 | sequence | | | | |
| | of | | | | |
| SEQ ID | Amino acid | Transporter | T121_NnuPUP3_9 | From | Nelumbo nucifera |
| NO: 485 | sequence | | | | |
| | of | | | | |
| SEQ ID | DNA coding | Transporter | T121_NnuPUP3_9 | From | Nelumbo nucifera |
| NO: 486 | sequence | | | | |
| | of | | | | |
| SEQ ID | Amino acid | Transporter | T122_PsoPUP3_17 | From | Papaver |
| NO: 487 | sequence | | | | somniferum |
| | of | | | | |
| SEQ ID | DNA coding | Transporter | T122_PsoPUP3_17 | From | Papaver |
| NO: 488 | sequence | | | | somniferum |
| | of | | | | |
| SEQ ID | Amino acid | Transporter | T123_PsoPUP3_25 | From | Papaver |
| NO: 489 | sequence | | | | somniferum |
| | of | | | | |
| SEQ ID | DNA coding | Transporter | T123_PsoPUP3_25 | From | Papaver |
| NO: 490 | sequence | | | | somniferum |
| | of | | | | |
| SEQ ID | Amino acid | Transporter | T124_PsoPUP3_33 | From | Papaver |
| NO: 491 | sequence | | | | somniferum |
| | of | | | | |
| SEQ ID | DNA coding | Transporter | T124_PsoPUP3_33 | From | Papaver |
| NO: 492 | sequence | | | | somniferum |
| | of | | | | |
| SEQ ID | Amino acid | Transporter | T125_JcuPUP3_41 | From | Jatropha curcas |
| NO: 493 | sequence | | | | |
| | of | | | | |
| SEQ ID | DNA coding | Transporter | T125_JcuPUP3_41 | From | Jatropha curcas |
| NO: 494 | sequence | | | | |
| | of | | | | |
| SEQ ID | Amino acid | Transporter | T126_CpePUP3_49 | From | Cucurbita pepo |
| NO: 495 | sequence | | | | subsp. pepo |
| | of | | | | |
| SEQ ID | DNA coding | Transporter | T126_CpePUP3_49 | From | Cucurbita pepo |
| NO: 496 | sequence | | | | subsp. pepo |
| | of | | | | |
| SEQ ID | Amino acid | Transporter | T127_LsaPUP3_57 | From | Lactuca sativa |
| NO: 497 | sequence | | | | |
| | of | | | | |
| SEQ ID | DNA coding | Transporter | T127_LsaPUP3_57 | From | Lactuca sativa |
| NO: 498 | sequence | | | | |
| | of | | | | |
| SEQ ID | Amino acid | Transporter | T128_PsoPUP3_65 | From | Papaver |
| NO: 499 | sequence | | | | somniferum |
| | of | | | | |
| SEQ ID | DNA coding | Transporter | T128_PsoPUP3_65 | From | Papaver |
| NO: 500 | sequence | | | | somniferum |
| | of | | | | |
| SEQ ID | Amino acid | Transporter | T129_PsoPUP3_73 | From | Papaver |
| NO: 501 | sequence | | | | somniferum |
| | of | | | | |
| SEQ ID | DNA coding | Transporter | T129_PsoPUP3_73 | From | Papaver |
| NO: 502 | sequence | | | | somniferum |
| | of | | | | |
| SEQ ID | Amino acid | Transporter | T130_NdoPUP3_89 | From | Nandina domestica |
| NO: 503 | sequence | | | | |
| | of | | | | |
| SEQ ID | DNA coding | Transporter | T130_NdoPUP3_89 | From | Nandina domestica |
| NO: 504 | sequence | | | | |
| | of | | | | |
| SEQ ID | Amino acid | Transporter | T131_PbrPUP3_81 | From | Papaver bracteatum |
| NO: 505 | sequence | | | | |
| | of | | | | |
| SEQ ID | DNA coding | Transporter | T131_PbrPUP3_81 | From | Papaver bracteatum |
| NO: 506 | sequence | | | | |
| | of | | | | |
| SEQ ID | Amino acid | Transporter | T132_CmiPUP3_10 | From | Cinnamomum |
| NO: 507 | sequence | | | | micranthum f. |
| | of | | | | kanehirae |
| SEQ ID | DNA coding | Transporter | T132_CmiPUP3_10 | From | Cinnamomum |
| NO: 508 | sequence | | | | micranthum f. |
| | of | | | | kanehirae |
| SEQ ID | Amino acid | Transporter | T133_PsoPUP3_18 | From | Papaver |
| NO: 509 | sequence | | | | somniferum |
| | of | | | | |
| SEQ ID | DNA coding | Transporter | T133_PsoPUP3_18 | From | Papaver |
| NO: 510 | sequence | | | | somniferum |
| | of | | | | |
| SEQ ID | Amino acid | Transporter | T135_PsoPUP_34 | From | Papaver |
| NO: 511 | sequence | | | | somniferum |
| | of | | | | |
| SEQ ID | DNA coding | Transporter | T135_PsoPUP_34 | From | Papaver |
| NO: 512 | sequence | | | | somniferum |
| | of | | | | |
| SEQ ID | Amino acid | Transporter | T136_RchPUP3_42 | From | Rosa chinensis |
| NO: 513 | sequence | | | | |
| | of | | | | |
| SEQ ID | DNA coding | Transporter | T136_RchPUP3_42 | From | Rosa chinensis |
| NO: 514 | sequence | | | | |
| | of | | | | |
| SEQ ID | Amino acid | Transporter | T137_EguPUP3_50 | From | Erythranthe guttata |
| NO: 515 | sequence | | | | |
| | of | | | | |
| SEQ ID | DNA coding | Transporter | T137_EguPUP3_50 | From | Erythranthe guttata |
| NO: 516 | sequence | | | | |
| | of | | | | |
| SEQ ID | Amino acid | Transporter | T138_AduPUP3_58 | From | Arachis duranensis |
| NO: 517 | sequence | | | | |
| | of | | | | |
| SEQ ID | DNA coding | Transporter | T138_AduPUP3_58 | From | Arachis duranensis |
| NO: 518 | sequence | | | | |
| | of | | | | |
| SEQ ID | Amino acid | Transporter | T139_PsoPUP3_66 | From | Papaver |
| NO: 519 | sequence | | | | somniferum |
| | of | | | | |
| SEQ ID | DNA coding | Transporter | T139_PsoPUP3_66 | From | Papaver |
| NO: 520 | sequence | | | | somniferum |
| | of | | | | |
| SEQ ID | Amino acid | Transporter | T140_PalPUP3_74 | From | Papaver alpinum |
| NO: 521 | sequence | | | | |
| | of | | | | |
| SEQ ID | DNA coding | Transporter | T140_PalPUP3_74 | From | Papaver alpinum |
| NO: 522 | sequence | | | | |
| | of | | | | |
| SEQ ID | Amino acid | Transporter | T141_EcaPUP3_88 | From | Eschscholzia |
| NO: 523 | sequence | | | | californica |
| | of | | | | |
| SEQ ID | DNA coding | Transporter | T141_EcaPUP3_88 | From | Eschscholzia |
| NO: 524 | sequence | | | | californica |
| | of | | | | |
| SEQ ID | Amino acid | Transporter | T142_McoPUP3_4 | From | Macleaya cordata |
| NO: 525 | sequence | | | | |
| | of | | | | |
| SEQ ID | DNA coding | Transporter | T142_McoPUP3_4 | From | Macleaya cordata |
| NO: 526 | sequence | | | | |
| | of | | | | |
| SEQ ID | Amino acid | Transporter | T143_CmiPUP3_11 | From | Cinnamomum |
| NO: 527 | sequence | | | | micranthum f. |
| | of | | | | kanehirae |
| SEQ ID | DNA coding | Transporter | T143_CmiPUP3_11 | From | Cinnamomum |
| NO: 528 | sequence | | | | micranthum f. |
| | of | | | | kanehirae |
| SEQ ID | Amino acid | Transporter | T144_PsoPUP3_19 | From | Papaver |
| NO: 529 | sequence | | | | somniferum |
| | of | | | | |
| SEQ ID | DNA coding | Transporter | T144_PsoPUP3_19 | From | Papaver |
| NO: 530 | sequence | | | | somniferum |
| | of | | | | |
| SEQ ID | Amino acid | Transporter | T146_PsoPUP_35 | From | Papaver |
| NO: 531 | sequence | | | | somniferum |
| | of | | | | |
| SEQ ID | DNA coding | Transporter | T146_PsoPUP_35 | From | Papaver |
| NO: 532 | sequence | | | | somniferum |
| | of | | | | |
| SEQ ID | Amino acid | Transporter | T147_MesPUP3_43 | From | Manihot esculenta |
| NO: 533 | sequence | | | | |
| | of | | | | |
| SEQ ID | DNA coding | Transporter | T147_MesPUP3_43 | From | Manihot esculenta |
| NO: 534 | sequence | | | | |
| | of | | | | |
| SEQ ID | Amino acid | Transporter | T148_HimPUP3_51 | From | Handroanthus |
| NO: 535 | sequence | | | | impetiginosus |
| | of | | | | |
| SEQ ID | DNA coding | Transporter | T148_HimPUP3_51 | From | Handroanthus |
| NO: 536 | sequence | | | | impetiginosus |
| | of | | | | |
| SEQ ID | Amino acid | Transporter | T149_AcoPUP3_59 | From | Aquilegia coerulea |
| NO: 537 | sequence | | | | |
| | of | | | | |
| SEQ ID | DNA coding | Transporter | T149_AcoPUP3_59 | From | Aquilegia coerulea |
| NO: 538 | sequence | | | | |
| | of | | | | |
| SEQ ID | Amino acid | Transporter | T150_PsoPUP3_67 | From | Papaver |
| NO: 539 | sequence | | | | somniferum |
| | of | | | | |
| SEQ ID | DNA coding | Transporter | T150_PsoPUP3_67 | From | Papaver |
| NO: 540 | sequence | | | | somniferum |
| | of | | | | |
| SEQ ID | Amino acid | Transporter | T151_PatPUP3_75 | From | Papaver atlanticum |
| NO: 541 | sequence | | | | |
| | of | | | | |
| SEQ ID | DNA coding | Transporter | T151_PatPUP3_75 | From | Papaver atlanticum |
| NO: 542 | sequence | | | | |
| | of | | | | |
| SEQ ID | Amino acid | Transporter | T152_GflPUP3_87 | From | Glaucium Flavum |
| NO: 543 | sequence | | | | |
| | of | | | | |
| SEQ ID | DNA coding | Transporter | T152_GflPUP3_87 | From | Glaucium Flavum |
| NO: 544 | sequence | | | | |
| | of | | | | |
| SEQ ID | Amino acid | Transporter | T153_PsoPUP3_5 | From | Papaver |
| NO: 545 | sequence | | | | somniferum |
| | of | | | | |
| SEQ ID | DNA coding | Transporter | T153_PsoPUP3_5 | From | Papaver |
| NO: 546 | sequence | | | | somniferum |
| | of | | | | |
| SEQ ID | Amino acid | Transporter | T154_CmiPUP3_12 | From | Cinnamomum |
| NO: 547 | sequence | | | | micranthum f. |
| | of | | | | kanehirae |
| SEQ ID | DNA coding | Transporter | T154_CmiPUP3_12 | From | Cinnamomum |
| NO: 548 | sequence | | | | micranthum f. |
| | of | | | | kanehirae |
| SEQ ID | Amino acid | Transporter | T156_PsoPUP3_28 | From | Papaver |
| NO: 549 | sequence | | | | somniferum |
| | of | | | | |
| SEQ ID | DNA coding | Transporter | T156_PsoPUP3_28 | From | Papaver |
| NO: 550 | sequence | | | | somniferum |
| | of | | | | |
| SEQ ID | Amino acid | Transporter | T157_RchPUP_36 | From | Rosa chinensis |
| NO: 551 | sequence | | | | |
| | of | | | | |
| SEQ ID | DNA coding | Transporter | T157_RchPUP_36 | From | Rosa chinensis |
| NO: 552 | sequence | | | | |
| | of | | | | |
| SEQ ID | Amino acid | Transporter | T158_DziPUP3_44 | From | Durio zibethinus |
| NO: 553 | sequence | | | | |
| | of | | | | |
| SEQ ID | DNA coding | Transporter | T158_DziPUP3_44 | From | Durio zibethinus |
| NO: 554 | sequence | | | | |
| | of | | | | |
| SEQ ID | Amino acid | Transporter | T159_OeuPUP3_52 | From | Olea europaea var. |
| NO: 555 | sequence | | | | sylvestris |
| | of | | | | |
| SEQ ID | DNA coding | Transporter | T159_OeuPUP3_52 | From | Olea europaea var. |
| NO: 556 | sequence | | | | sylvestris |
| | of | | | | |
| SEQ ID | Amino acid | Transporter | T160_CeuPUP3_60 | From | Coffea eugenioides |
| NO: 557 | sequence | | | | |
| | of | | | | |
| SEQ ID | DNA coding | Transporter | T160_CeuPUP3_60 | From | Coffea eugenioides |
| NO: 558 | sequence | | | | |
| | of | | | | |
| SEQ ID | Amino acid | Transporter | T161_PsoPUP3_68 | From | Papaver |
| NO: 559 | sequence | | | | somniferum |
| | of | | | | |
| SEQ ID | DNA coding | Transporter | T161_PsoPUP3_68 | From | Papaver |
| NO: 560 | sequence | | | | somniferum |
| | of | | | | |
| SEQ ID | Amino acid | Transporter | T162_PmiPUP3_76 | From | Papaver |
| NO: 561 | sequence | | | | miyabeanum |
| | of | | | | |
| SEQ ID | DNA coding | Transporter | T162_PmiPUP3_76 | From | Papaver |
| NO: 562 | sequence | | | | miyabeanum |
| | of | | | | |
| SEQ ID | Amino acid | Transporter | T163_PbrPUP3_86 | From | Papaver bracteatum |
| NO: 563 | sequence | | | | |
| | of | | | | |
| SEQ ID | DNA coding | Transporter | T163_PbrPUP3_86 | From | Papaver bracteatum |
| NO: 564 | sequence | | | | |
| | of | | | | |
| SEQ ID | Amino acid | Transporter | T164_PsoPUP3_78 | From | Papaver |
| NO: 565 | sequence | | | | somniferum |
| | of | | | | |
| SEQ ID | DNA coding | Transporter | T164_PsoPUP3_78 | From | Papaver |
| NO: 566 | sequence | | | | somniferum |
| | of | | | | |
| SEQ ID | Amino acid | Transporter | T165_AcoPUP3_13 | From | Aquilegia coerulea |
| NO: 567 | sequence | | | | |
| | of | | | | |
| SEQ ID | DNA coding | Transporter | T165_AcoPUP3_13 | From | Aquilegia coerulea |
| NO: 568 | sequence | | | | |
| | of | | | | |
| SEQ ID | Amino acid | Transporter | T166_PsoPUP3_21 | From | Papaver |
| NO: 569 | sequence | | | | somniferum |
| | of | | | | |
| SEQ ID | DNA coding | Transporter | T166_PsoPUP3_21 | From | Papaver |
| NO: 570 | sequence | | | | somniferum |
| | of | | | | |
| SEQ ID | Amino acid | Transporter | T168_FvePUP3_37 | From | Fragaria vesca |
| NO: 571 | sequence | | | | subsp. vesca |
| | of | | | | |
| SEQ ID | DNA coding | Transporter | T168_FvePUP3_37 | From | Fragaria vesca |
| NO: 572 | sequence | | | | subsp. vesca |
| | of | | | | |
| SEQ ID | Amino acid | Transporter | T169_ZjuPUP3_45 | From | Ziziphus jujuba |
| NO: 573 | sequence | | | | |
| | of | | | | |
| SEQ ID | DNA coding | Transporter | T169_ZjuPUP3_45 | From | Ziziphus jujuba |
| NO: 574 | sequence | | | | |
| | of | | | | |
| SEQ ID | Amino acid | Transporter | T170_LsaPUP3_53 | From | Lactuca sativa |
| NO: 575 | sequence | | | | |
| | of | | | | |
| SEQ ID | DNA coding | Transporter | T170_LsaPUP3_53 | From | Lactuca sativa |
| NO: 576 | sequence | | | | |
| | of | | | | |
| SEQ ID | Amino acid | Transporter | T171_McoPUP3_61 | From | Macleaya cordata |
| NO: 577 | sequence | | | | |
| | of | | | | |
| SEQ ID | DNA coding | Transporter | T171_McoPUP3_61 | From | Macleaya cordata |
| NO: 578 | sequence | | | | |
| | of | | | | |
| SEQ ID | Amino acid | Transporter | T172_AcoPUP3_69 | From | Aquilegia coerulea |
| NO: 579 | sequence | | | | |
| | of | | | | |
| SEQ ID | DNA coding | Transporter | T172_AcoPUP3_69 | From | Aquilegia coerulea |
| NO: 580 | sequence | | | | |
| | of | | | | |
| SEQ ID | Amino acid | Transporter | T173_PnuPUP3_77 | From | Papaver nudicale |
| NO: 581 | sequence | | | | |
| | of | | | | |
| SEQ ID | DNA coding | Transporter | T173_PnuPUP3_77 | From | Papaver nudicale |
| NO: 582 | sequence | | | | |
| | of | | | | |
| SEQ ID | Amino acid | Transporter | T174_PbrPUP3_85 | From | Papaver bracteatum |
| NO: 583 | sequence | | | | |
| | of | | | | |
| SEQ ID | DNA coding | Transporter | T174_PbrPUP3_85 | From | Papaver bracteatum |
| NO: 584 | sequence | | | | |
| | of | | | | |
| SEQ ID | Amino acid | Transporter | T175_PsoPUP3_6 | From | Papaver |
| NO: 585 | sequence | | | | somniferum |
| | of | | | | |
| SEQ ID | DNA coding | Transporter | T175_PsoPUP3_6 | From | Papaver |
| NO: 586 | sequence | | | | somniferum |
| | of | | | | |
| SEQ ID | Amino acid | Transporter | T176_AcoPUP3_14 | From | Aquilegia coerulea |
| NO: 587 | sequence | | | | |
| | of | | | | |
| SEQ ID | DNA coding | Transporter | T176_AcoPUP3_14 | From | Aquilegia coerulea |
| NO: 588 | sequence | | | | |
| | of | | | | |
| SEQ ID | Amino acid | Transporter | T177_PsoPUP3_22 | From | Papaver |
| NO: 589 | sequence | | | | somniferum |
| | of | | | | |
| SEQ ID | DNA coding | Transporter | T177_PsoPUP3_22 | From | Papaver |
| NO: 590 | sequence | | | | somniferum |
| | of | | | | |
| SEQ ID | Amino acid | Transporter | T178_PsoPUP3_30 | From | Papaver |
| NO: 591 | sequence | | | | somniferum |
| | of | | | | |
| SEQ ID | DNA coding | Transporter | T178_PsoPUP3_30 | From | Papaver |
| NO: 592 | sequence | | | | somniferum |
| | of | | | | |
| SEQ ID | Amino acid | Transporter | T179_PyePUP3_38 | From | Prunus yedoensis |
| NO: 593 | sequence | | | | var. nudiflora |
| | of | | | | |
| SEQ ID | DNA coding | Transporter | T179_PyePUP3_38 | From | Prunus yedoensis |
| NO: 594 | sequence | | | | var. nudiflora |
| | of | | | | |
| SEQ ID | Amino acid | Transporter | T180_McoPUP3_46 | From | Macleaya cordata |
| NO: 595 | sequence | | | | |
| | of | | | | |
| SEQ ID | DNA coding | Transporter | T180_McoPUP3_46 | From | Macleaya cordata |
| NO: 596 | sequence | | | | |
| | of | | | | |
| SEQ ID | Amino acid | Transporter | T181_HanPUP3_54 | From | Helianthus annuus |
| NO: 597 | sequence | | | | |
| | of | | | | |
| SEQ ID | DNA coding | Transporter | T181_HanPUP3_54 | From | Helianthus annuus |
| NO: 598 | sequence | | | | |
| | of | | | | |
| SEQ ID | Amino acid | Transporter | T182_CpaPUP3_62 | From | Carica papaya |
| NO: 599 | sequence | | | | |
| | of | | | | |
| SEQ ID | DNA coding | Transporter | T182_CpaPUP3_62 | From | Carica papaya |
| NO: 600 | sequence | | | | |
| | of | | | | |
| SEQ ID | Amino acid | Transporter | T184_PraPUP3_79 | From | Papaver radicatum |
| NO: 601 | sequence | | | | |
| | of | | | | |
| SEQ ID | DNA coding | Transporter | T184_PraPUP3_79 | From | Papaver radicatum |
| NO: 602 | sequence | | | | |
| | of | | | | |
| SEQ ID | Amino acid | Transporter | T186_ScaPUP3_84 | From | Sanguinaria |
| NO: 603 | sequence | | | | canadensis |
| | of | | | | |
| SEQ ID | DNA coding | Transporter | T186_ScaPUP3_84 | From | Sanguinaria |
| NO: 604 | sequence | | | | canadensis |
| | of | | | | |
| SEQ ID | Amino acid | Transporter | T188_AcoPUP3_15 | From | Aquilegia coerulea |
| NO: 605 | sequence | | | | |
| | of | | | | |
| SEQ ID | DNA coding | Transporter | T188_AcoPUP3_15 | From | Aquilegia coerulea |
| NO: 606 | sequence | | | | |
| | of | | | | |
| SEQ ID | Amino acid | Transporter | T189_PsoPUP3_23 | From | Papaver |
| NO: 607 | sequence | | | | somniferum |
| | of | | | | |
| SEQ ID | DNA coding | Transporter | T189_PsoPUP3_23 | From | Papaver |
| NO: 608 | sequence | | | | somniferum |
| | of | | | | |
| SEQ ID | Amino acid | Transporter | T191_MdoPUP3_39 | From | Malus domestica |
| NO: 609 | sequence | | | | |
| | of | | | | |
| SEQ ID | DNA coding | Transporter | T191_MdoPUP3_39 | From | Malus domestica |
| NO: 610 | sequence | | | | |
| | of | | | | |
| SEQ ID | Amino acid | Transporter | T192_CmiPUP3_47 | From | Cinnamomum |
| NO: 611 | sequence | | | | micranthum f. |
| | of | | | | kanehirae |
| SEQ ID | DNA coding | Transporter | T192_CmiPUP3_47 | From | Cinnamomum |
| NO: 612 | sequence | | | | micranthum f. |
| | of | | | | kanehirae |
| SEQ ID | Amino acid | Transporter | T193_AanPUP3_55 | From | Artemisia annua |
| NO: 613 | sequence | | | | |
| | of | | | | |
| SEQ ID | DNA coding | Transporter | T193_AanPUP3_55 | From | Artemisia annua |
| NO: 614 | sequence | | | | |
| | of | | | | |
| SEQ ID | Amino acid | Transporter | T194_CchPUP3_63 | From | Capsicum chinense |
| NO: 615 | sequence | | | | |
| | of | | | | |
| SEQ ID | DNA coding | Transporter | T194_CchPUP3_63 | From | Capsicum chinense |
| NO: 616 | sequence | | | | |
| | of | | | | |
| SEQ ID | Amino acid | Transporter | T195_JcuPUP3_71 | From | Jatropha curcas |
| NO: 617 | sequence | | | | |
| | of | | | | |
| SEQ ID | DNA coding | Transporter | T195_JcuPUP3_71 | From | Jatropha curcas |
| NO: 618 | sequence | | | | |
| | of | | | | |
| SEQ ID | Amino acid | Transporter | T196_PtrPUP3_80 | From | Papaver trinifolium |
| NO: 619 | sequence | | | | |
| | of | | | | |
| SEQ ID | DNA coding | Transporter | T196_PtrPUP3_80 | From | Papaver trinifolium |
| NO: 620 | sequence | | | | |
| | of | | | | |
| SEQ ID | Amino acid | Transporter | T197_AcoT97_GA | From | Aquilegia coerulea |
| NO: 621 | sequence | | | | |
| | of | | | | |
| SEQ ID | DNA coding | Transporter | T197_AcoT97_GA | From | Aquilegia coerulea |
| NO: 622 | sequence | | | | |
| | of | | | | |
| SEQ ID | Amino acid | Transporter | T198_AcoT97_GA | From | Aquilegia coerulea |
| NO: 623 | sequence | | | | |
| | of | | | | |
| SEQ ID | DNA coding | Transporter | T198_AcoT97_GA | From | Aquilegia coerulea |
| NO: 624 | sequence | | | | |
| | of | | | | |
| SEQ ID | Amino acid | Transporter | T199_NnuT97_GA | From | Nelumbo nucifera |
| NO: 625 | sequence | | | | |
| | of | | | | |
| SEQ ID | DNA coding | Transporter | T199_NnuT97_GA | From | Nelumbo nucifera |
| NO: 626 | sequence | | | | |
| | of | | | | |
| SEQ ID | Amino acid | Transporter | T200_T97_GA | From | Prunus yedoensis |
| NO: 627 | sequence | | | | var. nudiflora |
| | of | | | | |
| SEQ ID | DNA coding | Transporter | T200_T97_GA | From | Prunus yedoensis |
| NO: 628 | sequence | | | | var. nudiflora |
| | of | | | | |
| SEQ ID | Amino acid | Transporter | T201_HarPUP3_GA | From | Helicoverpa |
| NO: 629 | sequence | | | | armigera |
| | of | | | | |
| SEQ ID | DNA coding | Transporter | T201_HarPUP3_GA | From | Helicoverpa |
| NO: 630 | sequence | | | | armigera |
| | of | | | | |
| SEQ ID | Amino acid | Transporter | T202_PgoPUP3_GA | From | Pectinophora |
| NO: 631 | sequence | | | | gossypiella |
| | of | | | | |
| SEQ ID | DNA coding | Transporter | T202_PgoPUP3_GA | From | Pectinophora |
| NO: 632 | sequence | | | | gossypiella |
| | of | | | | |
| SEQ ID | Amino acid | Transporter | T203_HarPUP3_GA | From | Helicoverpa |
| NO: 633 | sequence | | | | armigera |
| | of | | | | |
| SEQ ID | DNA coding | Transporter | T203_HarPUP3_GA | From | Helicoverpa |
| NO: 634 | sequence | | | | armigera |
| | of | | | | |
| SEQ ID | Amino acid | Transporter | T204_RcoPUP3_GA | From | Ricinus communis |
| NO: 635 | sequence | | | | |
| | of | | | | |
| SEQ ID | DNA coding | Transporter | T204_RcoPUP3_GA | From | Ricinus communis |
| NO: 636 | sequence | | | | |
| | of | | | | |
| SEQ ID | Amino acid | Transporter | T205_HviPUP3_GA | From | Heliothis virescens |
| NO: 637 | sequence | | | | |
| | of | | | | |
| SEQ ID | DNA coding | Transporter | T205_HviPUP3_GA | From | Heliothis virescens |
| NO: 638 | sequence | | | | |
| | of | | | | |
| SEQ ID | Amino acid | Transporter | T206_VviPUP3_3_GA | From | Vitis vinifera |
| NO: 639 | sequence | | | | |
| | of | | | | |
| SEQ ID | DNA coding | Transporter | T206_VviPUP3_3_GA | From | Vitis vinifera |
| NO: 640 | sequence | | | | |
| | of | | | | |
| SEQ ID | Amino acid | Transporter | T207_MprPUP3_GA | From | Mucuna pruriens |
| NO: 641 | sequence | | | | |
| | of | | | | |
| SEQ ID | DNA coding | Transporter | T207_MprPUP3_GA | From | Mucuna pruriens |
| NO: 642 | sequence | | | | |
| | of | | | | |
| SEQ ID | Amino acid | Transporter | T208_McoPUP3_GA | From | Macleaya cordata |
| NO: 643 | sequence | | | | |
| | of | | | | |
| SEQ ID | DNA coding | Transporter | T208_McoPUP3_GA | From | Macleaya cordata |
| NO: 644 | sequence | | | | |
| | of | | | | |
| SEQ ID | Amino acid | Transporter | T209_RcoPUP3_GA | From | Ricinus communis |
| NO: 645 | sequence | | | | |
| | of | | | | |
| SEQ ID | DNA coding | Transporter | T209_RcoPUP3_GA | From | Ricinus communis |
| NO: 646 | sequence | | | | |
| | of | | | | |
| SEQ ID | Amino acid | Transporter | T210_NnuPUP3_GA | From | Nelumbo nucifera |
| NO: 647 | sequence | | | | |
| | of | | | | |
| SEQ ID | DNA coding | Transporter | T210_NnuPUP3_GA | From | Nelumbo nucifera |
| NO: 648 | sequence | | | | |
| | of | | | | |
| SEQ ID | Amino acid | Transporter | T211_HarPUP3_GA | From | Helicoverpa |
| NO: 649 | sequence | | | | armigera |
| | of | | | | |
| SEQ ID | DNA coding | Transporter | T211_HarPUP3_GA | From | Helicoverpa |
| NO: 650 | sequence | | | | armigera |
| | of | | | | |
| SEQ ID | Amino acid | Transporter | T212_HarPUP3_GA | From | Helicoverpa |
| NO: 651 | sequence | | | | armigera |
| | of | | | | |
| SEQ ID | DNA coding | Transporter | T212_HarPUP3_GA | From | Helicoverpa |
| NO: 652 | sequence | | | | armigera |
| | of | | | | |
| SEQ ID | Amino acid | Transporter | T213_HarPUP3_GA | From | Helicoverpa |
| NO: 653 | sequence | | | | armigera |
| | of | | | | |
| SEQ ID | DNA coding | Transporter | T213_HarPUP3_GA | From | Helicoverpa |
| NO: 654 | sequence | | | | armigera |
| | of | | | | |
| SEQ ID | Amino acid | Transporter | T214_HarPUP3_GA | From | Helicoverpa |
| NO: 655 | sequence | | | | armigera |
| | of | | | | |
| SEQ ID | DNA coding | Transporter | T214_HarPUP3_GA | From | Helicoverpa |
| NO: 656 | sequence | | | | armigera |
| | of | | | | |
| SEQ ID | Amino acid | Transporter | T215_HarPUP3_GA | From | Helicoverpa |
| NO: 657 | sequence | | | | armigera |
| | of | | | | |
| SEQ ID | DNA coding | Transporter | T215_HarPUP3_GA | From | Helicoverpa |
| NO: 658 | sequence | | | | armigera |
| | of | | | | |
| SEQ ID | Amino acid | Transporter | T216_HarPUP3_GA | From | Helicoverpa |
| NO: 659 | sequence | | | | armigera |
| | of | | | | |
| SEQ ID | DNA coding | Transporter | T216_HarPUP3_GA | From | Helicoverpa |
| NO: 660 | sequence | | | | armigera |
| | of | | | | |
| SEQ ID | Amino acid | Transporter | T217_AcoPUP3_GA | From | Aquilegia coerulea |
| NO: 661 | sequence | | | | |
| | of | | | | |
| SEQ ID | DNA coding | Transporter | T217_AcoPUP3_GA | From | Aquilegia coerulea |
| NO: 662 | sequence | | | | |
| | of | | | | |
| SEQ ID | Amino acid | Transporter | T65_IjaNPF_GA | From | Lonicera japonica |
| NO: 733 | sequence | | | | |
| | of | | | | |
| SEQ ID | DNA coding | Transporter | T65_IjaNPF_GA | From | Lonicera japonica |
| NO: 734 | sequence | | | | |
| | of | | | | |
| SEQ ID | Amino acid | Transporter | T94_EcrPOT_GA | From | Emmonsia crescens |
| NO: 735 | sequence | | | | |
| | of | | | | |
| SEQ ID | DNA coding | Transporter | T94_EcrPOT_GA | From | Emmonsia crescens |
| NO: 736 | sequence | | | | |
| | of | | | | |
| SEQ ID | Amino acid | ADHS | ADH5 Dehydrogenase | From | Saccharomyces |
| NO: 663 | sequence | Dehydrogenase | | | cerevisiae |
| | of | | | | |
| SEQ ID | DNA coding | ADH5 | ADH5 Dehydrogenase | From | Saccharomyces |
| NO: 664 | sequence | Dehydrogenase | | | cerevisiae |
| | of | | | | |
| SEQ ID | Amino acid | ADH6 | ADH6 Dehydrogenase | From | Saccharomyces |
| NO: 665 | sequence | Dehydrogenase | | | cerevisiae |
| | of | | | | |
| SEQ ID | DNA coding | ADH6 | ADH6 Dehydrogenase | From | Saccharomyces |
| NO: 666 | sequence | Dehydrogenase | | | cerevisiae |
| | of | | | | |
| SEQ ID | Amino acid | ADH7 | ADH7 Dehydrogenase | From | Saccharomyces |
| NO: 667 | sequence | Dehydrogenase | | | cerevisiae |
| | of | | | | |
| SEQ ID | DNA coding | ADH7 | ADH7 Dehydrogenase | From | Saccharomyces |
| NO: 668 | sequence | Dehydrogenase | | | cerevisiae |
| | of | | | | |
| SEQ ID | Amino acid | YPR127W | YPR127W Dehydrogenase | From | Saccharomyces |
| NO: 669 | sequence | Dehydrogenase | | | cerevisiae |
| | of | | | | |
| SEQ ID | DNA coding | YPR127W | YPR127W Dehydrogenase | From | Saccharomyces |
| NO: 670 | sequence | Dehydrogenase | | | cerevisiae |
| | of | | | | |
| SEQ ID | Amino acid | AAD3 | AAD3 Dehydrogenase | From | Saccharomyces |
| NO: 671 | sequence | Dehydrogenase | | | cerevisiae |
| | of | | | | |
| SEQ ID | DNA coding | AAD3 | AAD3 Dehydrogenase | From | Saccharomyces |
| NO: 672 | sequence | Dehydrogenase | | | cerevisiae |
| | of | | | | |
| SEQ ID | Amino acid | AAD4 | AAD4 Dehydrogenase | From | Saccharomyces |
| NO: 673 | sequence | Dehydrogenase | | | cerevisiae |
| | of | | | | |
| SEQ ID | DNA coding | AAD4 | AAD4 Dehydrogenase | From | Saccharomyces |
| NO: 674 | sequence | Dehydrogenase | | | cerevisiae |
| | of | | | | |
| SEQ ID | Amino acid | ADH3 | ADH3 Dehydrogenase | From | Saccharomyces |
| NO: 675 | sequence | Dehydrogenase | | | cerevisiae |
| | of | | | | |
| SEQ ID | DNA coding | ADH3 | ADH3 Dehydrogenase | From | Saccharomyces |
| NO: 676 | sequence | Dehydrogenase | | | cerevisiae |
| | of | | | | |
| SEQ ID | Amino acid | ADH4 | ADH4 Dehydrogenase | From | Saccharomyces |
| NO: 677 | sequence | Dehydrogenase | | | cerevisiae |
| | of | | | | |
| SEQ ID | DNA coding | ADH4 | ADH4 Dehydrogenase | From | Saccharomyces |
| NO: 678 | sequence | Dehydrogenase | | | cerevisiae |
| | of | | | | |
| SEQ ID | Amino acid | ALD6 | ALD6 Dehydrogenase | From | Saccharomyces |
| NO: 679 | sequence | Dehydrogenase | | | cerevisiae |
| | of | | | | |
| SEQ ID | DNA coding | ALD6 | ALD6 Dehydrogenase | From | Saccharomyces |
| NO: 680 | sequence | Dehydrogenase | | | cerevisiae |
| | of | | | | |
| SEQ ID | Amino acid | BDH1 | BDH1 Dehydrogenase | From | Saccharomyces |
| NO: 681 | sequence | Dehydrogenase | | | cerevisiae |
| | of | | | | |
| SEQ ID | DNA coding | BDH1 | BDH1 Dehydrogenase | From | Saccharomyces |
| NO: 682 | sequence | Dehydrogenase | | | cerevisiae |
| | of | | | | |
| SEQ ID | Amino acid | BDH2 | BDH2 Dehydrogenase | From | Saccharomyces |
| NO: 683 | sequence | Dehydrogenase | | | cerevisiae |
| | of | | | | |
| SEQ ID | DNA coding | BDH2 | BDH2 Dehydrogenase | From | Saccharomyces |
| NO: 684 | sequence | Dehydrogenase | | | cerevisiae |
| | of | | | | |
| SEQ ID | Amino acid | FOX2 | FOX2 Dehydrogenase | From | Saccharomyces |
| NO: 685 | sequence | Dehydrogenase | | | cerevisiae |
| | of | | | | |
| SEQ ID | DNA coding | FOX2 | FOX2 Dehydrogenase | From | Saccharomyces |
| NO: 686 | sequence | Dehydrogenase | | | cerevisiae |
| | of | | | | |
| SEQ ID | Amino acid | GCY1 | GCY1 Dehydrogenase | From | Saccharomyces |
| NO: 687 | sequence | Dehydrogenase | | | cerevisiae |
| | of | | | | |
| SEQ ID | DNA coding | GCY1 | GCY1 Dehydrogenase | From | Saccharomyces |
| NO: 688 | sequence | Dehydrogenase | | | cerevisiae |
| | of | | | | |
| SEQ ID | Amino acid | GPD1 | GPD1 Dehydrogenase | From | Saccharomyces |
| NO: 689 | sequence | Dehydrogenase | | | cerevisiae |
| | of | | | | |
| SEQ ID | DNA coding | GPD1 | GPD1 Dehydrogenase | From | Saccharomyces |
| NO: 690 | sequence | Dehydrogenase | | | cerevisiae |
| | of | | | | |
| SEQ ID | Amino acid | HIS4 | HIS4 Dehydrogenase | From | Saccharomyces |
| NO: 691 | sequence | Dehydrogenase | | | cerevisiae |
| | of | | | | |
| SEQ ID | DNA coding | HIS4 | HIS4 Dehydrogenase | From | Saccharomyces |
| NO: 692 | sequence | Dehydrogenase | | | cerevisiae |
| | of | | | | |
| SEQ ID | Amino acid | IPD1 | IPD1 Dehydrogenase | From | Saccharomyces |
| NO: 693 | sequence | Dehydrogenase | | | cerevisiae |
| | of | | | | |
| SEQ ID | DNA coding | IPD1 | IPD1 Dehydrogenase | From | Saccharomyces |
| NO: 694 | sequence | Dehydrogenase | | | cerevisiae |
| | of | | | | |
| SEQ ID | Amino acid | LYS12 | LYS12 Dehydrogenase | From | Saccharomyces |
| NO: 695 | sequence | Dehydrogenase | | | cerevisiae |
| | of | | | | |
| SEQ ID | DNA coding | LYS12 | LYS12 Dehydrogenase | From | Saccharomyces |
| NO: 696 | sequence | Dehydrogenase | | | cerevisiae |
| | of | | | | |
| SEQ ID | Amino acid | SER33 | SER33 Dehydrogenase | From | Saccharomyces |
| NO: 697 | sequence | Dehydrogenase | | | cerevisiae |
| | of | | | | |
| SEQ ID | DNA coding | SER33 | SER33 Dehydrogenase | From | Saccharomyces |
| NO: 698 | sequence | Dehydrogenase | | | cerevisiae |
| | of | | | | |
| SEQ ID | Amino acid | ZWF1 | ZWF1 Dehydrogenase | From | Saccharomyces |
| NO: 699 | sequence | Dehydrogenase | | | cerevisiae |
| | of | | | | |
| SEQ ID | DNA coding | ZWF1 | ZWF1 Dehydrogenase | From | Saccharomyces |
| NO: 700 | sequence | Dehydrogenase | | | cerevisiae |
| | of | | | | |
| SEQ ID | Amino acid | YPL088W | YPL088W Dehydrogenase | From | Saccharomyces |
| NO: 701 | sequence | Dehydrogenase | | | cerevisiae |
| | of | | | | |
| SEQ ID | DNA coding | YPL088W | YPL088W Dehydrogenase | From | Saccharomyces |
| NO: 702 | sequence | Dehydrogenase | | | cerevisiae |
| | of | | | | |
| SEQ ID | Amino acid | ARA1 | ARA1 Dehydrogenase | From | Saccharomyces |
| NO: 703 | sequence | Dehydrogenase | | | cerevisiae |
| | of | | | | |
| SEQ ID | DNA coding | ARA1 | ARA1 Dehydrogenase | From | Saccharomyces |
| NO: 704 | sequence | Dehydrogenase | | | cerevisiae |
| | of | | | | |
| SEQ ID | Amino acid | HFD1 | HFD1 Dehydrogenase | From | Saccharomyces |
| NO: 705 | sequence | Dehydrogenase | | | cerevisiae |
| | of | | | | |
| SEQ ID | DNA coding | HFD1 | HFD1 Dehydrogenase | From | Saccharomyces |
| NO: 706 | sequence | Dehydrogenase | | | cerevisiae |
| | of | | | | |
| SEQ ID | Amino acid | YPR1 Reductase | YPR1 Reductase | From | Saccharomyces |
| NO: 707 | sequence | | | | cerevisiae |
| | of | | | | |
| SEQ ID | DNA coding | YPR1 Reductase | YPR1 Reductase | From | Saccharomyces |
| NO: 708 | sequence | | | | cerevisiae |
| | of | | | | |
| SEQ ID | Amino acid | ALD4 Reductase | ALD4 Reductase | From | Saccharomyces |
| NO: 709 | sequence | | | | cerevisiae |
| | of | | | | |
| SEQ ID | DNA coding | ALD4 Reductase | ALD4 Reductase | From | Saccharomyces |
| NO: 710 | sequence | | | | cerevisiae |
| | of | | | | |
| SEQ ID | Amino acid | GOR1 Reductase | GOR1 Reductase | From | Saccharomyces |
| NO: 711 | sequence | | | | cerevisiae |
| | of | | | | |
| SEQ ID | DNA coding | GOR1 Reductase | GOR1 Reductase | From | Saccharomyces |
| NO: 712 | sequence | | | | cerevisiae |
| | of | | | | |
| SEQ ID | Amino acid | GRE2 Reductase | GRE2 Reductase | From | Saccharomyces |
| NO: 713 | sequence | | | | cerevisiae |
| | of | | | | |
| SEQ ID | DNA coding | GRE2 Reductase | GRE2 Reductase | From | Saccharomyces |
| NO: 714 | sequence | | | | cerevisiae |
| | of | | | | |
| SEQ ID | Amino acid | GRE3 Reductase | GRE3 Reductase | From | Saccharomyces |
| NO: 715 | sequence | | | | cerevisiae |
| | of | | | | |
| SEQ ID | DNA coding | GRE3 Reductase | GRE3 Reductase | From | Saccharomyces |
| NO: 716 | sequence | | | | cerevisiae |
| | of | | | | |
| SEQ ID | Amino acid | YDR541C Reductase | YDR541C Reductase | From | Saccharomyces |
| NO: 717 | sequence | | | | cerevisiae |
| | of | | | | |
| SEQ ID | DNA coding | YDR541C Reductase | YDR541C Reductase | From | Saccharomyces |
| NO: 718 | sequence | | | | cerevisiae |
| | of | | | | |
| SEQ ID | Amino acid | YLR460C Reductase | YLR460C Reductase | From | Saccharomyces |
| NO: 719 | sequence | | | | cerevisiae |
| | of | | | | |
| SEQ ID | DNA coding | YLR460C Reductase | YLR460C Reductase | From | Saccharomyces |
| NO: 720 | sequence | | | | cerevisiae |
| | of | | | | |
| SEQ ID | Amino acid | ARI1 Reductase | ARI1 Reductase | From | Saccharomyces |
| NO: 721 | sequence | | | | cerevisiae |
| | of | | | | |
| SEQ ID | DNA coding | ARI1 Reductase | ARI1 Reductase | From | Saccharomyces |
| NO: 722 | sequence | | | | cerevisiae |
| | of | | | | |
| SEQ ID | Amino acid | YGL039W Reductase | YGL039W Reductase | From | Saccharomyces |
| NO: 723 | sequence | | | | cerevisiae |
| | of | | | | |
| SEQ ID | DNA coding | YGL039W Reductase | YGL039W Reductase | From | Saccharomyces |
| NO: 724 | sequence | | | | cerevisiae |
| | of | | | | |
| SEQ ID | Amino acid | YCR102C Reductase | YCR102C Reductase | From | Saccharomyces |
| NO: 725 | sequence | | | | cerevisiae |
| | of | | | | |
| SEQ ID | DNA coding | YCR102C Reductase | YCR102C Reductase | From | Saccharomyces |
| NO: 726 | sequence | | | | cerevisiae |
| | of | | | | |
| SEQ ID | Amino acid | HMG1 Reductase | HMG1 Reductase | From | Saccharomyces |
| NO: 727 | sequence | | | | cerevisiae |
| | of | | | | |
| SEQ ID | DNA coding | HMG1 Reductase | HMG1 Reductase | From | Saccharomyces |
| NO: 728 | sequence | | | | cerevisiae |
| | of | | | | |
| SEQ ID | Amino acid | PHA2 Dehydratase | PHA2 Dehydratase | From | Saccharomyces |
| NO: 729 | sequence | | | | cerevisiae |
| | of | | | | |
| SEQ ID | DNA coding | PHA2 Dehydratase | PHA2 Dehydratase | From | Saccharomyces |
| NO: 730 | sequence | | | | cerevisiae |
| | of | | | | |
| SEQ ID | Amino acid | TRP3 Synthase | TRP3 Synthase | From | Saccharomyces |
| NO: 731 | sequence | | | | cerevisiae |
| | of | | | | |
| SEQ ID | DNA coding | TRP3 Synthase | TRP3 Synthase | From | Saccharomyces |
| NO: 732 | sequence | | | | cerevisiae |
| | of | | | | |
| SEQ ID | DNA coding | HEME cofactor | HEM2 | From | Saccharomyces |
| NO: 737 | sequence | | | | cerevisiae |
| | of | | | | |
| SEQ ID | Amino acid | HEME cofactor | HEM2 | From | Saccharomyces |
| NO: 738 | sequence | | | | cerevisiae |
| | of | | | | |
| SEQ ID | DNA coding | HEME cofactor | HEM3 | From | Saccharomyces |
| NO: 739 | sequence | | | | cerevisiae |
| | of | | | | |
| SEQ ID | Amino acid | HEME cofactor | HEM3 | From | Saccharomyces |
| NO: 740 | sequence | | | | cerevisiae |
| | of | | | | |
| SEQ ID | DNA coding | HEME cofactor | HEM12 | From | Saccharomyces |
| NO: 741 | sequence | | | | cerevisiae |
| | of | | | | |
| SEQ ID | Amino acid | HEME cofactor | HEM12 | From | Saccharomyces |
| NO: 742 | sequence | | | | cerevisiae |
| | of | | | | |
| SEQ ID | DNA coding | HEME cofactor | HMX1 | From | Saccharomyces |
| NO: 743 | sequence | | | | cerevisiae |
| | of | | | | |
| SEQ ID | Amino acid | HEME cofactor | HMX1 | From | Saccharomyces |
| NO: 744 | sequence | | | | cerevisiae |
| | of | | | | |
| SEQ ID | DNA coding | P450 chaperone | KAR2 | From | Saccharomyces |
| NO: 745 | sequence | | | | cerevisiae |
| | of | | | | |
| SEQ ID | Amino acid | P450 chaperone | KAR2 | From | Saccharomyces |
| NO: 746 | sequence | | | | cerevisiae |
| | of | | | | |
| SEQ ID | DNA coding | P450 chaperone | HSP82 | From | Saccharomyces |
| NO: 747 | sequence | | | | cerevisiae |
| | of | | | | |
| SEQ ID | Amino acid | P450 chaperone | HSP82 | From | Saccharomyces |
| NO: 748 | sequence | | | | cerevisiae |
| | of | | | | |
| SEQ ID | DNA coding | P450 chaperone | CNE1 | From | Saccharomyces |
| NO: 749 | sequence | | | | cerevisiae |
| | of | | | | |
| SEQ ID | Amino acid | P450 chaperone | CNE1 | From | Saccharomyces |
| NO: 750 | sequence | | | | cerevisiae |
| | of | | | | |
| SEQ ID | DNA coding | P450 chaperone | SSA1 | From | Saccharomyces |
| NO: 751 | sequence | | | | cerevisiae |
| | of | | | | |
| SEQ ID | Amino acid | P450 chaperone | SSA1 | From | Saccharomyces |
| NO: 752 | sequence | | | | cerevisiae |
| | of | | | | |
| SEQ ID | DNA coding | P450 chaperone | CPR6 | From | Saccharomyces |
| NO: 753 | sequence | | | | cerevisiae |
| | of | | | | |
| SEQ ID | Amino acid | P450 chaperone | CPR6 | From | Saccharomyces |
| NO: 754 | sequence | | | | cerevisiae |
| | of | | | | |
| SEQ ID | DNA coding | P450 chaperone | FES1 | From | Saccharomyces |
| NO: 755 | sequence | | | | cerevisiae |
| | of | | | | |
| SEQ ID | Amino acid | P450 chaperone | FES1 | From | Saccharomyces |
| NO: 756 | sequence | | | | cerevisiae |
| | of | | | | |
| SEQ ID | DNA coding | P450 chaperone | HSP104 | From | Saccharomyces |
| NO: 757 | sequence | | | | cerevisiae |
| | of | | | | |
| SEQ ID | Amino acid | P450 chaperone | HSP104 | From | Saccharomyces |
| NO: 758 | sequence | | | | cerevisiae |
| | of | | | | |
| SEQ ID | DNA coding | P450 chaperone | STI1 | From | Saccharomyces |
| NO: 759 | sequence | | | | cerevisiae |
| | of | | | | |
| SEQ ID | Amino acid | P450 chaperone | STI1 | From | Saccharomyces |
| NO: 760 | sequence | | | | cerevisiae |
| | of | | | | |
| SEQ ID | DNA coding | P450 regulator | DAP1 | From | Saccharomyces |
| NO: 761 | sequence | | | | cerevisiae |
| | of | | | | |
| SEQ ID | Amino acid | P450 regulator | DAP1 | From | Saccharomyces |
| NO: 762 | sequence | | | | cerevisiae |
| | of | | | | |
| SEQ ID | DNA coding | P450 regulator | HAC1 | From | Saccharomyces |
| NO: 763 | sequence | | | | cerevisiae |
| | of | | | | |
| SEQ ID | Amino acid | P450 regulator | HAC1 | From | Saccharomyces |
| NO: 764 | sequence | | | | cerevisiae |
| | of | | | | |
| SEQ ID | DNA coding | NADPH cofactor | ZWF1 | From | Saccharomyces |
| NO: 765 | sequence | | | | cerevisiae |
| | of | | | | |
| SEQ ID | Amino acid | NADPH cofactor | ZWF1 | From | Saccharomyces |
| NO: 766 | sequence | | | | cerevisiae |
| | of | | | | |
| SEQ ID | DNA coding | NADPH cofactor | GND1 | From | Saccharomyces |
| NO: 767 | sequence | | | | cerevisiae |
| | of | | | | |
| SEQ ID | Amino acid | NADPH cofactor | GND1 | From | Saccharomyces |
| NO: 768 | sequence | | | | cerevisiae |
| | of | | | | |
| SEQ ID | DNA coding | formadehyde | SFA1 | From | Saccharomyces |
| NO: 769 | sequence | toxicity regulator | | | cerevisiae |
| | of | | | | |
| SEQ ID | Amino acid | formadehyde | SFA1 | From | Saccharomyces |
| NO: 770 | sequence | toxicity regulator | | | cerevisiae |
| | of | | | | |
| SEQ ID | DNA coding | Moth P450 | Hv_CYP_A0A2A4JAM9_A110N + H242P + V224I_co6 | From | Artificial |
| NO: 771 | sequence | (demethylase) | | | |
| | of | mutant codon | | | |
| | | optimized | | | |
| SEQ ID | Amino acid | Moth P450 | Hv_CYP_A0A2A4JAM9_A110N + H242P + V224I_co6 | From | Artificial |
| NO: 772 | sequence | (demethylase) | | | |
| | of | mutant codon | | | |
| | | optimized | | | |
| SEQ ID | DNA coding | Transporter codon | T149_AcPUP3_59_co2 | From | Artificial |
| NO: 773 | sequence | optimised | | | |
| | of | | | | |
| SEQ ID | Amino acid | Transporter | T149_AcPUP3_59_co2 | From | Aquilegia coerulea |
| NO: 774 | sequence | | | | |
| | of | | | | |
| SEQ ID | DNA coding | Transporter codon | T149_AcPUP3_59_co3 | From | Artificial |
| NO: 775 | sequence | optimised | | | |
| | of | | | | |
| SEQ ID | Amino acid | Transporter | T149_AcPUP3_59_co3 | From | Aquilegia coerulea |
| NO: 776 | sequence | | | | |
| | of | | | | |
| SEQ ID | DNA coding | Transporter codon | T149_AcPUP3_59_co4 | From | Artificial |
| NO: 777 | sequence | optimised | | | |
| | of | | | | |
| SEQ ID | Amino acid | Transporter | T149_AcPUP3_59_co4 | From | Aquilegia coerulea |
| NO: 778 | sequence | | | | |
| | of | | | | |
| SEQ ID | DNA coding | Transporter codon | T180_McPUP3_46_co2 | From | Artificial |
| NO: 779 | sequence | optimised | | | |
| | of | | | | |
| SEQ ID | Amino acid | Transporter | T180_McPUP3_46_co2 | From | Momordica |
| NO: 780 | sequence | | | | charantia |
| | of | | | | |
| SEQ ID | DNA coding | Transporter codon | T180_McPUP3_46_co3 | From | Artificial |
| NO: 781 | sequence | optimised | | | |
| | of | | | | |
| SEQ ID | Amino acid | Transporter | T180_McPUP3_46_co3 | From | Momordica |
| NO: 782 | sequence | | | | charantia |
| | of | | | | |
| SEQ ID | DNA coding | Transporter codon | T180_McPUP3_46_co4 | From | Artificial |
| NO: 783 | sequence | optimised | | | |
| | of | | | | |
| SEQ ID | Amino acid | Transporter | T180_McPUP3_46_co4 | From | Momordica |
| NO: 784 | sequence | | | | charantia |
| | of | | | | |
| SEQ ID | DNA coding | Transporter codon | T180_McPUP3_46_co6 | From | Artificial |
| NO: 785 | sequence | optimised | | | |
| | of | | | | |
| SEQ ID | Amino acid | Transporter | T180_McPUP3_46_co6 | From | Momordica |
| NO: 786 | sequence | | | | charantia |
| | of | | | | |
| SEQ ID | DNA coding | Transporter codon | T193_AanPUP3_55_co2 | From | Artificial |
| NO: 787 | sequence | optimised | | | |
| | of | | | | |
| SEQ ID | Amino acid | Transporter | T193_AanPUP3_55_co2 | From | Artemisia annua |
| NO: 788 | sequence | | | | |
| | of | | | | |
| SEQ ID | DNA coding | Transporter codon | T193_AanPUP3_55_co3 | From | Artificial |
| NO: 789 | sequence | optimised | | | |
| | of | | | | |
| SEQ ID | Amino acid | Transporter | T193_AanPUP3_55_co3 | From | Artemisia annua |
| NO: 790 | sequence | | | | |
| | of | | | | |
| SEQ ID | DNA coding | Transporter codon | T193_AanPUP3_55_co5 | From | Artificial |
| NO: 791 | sequence | optimised | | | |
| | of | | | | |
| SEQ ID | Amino acid | Transporter | T193_AanPUP3_55_co5 | From | Artemisia annua |
| NO: 792 | sequence | | | | |
| | of | | | | |
| SEQ ID | DNA coding | Transporter codon | T193_AanPUP3_55_co6 | From | Artificial |
| NO: 793 | sequence | optimised | | | |
| | of | | | | |
| SEQ ID | Amino acid | Transporter | T193_AanPUP3_55_co6 | From | Artemisia annua |
| NO: 794 | sequence | | | | |
| | of | | | | |
| SEQ ID | Amino acid | Transporter | T218_HviENT3_GA | From | Heliothis virescens |
| NO: 795 | sequence | | | | |
| | of | | | | |
| SEQ ID | DNA coding | Transporter | T218_HviENT3_GA | From | Heliothis virescens |
| NO: 796 | sequence | | | | |
| | of | | | | |
| SEQ ID | Amino acid | Transporter | T220_CsuENT3_GA | From | Chilo suppressalis |
| NO: 797 | sequence | | | | |
| | of | | | | |
| SEQ ID | DNA coding | Transporter | T220_CsuENT3_GA | From | Chilo suppressalis |
| NO: 798 | sequence | | | | |
| | of | | | | |
| SEQ ID | Amino acid | Transporter | T221_BmoENT3_GA | From | Bombyx mori |
| NO: 799 | sequence | | | | |
| | of | | | | |
| SEQ ID | DNA coding | Transporter | T221_BmoENT3_GA | From | Bombyx mori |
| NO: 800 | sequence | | | | |
| | of | | | | |
| SEQ ID | Amino acid | Transporter | T227_AcuENT3_GA | From | Anopheles |
| NO: 801 | sequence | | | | culicifacies |
| | of | | | | |
| SEQ ID | DNA coding | Transporter | T227_AcuENT3_GA | From | Anopheles |
| NO: 802 | sequence | | | | culicifacies |
| | of | | | | |
| SEQ ID | Amino acid | Transporter | T234_CsuENT3_GA | From | Chilo suppressalis |
| NO: 803 | sequence | | | | |
| | of | | | | |
| SEQ ID | DNA coding | Transporter | T234_CsuENT3_GA | From | Chilo suppressalis |
| NO: 804 | sequence | | | | |
| | of | | | | |
| SEQ ID | Amino acid | Transporter | T237_PxuENT3_GA | From | Papilio xuthus |
| NO: 805 | sequence | | | | |
| | of | | | | |
| SEQ ID | DNA coding | Transporter | T237_PxuENT3_GA | From | Papilio xuthus |
| NO: 806 | sequence | | | | |
| | of | | | | |
| SEQ ID | Amino acid | Transporter | T238_HviENT3_GA | From | Heliothis virescens |
| NO: 807 | sequence | | | | |
| | of | | | | |
| SEQ ID | DNA coding | Transporter | T238_HviENT3_GA | From | Heliothis virescens |
| NO: 808 | sequence | | | | |
| | of | | | | |
| SEQ ID | Amino acid | Transporter | T239_CmePUP3_GA | From | Cucumis melo var. |
| NO: 809 | sequence | | | | makuwa |
| | of | | | | |
| SEQ ID | DNA coding | Transporter | T239_CmePUP3_GA | From | Cucumis melo var. |
| NO: 810 | sequence | | | | makuwa |
| | of | | | | |
| SEQ ID | Amino acid | Transporter | T240_PpePUP3_GA | From | Prunus persica |
| NO: 811 | sequence | | | | |
| | of | | | | |
| SEQ ID | DNA coding | Transporter | T240_PpePUP3_GA | From | Prunus persica |
| NO: 812 | sequence | | | | |
| | of | | | | |
| SEQ ID | Amino acid | Transporter | T242_AchPUP3_GA | From | Actinidia chinensis |
| NO: 813 | sequence | | | | var. chinensis |
| | of | | | | |
| SEQ ID | DNA coding | Transporter | T242_AchPUP3_GA | From | Actinidia chinensis |
| NO: 814 | sequence | | | | var. chinensis |
| | of | | | | |
| SEQ ID | Amino acid | Transporter | T243_EguPUP3_GA | From | Erythranthe guttata |
| NO: 815 | sequence | | | | |
| | of | | | | |
| SEQ ID | DNA coding | Transporter | T243_EguPUP3_GA | From | Erythranthe guttata |
| NO: 816 | sequence | | | | |
| | of | | | | |
| SEQ ID | Amino acid | Transporter | T244_CcaPUP3_GA | From | Corchorus capsularis |
| NO: 817 | sequence | | | | |
| | of | | | | |
| SEQ ID | DNA coding | Transporter | T244_CcaPUP3_GA | From | Corchorus capsularis |
| NO: 818 | sequence | | | | |
| | of | | | | |
| SEQ ID | Amino acid | Transporter | T245_CcaPUP3_GA | From | Handroanthus |
| NO: 819 | sequence | | | | impetiginosus |
| | of | | | | |
| SEQ ID | DNA coding | Transporter | T245_CcaPUP3_GA | From | Handroanthus |
| NO: 820 | sequence | | | | impetiginosus |
| | of | | | | |
| SEQ ID | Amino acid | Transporter | T248_McoPUP3_GA | From | Macleaya cordata |
| NO: 821 | sequence | | | | |
| | of | | | | |
| SEQ ID | DNA coding | Transporter | T248_McoPUP3_GA | From | Macleaya cordata |
| NO: 822 | sequence | | | | |
| | of | | | | |
| SEQ ID | Amino acid | Transporter | T253_AanPUP3_GA | From | Artemisia annua |
| NO: 823 | sequence | | | | |
| | of | | | | |
| SEQ ID | DNA coding | Transporter | T253_AanPUP3_GA | From | Artemisia annua |
| NO: 824 | sequence | | | | |
| | of | | | | |
| SEQ ID | Amino acid | Transporter | T254_CcaPUP3_GA | From | Cynara cardunculus |
| NO: 825 | sequence | | | | var. scolymus |
| | of | | | | |
| SEQ ID | DNA coding | Transporter | T254_CcaPUP3_GA | From | Cynara cardunculus |
| NO: 826 | sequence | | | | var. scolymus |
| | of | | | | |
| SEQ ID | Amino acid | P450 (demethylase) | A0A286QUG7 | From | Spodoptera exigua |
| NO: 827 | sequence | | | | |
| | of | | | | |
| SEQ ID | DNA coding | P450 (demethylase) | A0A286QUG7 | From | Spodoptera exigua |
| NO: 828 | sequence | | | | |
| | of | | | | |
| SEQ ID | Amino acid | P450 (demethylase) | D5L0M5 | From | Manduca sexta |
| NO: 829 | sequence | | | | |
| | of | | | | |
| SEQ ID | DNA coding | P450 (demethylase) | DSL0M5 | From | Manduca sexta |
| NO: 830 | sequence | | | | |
| | of | | | | |
| SEQ ID | Amino acid | P450 (demethylase) | XP026740610 | From | Trichoplusia ni |
| NO: 831 | sequence | | | | |
| | of | | | | |
| SEQ ID | DNA coding | P450 (demethylase) | XP026740610 | From | Trichoplusia ni |
| NO: 832 | sequence | | | | |
| | of | | | | |
| SEQ ID | Amino acid | P450 (demethylase) | W5W4U7 | From | Lymantria dispar |
| NO: 833 | sequence | | | | |
| | of | | | | |
| SEQ ID | DNA coding | P450 (demethylase) | W5W4U7 | From | Lymantria dispar |
| NO: 834 | sequence | | | | |
| | of | | | | |
| SEQ ID | Amino acid | P450 (demethylase) | ACF17813 | From | Ostrinia furnacalis |
| NO: 835 | sequence | | | | |
| | of | | | | |
| SEQ ID | DNA coding | P450 (demethylase) | ACF17813 | From | Ostrinia furnacalis |
| NO: 836 | sequence | | | | |
| | of | | | | |
| SEQ ID | Amino acid | P450 (demethylase) | A0A4C1YMA7 | From | Eumeta variegata |
| NO: 837 | sequence | | | | |
| | of | | | | |
| SEQ ID | DNA coding | P450 (demethylase) | A0A4C1YMA7 | From | Eumeta variegata |
| NO: 838 | sequence | | | | |
| | of | | | | |
| SEQ ID | Amino acid | P450 (demethylase) | MsCPR_XP_030039194 | From | Manduca sexta |
| NO: 839 | sequence | | | | |
| | of | | | | |
| SEQ ID | DNA coding | P450 (demethylase) | MSCPR_XP_030039194 | From | Manduca sexta |
| NO: 840 | sequence | | | | |
| | of | | | | |
| SEQ ID | Amino acid | P450 (demethylase) | HvCPR_A0A2A4IYH3 | From | Heliothis virescens |
| NO: 841 | sequence | | | | |
| | of | | | | |
| SEQ ID | DNA coding | P450 (demethylase) | HvCPR_A0A2A4IYH3 | From | Heliothis virescens |
| NO: 842 | sequence | | | | |
| | of | | | | |
| SEQ ID | Amino acid | P450 (demethylase) | HaCYP6AE15v2_t | From | Helicoverpa |
| NO: 843 | sequence | | | | armigera |
| | of | | | | |
| SEQ ID | DNA coding | P450 (demethylase) | HaCYP6AE15v2_t | From | Helicoverpa |
| NO: 844 | sequence | | | | armigera |
| | of | | | | |
| SEQ ID | Amino acid | P450 (demethylase) | NMCH-HaCYP6AE15v2_t (Amino acid 1-25 −> | From | Helicoverpa |
| NO: 845 | sequence | | NMCH N-terminal signal peptide) | | armigera |
| | of | | | | |
| SEQ ID | DNA coding | P450 (demethylase) | NMCH-HaCYP6AE15v2_t | From | Helicoverpa |
| NO: 846 | sequence | | | | armigera |
| | of | | | | |
| SEQ ID | Amino acid | P450 (demethylase) | EcCFS-SP-HaCYP6AE15v2_t (Amino acid 1-22 −> | From | Helicoverpa |
| NO: 847 | sequence | | EcCFS N-terminal signal peptide) | | armigera |
| | of | | | | |
| SEQ ID | DNA coding | P450 (demethylase) | EcCFS-SP-HaCYP6AE15v2_t | From | Helicoverpa |
| NO: 848 | sequence | | | | armigera |
| | of | | | | |
| SEQ ID | Amino acid | P450 (demethylase) | HaCYP6AE15v2_A316G_t | From | Helicoverpa |
| NO: 849 | sequence | | | | armigera |
| | of | | | | |
| SEQ ID | DNA coding | P450 (demethylase) | HaCYP6AE15v2_A316G_t | From | Helicoverpa |
| NO: 850 | sequence | | | | armigera |
| | of | | | | |
| SEQ ID | Amino acid | P450 (demethylase) | NMCH-HaCYP6AE15v2_A316G_t (Amino acid 1-25 −> | From | Helicoverpa |
| NO: 851 | sequence | | NMCH N-terminal signal peptide) | | armigera |
| | of | | | | |
| SEQ ID | DNA coding | P450 (demethylase) | NMCH-HaCYP6AE15v2_A316G_t | From | Helicoverpa |
| NO: 852 | sequence | | | | armigera |
| | of | | | | |
| SEQ ID | Amino acid | P450 (demethylase) | EcCFS-SP-HaCYP6AE15v2_A316G_t (Amino acid 1-22 −> | From | Helicoverpa |
| NO: 853 | sequence | | EcCFS N-terminal signal peptide) | | armigera |
| | of | | | | |
| SEQ ID | DNA coding | P450 (demethylase) | EcCFS-SP-HaCYP6AE15v2_A316G_t | From | Helicoverpa |
| NO: 854 | sequence | | | | armigera |
| | of | | | | |
| SEQ ID | Amino acid | P450 (demethylase) | HaCYP6AE15v2_D392E_t | From | Helicoverpa |
| NO: 855 | sequence | | | | armigera |
| | of | | | | |
| SEQ ID | DNA coding | P450 (demethylase) | HaCYP6AE15v2_D392E_t | From | Helicoverpa |
| NO: 856 | sequence | | | | armigera |
| | of | | | | |
| SEQ ID | Amino acid | P450 (demethylase) | NMCH-HaCYP6AE15v2_D392E_t (Amino acid 1-25 −> | From | Helicoverpa |
| NO: 857 | sequence | | NMCH N-terminal signal peptide) | | armigera |
| | of | | | | |
| SEQ ID | DNA coding | P450 (demethylase) | NMCH-HaCYP6AE15v2_D392E_t | From | Helicoverpa |
| NO: 858 | sequence | | | | armigera |
| | of | | | | |
| SEQ ID | Amino acid | P450 (demethylase) | EcCFS-SP-HaCYP6AE15v2_D392E_t (Amino acid 1-22 −> | From | Helicoverpa |
| NO: 859 | sequence | | EcCFS N-terminal signal peptide) | | armigera |
| | of | | | | |
| SEQ ID | DNA coding | P450 (demethylase) | EcCFS-SP-HaCYP6AE15v2_D392E_t | From | Helicoverpa |
| NO: 860 | sequence | | | | armigera |
| | of | | | | |
| SEQ ID | Amino acid | P450 (demethylase) | Hv_CYP_A0A2A4JAM9_t | From | Heliothis virescens |
| NO: 861 | sequence | | | | |
| | of | | | | |
| SEQ ID | DNA coding | P450 (demethylase) | Hv_CYP_A0A2A4JAM9_t | From | Heliothis virescens |
| NO: 862 | sequence | | | | |
| | of | | | | |
| SEQ ID | Amino acid | P450 (demethylase) | NMCH-Hv_CYP_A0A2A4JAM9_t (Amino acid 1-25 −> | From | Heliothis virescens |
| NO: 863 | sequence | | NMCH N-terminal signal peptide) | | |
| | of | | | | |
| SEQ ID | DNA coding | P450 (demethylase) | NMCH-Hv_CYP_A0A2A4JAM9_t | From | Heliothis virescens |
| NO: 864 | sequence | | | | |
| | of | | | | |
| SEQ ID | Amino acid | P450 (demethylase) | EcCFS-SP-Hv_CYP_A0A2A4JAM9_t (Amino acid 1-22 −> | From | Heliothis virescens |
| NO: 865 | sequence | | EcCFS N-terminal signal peptide) | | |
| | of | | | | |
| SEQ ID | DNA coding | P450 (demethylase) | EcCFS-SP-Hv_CYP_A0A2A4JAM9_t | From | Heliothis virescens |
| NO: 866 | sequence | | | | |
| | of | | | | |
| SEQ ID | Amino acid | P450 (demethylase) | EcCFS (CYP719A5) (Amino acid 1-22 −> | From | Eschscholzia |
| NO: 867 | sequence | | N-terminal signal peptide) | | californica |
| | of | | | | |
| SEQ ID | DNA coding | P450 (demethylase) | EcCFS (CYP719A5) | From | Eschscholzia |
| NO: 868 | sequence | | | | californica |
| | of | | | | |
| SEQ ID | Amino acid | P450 (demethylase) | EcNMCH (CYP80B2) (Amino acid 1-25 −> | From | Eschscholzia |
| NO: 869 | sequence | | N-terminal signal peptide) | | californica |
| | of | | | | |
| SEQ ID | DNA coding | P450 (demethylase) | EcNMCH (CYP80B2) | From | Eschscholzia |
| NO: 870 | sequence | | | | californica |
| | of | | | | |

### ITEMS OF THE INVENTION

The present invention further provides the following itemized embodiments:
1. A genetically modified host cell comprising a pathway having enhanced production of one or more benzylisoquinoline alkaloids wherein the cell comprises one or more features selected from:
   a) expression of one or more heterologous genes encoding one or more demethylases capable of converting thebaine into northebaine, thebaine into oripavine, thebaine into nororipavine and/or oripavine into nororipavine;
   b) expression of one or more heterologous genes encoding a tyrosine hydroxylase (TH) converting L-tyrosine into L-dopa, wherein the TH has at least 70% identity to the TH comprised in 7, 9, 11, 13, 15, 17, 19, 21, 23, 25, 27, 29, 31, 33, 35, 37, 39, 41, 43, 45, 47, 49, 51, 53, 55, 57, 59, 61, 63 or 65;
   c) reduction or elimination of activity of one or more dehydrogenases native to the host cell comprised in SEQ ID NO: 663, 665, 667, 669, 671, 673, 675, 677, 679, 681, 683, 685, 687, 689, 691, 693, 695, 697, 699, 701, 703 or 705;
   d) reduction or elimination of activity of one or more reductases native to the host cell comprised in SEQ ID NO: 707, 709, 711, 713, 715, 717, 719, 721, 723, 725, 727, 729 or 731;
   e) expression of one or more heterologous genes encoding a norcoclaurine synthase (NCS) converting Dopamine and 4-HPAA into (S)-norcoclaurine, wherein the NCS has at least 70% identity to the NCS comprised in SEQ ID NO: 73 OR 76;
   f) expression of one or more heterologous genes encoding
      i) a fused 1,2-dehydroreticuline synthase-1,2-dehydroreticuline reductase (DRS-DRR) converting (S)-Reticuline into (R)-reticuline, wherein
         ia) the DRS-DDR has at least 70% identity to the DRS-DRR comprised in SEQ ID NO: 92, 94, 96; or
         ib) the DRS moiety has at least 70%, identity to the DRS comprised in SEQ ID NO: 98, 100, 102, 104 or 106; and the DRR moiety has at least 70% identity to the DRR comprised in SEQ ID NO: 108 or 110; or
      ii) a DRS having at least 70% identity to the DRS comprised in SEQ ID NO: 98, 100, 102, 104 or 106; and a DRR having at least 70% identity to the DRR comprised in SEQ ID NO: 108 or 110;
      iii) a fused 1,2-dehydroreticuline synthase-1,2-dehydroreticuline reductase (DRS-DRR) converting (S)-Reticuline into (R)-reticuline selected from DRS-DDR's having at least 70% identity to the DRS-DRR comprised in SEQ ID NO: 92, 94, 96; and/or
      iv) a 1,2-dehydroreticuline synthase (DRS) selected from DRSs having at least 70% identity to the DRS comprised in SEQ ID NO: 98, 100, 102, 104 or 106; and a 1,2-dehydroreticuline reductases (DDR) selected from DDR's having at least 70% identity to the DRR comprised in SEQ ID NO: 108 or 110;
   g) expression of one or more heterologous genes encoding a thebaine synthase (THS) converting 7-O-acetylsalutaridinol or 7-O-acetylsalutaridinol acetate into thebaine, wherein the THS has at least 70% identity to the THS comprised in SEQ ID NO: 126, 127, 128, 129, 131, 133, 134, 136 or 138; and
   h) expression of one or more heterologous genes encoding a transporter protein capable of increasing uptake or export in the host cell of a reticuline derivative selected from transporter proteins having at least 70% identity to the transporter protein comprised in SEQ ID NO: 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 733, 735, 795, 797, 799, 801, 803, 805, 807, 809, 811, 813, 815, 817, 819, 821, 823 or 825.
2. The cell of item 1, wherein the sequence identity to any sequence is at least 90%.
3. The cell of items 1 to 2, wherein the one or more demethylase is an insect demethylase.
4. The cell of item 3, wherein the insect demethylases have a product:by-product molar ratio of at least 2.0, such as at least 2.25, such as at least 2.5, such as at least 2.75, such as at least 3.0, such as at least 3.25, such as at least 3.5, such as at least 3.75, such as at least 4.0, such as at least 4.5, such as at least 5.0, such as at least 10.0 and wherein when the product is northebaine then the by-product is thebaine N-oxide and/or northebaine oxaziridine and when the product is nororipavine then the by-product is oripavine N-oxide and/or nororipavine oxaziridine.
5. The cell of items 3 to 4, wherein the insect demethylases have N-demethylation activity and/or 0-demethylation activity.
6. The cell of items 3 to 5, wherein the insect demethylases are is of family CYP6.
7. The cell of items 3 to 6, wherein the insect is of the order Lepidoptera.
8. The cell of item 7, wherein the insect is of the genus Helicoverpa.
9. The cell of item 8, wherein the insect is of the species Helicoverpa armigera.
10. The cell of item 7, wherein the insect is of the genus Heliothis.
11. The cell of item 10, wherein the insect is of the species Heliothis virescens.
12. The cell of item 7, wherein the insect is of the genus Spodoptera.
13. The cell of item 12, wherein the insect is of the species Spodoptera exigua.
14. The cell of items 3 to 13, wherein the insect demethylase comprises a polypeptide selected from the group consisting of:
   a) a demethylase which is at least 70% identical to the insect demethylase comprised in any one of SEQ ID NO: 140, 142, 144, 146, 148, 150, 152, 154, 156, 158, 160, 162, 164, 166, 168, 170, 172, 174, 176, 178, 180, 182, 184, 186, 188, 190, 192, 194, 196, 827, 829, 831, 833, 835, 837, 839, 841, 843, 845, 847, 849, 851, 853, 855, 857, 859, 861, 863, 865, 867 and 869;
   b) a demethylase encoded by a polynucleotide which is at least 70% identical to the polynucleotide comprised in any one of SEQ ID NO: 141, 143, 145, 147, 149, 151, 153, 155, 157, 159, 161, 163, 165, 167, 169, 171, 173, 175, 177, 179, 181, 183, 185, 187, 189, 191, 193, 195, 197, 828, 830, 832, 834, 836, 838, 840, 842, 844, 846, 848, 850, 852, 854, 856, 858, 860, 862, 864, 866, 868 and 870 or genomic DNA thereof; and
   c) a functional variant of the insect demethylase of (a) or (b) capable of converting thebaine into northebaine, thebaine into oripavine, thebaine into nororipavine and/or oripavine into nororipavine.
15. The cell of item 14, wherein the insect demethylase is
   a) the demethylase comprised in any one of SEQ ID NO: 140, 142, 144, 146, 148, 150, 152, 154, 156, 158, 160, 162, 164, 166, 168, 170, 172, 174, 176, 178, 180, 182, 184, 186, 188, 190, 192, 194, 196, 827, 829, 831, 833, 835, 837, 839, 841, 843, 845, 847, 849, 851, 853, 855, 857, 859, 861, 863, 865, 867 and 869; or
   b) the demethylase encoded by a polynucleotide comprised in any one of SEQ ID NO: 141, 143, 145, 147, 149, 151, 153, 155, 157, 159, 161, 163, 165, 167, 169, 171, 173, 175, 177, 179, 181, 183, 185, 187, 189, 191, 193, 195, 197, 828, 830, 832, 834, 836, 838, 840, 842, 844, 846, 848, 850, 852, 854, 856, 858, 860, 862, 864, 866, 868 and 870 or genomic DNA thereof.
16. The cell of any preceding item, wherein the demethylases are artificial mutants comprising one or more mutations in a signal sequence.
17. The cell of item 16, wherein the signal sequence of the demethylases has been wholly or partially been replaced by a signal sequence from another enzyme.
18. The cell of item 17, wherein the demethylases are artificial mutants having least 70%, such as at least 80%, such as at least 90%, such as at least 95%, such as at least 96%, such as at least 97%, such as at least 98%, such as at least 99%, such as 100% identity to the demethylase comprised in SEQ ID NO: 845, 847, 851, 853, 857, 859, 863, 865, 867 or 869.
19. The cell of item 15, wherein the demethylases are artificial mutants having least 70%, such as at least 80%, such as at least 90%, such as at least 95%, such as at least 96%, such as at least 97%, such as at least 98%, such as at least 99%, such as 100% identity to the demethylase comprised in SEQ ID NO: 152 and comprises one or more mutations corresponding to A110X, H242X, and/or V224X, such as A110N, H242P and/or V224I.
20. The cell of item 15, wherein the demethylases are artificial mutants having at least 70%, such as at least 80%, such as at least 90%, such as at least 95%, such as at least 96%, such as at least 97%, such as at least 98%, such as at least 99%, such as 100% identity to the demethylase comprised in SEQ ID NO: 140 and comprises one or more mutations corresponding to A316X and/or D392X, such as A316G and/or D392E.
21. The cell of any preceding item, wherein the demethylase comprises one or more conserved amino acids corresponding to positions G103, H111, K167, E198, R219, L223, I256, A259, L273, V284, I309, L314, Q517, L160, N216, R443 of SEQ ID NO: 152 or conservative substitutions thereof
22. The cell of item 21, wherein the demethylase comprises a polypeptide which is at least 60% identical to the insect demethylase comprised in SEQ ID NO: 152.
23. The cell of item 21, wherein the selected one or more conserved amino acid is/are in or near the active site of the demethylase, optionally corresponding to positions G103, H111 and L314 of SEQ ID NO: 152 or conservative substitutions thereof
24. The cell of items 1 to 2, wherein the demethylase is a fungal demethylase.
25. The cell of item 24, wherein the fungus is of a genus selected from Rhizopus, Lichtheimia, Syncephalastrum, Cunninghamella, Mucor, Parasitella, Absidia, Choanephora, Bifiguratus and Choanephora.
26. The cell of item 25, wherein the fungus is of a species selected from Rhizopus microspores, Rhizopus azygosporus, Rhizopus stolonifera, Rhizopus oryzae, Rhizopus delemar, Lichtheimia corymbifera, Lichtheimia ramose, Syncephalastrum racemosum, Cunninghamella echinulate, Mucor circinelloides, Mucor ambiguous, Parasitella parasitica, Absidia repens, Absidia glauca, Choanephora cucurbitarum, Bifiguratus adelaidae and Choanephora cucurbitarum.
27. The cell of items 24 to 26, wherein the demethylase comprises a polypeptide selected from the group consisting of:
   a) a polypeptide which is at least 70% identical to the demethylase comprised in any one of SEQ ID NO: 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288 or 290;
   b) a polypeptide encoded by a polynucleotide which is at least 70% identical to the polynucleotide comprised in any one of SEQ ID NO: 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289 or 291 or genomic DNA thereof; and
   c) a functional variant of the demethylase of (a) or (b) capable of converting thebaine into northebaine, thebaine into oripavine, thebaine into nororipavine and/or oripavine into nororipavine.
28. The cell of item 27, wherein the demethylase is
   a) a polypeptide which is the demethylase comprised in any one ofSEQ ID NO: 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288 or 290; or
   b) a polypeptide encoded by a polynucleotide comprised in any one of SEQ ID NO: 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289 or 291 or genomic DNA thereof.
29. The cell of items 27 to 28, wherein the demethylase comprises an amino acid which is not one or more of the amino acids selected from:
   a) Valine at a position corresponding to V75 of SEQ ID NO: 290;
   b) Isoleucine at a position corresponding to I79 of SEQ ID NO: 290;
   c) Isoleucine at a position corresponding to V83 of SEQ ID NO: 290;
   d) Asparagine at a position corresponding to N84 of SEQ ID NO: 290;
   e) Arginine at a position corresponding to R86 of SEQ ID NO: 290;
   f) Aspartic acid at a position corresponding to D87 of SEQ ID NO: 290;
   g) Glutamic acid at a position corresponding to E126 of SEQ ID NO: 290;
   h) Threonine at a position corresponding to T145 of SEQ ID NO: 290;
   i) Asparagine at a position corresponding to N172 of SEQ ID NO: 290;
   j) Threonine at a position corresponding to T193 of SEQ ID NO: 290;
   k) Glycine at a position corresponding to G218 of SEQ ID NO: 290;
   l) Isoleucine at a position corresponding to I236 of SEQ ID NO: 290;
   m) Alanine at a position corresponding to A258 of SEQ ID NO: 290;
   n) Methionine at a position corresponding to M259 of SEQ ID NO: 290;
   o) Aspartic acid at a position corresponding to D298 of SEQ ID NO: 290;
   p) Leucine at a position corresponding to L430 of SEQ ID NO: 290;
   q) Histidine at a position corresponding to H448 of SEQ ID NO: 290;
   r) Asparagine at a position corresponding to N503 of SEQ ID NO: 290;
   s) Proline at a position corresponding to P506 of SEQ ID NO: 290;
   t) Phenylalanine at a position corresponding to F507 of SEQ ID NO: 290;
   u) Asparagine at a position corresponding to N508 of SEQ ID NO: 290; and
   v) Valine at a position corresponding to V509 of SEQ ID NO: 290.
30. The cell of items 29, wherein the demethylase comprises an amino acid which is not histidine at a position corresponding to H448 of SEQ ID NO: 290, an amino acid which is not asparagine at a position corresponding to H508 of SEQ ID NO: 290 and/or an amino acid which is not valine at a position corresponding to H509 of SEQ ID NO: 290.
31. The cell of item 30, wherein the demethylase comprises tyrosine at the position corresponding to position 448 of SEQ ID NO: 290, threonine at the position corresponding to position corresponding to H508 of SEQ ID NO: 290 and/or glycine at the position corresponding to position corresponding to H509 of SEQ ID NO: 290.
32. The cell of items 3 to 31, further comprising a demethylase-CPR capable of reducing and/or regenerating the demethylase enzyme.
33. The cell of item 32, wherein the demethylase-CPR is heterologous to the cell.
34. The cell of items 32 to 33, wherein the demethylase-CPR is derived from an insect.
35. The cell of item 34, wherein the insect demethylase-CPR is from an insect of the order Lepidoptera.
36. The cell of item 35, wherein the insect is of the genus Helicoverpa.
37. The cell of item 36, wherein the insect is of the species Helicoverpa armigera.
38. The cell of item 35, wherein the insect is of the genus Heliothis.
39. The cell of item 38, wherein the insect is of the species Heliothis virescens.
40. The cell of item 35, wherein the insect is of the genus Spodoptera.
41. The cell of item 40, wherein the insect is of the species Spodoptera exigua.
42. The cell of items 34 to 41, wherein the demethylase-CPR comprises a polypeptide selected from the group consisting of:
   a) a polypeptide which is at least 70% identical to the demethylase-CPR comprised in SEQ ID NO: 292, 294, 296, 298, 300 or 302;
   b) a polypeptide encoded by a polynucleotide which is at least 70% identical to the polynucleotide comprised in SEQ ID NO: 293, 295, 297, 299, 301, 303 or 304 or genomic DNA thereof; an
   c) a functional variant of the demethylase-CPR of (a) or (b) capable of reducing/regenerating the demethylase.
43. The cell of items 32 to 33, wherein the demethylase-CPR is a fungal demethylase-CPR.
44. The cell of item 43, wherein the demethylase-CPR comprises a polypeptide selected from the group consisting of:
   a) a polypeptide which is at least 70% identical to the demethylase-CPR comprised in any one of SEQ ID NO: 305;
   b) a polypeptide encoded by a polynucleotide which is at least 70% identical to the polynucleotide comprised in any one of SEQ ID NO: 306 or genomic DNA thereof; and
   c) a functional variant of the demethylase-CPR of (a) or (b) capable of reducing/regenerating the demethylase enzyme.
45. The cell of any preceding item, further expressing one or more genes encoding polypeptides selected from:
   a) a 3-deoxy-D-arabino-2-heptulosonic acid 7-phosphate synthase (DAHP synthase) converting PEP and E4P into DAHP;
   b) a 5-enolpyruvylshikimate-3-phosphate (EPSP) synthase (aro1) converting 3-phosphoshikimate and PEP into EPSP;
   c) an aro1 polypeptide converting DHAP and PEP into EPSP;
   d) a chorismate synthase converting EPSP into Chorismate;
   e) a chorismate mutase converting Chorismate into prephenate;
   f) a prephenate dehydrogenase (Tyr1) converting prephenate into 4-HPP;
   g) an aromatic aminotransferase converting 4-HPP into L-Tyrosine;
   h) a TH-CPR capable of reducing TH;
   i) a L-dopa decarboxylase (DODC) converting L-dopa into dopamine;
   j) a Tyrosine decarboxylase (TYDC) converting L-dopa into dopamine;
   k) a hydroxyphenylpyruvate decarboxylase (HPPDC) converting 4-HPP into 4-HPPA;
   l) a monoamine oxidase converting dopamine into 3,4-DHPAA;
   m) a 6-O-methyltransferase (6-OMT) converting (S)-norcoclaurine into (S)-Coclaurine and/or norlaudanosoline into (S)-3′-Hydroxy-coclaurine;
   n) a Coclaurine-N-methyltransferase (CNMT) converting (S)-Coclaurine into (S)—N-methylcoclaurine and/or (S)-3′-hydroxycoclaurine into (S)-3′-hydroxy-N-methyl-coclaurine;
   o) a N-methylcoclaurine hydroxylase (NMCH) converting (S)-Coclaurine into (S)-3′-hydroxycoclaurine and/or (S)—N-Methylcoclaurine into (S)-3′-Hydroxy-N-Methylcoclaurine;
   p) a 3′-hydroxy-N-methyl-(S)-coclaurine 4′-O-methyltransferase (4′-OMT) converting (S)-3′-Hydroxy-N-Methylcoclaurine into (S)-Reticuline;
   q) a DRS-CPR capable of reducing DRS-DRR;
   r) a salutaridine synthase (SAS) converting (R)-reticuline into Salutaridine;
   s) a salutaridine reductase (SAR) converting Salutaridine to Salutaridinol; and
   t) a salutaridinol 7-O-acetyltransferase (SAT) converting Salutaridinol into 7-O-acetylsalutaridinol.
46. The cell of item 45, wherein the corresponding:
   a) DAHP synthase has at least 70% identity to the DAHP synthase comprised in SEQ ID NO: 1
   b) chorismate mutase has at least 70% identity to the chorismate synthase comprised in SEQ ID NO: 3;
   c) TH-CPR has at least 70% identity to the TH-CPR comprised in SEQ ID NO: 67;
   d) DODC has at least 70% identity to the DODC comprised in SEQ ID NO: 69 or 71;
   e) 6-OMT has at least 70% identity to the 6-OMT comprised in SEQ ID NO: 79 or 81;
   f) CNMT has at least 70% identity to the CNMT comprised in SEQ ID NO: 82 or 84;
   g) NMCH has at least 70% identity to the NMCH comprised in SEQ ID NO: 85 OR 87;
   h) 4′-OMT has at least 70% identity to the 4′-OMT comprised in SEQ ID NO: 89 or 91;
   i) demethylase-CPR has at least 70% identity to the demethylase-CPR comprised in SEQ ID NO: 112 or 114;
   j) SAS has at least 70% identity to the SAS comprised in SEQ ID NO: 116 or 118;
   k) SAR has at least 70% identity to the SAR comprised in SEQ ID NO: 120 or 122;
   l) SAT has at least 70% identity to the SAT comprised in SEQ ID NO: 123 or 125; and
   m) ODM has at least 70% identity to the ODM comprised in SEQ ID NO: 218, 220, 222, 224, 226, 228, 236, 240, 250, 252, 254 and 268.
47. The cell of item any preceding item, wherein the cell is further modified to increase cytosolic levels of heme, optionally by
   a) overexpressing and/or co-expressing one or more rate-limiting proteins in the heme pathway, such as HEM 2, HEM3 and/or HEM12 optionally by increasing the number of copies of the genes integrated in the host cell and/or by linking the genes to a combination of stronger and weaker promoters, such as promoters selected from pPYK1, pSED1, pKEX2, pTEF1, pTDH3 and pPGK1, where pTEF1, pTDH3 and pPGK1; and/or
   b) disrupting, deleting and/or attenuating any heme-down regulating genes, such as HMX1.
48. The cell of item any preceding item, wherein the cell is further modified by overexpressing and/or co-expressing P450 helper genes, optionally selected from DAP1, HAC1, KAR2, HSP82, CNE1, SSA1, CPR6, FES1, HSP104 and STI1.
49. The cell of item any preceding item, wherein the cell is further modified by overexpressing and/or co-expressing one or more genes in the pentose metabolic pathway, optionally selected from ZWF1 and GND1.
50. The cell of claim any preceding claim, wherein the cell is further modified by overexpressing and/or co-expressing one or more genes encoding factors lowering and/or detoxifying cytosolic formaldehyde, optionally selected from SFA1.
51. The cell of any preceding item expressing one or more polynucleotides selected from the group of:
   a) one or more polynucleotides which is at least 70% identical to the DAHP synthase encoding polynucleotide comprised in SEQ ID NO: 2 or genomic DNA thereof;
   b) one or more polynucleotides which is at least 70% identical to the chorismate mutase encoding polynucleotide comprised in SEQ ID NO: 4 or genomic DNA thereof;
   c) one or more polynucleotides which is at least 70% identical to the TH encoding polynucleotide comprised in SEQ ID NO: 8, 10, 12, 14, 16, 18, 20, 22, 24, 26, 28, 30, 32, 34, 36, 38, 40, 42, 44, 46, 48, 50, 52, 54, 56, 58, 60, 62, 64 or 66 or genomic DNA thereof;
   d) one or more polynucleotides which is at least 70% identical to the TH-CPR encoding polynucleotide comprised in SEQ ID NO: 68 or genomic DNA thereof;
   e) one or more polynucleotides which is at least 70% identical to the DODC encoding polynucleotide comprised in SEQ ID NO: 70 or 72 or genomic DNA thereof;
   f) one or more polynucleotides which is at least 70% identical to the NCS encoding polynucleotide comprised in SEQ ID NO: 74 or 77 or genomic DNA thereof;
   g) one or more polynucleotides which is at least 70% identical to the 6-OMT encoding polynucleotide comprised in SEQ ID NO: 80 or genomic DNA thereof;
   h) one or more polynucleotides which is at least 70% identical to the CNMT encoding polynucleotide comprised in SEQ ID NO: 83 or genomic DNA thereof;
   i) one or more polynucleotides which is at least 70% identical to the NMCH encoding polynucleotide comprised in SEQ ID NO: 86 or 88 or genomic DNA thereof;
   j) one or more polynucleotides which is at least 70% identical to the 4′-OMT encoding polynucleotide comprised in SEQ ID NO: 90 or genomic DNA thereof;
   k) one or more polynucleotides which is at least 70% identical to the DRS-DRR encoding polynucleotide comprised in SEQ ID NO: 93, 95 or 97 or genomic DNA thereof;
   l) one or more polynucleotides which is at least 70% identical to the DRS encoding polynucleotide comprised in SEQ ID NO: 99, 101, 103, 105 or 107 or genomic DNA thereof;
   m) one or more polynucleotides which is at least 70% identical to the DRR encoding polynucleotide comprised in SEQ ID NO: 109 or 111 or genomic DNA thereof;
   n) one or more polynucleotides which is at least 70% identical to the demethylase-CPR encoding polynucleotide comprised in SEQ ID NO: 113 or 115 or genomic DNA thereof;
   o) one or more polynucleotides which is at least 70% identical to the SAS encoding polynucleotide comprised in SEQ ID NO: 117 or 119 or genomic DNA thereof;
   p) one or more polynucleotides which is at least 70% identical to the SAR encoding polynucleotide comprised in SEQ ID NO: 121 or genomic DNA thereof;
   q) one or more polynucleotides which is at least 70% identical to the SAT encoding polynucleotide comprised in SEQ ID NO: 124 or genomic DNA thereof;
   r) one or more polynucleotides which is at least 70% identical to the THS encoding polynucleotide comprised in SEQ ID NO: 130, 132, 135, 137 or 139 or genomic DNA thereof;
   s) one or more polynucleotides which is at least 70% identical to the ODM encoding polynucleotide comprised in SEQ ID NO: 219, 221, 223, 225, 227, 229, 237, 241, 251, 253, 255 and 267 or genomic DNA thereof;
   t) one or more polynucleotides which is at least 70% identical to the demethylase encoding polynucleotide comprised in any one of SEQ ID NO: 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 828, 830, 832, 834, 836, 838, 840, 842, 844, 846, 848, 850, 852, 854, 856, 858, 860, 862, 864, 866, 868 and 870 or genomic DNA thereof;
   u) one or more polynucleotides which is at least 70% identical to the demethylase-CPR encoding polynucleotide comprised in any one of SEQ ID NO: 293, 295, 297, 299, 301, 303, 304 or 306 or genomic DNA thereof; and
   v) one or more polynucleotides which is at least 70% identical to the transporter encoding polynucleotide comprised in SEQ ID NO: 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 606, 608, 610, 612, 614, 616, 618, 620, 622, 624, 626, 628, 630, 632, 634, 636, 638, 640, 642, 644, 646, 648, 650, 652, 654, 656, 658, 660, 662, 734 or 736 or genomic DNA thereof.
52. The cell of any preceding item wherein the cell is eukaryote selected from the group consisting of mammalian, insect, plant, or fungal cells.
53. The cell of item 52 wherein the cell is a plant cell of the genus Physcomitrella or Papaver or Nicotiana.
54. The cell of item 53 wherein the cell is a plant cell of the species Papaver soniferum or Nicotiana benthamiana.
55. The cell of item 52 wherein the cell is a fungal cell selected from the phylas consisting of Ascomycota, Basidiomycota, Neocallimastigomycota, Glomeromycota, Blastocladiomycota, Chytridiomycota, Zygomycota, Oomycota and Microsporidia.
56. The cell of item 55 wherein the fungal cell is a yeast selected from the group consisting of ascosporogenous yeast (Endomycetales), basidiosporogenous yeast, and Fungi Imperfecti yeast (Blastomycetes).
57. The cell of item 56 wherein the yeast cell is selected from the genera consisting of Saccharomyces, Kluveromyces, Candida, Pichia, Debaromyces, Hansenula, Yarrowia, Zygosaccharomyces, and Schizosaccharomyces.
58. The cell of item 57 wherein the yeast cell is selected from the species consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica.
59. The cell of item 55 wherein the fungal cell is a filamentous fungus.
60. The cell of item 59 wherein the filamentous fungal cell is selected from the phylas consisting of Ascomycota, Eumycota and Oomycota.
61. The cell of item 60 wherein the filamentous fungal cell is selected from the genera consisting of Acremonium, Aspergillus, Aureobasidium, Bjerkandera, Ceriporiopsis, Chrysosporium, Coprinus, Corio/us, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma
62. The cell of item 61 wherein the filamentous fungal cell is selected from the species consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.
63. The cell of any preceding item, wherein one or more further native or endogenous genes of the cell is attenuated, disrupted and/or deleted.
64. The cell of any preceding item, further comprising at least 2 copies of one or more genes in the benzylisoquinoline alkaloid pathway.
65. The cell of any preceding item, wherein one or more genes of the benzylisoquinoline alkaloid pathway are overexpressed.
66. The cell of any preceding item further genetically modified to provide an increased amount of a substrate for at least one polypeptide of the benzylisoquinoline alkaloid pathway.
67. The cell of any preceding item further genetically modified to exhibit increased tolerance towards one or more substrates, intermediates, or product molecules from the benzylisoquinoline alkaloid pathway.
68. A polynucleotide construct comprising a polynucleotide sequence encoding a heterologous enzymes or transporter protein of any preceding item operably linked to one or more control sequences.
69. The polynucleotide construct of item 68 wherein the control sequence is heterologous to the polynucleotide.
70. The polynucleotide construct of item 69, wherein the construct is an expression vector.
71. The cell of any preceding item comprising the polynucleotide construct of items 68 to 70.
72. A cell culture, comprising the cell of any preceding item and a growth medium.
73. A method for producing a benzylisoquinoline alkaloid comprising
   a) culturing the cell culture of item 72 at conditions allowing the cell to produce the benzylisoquinoline alkaloid; and
   b) optionally recovering and/or isolating the benzylisoquinoline alkaloid.
74. The method of item 73, wherein the recovering and/or isolation step comprises separating a liquid phase of the cell or cell culture from a solid phase of the cell or cell culture to obtain a supernatant comprising the benzylisoquinoline alkaloid and subjecting the supernatant to one or more steps selected from:
   a) contacting the supernatant with one or more adsorbent resins in order to obtain at least a portion of the produced benzylisoquionoline alkaloid, then optionally recovering the benzylisoquionoline alkaloid from the resin in a concentrated solution prior to precipitation or crystallisation of the benzylisoquionoline alkaloid;
   b) contacting the supernatant with one or more ion exchange or reversed-phase chromatography columns in order to obtain at least a portion of the benzylisoquionoline alkaloid, then optionally recovering the benzylisoquionoline alkaloid from the resin in a concentrated solution prior to precipitation or crystallisation of the benzylisoquionoline alkaloid;
   c) extracting the benzylisoquionoline alkaloid from the supernatant, such as by liquid-liquid extraction into an immisible solvent, then optionally evaporating the solvent to concentrate and precipitate the benzylisoquionoline alkaloid or performing further liquid-liquid extraction to recover and concentrate benzylisoquionoline alkaloid prior to crystallisation or precipitation or in order to directly perform a further chemical reaction on benzylisoquionoline alkaloid; and
   thereby recovering and/or isolating the benzylisoquinoline alkaloid.
75. The method of items 73 to 74, further comprising one or more elements selected from:
   a) culturing the cell culture in a nutrient medium;
   b) culturing the cell culture under aerobic or anaerobic conditions
   c) culturing the cell culture under agitation;
   d) culturing the cell culture at a temperature of between 25 to 50° C.;
   e) culturing the cell culture at a pH of between 3-9; and
   f) culturing the cell culture for between 10 hours to 30 days.
76. The method of items 73 to 75, wherein one or more steps of producing the benzylisoquinoline alkaloid is performed in vitro.
77. The method of items 73 to 76, comprising converting thebaine to northebaine in the cell, wherein the conversion is performed at a pH from 6 to 8, such as from 6.5 to 7.5, such as about 7.0.
78. The method of item s 73 to 76, comprising converting oripavine to nororipavine in the cell, wherein the conversion is performed at a pH from 3.5 to 5.5, such as from 3.0 to 5.0, such as about 4.5.
79. The method of item 73 to 78, comprising feeding the cell culture with one or more exogenous benzylisoquinoline alkaloid precursors.
80. The method of item 79, wherein the exogenous benzylisoquinoline alkaloid precursor is thebaine and/or oripavine.
81. The method of item 79, wherein the exogenous benzylisoquinoline alkaloid precursor is comprised in a plant extract.
82. The method of items 73 to 81, wherein the benzylisoquinoline alkaloid is selected from one or more of thebaine, northebaine, oripavine and nororipavine.
83. The method of items 73 to 82, wherein the benzylisoquinoline alkaloid is of the general formula R1-V-H (V):

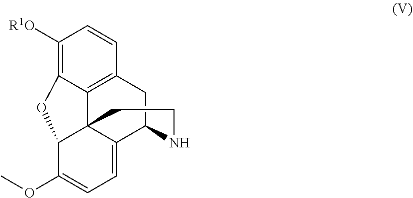

or a salt thereof.
84. The method of item 84, wherein the benzylisoquinoline alkaloid is a nororipavine, HO—V—H (VI), of the general formula:

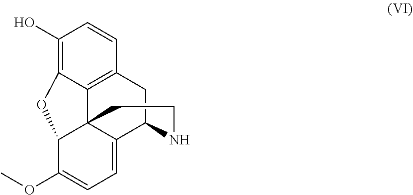

or a salt thereof.
85. The method of item 84 to 84, further comprising chemically or biologically modifying the benzylisoquinoline alkaloid.
86. The method of item 85, wherein the modified benzylisoquinoline alkaloid is selected from one or more of buprenorphine, naltrexone, naloxone and nalbuphine.
87. The method of item 85 or 86, wherein the benzylisoquinoline alkaloid to be modified is one or more of thebaine, northebaine, oripavine or nororipavine and the method further comprises subjecting the benzylisoquinoline alkaloid in sequence to a bis-benzylation step, a Diels-Alder step and a Grignard step converting the benzylisoquinoline alkaloid into buprenorphine.
88. The method of item 87, wherein the benzylisoquinoline alkaloid to be modified is HO—VI-H (VI).
89. The method of item 88, further comprising:
   a) in a first solvent system S-1 comprising a polar protic solvent, reacting the compound HO—VI-H (VI), with benzyl halide, benzyl sulfonate, or activated benzyl alcohol to provide a compound BnO—VI-Bn (VII) of the general formula:

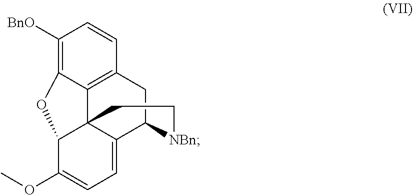

b) in a second solvent system 5-2 comprising a polar protic solvent, reacting compound BnO—VI-Bn (VII) with methyl vinyl ketone to provide a compound BnO-VII-Bn (VIII) of the general formula:

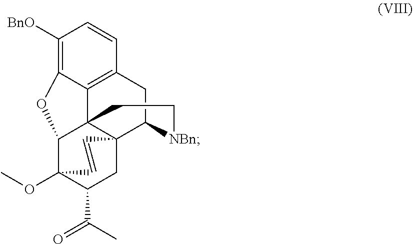

c) in a third solvent system S-3 comprising a nonpolar solvent, reacting compound BnO—VII-Bn (VIII) with a tert-butylmagnesium compound to provide a compound BnO-VIIIA-Bn (IX) of the general formula:

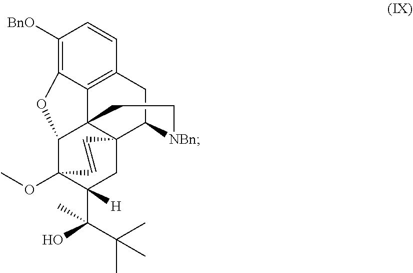

d) reacting Compound BnO-VIIIA-Bn (IX) with H2 in the presence of a hydrogenation catalyst to provide a compound HO—IX—H (X) of the general formula:

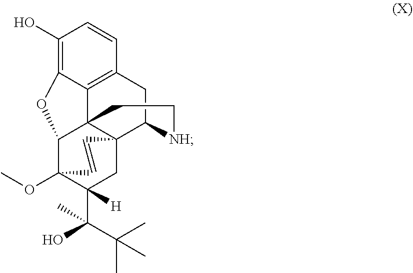

e) reacting Compound HO—IX—H (X) with
   i. cyclopropane carboxaldehyde followed by a hydride source; or:
   ii. cyclopropanecarboxylic acid halide followed by a reducing agent; or
   iii. cyclopropylmethyl halide or activated cyclopropane methanol;
      to provide buprenorphine.
90. The method of item 89, wherein S-1 comprises at least one protic solvent having a dielectric constant of at least about 12, or at least about 14, or at least about 16.
91. The method of item 90, wherein S-1 comprises at least about 50 vol. %, or at least about 75 vol. %, or at least about 90 vol. % of the at least one protic solvent having a dielectric constant of at least about 12 (e.g. at least 14, or at least 16).
92. The method of item 89, wherein S-1 comprises at least one protic solvent having a polarity index of at least about 3, or at least about 3.5, or at least about 4.
93. The method of item 92, wherein S-1 comprises at least about 50 vol. %, or at least about 75 vol. %, or at least about 90 vol. % of the at least one protic solvent having a polarity index of at least about 3, e.g., at least 3.5, or at least 4.
94. The method of items 89 to 93, wherein S-2 comprises at least one protic solvent having a dielectric constant of at least about 12, or at least about 14, or at least about 16.
95. The method of item 94, wherein S-2 comprises at least about 50 vol. %, or at least about 75 vol. %, or at least about 90 vol. % of the at least one protic solvent having a dielectric constant of at least about 12, e.g. at least 14, or at least 16.
96. The method of items 89 to 93, wherein S-2 comprises at least one protic solvent having a polarity index of at least about 3, or at least about 3.5, or at least about 4.
97. The method of item 96, wherein S-2 comprises at least about 50 vol. %, or at least about 75 vol. %, or at least about 90 vol. % of the at least one protic solvent having a polarity index of at least about 3, e.g. at least 3.5, or at least 4.
98. The method of items 89 to 97, wherein S-1 comprises isopropanol and optionally water.
99. The method of items 89 to 98, wherein S-2 comprises isopropanol and optionally water.
100. The method of items 98 or 99, wherein S-1 and/or 5-2 comprises about 50-100 vol. % isopropanol and 0 to about 50 vol. % water.
101. The method of items 89 to 100, wherein step 89.b) is conducted in the presence of oxygen.
102. The method of items 89 to 101, wherein the methyl vinyl ketone of step 89.b) is added to a crude reaction product of step 89.a), the crude reaction product comprising solvent S-1 and compound BnO-II-Bn (VII).
103. The method of items 89 to 102, wherein S-3 comprises at least one nonpolar solvent having a dielectric constant of at most about 6, or at most about 5, or at most about 4.
104. The method of item 103, wherein S-3 comprises at least about 50 vol. %, or at least about 75 vol. %, or at least about 90 vol. % of the at least one nonpolar solvent having a dielectric constant of at most 6, e.g. at most 5, or at most 4.
105. The method of items 89 to 102, wherein S-3 comprises at least one nonpolar solvent having a polarity index of less than 3, or less than 2, or less than 1.
106. The method of item 105, wherein S-3 comprises at least about 50 vol. %, or at least about 75 vol. %, or at least about 90 vol. % of the at least one nonpolar solvent having a polarity index of less than 3, e.g. less than 2, or less than 1.
107. The method of items 89 to 106, wherein 5-3 comprises less than about 10 vol. %, or less than about 5 vol. %, or less than about 2 vol. %, or less than about 1 vol. % of a total amount of solvents having a dielectric constant of greater than 6.
108. The method of items 89 to 106, wherein S-3 comprises less than 10 vol. %, or less than 5 vol. %, or less than 2 vol. %, or less than 1 vol. % of total amount of solvents having a polarity index of 3 or greater.
109. The method of items 89 to 108, wherein S-3 comprises 30-90 vol. % of one or more alkanes and/or cycloalkanes.
110. The method of item 109, wherein the one or more alkanes and/or cycloalkanes comprises, e.g. is cyclohexane.
111. The method of items 89 to 110, wherein S-3 comprises 10-50 vol. % toluene, 30-90 vol. % cyclohexane, and up to 30 vol. % tetrahydrofuran.
112. The method of items 89 to 111, wherein the tert-butylmagnesium compound comprises one or both of a tert-butylmagnesium halide and di-tert-butylmagnesium.
113. The method of items 89 to 111, wherein the tert-butylmagnesium compound comprises a tert-butylmagnesium halide and di-tert-butylmagnesium.
114. A fermentation composition comprising the cell culture of item 72 and the benzylisoquinoline alkaloid comprised therein.
115. The fermentation composition of item 114, wherein at least 10%, 25%, 50%, such as at least 75%, such as at least 95%, such as at least 99% of the cells are lysed.
116. The fermentation composition of items 114 to 115, wherein at least 10%, 25%, 50%, such as at least 75%, such as at least 95%, such as at least 99% of solid cellular material has separated from the liquid.
117. The fermentation composition of item 114 to 116, further comprising one or more compounds selected from trace metals, vitamins, salts, yeast nitrogen base, carbon source, YNB, and/or amino acids of the fermentation; wherein the concentration of the benzylisoquinoline alkaloid is at least 1 mg/kg composition.
118. A composition comprising the fermentation composition of any preceding item and one or more carriers, agents, additives and/or excipients.
119. A pharmaceutical composition comprising the fermentation composition of any preceding item and one or more pharmaceutical grade excipient, additives and/or adjuvants.
120. The pharmaceutical composition of item 119, wherein the pharmaceutical preparation is in form of a powder, tablet or a capsule.
121. The pharmaceutical composition of item 119, wherein the pharmaceutical preparation is in form of a pharmaceutical solution, suspension, lotion or ointment.
122. The pharmaceutical composition of items 119 to 121 for use as a medicament for prevention, treatment and/or relief of a disease in a mammal.
123. The pharmaceutical composition of item 122 for use in the prevention, treatment and/or relief of pain, infections, tussive conditions, parasitic conditions, cytotoxic conditions, opiate poisoning conditions and/or cancerous conditions in a mammal.
124. A method for preparing the pharmaceutical composition of item 119 to 123 comprising mixing the fermentation composition of items 114 to 117 with one or more pharmaceutical grade excipient, additives and/or adjuvants.
125. A method for preventing, treating and/or relieving a disease comprising administering a therapeutically effective amount of the pharmaceutical composition of items 119 to 121 to a mammal.
126. The method of item 125, wherein the disease is pain, infections, tussive conditions, parasitic conditions, cytotoxic conditions, opiate poisoning conditions and/or cancerous conditions.
127. A mutant insect demethylase comprising one or more mutations in the signal sequence of the naturally occurring insect demethylase.
128. The mutant demethylase of item XX, wherein the signal sequence of the demethylase has been wholly or partially been replaced by a signal sequence from another enzyme.
129. The mutant demethylase of item XX, wherein the demethylase has least 70%, such as at least 80%, such as at least 90%, such as at least 95%, such as at least 96%, such as at least 97%, such as at least 98%, such as at least 99%, such as 100% identity to the demethylase comprised in SEQ ID NO: 845, 847, 851, 853, 857, 859, 863, 865, 867 or 869.
130. A mutant insect demethylase having least 70%, such as at least 80%, such as at least 90%, such as at least 95%, such as at least 96%, such as at least 97%, such as at least 98%, such as at least 99%, such as 100% identity to the demethylase comprised in SEQ ID NO: 152 and comprising one or more mutations corresponding to A110X, H242X, and/or V224X, optionally A110N, H242P and/or V224I.
131. A mutant insect demethylase having at least 70%, such as at least 80%, such as at least 90%, such as at least 95%, such as at least 96%, such as at least 97%, such as at least 98%, such as at least 99%, such as 100% identity to the demethylase comprised in SEQ ID NO: 140 and comprising one or more mutations corresponding to A316X and/or D392X, optionally A316G and/or D392E.

### EXAMPLES

### Materials and Methods

Chemicals used in the examples herein, e.g. for buffers and substrates, are commercial products of at least reagent grade. Water utilized in the examples was de-ionized, MilliQ water.

For examples 1 through 6, Saccharomyces cerevisiae yeast strains were constructed in strain background sOD157 (MATa his3 Δ0 leu2Δ0 ura3Δ0 CATS-91Met GAL2 ho MIP1-661Thr SAL1-1). Strain sOD157 with the said genotype corresponds to strain S288C (genotype MATa his3 Δ0 leu2 Δ0 ura3 Δ0) which is a publicly available widely used laboratory strain (see the Saccharomyces Genome Database (SGD)). Accordingly, similar results can be reached by using strain S288C as the results demonstrated below using strain sOD157 as base strain for modification, background and/or control. All strain transformations with relevant plasmids was done using the lithium acetate method (Gietz et al. 2007).

For examples 7 through 21, Saccharomyces cerevisiae yeast strains were constructed in strain background EVST25898 (genotype MATalpha his3 Δ0 leu2 Δ0 ura3 Δ0 aro3Δ::pTEF1-ARO4(K229L)-tCYC1::pPGK1-ARO7(T266L)-tADH1::KI CAT5-91Met GAL2 ho MIP1-661Thr SAL1-1 YORWΔ22::npBIO1nt20npBIO6nt). The EVST25898 with the genotype above corresponds to S288C (genotype MATalpha his3 Δ0 leu2 Δ0 ura3 Δ0). S288C is a publicly available widely used laboratory strain (see the Saccharomyces Genome Database (SGD)). As is known from other works, one would get similar results by use of EVST25898 with genotype above or by use of S288C (genotype MATalpha his3 Δ0 leu2 Δ0 ura3 Δ0) as background/control strains, since these two host phenotypes are substantially identical.

For examples 22 through 25, Saccharomyces cerevisiae yeast strain BY4741 was constructed from S288C, which is a publicly available widely used laboratory strain (see the Saccharomyces Genome Database (SGD) eg. available from http://www.euroscarf.de.

Promoters and plasmids used throughout the examples were, unless otherwise characterized, standard promoters and plasmids abundantly know to the skilled person.

### Example 1—Modification of Base Strain to Express Moth Demethylase Enzymes

For testing the N-demethylation of thebaine to northebaine and N-demethylation of oripavine to nororipavine by expression of a moth demethylase, a strain of sOD157 was transformed with a plasmid containing a demethylase-CPR Ha_CPR E0A3A7 (SEQ ID NO: 292) from Helicoverpa armigera (pOD1184) and a permease T102_PsoPUP3_1 (SEQ ID NO: 466) from Papaver somniferum (pOD635) in combination with a selection of representative insect demethylase selected from several moth species. All sequences tested were codon optimized for expression in S. cerevisiae. Genes were inserted and expressed using either plasmids P413TEF, P415TEF or p416TEF, all described by (Mumberg, Müller and Funk 1995). The control strain was constructed by transforming the sOD157 strain with plasmid pOD1184 designed according to table 1-1 below, as well as an empty plasmid: p415TEF and transformants were selected in synthetic complete (SC) agar plates lacking histidine, leucine and uracil. Transformation plates were incubated for 3-4 days at 30° C. until visible colonies were obtained.

TABLE 1-1
Plasmids introduced in the corresponding yeast strains
for heterologous expression of moth demethylase.

| | | | Yeast Selection | |
|---|---|---|---|---|
| Vector Name | Backbone | Promoter-Gene-Terminator | Marker | Description |
| pOD1184 | P413TEF | pTEF1-Ha_CPR_E0A3A7- | HIS3 | Ha_CPR_E0A3A7 from |
| | | tCYC1 | | Helicoverpa armigera |
| Empty | P415TEF | No gene inserted | LEU2 | (Mumberg, Müller and |
| | | | | Funk 1995) |
| pOD635 | P416TEF | pTEF1- | URA3 | PsoPUP3_1 from |
| | | T102_PsoPUP3_1-tCYC1 | | Papaver somniferum |

### Example 2—Modification of Base Strain to Express Fungal Demethylase Enzymes

For testing the N-demethylation of thebaine to northebaine, O-demethylation of thebaine to oripavine and N-demethylation oripavine to nororipavine by expression of a fungal demethylase, a sOD157 strain was co-transformed with a plasmid containing a Demethylase-CPR Cel_CPR from Cunninghamella elegans (SEQ ID NO: 305) (pOD13) and a permease T14_PsoNPF3_GA (SEQ ID NO: 328) in combination with a selection of fungal demethylase. All sequences tested were codon optimized for expression in S. cerevisiae. Genes were inserted and expressed using either P413TEF, P415TEF or p416TEF, all described by (Mumberg, Müller and Funk 1995). The control strain was constructed by transforming the sOD157 strain with plasmid pOD13 designed according to table 2-1 below as well as an empty plasmid: p415TEF, and transformants were selected as described above for testing of insect demethylase.

TABLE 2-1
Plasmids introduced in the corresponding yeast strains
for heterologous expression of fungal demethylase.

| | | | Yeast Selection | |
|---|---|---|---|---|
| Vector Name | Backbone | Promoter-Gene-Terminator | Marker | Description |
| pOD13 | P413TEF | pTEF1-Cel_CPR-tCYC1 | HIS3 | Cel_CPR from |
| | | | | Cunninghamella elegans |
| Empty | P415TEF | No gene inserted | LEU2 | (Mumberg, Müller and |
| | | | | Funk 1995) |
| pOD353 | P416TEF | pTEF1-T14_PsNPF3-tCYC1 | URA3 | T14_PsNPF3 from |
| | | | | Papaver alpinum |

### Example 3—Cultivation and Harvest of Yeast Strains

### Cultivation

Yeast strains were cultivated in 96-deep-well-plate (DWP) format. Cells were grown in 0.5 ml SC-His-Leu-Ura medium at 30° C. with shaking at 250 rpm in ISF1-X Kuhner shaker for 20-24 hours and utilized as pre-cultures for in vivo bioconversion assays.

50 μl of the overnight cell cultures were grown in 450 μl DELFT minimal medium (pH 7.0 or pH 4.5) containing 0.1 M potassium phosphate buffer.

Thebaine (or oripavine) were added via a 25 mM stock solution in DMSO. Cells were grown for 72 hours with shaking at 250 rpm.

### Harvest.

60 μl of cell cultures were transferred to a new 96-deep-well-plate containing 50 μl of MilliQ water with 0.1% of formic acid. The harvested 96 well plate was incubated at 80° C. for 10 minutes. Plate was then centrifugated for 10 minutes at 4000 rpm. The supernatants were then taken for analysis with samples having a final dilution of 1:1. Thebaine, northebaine, oripavine, northebaine-oxaziridine, thebaine N-oxide, nororipavine, nororipavine-oxaziridine, and oripavine N-oxide contents were analyzed by HPLC.

### Example 4—HPLC Analysis

For all compounds (thebaine, northebaine, oripavine and nororipavine) stock solutions were prepared in DMSO at a concentration of 10 mM. Standard solutions were prepared at concentrations of 50 μM, 100 μM, 250 μM and 500 μM from the stock solutions. Samples were injected into an Agilent 1290 Infinity|UHPLC with a binary pump (Agilent Technologies, Palo Alto, CA, USA). Separation was achieved on a Kinetex F5 column (100×2.1 mm, 1.7 μm, 100 Å, Phenomenex, Torrance, CA, USA) using 0.05% (v/v) formic acid in H₂O and 0.05% (v/v) formic acid in acetonitrile as mobile phases A and B, respectively using the time-gradient as shown in table 4-1.

TABLE 4-1
Gradient for HPLC

| Time (min) | % B |
|---|---|
| 0.0-3.6 | 2-30 |
| 3.6-4.1 | 30-100 |
| 4.1-5.1 | 100 |
| 5.1-5.5 | 100-2 |
| 1 | 2 |

The injection volume was 1 μL and the mobile phase flow rate was 600 μL/min. The column temperature was maintained at 30° C. The liquid chromatography system was coupled to an Agilent 1290 diode array detector (Agilent Technologies, Palo Alto, CA, USA). UV-spectra were acquired at 220, 254 and 285 nm with 285 nm used for quantification of nororipavine, oripavine, northebaine and thebaine.

### Example 5—Identification of Performing Moth Demethylases for the Conversion of Thebaine and Oripavine

Rates for a select group of insect demethylase for converting thebaine into northebaine or by-products northebaine-Oxaziridine or thebaine N-oxide are shown in tables 5-1 and 5-2, while conversion rates for the select group of insect demethylase for converting oripavine into nororipavine or by-products nororipavine-oxaziridine or oripavine N-oxide are shown in tables 5-3 and 5-4.

TABLE 5-1
Bioconversion of thebaine to northebaine in strains expressing different possible
demethylase enzymes from insect, co-expressed with a demethylase-CPR from
Helicoverpa armigera (HaCPR_E0A3A7) and a permease from Papaver somniferum
(T102_PsoPUP3_1), and grown in DELFT minimal medium at pH 7.0.

| | SEQ | Northebaine | Thebaine | Northebaine-Oxaziridine | Thebaine N-oxide |
|---|---|---|---|---|---|
| Demethylase | ID NO | (%) | (%) | (%) | (%) |
| Hv_CYP_A0A2A4JAM9 | 152 | 44.33 | 50.91 | Below detection | 4.76 |
| | | | | limit | |
| HaCYP6AE15v2 | 140 | 30.74 | 62.83 | 3.31 | 3.11 |
| Se_CYP6AE68 | 156 | 27.26 | 69.91 | Below detection | 2.83 |
| | | | | limit | |
| Hv_CYP_A0A2A4JAK3 | 154 | 22.62 | 77.38 | Below detection | Below detection |
| | | | | limit | limit |
| Sf_A0A2H1WID4 | 168 | 18.39 | 81.61 | Below detection | Below detection |
| | | | | limit | limit |
| HaCYP6AE19 | 142 | 15.06 | 81.31 | Below detection | 3.63 |
| | | | | limit | |
| Sf_CYP6AE44 | 174 | 14.43 | 82.96 | Below detection | 2.61 |
| | | | | limit | |
| Ha_CYP6AE12 | 172 | 13.99 | 85.19 | Below detection | 0.82 |
| | | | | limit | |
| Hv_CYP_A0A2A4J7V4 | 158 | 10.24 | 89.76 | Below detection | Below detection |
| | | | | limit | limit |
| HaCYP6AE17 | 146 | 6.31 | 93.69 | Below detection | Below detection |
| | | | | limit | limit |
| DpCYP_Q7YZS2 | 178 | 6.16 | 93.84 | Below detection | Below detection |
| | | | | limit | limit |
| HaCYP6AE_A0A068F0X7 | 176 | 5.50 | 94.50 | Below detection | Below detection |
| | | | | limit | limit |
| HaCYP6AE24 | 148 | 3.02 | 96.97 | Below detection | Below detection |
| | | | | limit | limit |
| HaCYP6AE11 | 144 | 2.57 | 97.42 | Below detection | Below detection |
| | | | | limit | limit |
| Bm_CYP6AE9 | 352 | 4.88456 | 90.23088 | Below detection | Below detection |
| | | | | limit | limit |
| Control plasmids | — | Below detection | 100 | Below detection | Below detection |
| | | limit | | limit | limit |

TABLE 5-2
Bioconversion of thebaine to northebaine in strains co-expressing different possible demethylase
enzymes from insect and different possible demethylase-CPR enzymes from insect with a permease from
Papaver somniferum (T102_PsoPUP3_1), and grown in DELFT minimal medium at pH 7.0.

| | | | | | Northebaine- | Thebaine |
|---|---|---|---|---|---|---|
| | | SEQ | Northebaine | Thebaine | Oxaziridine | N-oxide |
| Demethylase | Demethylase-CPR | ID NO | (%) | (%) | (%) | (%) |
| CmCYP6_A0A0C5CGV6 | CmCPR_A0A1S5ZY34 | 160/302 | 19.05 | 80.41 | Below | 0.54 |
| | | | | | detection | |
| | | | | | limit | |
| Se_CYP6AE68 | Se_CPR_F1DI27 | 156/294 | 16.00 | 81.91 | Below | 2.09 |
| | | | | | detection | |
| | | | | | limit | |
| BmCYP6AE9_A9QW15 | BmCPR_A0FGR6 | 162/298 | 15.32 | 84.46 | 0.23 | Below |
| | | | | | | detection |
| | | | | | | limit |
| ZfCYP6AE27_D2JLK6 | ZfCPR_A0A346M705 | 186/300 | 14.42 | 85.58 | Below | Below |
| | | | | | detection | detection |
| | | | | | limit | limit |
| Sf_CYP6AE44 | Se_CPR_F1DI27 | 174/294 | 11.28 | 86.25 | Below | 2.47 |
| | | | | | detection | |
| | | | | | limit | |
| BmCYP6AE2_L0N7C5 | Bm_CPR_Q9NKV3 | 182/296 | 11.01 | 88.99 | Below | Below |
| | | | | | detection | detection |
| | | | | | limit | limit |
| Sf_A0A2H1WID4 | Se_CPR_F1DI27 | 168/294 | 9.55 | 88.62 | Below | 1.82 |
| | | | | | detection | |
| | | | | | limit | |
| BmCYP6AE9_A5HKM1 | Bm_CPR_Q9NKV3 | 196/296 | 7.56 | 92.44 | Below | Below |
| | | | | | detection | detection |
| | | | | | limit | limit |
| CmCYP6_A0A0C5C1I6 | CmCPR_A0A1S5ZY34 | 192/302 | 5.93 | 94.07 | Below | Below |
| | | | | | detection | detection |
| | | | | | limit | limit |
| BmCYP_C1KJL7 | BmCPR_A0FGR6 | 184/298 | 5.91 | 94.10 | Below | Below |
| | | | | | detection | detection |
| | | | | | limit | limit |
| BmCyp6AE21_B6VFR9 | Bm_CPR_Q9NKV3 | 188/296 | 5.72 | 94.28 | Below | Below |
| | | | | | detection | detection |
| | | | | | limit | limit |
| Sf_CYP_A0A2H1V0E7 | Se_CPR_F1DI27 | 170/294 | 5.50 | 92.15 | Below | 2.35 |
| | | | | | detection | |
| | | | | | limit | |
| BmCYP6AE7_A4GUB8 | Bm_CPR_Q9NKV3 | 190/296 | 5.48 | 94.52 | Below | Below |
| | | | | | detection | detection |
| | | | | | limit | limit |
| SeCYP6_A0A248QEH8 | SeCPR_F1DI27 | 194/294 | 4.24 | 95.76 | Below | Below |
| | | | | | detection | detection |
| | | | | | limit | limit |
| Control plasmids | | — | Below | 100 | Below | Below |
| | | | detection | | detection | detection |
| | | | limit | | limit | limit |

TABLE 5-3
Bioconversion of oripavine to nororipavine in strains expressing different possible demethylase
enzymes from insect, co-expressed with a demethylase-CPR from H. armigera (HaCPR_E0A3A7) and a
permease from Papaver somniferum (T102_PsoPUP3_1), and grown in DELFT minimal medium at pH 4.5.

| | SEQ | Nororipavine | Oripavine | Nororipavine-oxaziridine | Oripavine N-oxide |
|---|---|---|---|---|---|
| Demethylase | ID NO | (%) | (%) | (%) | (%) |
| Hv_CYP_A0A2A4JAM9 | 152 | 35.83 | 60.64 | 1.576380207 | 1.95 |
| HaCYP6AE15v2 | 140 | 26.98 | 71.60 | 0.38907368 | 1.02 |
| CmCYP6_A0A0C5CGV6 | 160 | 17.23 | 82.04 | Below detection | 0.72 |
| | | | | limit | |
| BmCYP6AE9_A9QW15 | 162 | 21.40 | 77.51 | Below detection | 1.09 |
| | | | | limit | |
| HaCYP6AE19 | 142 | 11.17 | 88.21 | Below detection | 0.62 |
| | | | | limit | |
| Hv_CYP_A0A2A4JAK3 | 154 | 6.52 | 91.81 | Below detection | 1.68 |
| | | | | limit | |
| Se_CYP6AE68 | 156 | 5.67 | 92.80 | Below detection | 1.53 |
| | | | | limit | |
| Hv_CYP_A0A2A4J7V4 | 158 | 4.51 | 94.38 | Below detection | 0.67 |
| | | | | limit | |
| Bm_CYP6AE9 | 165 | 4.282177 | 93.88658 | Below detection | 1.83124 |
| | | | | limit | |
| Control plasmids | — | Below detection | 100 | Below detection | Below detection |
| | | limit | | limit | limit |

TABLE 5-4
Bioconversion of oripavine to nororipavine in strains co-expressing different possible demethylase
enzymes from insect and different demethylase-CPR enzymes from insect with a permease from
Papaver somniferum (T102_PsoPUP3_1), and grown in DELFT minimal medium at pH 4.5.

| | | | | | Nororipavine- | Oripavine |
|---|---|---|---|---|---|---|
| | | SEQ | Nororipavine | Oripavine | Oxaziridine | N-oxide |
| Demethylase | Demethylase-CPR | ID NO | (%) | (%) | (%) | (%) |
| CmCYP6_A0A0C5CGV6 | CmCPR_A0A1S5ZY34 | 160/302 | 17.07 | 82.68 | Below | 0.25 |
| | | | | | detection | |
| | | | | | limit | |
| Se_CYP6AE68 | Se_CPR_F1DI27 | 156/294 | 16.91 | 79.93 | Below | 3.16 |
| | | | | | detection | |
| | | | | | limit | |
| BmCYP6AE9_A9QW15 | BmCPR_A0FGR6 | 162/298 | 12.60 | 87.40 | Below | Below |
| | | | | | detection | detection |
| | | | | | limit | limit |
| Sf_A0A2H1WID4 | Se_CPR_F1DI27 | 168/294 | 10.79 | 88.37 | Below | 0.83 |
| | | | | | detection | |
| | | | | | limit | |
| CmCYP6_A0A0C5CGV6 | CmCPR_A0A1S5ZY34 | 160/302 | 8.80 | 91.20 | Below | Below |
| | | | | | detection | detection |
| | | | | | limit | limit |
| Sf_CYP_A0A2H1V0E7 | Se_CPR_F1DI27 | 170/294 | 8.17 | 91.83 | Below | Below |
| | | | | | detection | detection |
| | | | | | limit | limit |
| Se_CYP6AE10 | Se_CPR_F1DI27 | 166/294 | 5.58 | 92.36 | Below | 2.05 |
| | | | | | detection | |
| | | | | | limit | |
| Bm_CYP6AE9 | Bm_CPR_Q9NKV3 | 196/296 | 4.28 | 93.89 | Below | 1.83 |
| | | | | | detection | |
| | | | | | limit | |
| Control plasmids | | — | Below | 100 | Below | Below |
| | | | detection | | detection | detection |
| | | | limit | | limit | limit |

Surprisingly at was found that insect demethylase can actually be expressed in yeast and that they are capable of in vivo converting thebaine and/or oripavine to Northebaine and/or nororipavine with high efficiency and with production of very little by-products. Expression of demethylase gene HaCYP6AE15v2 (SEQ ID NO: 141) from H. armigera in a strain containing the demethylase-CPR gene HaCPR_E0A3A7 (SEQ ID NO: 293) from H. armigera, exhibited an N-demethylation of thebaine to northebaine of approximately 31% and N-demethylation of oripavine to nororipavine of approximately 27%, without significant presence of oxaziridines or N-oxides. Expression of demethylase gene Hv_CYP_A0A2A4JAM9 (SEQ ID NO: 153) from Heliothis virescens in a strain containing the demethylase-CPR gene HaCPR_E0A3A7 (SEQ ID NO: 293) from H. armigera, exhibited an N-demethylation of thebaine to northebaine of approximately 44% and N-demethylation of oripavine to nororipavine of approximately 36%, without significant presence of oxaziridines or N-oxides. In fact, expression of demethylase Hv_CYP_A0A2A4JAM9 gave approximately 279% more conversion of oripavine to nororipavine when compared to the best fungal demethylase (CYPDN_92—SEQ ID NO: 252) described in example 6 below—which is a remarkable yield improvement. Additionally, expression of demethylase Hv_CYP_A0A2A4JAM9 gave approximately 5.4% more conversion of thebaine to northebaine with significantly less by-products when compared to the best fungal demethylase (CYPDN_92—SEQ ID NO: 252) described in example 6 below—which is also a remarkable yield improvement.

For testing the N-demethylation of thebaine to northebaine and N-demethylation of oripavine to nororipavine by expression of a moth cytochrome P450, a yeast strain was transformed with a plasmid containing an NADPH-cytochrome P450 reductase and an uptake transporter in combination with the various putative moth cytochrome P450 proteins.

TABLE 5-5
Bioconversion of thebaine to northebaine in strains expressing different candidate demethylase
enzymes from insect, co-expressed with a demethylase-CPR from Helicoverpa armigera (SEQ ID
NO 293) and a permease from Papaver somniferum (SEQ ID NO 466) (T102_PsoPUP3_1),
and grown in DELFT minimal medium at pH 7.0 with 500 μM of thebaine.

| Cytochrome | SEQ | Thebaine | Northebaine | Northebaine-Oxaziridine | Thebaine N-oxide |
|---|---|---|---|---|---|
| P450 | ID NO | (%) | (%) | (%) | (%) |
| A0A286QUG7 | 827 | 87.63 | 12.37 | Below detection | Below detection |
| | | | | limit | limit |
| W5W4U7 | 833 | 94.72 | 5.28 | Below detection | Below detection |
| | | | | limit | limit |
| D5L0M5 | 829 | 94.17 | 5.83 | Below detection | Below detection |
| | | | | limit | limit |
| XP026740610 | 831 | 93.14 | 6.86 | Below detection | Below detection |
| | | | | limit | limit |
| ACF17813 | 835 | 89.15 | 10.85 | Below detection | Below detection |
| | | | | limit | limit |
| A0A4C1YMA7 | 837 | 93.03 | 6.97 | Below detection | Below detection |
| | | | | limit | limit |

TABLE 5-6
Bioconversion of thebaine to northebaine in strains co-expressing different
possible demethylase enzymes from insect and different possible demethylase-CPR
enzymes from insect with a permease from Papaver somniferum (SEQ ID NO 466),
and grown in DELFT minimal medium at pH 7.0 with 500 μM of thebaine.

| | | | | | Northebaine- | Thebaine |
|---|---|---|---|---|---|---|
| Cytochrome | NADPH-cytochrome | SEQ | Thebaine | Northebaine | Oxaziridine | N-oxide |
| P450 | P450 reductase | ID NO | (%) | (%) | (%) | (%) |
| A0A286QUG7 | Se_CPR_F1DI27 | 827/294 | 89.59 | 10.41 | Below | Below |
| | | | | | detecttion | detecttion |
| | | | | | limit | limit |
| D5L0M5 | MsCPR_XP_030039194 | 829/839 | 95.24 | 4.76 | Below | Below |
| | | | | | detecttion | detecttion |
| | | | | | limit | limit |
| A0A4C1YMA7 | HvCPR_A0A2A4IYH3 | 837/841 | 96.40 | 3.60 | Below | Below |
| | | | | | detecttion | detecttion |
| | | | | | limit | limit |

TABLE 5-7
Bioconversion of oripavine to nororipavine in strains expressing different possible
demethylase enzymes from insect, co-expressed with a demethylase-CPR from Helicoverpa armigera
(SEQ ID NO 293) and a permease from Papaver somniferum (SEQ ID NO 466), and grown
in DELFT minimal medium at pH 4.5 with 500 μM of oripavine.

| Cytochrome | SEQ | Oripavine | Nororipavine | Nororipavine-Oxaziridine | Oripavine N-oxide |
|---|---|---|---|---|---|
| P450 | ID NO | (%) | (%) | (%) | (%) |
| A0A286QUG7 | 827 | 87.43 | 12.57 | Below detection | Below detection |
| | | | | limit | limit |
| D5L0M5 | 829 | 95.73 | 4.27 | Below detection | Below detection |
| | | | | limit | limit |
| XP026740610 | 831 | 82.96 | 17.04 | Below detection | Below detection |
| | | | | limit | limit |

TABLE 5-8
Bioconversion of oripavine to nororipavine in strains co-expressing different
potential demethylase enzymes from insect and different candidate demethylase-CPR
enzymes from insect with a permease from Papaver somniferum (SEQ ID NO 466),
and grown in DELFT minimal medium at pH 4.5 with 500 μM of oripavine.

| | | | | | Northebaine- | Thebaine |
|---|---|---|---|---|---|---|
| Cytochrome | NADPH-cytochrome | SEQ | Oripavine | Nororipavine | Oxaziridine | N-oxide |
| P450 | P450 reductase | ID NO | (%) | (%) | (%) | (%) |
| A0A286QUG7 | Se_CPR_F1DI27 | 827/294 | 86.79 | 13.21 | Below | Below |
| | | | | | detecttion | detecttion |
| | | | | | limit | limit |
| D5L0M5 | MsCPR_XP_030039194 | 829/839 | 95.07 | 4.93 | Below | Below |
| | | | | | detecttion | detecttion |
| | | | | | limit | limit |

### Example 6—Identification of Fungal Demethylases for the Bioconversion of Thebaine and Oripavine

Rates for the select group of fungal demethylase of converting thebaine into northebaine or by-products northebaine-Oxaziridine or thebaine N-oxide are shown in table 6-1 below, while conversion rates for the select group of fungal demethylase of oripavine into nororipavine or by-product oripavine N-oxide are shown in table 6-2.

TABLE 6-1
Bioconversion of thebaine to northebaine or thebaine to oripavine in strains
expressing a select group of fungal demethylase enzymes and grown in
DELFT minimal medium at pH 7.0 in presence of 0.5 mM of thebaine.

| | SEQ | Northebaine | Oripavine | Northebaine-oxaziridine | Thebaine N-oxide |
|---|---|---|---|---|---|
| Demethylase | ID NO: | (%) | (%) | (%) | (%) |
| CYPDN_8 | 290 | 15.42 | 2.15 | 3.47 | 5.06 |
| (control | | | | | |
| demethylase) | | | | | |
| CYPDN_39 | 198 | 13.56 | 6.06 | Below detection | 1.78 |
| | | | | limit | |
| CYPDN_41 | 200 | 15.45 | Below detection | Below detection | Below detection |
| | | | limit | limit | limit |
| CYPDN_43 | 202 | 8.05 | Below detection | 1.97 | 4.56 |
| | | | limit | | |
| CYPDN_45 | 204 | 8.04 | Below detection | Below detection | 3.54 |
| | | | limit | limit | |
| CYPDN_50 | 208 | 3.74 | Below detection | Below detection | Below detection |
| | | | limit | limit | limit |
| CYPDN_51 | 210 | 16.33 | Below detection | 3.27 | 6.40 |
| | | | limit | | |
| CYPDN_57 | 212 | 7.12 | Below detection | Below detection | Below detection |
| | | | limit | limit | limit |
| CYPDN_59 | 214 | 4.97 | Below detection | Below detection | 2.01 |
| | | | limit | limit | |
| CYPDN_61 | 216 | 2.20 | 1.55 | Below detection | Below detection |
| | | | | limit | limit |
| CYPDN_62 | 218 | 2.12 | 2.82 | Below detection | Below detection |
| | | | | limit | limit |
| CYPDN_63 | 220 | 2.12 | 2.63 | Below detection | Below detection |
| | | | | limit | limit |
| CYPDN_64 | 222 | 2.32 | 11.72 | Below detection | Below detection |
| | | | | limit | limit |
| CYPDN_65 | 224 | 2.16 | 6.85 | Below detection | Below detection |
| | | | | limit | limit |
| CYPDN_67 | 226 | 4.54 | 1.87 | Below detection | 1.14 |
| | | | | limit | |
| CYPDN_68 | 228 | 2.31 | 4.19 | Below detection | Below detection |
| | | | | limit | limit |
| CYPDN_69 | 230 | 3.73 | Below detection | Below detection | 1.80 |
| | | | limit | limit | |
| CYPDN_70 | 232 | 7.16 | Below detection | Below detection | 1.99 |
| | | | limit | limit | |
| CYPDN_74 | 234 | 0.27 | Below detection | Below detection | Below detection |
| | | | limit | limit | limit |
| CYPDN_75 | 236 | 2.81 | 7.733 | Below detection | Below detection |
| | | | | limit | limit |
| CYPDN_77 | 238 | 1.16 | Below detection | Below detection | Below detection |
| | | | limit | limit | limit |
| CYPDN_80 | 240 | 3.16 | 3.88 | Below detection | Below detection |
| | | | | limit | limit |
| CYPDN_82 | 242 | 1.14 | Below detection | Below detection | Below detection |
| | | | limit | limit | limit |
| CYPDN_84 | 244 | 0.65 | Below detection | Below detection | Below detection |
| | | | limit | limit | limit |
| CYPDN_85 | 246 | 6.01 | Below detection | Below detection | 2.20 |
| | | | limit | limit | |
| CYPDN_86 | 248 | 5.19 | Below detection | Below detection | Below detection |
| | | | limit | limit | limit |
| CYPDN_91 | 250 | 31.99 | 6.48 | 8.25 | 13.96 |
| CYPDN_92 | 252 | 33.87 | 5.14 | 8.03 | 15.09 |
| CYPDN_93 | 254 | 14.44 | 3.58 | 3.22 | 8.86 |
| CYPDN_95 | 256 | 3.73 | Below detection | Below detection | Below detection |
| | | | limit | limit | limit |
| CYPDN_98 | 258 | 1.17 | Below detection | Below detection | 1.78 |
| | | | limit | limit | |
| CYPDN_100 | 260 | 1.30 | Below detection | Below detection | 2.39 |
| | | | limit | limit | |
| CYPDN_101 | 262 | 0.93 | Below detection | Below detection | Below detection |
| | | | limit | limit | limit |
| CYPDN_103 | 264 | 1.31 | Below detection | Below detection | Below detection |
| | | | limit | limit | limit |
| CYPDN_104 | 266 | 2.47 | Below detection | Below detection | 5.43 |
| | | | limit | limit | |
| CYPDN_105 | 268 | 8.09 | 7.04 | Below detection | Below detection |
| | | | | limit | limit |
| CYPDN_108 | 270 | 0.43 | Below detection | Below detection | Below detection |
| | | | limit | limit | limit |
| CYPDN_109 | 272 | 3.67 | Below detection | Below detection | 1.92 |
| | | | limit | limit | |
| CYPDN_110 | 274 | 6.11 | Below detection | Below detection | 2.22 |
| | | | limit | limit | |
| CYPDN_112 | 276 | 9.56 | Below detection | 1.71 | 6.39 |
| | | | limit | | |
| CYPDN_115 | 278 | 0.45 | Below detection | Below detection | Below detection |
| | | | limit | limit | limit |
| CYPDN_118 | 282 | 2.75 | Below detection | 1.02 | 2.90 |
| | | | limit | | |
| CYPDN_119 | 284 | 1.94 | Below detection | Below detection | 2.42 |
| | | | limit | limit | |
| CYPDN_120 | 286 | 1.50 | Below detection | Below detection | 2.14 |
| | | | limit | limit | |
| CYPDN_123 | 288 | 9.79 | Below detection | Below detection | 3.14 |
| | | | limit | limit | |
| Plasmids control | — | Below detection | Below detection | Below detection | Below detection |
| | | limit | limit | limit | limit |

TABLE 6-2
Bioconversion of oripavine to nororipavine in strains expressing
a select group of fungal demethylase enzymes and grown in DELFT
minimal medium at pH 4.5 in presence of 0.5 mM of oripavine.

| Demethylase | Nororipavine (%) | Oripavine N-oxide (%) |
|---|---|---|
| CYPDN_8 | 5.15 | 1.81 |
| CYPDN_43 | 7.79 | 3.38 |
| CYPDN_45 | 1.44 | Below detection limit |
| CYPDN_46 | 0.37 | Below detection limit |
| CYPDN_51 | 8.62 | Below detection limit |
| CYPDN_59 | 0.84 | Below detection limit |
| CYPDN_67 | 1.55 | Below detection limit |
| CYPDN_69 | 1.13 | Below detection limit |
| CYPDN_70 | 3.64 | Below detection limit |
| CYPDN_74 | 1.79 | Below detection limit |
| CYPDN_80 | 1.50 | Below detection limit |
| CYPDN_82 | 1.20 | Below detection limit |
| CYPDN_84 | 1.52 | Below detection limit |
| CYPDN_85 | 1.33 | Below detection limit |
| CYPDN_86 | 2.93 | Below detection limit |
| CYPDN_91 | 9.42 | Below detection limit |
| CYPDN_92 | 9.68 | 3.03 |
| CYPDN_93 | 4.90 | Below detection limit |
| CYPDN_98 | 1.93 | 3.30 |
| CYPDN_100 | 1.82 | 1.09 |
| CYPDN_101 | 1.90 | 3.00 |
| CYPDN_103 | 1.54 | Below detection limit |
| CYPDN_104 | 2.84 | 9.25 |
| CYPDN_105 | 1.82 | Below detection limit |
| CYPDN_108 | 0.97 | Below detection limit |
| CYPDN_109 | 3.41 | 2.94 |
| CYPDN_110 | 2.52 | 2.09 |
| CYPDN_112 | 6.86 | 4.31 |
| CYPDN_117 | 0.96 | 0.99 |
| CYPDN_118 | 1.70 | 3.07 |
| CYPDN_119 | 1.46 | Below detection limit |
| CYPDN_123 | 2.07 | 2.08 |
| Plasmids control | 0.17 | Below detection limit |

Surprisingly, fungal demethylase were found capable of in vivo converting thebaine and/or oripavine to Northebaine and/or nororipavine with even higher efficiency and with production of less by-products as compared to know demethylase used for this conversion. Expression of demethylase genes CYPDN_91 or CYPDN_92 in a strain containing demethylase-CPR Cel_CPR from C. elegans gave a remarkable improvement in N-demethylation of thebaine to northebaine. The strains with CYPDN_91 or CYPDN_92 exhibited a N-demethylation of thebaine to northebaine of approximately 32-34% when strains were grown in DELFT minimal medium at pH 7.0 in presence of 0.5 mM thebaine. Moreover, strains with CYPDN_64, CYPDN_65 or CYPDN_75 exhibited O-demethylation of thebaine to oripavine of 6-11% when strains were grown in DELFT minimal medium at pH 7.0 in presence of 0.5 mM thebaine.

In fact, expression of demethylase genes CYPDN_91 or CYPDN92 in a yeast strain that contains the demethylase-CPR Cel_CPR, results in an improvement of N-demethylation of thebaine to northebaine of 107-120% in comparison to the best prior art control strain (CYPDN_8), while the demethylase genes CYPDN_64, CYPDN_65 and CYPDN_75 when individually expressed in a yeast strain that contains demethylase-CPR Cel_CPR, exhibit a specific 0-demethylase activity with a yield of bioconversion of thebaine to oripavine of 6-11%.

### Example 7: Strain Engineering for Expression of Heterologous Demethylase in Combination with transporters

Demethylase CYPDN8 from Rhizopus microspores is shown as SEQ ID NO. 290 and also known in the art such as from WO2018/229306, which also describes other herein relevant technical details such as about pOD75 and pOD13 plasmids as referred to herein. Accordingly, based on the technical disclosure herein and the technical disclosure of WO2018/229306 hereby incorporated herein by reference, the skilled person is able to routinely carry out and practice the examples of the invention as included herein.

### Plasmid Based Gene Expression

Strains EVST25898 and sOD157 were transformed with relevant plasmids using the lithium acetate method (Gietz et al. 2002. Methods Enzymol. Vol 350, p87-96). EVST25898 was used as the tester strain in Example 10 and 11. sOD157 was used as tester strain from Example 13 and onwards. The only difference between these two strains was that sOD157 (parental strain: EVST25898) contains additional elements to facilitate cloning. For testing the impact of possible transporter proteins on the bioconversion of thebaine to northebaine, the host yeast strain was transformed with a plasmid containing demethylase gene CYPDN8 (pOD75) along with a plasmid containing Gel_CPR (co) from Cunninghamella elegans (pOD13) in combination with the various possible transporter proteins. Genes were inserted and expressed using either P413TEF, P415TEF or p416TEF, all described by Mumberg et al., 1995. Gene. April 14; 156(1):119-22. The control strain was constructed by transforming strains EVST25898 or sOD157 with pOD75, pOD13 as well as an empty plasmid: p416TEF.

Table 7-1 describes the plasmids that were expressed with the yeast strains. Transformants were selected in synthetic complete (SC) agar plates lacking histidine, leucine and uracil. Transformation plates were incubated for 3-4 days at 30° C. until visible colonies were obtained.

TABLE 7-1
Plasmids expressed in the corresponding yeast strains

| | | | Yeast Selection | |
|---|---|---|---|---|
| Vector Name | Backbone | Promoter-Gene-Terminator | Marker | Description |
| pOD13 | P413TEF | pTEF1-Cel_CPR_co-tCYC1 | HIS3 | Cel_CPR (co) from |
| | | | | Cunninghamella elegans |
| | | | | (SEQ ID NO: 306) |
| pOD75 | P415TEF | pTEF1-CYPDN8-tCYC1 | LEU2 | A0A0C7AZL4 (co) from |
| | | | | Rhizopus Microsporus |
| | | | | (SEQ ID NO: 290) |
| p416TEF | | No gene inserted | URA3 | Mumberg et al., 1995. Gene. |
| | | | | Apr 14; 156(1): 119-22 |
| pOD470 | P415TEF | pTEF1_CYPDN43_tCYC1 | LEU2 | Demethylase from |
| | | | | Lichtheimia corymbifera |
| | | | | (SEQ ID NO: 202) |
| pOD1034 | P415TEF | pTEF1-HaCYP6AE15v2-tCYC1 | LEU2 | Demethylase from |
| | | | | Helicoverpa armigera |
| | | | | (SEQ ID NO: 140) |
| pOD1357 | P415TEF | pTEF1-Hv_CYP_A0A2A4JAM9-tCYC1 | LEU2 | Demethylase from |
| | | | | Heliothis virescens |
| | | | | (SEQ ID NO: 152) |
| pOD1184 | P413TEF | pTEF1- HaCPR_E7E2N6 -tCYC1 | HIS3 | Demethylase-CPR (co) from |
| | | | | Helicoverpa armigera |
| | | | | (SEQ ID NO: 292) |
| pOD1621 | P415TEF | pTEF1-Hv_CYP_A0A2A4JAM9_A110S- | LEU2 | Demethylase from |
| | | tCYC1 | | Heliothis virescens |
| | | | | with single mutation at |
| | | | | amino acid residue 110 |
| | | | | (SEQ ID NO: XXX) |
| pOD1736 | P415TEF | pTEF1- | LEU2 | Demethylase from |
| | | Hv_CYP_A0A2A4JAM9_A110N + | | Heliothis virescens |
| | | H242P-tCYC1 | | with double mutations |
| | | | | at amino acid residues |
| | | | | 110 and 242 |
| | | | | (SEQ ID NO: XXX) |
| pOD1738 | P415TEF | pTEF1-Hv_CYP_A0A2A4JAM9_{—} | LEU2 | Demethylase from |
| | | A110N + R112K + H242P-tCYC1 | | Heliothis virescens |
| | | | | with triple mutations |
| | | | | at amino acid residues |
| | | | | 110, 112 and 242 |
| | | | | (SEQ ID NO: XXX) |
| pOD1740 | P415TEF | PTEF1-HV_CYP_A0A2A4JAM9_{—} | LEU2 | Demethylase from |
| | | A110N + H242P + V224I-tCYC1 | | Heliothis virescens |
| | | | | with triple mutations |
| | | | | at amino acid residues |
| | | | | 110, 224 and 242 |
| | | | | (SEQ ID NO: XXX) |

### Gene Expression by USER Integration.

Strain EVST25898 was further modified by genomic integration using the Saccharomyces cerevisiae gene integration and expression system developed by Mikkelsen, M D et al. (Metab. Eng. 14, Issue 2, 104-111 (2012)). The demethylase gene CYPDN8 was expressed using the well-known Saccharomyces cerevisiae TEF1 promoter, and the Gel_GPR (co) from Cunninghamella elegans was expressed using the Saccharomyces cerevisiae PGK1 promoter. The expression cassette was integrated in site XII-5 using the Kluyveromyces lactis URA3 marker as selection marker for growth on media lacking uracil (described by Mikkelsen, M D et al. (Metab. Eng. 14, Issue 2, 104-111 (2012)). Subsequently, the transporter genes T11_AthGTR1_GA (SEQ ID NO: 324), T52_BmePTR2_GA (SEQ ID NO: 398), T14_PsoNPF3_GA (SEQ ID NO: 328), T60_AmeNPF2_GA (SEQ ID NO: 412), T1_CjaMDR1_GA (SEQ ID NO: 308) and T70_CmaNPF_GA (SEQ ID NO: 430) were integrated into the site XI-5 of the Saccharomyces cerevisiae strain using the Saccharomyces cerevisiae TEF1, PGK1, TEF2, TDH3, TPI1, and PDC1 promoters respectively. Selection for transformants was done using the well-known Kluyveromyces lactis LEU2 marker available e.g. from EUROSCARF (http://www.euroscarf.de) and growth on media lacking leucine. After that, plasmid pOD13 (see Table 7-1) was transformed with the resulting strain in order to make the strain prototrophic by selecting on media lacking histidine. Transformation plates were incubated for 3-4 days at 30° C. until visible colonies were obtained.

### Gene Expression by Ty Integration.

Multiple copies of demethylase and best transporter combination were integrated into the previous mentioned strain background by Ty integration. Method of Ty genomic integration was modified based on system developed by Maury, J et al. (PLoS One 11(3):e0150394 PMID:26934490). The best demethylase genes were expressed using the well-known Saccharomyces cerevisiae TEF2 promoter, and best suitable transporter genes were expressed using the Saccharomyces cerevisiae TDH3 promoter. Ty expression of the genes was integrated by using the Kluyveromyces lactis URA3 marker as selection marker for growth on media lacking uracil (described by Mikkelsen, M D et al. (Metab. Eng. 14, Issue 2, 104-111 (2012)). Ty expression of the genes was also integrated by using the Kluyveromyces lactis LEU2 marker as selection marker for growth on media lacking leucine. Ty expression of the genes can also be integrated by using the Schizosaccharomyces pombe HIS5 marker as selection marker for growth on media lacking histidine. The strains were made prototrophic by integrating the gene encoding demethylase-CPR such as HaCPR_E7E2N6 (and additional copies of best transporters by USER integration as previously described. Genomic integration by USER was performed and selected using the well-known Kluyveromyces lactis LEU2 marker available e.g. from EUROSCARF (http://www.euroscarf.de) and growth on media lacking leucine. Genomic integration by USER was also performed and selected using the well-known Schizosaccharomyces pombe HIS5 marker available e.g. from EUROSCARF (http://www.euroscarf.de) and growth on media lacking histidine. Transformation plates were incubated for 3-4 days at 30° C. until visible colonies were obtained.

### Example 8. Cultivation and Harvest of Yeast Strains

### Cultivation

Yeast strains of example 7 were cultivated in 96-deep-well-plate (DWP) format. Cells were grown in 0.5 ml SC-His-Leu-Ura medium at 30° C. with shaking at 250 rpm in ISF1-X Kuhner shaker for 20-24 hours and utilized as precultures for in vivo bioconversion assays. For Example 10 and Example 11, 50 μl of the overnight cell cultures were grown in 450 μl Synthetic complete (SC)-His-Leu-Ura medium (pH 7) or DELFT minimal medium (pH 7) containing 0.5 mM thebaine or oripavine. Both media contain 0.1 M potassium phosphate buffer. Thebaine (or Oripavine) were added via a 25 mM stock solution in DMSO. Cells were grown for 72 hours with shaking at 250 rpm. From Example 13 and onwards, cultivation of the cells fed with thebaine was as the same as previously mentioned. As for cultivation of cells fed with oripavine, 50 μl of the overnight cell cultures were grown in 450 μl of DELFT minimal medium (pH 4.5) containing 0.5 mM oripavine. The media was not buffered with potassium phosphate buffer.

### Harvest.

LC-MS analysis (see for example 10 to 12): 50 μl of cell cultures were transferred to a new 96-deep-well-plate containing 50 μl of MilliQ water with 0.1% of formic acid. The harvested 96 well plate was incubated at 80° C. for 10 minutes. Plate was then centrifugated for 10 minutes at 4000 rpm. The supernatants were then diluted in MilliQ water with 0.1% of formic acid to reach a final dilution of 1:100. Thebaine, northebaine, oripavine and nororipavine contents were analyzed by LC-MS.

HPLC analysis (for example 13 to 21): 60 dl of cell cultures were transferred to a new 96-deep-well-plate containing 60 l of MilliQ water with 0.1% of formic acid (1:1 dilution). The harvested 96 well plate was incubated at 80° C. for 10 minutes. Plate was then centrifugated for 10 minutes at 4000 rpm. For cells that were fed with 0.5 mM thebaine or oripavine, 100 μl of the supernatants were transferred to a new plate for HPLC analysis. For cells that were fed with higher concentration of thebaine or oripavine, dilution rate was increased accordingly.

### Example 9. Analytical Procedures

For examples 10 to 14 below results were evaluated by LC-MS as follows: For all compounds (thebaine, northebaine, oripavine and nororipavine) stock solutions were prepared in DMSO at a concentration of 10 mM. Standard solutions were prepared at concentrations of 6 μM, 4 μM, 2 μM, 1 μM, 500 nM, 200 nM, 100 nM, 50 nM, 20 nM and 10 nM from the stock solutions. Samples were injected into the Agilent 1290 UPLC coupled to an Ultivo Triple Quadrupole. The LC-MS method was as follows: Mobile Phase A. H₂O+0.1% Formic acid; Mobile Phase B: Acetonitrile+0.1% Formic acid; Column: Phenomenex Kinetex 1.7 μm XB—C18 100 Å, 2.1×100 mm. The elution gradient is shown in Table 9-1 and the LC-MS conditions are given in Table 9-2. Table 9-3 shows the mass spectrometer source and detector parameters and Table 9-4 shows the target compounds, their retention times, their parent ion, transition ions (MRM) as well as dwell times, cone voltages and collision energies used.

TABLE 9-1
Gradient for LC-MS

| Time (min) | % B |
|---|---|
| 0 | 2 |
| 0.30 | 2 |
| 4.00 | 30 |
| 4.40 | 100 |
| 4.90 | 100 |
| 5 | 2 |
| 6 | 2 |

TABLE 9-2
LC-MS conditions

| Parameter | Value | |
|---|---|---|
| Injection volume | 2 | μl |
| Column Temperature | 30° C. ± 4° C. | |
| Injection method | Flow through needle | |
| Flow | 0.4 | ml/min |

| | |
|---|---|
| Auto sampler temperature | 10° C. ± 2° C. |
| Reconditioning wash | 2% Acetonitrile (in H₂O), 5 sec |
| Weak wash | 20% Methanol (in H₂O), 5 sec |
| Strong wash | 30% Acetonitrile, 30% Methanol, |
| | 30% 2-Propanol, 10% H₂O, 10 sec |
| Seal wash | 20% 2-Propanol (in H₂O) |

TABLE 9-13
Mass spectrometer source and detector
parameters (Ultivo Triple Quadrupole)

| Source Parameter | Value | |
|---|---|---|
| Ion Source | Electrospray Positive Mode (ESI+) | |
| Capillary Voltage | 3.5 | kV |
| Nozzle Voltage | 500 | V |
| Source Gas Temperature | 290° | C. |
| Source Gas Flow | 12 | L/min |
| Source Sheath Gas Temperature | 380° | C. |
| Source Sheath Gas Flow | 12 | L/min |
| Nebulizer | 30 | psi |

| | |
|---|---|
| Mode | MS/MS |
| Collision | See Table 14 |

TABLE 9-4
Multiple reaction monitoring targets and conditions (ESI+)

| Target | Retention | Parent | Daughter | Dwell | Fragmentor | Collision |
|---|---|---|---|---|---|---|
| compound | time (min) | ion (m/z) | ion (m/z) | time (ms) | voltage (V) | energy (V) |
| Northebaine | 3.53 | 298 | 249 | 55.03 | 100 | 20 |
| Thebaine | 3.6 | 312 | 58 | 61.53 | 110 | 10 |
| Oripavine | 2.59 | 298 | 237 | 64.05 | 110 | 5 |
| Nororipavine | 2.54 | 284 | 218 | 70.30 | 110 | 10 |

For examples 15 to 21 below results were evaluated by HPLC as described in example 4

### Example 10. Transporters Capable of Improving Bioconversion of Thebaine and/or Derivatives Thereof

### Bioconversion

Expression of transporter genes in a strain containing demethylase gene CYPDN8 and demethylase-CPR Cel_CPR (co) gave remarkable improvement in bioconversion of thebaine to northebaine for some of the transporter genes, where some exhibited a significant improved bioconversion when strains were grown in presence of 0.5 mM thebaine.

TABLE 10-1
Bioconversion of thebaine to northebaine in strains expressing different
possible transporter enzymes and improvement in the bioconversion as compared
to control strain not expressing any heterologous transporter genes.

| | | Percentage | Improvement | |
|---|---|---|---|---|
| | | bioconversion | in Thebaine to | |
| | SEQ | of Thebaine to | Northebaine | |
| | ID | Northebaine | bioconversion | |
| Transporter genes | NO: | (%) | (%) | Growth medium |
| T1_CjaMDR1_GA | 308 | 12.0 | 45 | SC-his-leu-ura |
| T3_NcaNPF_GA | 310 | 6.7 | −19 | SC-his-leu-ura |
| T4_EsaGTR_GA | 312 | 11.3 | 36 | SC-his-leu-ura |
| T5_AlyPOT_GA | 314 | 6.1 | −27 | SC-his-leu-ura |
| T6_CruGTR_GA | 316 | 6.4 | −23 | SC-his-leu-ura |
| T7_PtrPOT_GA | 318 | 13.5 | 63 | SC-his-leu-ura |
| T8_BnaMFS_GA | 320 | 4.2 | −49 | SC-his-leu-ura |
| T10_BolGTR_GA | 322 | 6.0 | −28 | SC-his-leu-ura |
| T11_AthGTR1_GA | 324 | 9.7 | 17 | SC-his-leu-ura |
| T12_PsoNPF1_GA | 326 | 6.7 | −19 | SC-his-leu-ura |
| T14_PsoNPF3_GA | 328 | 10.3 | 24 | SC-his-leu-ura |
| T17_PsoNPF6_GA | 330 | 5.1 | −39 | SC-his-leu-ura |
| Control SC-his-leu-ura | 332 | 8.3 | 0.0 | SC-his-leu-ura |
| T18_PsoNPF7_GA | 334 | 10.0 | 2 | DELFT minimal medium |
| T19_RmiPTR2_GA | 336 | 9.5 | 13 | DELFT minimal medium |
| T20_RmiPTR2_v2_GA | 338 | 7.9 | 6 | DELFT minimal medium |
| T21_RalPTR2_GA | 340 | 7.3 | −13 | DELFT minimal medium |
| T22_CanPOT_GA | 342 | 4.4 | −48 | DELFT minimal medium |
| T23_ArePOT_GA | 344 | 4.6 | −45 | DELFT minimal medium |
| T24_SlyPTR2_GA | 346 | 4.0 | −52 | DELFT minimal medium |
| T25_AorPOT_GA | 348 | 4.1 | −51 | DELFT minimal medium |
| T26_NfuPOT_GA | 350 | 4.0 | −52 | DELFT minimal medium |
| T28_MciPOT_GA | 354 | 4.2 | −50 | DELFT minimal medium |
| T29_AcaPOT_GA | 356 | 5.1 | −39 | DELFT minimal medium |
| T30_MlyPOT_GA | 358 | 5.6 | −33 | DELFT minimal medium |
| T31_TgaPOT_GA | 360 | 4.4 | −48 | DELFT minimal medium |
| T32_AarPOT_GA | 362 | 5.1 | −39 | DELFT minimal medium |
| T33_CcuPTR2_GA | 364 | 4.6 | −45 | DELFT minimal medium |
| T34_HvePOT_GA | 366 | 5.5 | −35 | DELFT minimal medium |
| T35_EcuPOT_GA | 368 | 7.8 | −7 | DELFT minimal medium |
| T36_RnePOT_GA | 370 | 4.3 | −49 | DELFT minimal medium |
| T37_OcoPOT_GA | 372 | 4.8 | −45 | DELFT minimal medium |
| T38_ScuPTR2_GA | 374 | 9.9 | 18 | DELFT minimal medium |
| T39_CgrPOT_GA | 376 | 5.6 | −33 | DELFT minimal medium |
| T40_EdePOT_GA | 378 | 6.1 | −27 | DELFT minimal medium |
| T41_CalPTR2_GA | 380 | 5.7 | −32 | DELFT minimal medium |
| T44_CcaMFS_GA | 382 | 4.4 | −48 | DELFT minimal medium |
| T45_PanPOT_GA | 384 | 9.8 | 0 | DELFT minimal medium |
| T46_RchPOT_GA | 386 | 8.1 | −4 | DELFT minimal medium |
| T47_PbeNPF_GA | 388 | 4.6 | −45 | DELFT minimal medium |
| T48_CcaPOT_GA | 390 | 9.7 | −1 | DELFT minimal medium |
| T49_HanPOT_GA | 392 | 7.7 | −8 | DELFT minimal medium |
| T51_TorPOT_GA | 396 | 5.5 | −35 | DELFT minimal medium |
| T52_BmePTR2_GA | 398 | 11.7 | 19 | DELFT minimal medium |
| T53_EhePOT_GA | 400 | 7.3 | −13 | DELFT minimal medium |
| T54_MelPOT_GA | 402 | 10.9 | 11 | DELFT minimal medium |
| T55_NsyNPF_GA | 404 | 3.2 | −62 | DELFT minimal medium |
| T56_CanNPF_GA | 406 | 8.4 | 0 | DELFT minimal medium |
| T57_AcoNPF_GA | 408 | 11.7 | 19 | DELFT minimal medium |
| T59_AmeNPF1_GA | 410 | 5.3 | −37 | DELFT minimal medium |
| T60_AmeNPF2_GA | 412 | 11.9 | 21 | DELFT minimal medium |
| T61_TwiNPF_GA | 414 | 8.1 | −4 | DELFT minimal medium |
| T62_SmaNPF_GA | 416 | 7.5 | −11 | DELFT minimal medium |
| T63_CfoNPF_GA | 418 | 7.4 | −12 | DELFT minimal medium |
| T64_XsiNPF_GA | 420 | 6.9 | −18 | DELFT minimal medium |
| T66_TelNPF_GA | 422 | 8.3 | −1 | DELFT minimal medium |
| T69_PhoNPF_GA | 428 | 5.4 | −36 | DELFT minimal medium |
| T70_CmaNPF_GA | 430 | 9.1 | 8 | DELFT minimal medium |
| T72_TcoNPF_GA | 434 | 8.4 | 0 | DELFT minimal medium |
| T73_PbrNPF1_GA | 436 | 5.8 | −31 | DELFT minimal medium |
| T74_PbrNPF2_GA | 438 | 6.6 | −21 | DELFT minimal medium |
| T75_PbrNPF3_GA | 440 | 7.7 | −8 | DELFT minimal medium |
| T76_AhuNPF_GA | 442 | 4.9 | −42 | DELFT minimal medium |
| T77_PocNPF_GA | 444 | 5.5 | −35 | DELFT minimal medium |
| T78_VofNPF_GA | 446 | 8.5 | 1 | DELFT minimal medium |
| T79_EcaNPF_GA | 448 | 7.6 | −10 | DELFT minimal medium |
| T80_CroNPF_GA | 450 | 9.8 | 0 | DELFT minimal medium |
| T82_NsaNPF_GA | 452 | 8.8 | −10 | DELFT minimal medium |
| Control DELFT | — | 8.4 | 0.0 | DELFT minimal medium |
| Control DELFT | — | 9.8 | 0.0 | DELFT minimal medium |

Numbers in Italic are relative to Control DELFT of 9.8.

### Improvement of Bioconversion:

Expression of one of the transporter genes T14_PsoNPF3_GA, T1_CjaMDR1_GA, T4_EsaGTR_GA or T7_PtrPOT_GA in a yeast strain that contains demethylase gene CYPDN8 and demethylase-CPR Cel_CPR (co), results in improved bioconversion of thebaine to northebaine in the range of 24-63% in comparison to the control strain.

Further, significant improvement was also seen for the transporter genes T60_AmeNPF2_GA, T57_AcoNPF_GA, T52_BmePTR2_GA, T38_ScuPTR2_GA, T11_AthGTR1_GA, T19_RmiPTR2_GA, T70_CmaNPF_GA or T54_MelPOT_GA.

### Conclusions

The results of this Example demonstrate expression of one of the transporter genes T14_PsoNPF3_GA, T1_CjaMDR1_GA, T4_EsaGTR_GA or T7_PtrPOT_GA in a yeast strain that contains demethylase gene CYPDN8 and demethylase-CPR Cel_CPR (co), results in improved bioconversion of thebaine to northebaine in the range of 24-63% in comparison to the control strain. Further, significant improvement was also seen for the transporter genes T60_AmeNPF2_GA, T57_AcoNPF_GA, T52_BmePTR2_GA, T38_ScuPTR2_GA, T11_AthGTR1_GA, T19_RmiPTR2_GA, T70_CmaNPF_GA or T54_MelPOT_GA.

Further, transporters were tested for improvement in conversion of the thebaine derivative oripavine to nororipavine.

### Bioconversion.

Expression of transporter gene T14_PsoNPF3_GA from Papaver somniferum in a strain containing demethylase gene CYPDN8 and demethylase-CPR Cel_CPR (co) showed remarkable improvement in bioconversion of oripavine to nororipavine. In an assay where a strain was grown in presence of 0.5 mM oripavine, the strain containing T14_PsoNPF3_GA exhibited 2.3% bioconversion of the oripavine to nororipavine, which corresponds to an improvement in bioconversion of oripavine to nororipavine by 64% in comparison to the control strain.

TABLE 10-2
Bioconversion and improvement in oripavine to
nororipavine bioconversion compared to the control
strain, observed when growing strains expressing
different possible transporter proteins.

| | | | Improvement of |
|---|---|---|---|
| | SEQ | Bioconversion of | oripavine to nororipavine |
| | ID | oripavine to | bioconversion as |
| Transporter genes | NO: | nororipavine (%) | compared to control (%) |
| T14_PsoNPF3_GA | 328 | 2.3 | 64 |
| Control | — | 1.4 | 0 |

### Conclusions

The result of this Example demonstrates that expression of transporter gene T14_PsoNPF3_GA gave around 64% more bioconversion of oripavine to nororipavine—which is a remarkable yield improvement.

### Example 11. Further Transporters Capable of Improving Bioconversion of Thebaine and/or Derivatives Thereof

This Example 11 discusses transporter genes that are not explicitly mentioned in corresponding Example 10 above.

### Bioconversion.

In bioconversion experiments similar to Example 10 above—3 additional transporters have shown to improve bioconversion of thebaine to northebaine. As shown in Table 11-1 below, T65_ljaNPF_GA, T94_EcrPOT_GA and T97_ScaT14_GA are able to improve bioconversion of thebaine to northebaine by 29.9%, 31.9% and 21.8%, respectively, when compared to a control strain.

Table 11-1 also shows a yeast strain which genes CYPDN8 from Rhizopus microspores and Cel_CPR_co from Cunninghamella elegans have been integrated into host strain EVST25898 (Example 7) at Chromosome XII-5 with URA3 from Kluyveromyces lactis as selection marker. Subsequently, 6 different transporters T11_AthGTR1_GA, T52_BmePTR2_GA, T14_PsoNPF3_GA, T60_AmeNPF2_GA, T1_CjaMDR1_GA, and T70_CmaNPF_GA were expressed in the same strain at Chromosome XI-5 with LEU2 from Kluyveromyces lactis as selection marker. Plasmid pOD13 (Table 7-1) was also expressed in the same strain to make the strain prototrophic. An indication of improvement in the bioconversion of thebaine to northebaine when multiple copies of various transporters were expressed in the same strain.

TABLE 11-1
Bioconversion of thebaine to northebaine in strains expressing different
possible transporter enzymes and improvement in the bioconversion as compared
to control strain not expressing any heterologous transporter genes.

| | | Percentage | Improvement | |
|---|---|---|---|---|
| | SEQ | bioconversion of | in thebaine to | |
| | ID | thebaine to | northebaine | |
| Transporter genes | NO: | northebaine (%) | bioconversion (%) | Growth medium |
| T65_IjaNPF_G | 734 | 10.9 | 29.9 | DELFT minimal medium |
| T94_EcrPOT_G | 736 | 11.1 | 31.9 | DELFT minimal medium |
| T97_ScaT14_GA | 462 | 10.2 | 21.8 | DELFT minimal medium |
| T11_AthGTR1_GA + | 324 | 11.3 | 34.6 | DELFT minimal medium |
| T52_BmePTR2_GA + | 398 | | | |
| T14_PsoNPF3_GA + | 328 | | | |
| T60_AmeNPF2_GA + | 412 | | | |
| T1_CjaMDR1_GA + | 308 | | | |
| T70_CmaNPF_GA | 430 | | | |
| Control DELFT | — | 8.4 | | DELFT minimal medium |

When multiple of different genes were expressed in the yeast cell, it is referred to as gene1+gene2, etc.

### Conclusions

In bioconversion experiments similar to Example 10 above—the results of this Example demonstrate that three additional transporters have shown to improve bioconversion of thebaine to northebaine. As shown in Table 11-1, T65_ljaNPF_GA, T94_EcrPOT_GA and T97_ScaT14_GA are able to improve bioconversion of thebaine to northebaine by 29.9%, 31.9% and 21.8%, respectively, when compared to a control strain.

Further, a strain comprising a combination of 6 transporter proteins discussed in Example 10 gave a very good improvement of thebaine to northebaine.

### Example 12 Further Transporters Tested for Improvement in Conversion of the Thebaine Derivative Oripavine to Nororipavine

### Bioconversion

In bioconversion experiments similar to Example 10 above—an additional transporter that is able to help improving bioconversion of oripavine to nororipavine has been identified.

As shown in Table 12-1 below, T97_ScaT14_GA from Sanguinaria canadensis is able to convert close to 5% of oripavine to nororipavine when fed with 0.5 mM oripavine. In comparison to the control strain, expression of T97_ScaT14_GA improves the bioconversion of oripavine to nororipavine by 254.4%.

TABLE 12-1
Bioconversion and improvement in oripavine to nororipavine
bioconversion compared to the control strain.

| | | | Improvement of |
|---|---|---|---|
| | SEQ | Bioconversion of | oripavine to nororipavine |
| | ID | oripavine to | bioconversion as |
| Transporter genes | NO: | nororipavine (%) | compared to control (%) |
| T97_ScaT14_GA | 462 | 4.96 | 254.4 |
| Control | — | 1.4 | 0 |

### Conclusions

In bioconversion experiments similar to Example 10 above, the results of this Example demonstrate an additional transporter able to help in improving bioconversion of oripavine to nororipavine has been identified.

As shown in Table 12-1, T97_ScaT14_GA from Sanguinaria canadensis is able to convert close to 5% of oripavine to nororipavine when fed with 0.5 mM oripavine. In comparison to the control strain, expression of T97_ScaT14_GA improves the bioconversion of oripavine to nororipavine by 254.4%.

### Example 13. Identification of Purine Uptake Permease (PUP) Transporters Capable of Improving Bioconversion of Thebaine

### Bioconversion

The impact of purine uptake permease transporter proteins on bioconversion of thebaine to northebaine was studied by transforming yeast strain with a plasmid containing a demethylase comparable to the above examples that was capable of acting on reticuline derivatives such as thebaine and/or oripavine using the backbone plasmid p415TEF. A plasmid containing demethylase-CPR (pOD13 from Example 7) was also expressed in combination with various candidate transporter proteins. Yeast strain construction and method of screening for PUP transporters were as previously described in Example 7. Table 13-1 shows the result of percentage bioconversion from thebaine to northebaine with the expression of various PUP transporters. Table 13-1 also presents the percentage improvement in the bioconversion when normalized for a control strain expressing demethylase but not expressing any heterologous transporter.

TABLE 13-1
Percentage demethylase-mediated bioconversion from Thebaine to
Northebaine with the expression of various transporters and percentage
improvements in the bioconversion as compared to a control strains
not expressing any heterologous transporters.

| | | | Percentage |
|---|---|---|---|
| | | Percentage | Improvement in |
| | SEQ | bioconversion of | Thebaine to |
| | ID | Thebaine to | Northebaine |
| PUP Transporters | NO: | Northebaine (%) | bioconversion (%) |
| T101_McoPUP3_1 | 464 | 7.0 | 6.7 |
| T102_PsoPUP3_1 | 466 | 8.6 | 29.8 |
| T103_PsoPUP3_2 | 468 | 7.1 | 7.9 |
| T104_PsoPUP3_3 | 470 | 7.4 | 11.4 |
| T105_PsoPUP-L | 472 | 9.2 | 39.8 |
| Control 1 | | 6.6 | — |
| T109_GflPUP3_83 | 474 | 6.4 | 55.0 |
| T122_PsoPUP3_17 | 488 | 6.1 | 48.4 |
| T130_NdoPUP3_89 | 504 | 4.9 | 19.9 |
| T131_PbrPUP3_81 | 506 | 4.9 | 20.4 |
| T132_CmiPUP3_10 | 508 | 6.6 | 60.2 |
| T133_PsoPUP3_18 | 510 | 5.9 | 42.7 |
| T136_RchPUP3_42 | 514 | 4.6 | 11.1 |
| T137_EguPUP3_50 | 516 | 5.1 | 24.7 |
| T138_AduPUP3_58 | 518 | 4.6 | 11.7 |
| T139_PsoPUP3_66 | 520 | 4.9 | 19.5 |
| T140_PalPUP3_74 | 522 | 5.4 | 30.5 |
| T141_EcaPUP3_88 | 524 | 6.8 | 64.7 |
| T142_McoPUP3_4 | 526 | 7.7 | 88.9 |
| T143_CmiPUP3_11 | 528 | 5.8 | 41.8 |
| T144_PsoPUP3_19 | 530 | 7.7 | 87.1 |
| T146_PsoPUP_35 | 532 | 4.6 | 13.4 |
| T147_MesPUP3_43 | 534 | 6.1 | 49.8 |
| T148_HimPUP3_51 | 536 | 5.0 | 21.4 |
| T149_AcoPUP3_59 | 538 | 6.9 | 69.1 |
| T150_PsoPUP3_67 | 540 | 5.9 | 43.6 |
| T151_PatPUP3_75 | 542 | 5.7 | 39.1 |
| T152_GflPUP3_87 | 544 | 8.0 | 94.0 |
| T153_PsoPUP3_5 | 546 | 4.9 | 19.1 |
| T154_CmiPUP3_12 | 548 | 7.1 | 74.2 |
| T157_RchPUP_36 | 552 | 5.8 | 42.1 |
| T159_OeuPUP3_52 | 556 | 5.8 | 41.8 |
| T160_CeuPUP3_60 | 558 | 5.4 | 30.9 |
| T161_PsoPUP3_68 | 560 | 6.2 | 51.9 |
| T162_PmiPUP3_76 | 562 | 6.4 | 56.1 |
| T163_PbrPUP3_86 | 564 | 5.1 | 24.8 |
| T164_PsoPUP3_78 | 566 | 5.2 | 27.2 |
| T165_AcoPUP3_13 | 568 | 6.5 | 57.9 |
| T166_PsoPUP3_21 | 570 | 6.6 | 61.9 |
| T168_FvePUP3_37 | 572 | 6.4 | 56.5 |
| T169_ZjuPUP3_45 | 574 | 6.6 | 60.6 |
| T170_LsaPUP3_53 | 576 | 6.7 | 62.6 |
| T171_McoPUP3_61 | 578 | 5.5 | 33.8 |
| T172_AcoPUP3_69 | 580 | 6.6 | 60.2 |
| T174_PbrPUP3_85 | 584 | 5.3 | 29.4 |
| T175_PsoPUP3_6 | 586 | 6.7 | 63.9 |
| T176_AcoPUP3_14 | 588 | 5.8 | 41.5 |
| T177_PsoPUP3_22 | 590 | 6.5 | 57.4 |
| T178_PsoPUP3_30 | 592 | 6.1 | 47.6 |
| T180_McoPUP3_46 | 596 | 5.5 | 35.0 |
| T181_HanPUP3_54 | 598 | 5.3 | 30.1 |
| T182_CpaPUP3_62 | 600 | 6.9 | 67.8 |
| T184_PraPUP3_79 | 602 | 5.2 | 27.9 |
| T186_ScaPUP3_84 | 604 | 7.0 | 69.8 |
| T188_AcoPUP3_15 | 606 | 4.7 | 14.7 |
| T189_PsoPUP3_23 | 608 | 4.7 | 14.8 |
| T191_MdoPUP3_39 | 610 | 5.2 | 26.5 |
| T192_CmiPUP3_47 | 612 | 5.5 | 35.0 |
| T193_AanPUP3_55 | 614 | 6.2 | 51.9 |
| T194_CchPUP3_63 | 616 | 5.7 | 39.1 |
| T195_JcuPUP3_71 | 618 | 5.3 | 29.8 |
| T196_PtrPUP3_80 | 620 | 5.7 | 39.1 |
| Control 2 | | 4.1 | — |

Note:
Control 1 is used as the control for T101_McoPUP3_1, T102_PsoPUP3_1, T103_PsoPUP3_2, T104_PsoPUP3_3 and T105_PsoPUP-L. Control 2 is used as control for the rest of the PUP transporters. This was done to compensate for any slight variations that may arise between different runs of LC-MS analysis.

### Improvement of Bioconversion.

When compared to a control strain without a heterologous transporter, several strains engineered with PUP transporters exhibited at least 50% greater bioconversion of the 0.5 mM thebaine fed in this assay. Amongst the PUP transporters examined, PUP transporters T152_GflPUP3_87, T149_AcoPUP3_59, T109_GflPUP3_83, T142_McoPUP3_4, T144_PsoPUP3_19, T141_EcaPUP3_88, T182_CpaPUP3_62, T193_AanPUP3_55 and T122_PsoPUP3_17 exhibited improvements in bioconversion of thebaine to northebaine in the range of 48 94% in comparison to the control strain without a heterologous transporter (Table 13-2). Expression of some PUP transporters, such as T152_GflPUP3_87 from Glaucium flavum, T149_AcoPUP3_59 from Aquilegia coerulea, and T142_McoPUP3_4 from Macleaya cordata, gave remarkable improvements in the demethylase-mediated bioconversion of thebaine to northebaine.

TABLE 13-2
Purine Uptake Permease transporters which have been demonstrated
herein to provide especially large improvements in the demethylase-
mediated bioconversion from Thebaine to Northebaine.

| PUP Transporters | Latin Name for Origin of Sourced Genes |
|---|---|
| T152_GflPUP3_87 | Glaucium flavum |
| T142_McoPUP3_4 | Macleaya cordata |
| T144_PsoPUP3_19 | Papaver somniferum |
| T149_AcoPUP3_59 | Aquilegia coerulea |
| T109_GflPUP3_83 | Glaucium flavum |
| T141_EcaPUP3_88 | Eschscholzia californica |
| T182_CpaPUP3_62 | Carica papaya |
| T193_AanPUP3_55 | Artemisia annua |
| T132_CmiPUP3_10 | Cinnamomum micranthum f. kanehirae |
| T186_ScaPUP3_84 | Sanguinaria canadensis |
| T175_PsoPUP3_6 | Papaver somniferum |
| T122_PsoPUP3_17 | Papaver somniferum |

### Conclusions

Table 13-2 shows some of the PUP transporters that have been herein demonstrated for the first time to shown very considerable improvements in the bioconversion from Thebaine to Northebaine by demethylase. In particular, the results of this Example demonstrate that expression of PUP transporters T152_GflPUP3_87 from Glaucium flavum, T149_AcoPUP3_59 from Aquilegia coerulea, T109_GflPUP3_83 from Glaucium flavum, T142_McoPUP3_4 from Macleaya cordata, T144_PsoPUP3_19 from Papaver somniferum, T141_EcaPUP3_88 from Eschscholzia californica, T182_CpaPUP3_62 from Carica papaya, T193_AanPUP3_55 from Artemisia annua, T132_CmiPUP3_10 from Cinnamomum micranthumf. kanehirae, T186_ScaPUP3_84 from Sanguinaria canadensis, T175_PsoPUP3_6 from Papaver somniferum and T122_PsoPUP3_17 from Papaver somniferum, each stimulated somewhere in the range of 48-94% more bioconversion of thebaine to northebaine. The improvements in yield shown herein are both unexpected and highly valuable given the nature of the opioid-related compounds produced.

### Example 14. Identification of Purine Uptake Permease (PUP) Transporters Capable of Improving Bioconversion of Oripavine to Nororipavine

### Bioconversion

The impact of purine uptake permease transporter proteins on bioconversion of oripavine to nororipavine was studied by transforming yeast with a plasmid containing a comparable demethylase that was capable of acting on reticuline derivatives such as thebaine and/or oripavine using the backbone plasmid p415TEF. A plasmid containing demethylase-CPR (pOD13 from Example 7) was also expressed in combination with various possible transporter proteins. Yeast strain construction and method of screening for PUP transporters were as previously described in Example 7. Table 14-1 shows the result of percentage bioconversion from oripavine to nororipavine with the expression of various PUP transporters. Table 14-1 also presents the percentage improvement in the bioconversion when normalized for a control strain expressing demethylase but not expressing any heterologous transporter.

### Improvement of Bioconversion.

The percentage bioconversion of strains displayed by several PUP transporters exhibited as high as 1600% and greater bioconversion of the 0.5 mM oripavine fed to the assay when compared to a control strain expressing demethylase but not expressing transporter. Amongst the transporters examined in this example, PUP transporters T149_AcoPUP3_59 T168_FvePUP3_37 T116_HanPUP3_56, T192_CmiPUP3_47, T109_GflPUP3_83, T180_McoPUP3_46, T193AanPUP3_55, T165_AcoPUP3_13 T195_JcuPUP3_71 and T143_CmiPUP3_11 exhibited improvements in the demethylase-mediated bioconversion of oripavine to nororipavine in the range of 1400-1662% in comparison to the control strain expressing demethylase but not expressing a heterologous transporter (Table 14-1). Expression of some PUP transporters, such as T149_AcoPUP3_59 from Aquilegia coeruea, T168_FvePUP3_37 from Fragaria vesca subsp. vesca, and T116_HanPUP3_56 from Helianthus annuus gave particularly remarkable improvements in the demethylase-mediated bioconversion of oripavine to nororipavine.

TABLE 14-1
Percentage of demethylase-mediated bioconversion from Oripavine
to Nororipavine with the expression of various transporters and
the percentage improvement in the bioconversion as compared to
a control strains not expressing any heterologous transporters.

| | Percentage | Percentage Improvement |
|---|---|---|
| | Bioconversion of | in Oripavine to |
| | Oripavine to | Nororipavine |
| PUP Transporters | Nororipavine (%) | bioconversion (%) |
| T101_McoPUP3_1 | 3.5 | 147.7 |
| T102_PsoPUP3_1 | 10.1 | 621.4 |
| T103_PsoPUP3_2 | 1.7 | 21.9 |
| T104_PsoPUP3_3 | 8.0 | 474.8 |
| T105_PsoPUP-L | 12.2 | 771.1 |
| Control 1 | 1.4 | — |
| T109_GflPUP3_83 | 15.5 | 1447.6 |
| T113_PsoPUP3_32 | 10.1 | 912.6 |
| T114_TorPUP3_40 | 5.9 | 486.0 |
| T115_CsaPUP3_48 | 11.7 | 1065.5 |
| T116_HanPUP3_56 | 17.5 | 1653.1 |
| T117_MacPUP3_64 | 4.2 | 317.4 |
| T121_NnuPUP3_9 | 1.5 | 47.1 |
| T122_PsoPUP3_17 | 12.5 | 1149.3 |
| T123_PsoPUP3_25 | 1.3 | 32.4 |
| T124_PsoPUP3_33 | 4.9 | 393.9 |
| T125_JcuPUP3_41 | 14.5 | 1346.2 |
| T126_CpePUP3_49 | 11.8 | 1077.1 |
| T127_LsaPUP3_57 | 5.4 | 441.7 |
| T128_PsoPUP3_65 | 4.8 | 383.9 |
| T129_PsoPUP3_73 | 6.3 | 532.3 |
| T130_NdoPUP3_89 | 14.2 | 1315.0 |
| T131_PbrPUP3_81 | 5.0 | 399.4 |
| T132_CmiPUP3_10 | 14.8 | 1383.1 |
| T133_PsoPUP3_18 | 14.5 | 1349.6 |
| T135_PsoPUP_34 | 1.7 | 73.4 |
| T136_RchPUP3_42 | 13.0 | 1197.8 |
| T137_EguPUP3_50 | 8.4 | 744.8 |
| T138_AduPUP3_58 | 14.5 | 1348.7 |
| T139_PsoPUP3_66 | 4.4 | 341.0 |
| T140_PalPUP3_74 | 3.6 | 264.7 |
| T141_EcaPUP3_88 | 11.3 | 1030.8 |
| T142_McoPUP3_4 | 15.4 | 1438.8 |
| T143_CmiPUP3_11 | 15.8 | 1483.1 |
| T144_PsoPUP3_19 | 15.1 | 1408.2 |
| T146_PsoPUP_35 | 5.8 | 478.0 |
| T147_MesPUP3_43 | 10.5 | 954.4 |
| T148_HimPUP3_51 | 7.7 | 674.8 |
| T149_AcoPUP3_59 | 17.4 | 1639.5 |
| T150_PsoPUP3_67 | 13.4 | 1240.3 |
| T151_PatPUP3_75 | 13.2 | 1223.8 |
| T152_GflPUP3_87 | 14.9 | 1394.9 |
| T153_PsoPUP3_5 | 6.8 | 583.2 |
| T154_CmiPUP3_12 | 11.4 | 1039.5 |
| T156_PsoPUP3_28 | 6.9 | 589.7 |
| T157_RchPUP_36 | 12.2 | 1123.8 |
| T158_DziPUP3_44 | 7.7 | 673.2 |
| T159_OeuPUP3_52 | 10.0 | 902.7 |
| T160_CeuPUP3_60 | 4.0 | 304.6 |
| T161_PsoPUP3_68 | 13.4 | 1237.9 |
| T162_PmiPUP3_76 | 14.1 | 1314.8 |
| T163_PbrPUP3_86 | 3.8 | 280.2 |
| T164_PsoPUP3_78 | 5.5 | 448.3 |
| T165_AcoPUP3_13 | 15.3 | 1429.8 |
| T166_PsoPUP3_21 | 10.3 | 931.0 |
| T168_FvePUP3_37 | 17.6 | 1662.4 |
| T169_ZjuPUP3_45 | 14.1 | 1310.9 |
| T170_LsaPUP3_53 | 14.7 | 1372.2 |
| T171_McoPUP3_61 | 3.5 | 251.3 |
| T172_AcoPUP3_69 | 12.3 | 1126.4 |
| T173_PnuPUP3_77 | 1.9 | 94.1 |
| T174_PbrPUP3_85 | 5.5 | 452.5 |
| T175_PsoPUP3_6 | 8.7 | 769.9 |
| T176_AcoPUP3_14 | 7.4 | 636.2 |
| T177_PsoPUP3_22 | 11.3 | 1029.5 |
| T178_PsoPUP3_30 | 15.0 | 1396.5 |
| T179_PyePUP3_38 | 4.4 | 344.5 |
| T180_McoPUP3_46 | 16.8 | 1580.6 |
| T181_HanPUP3_54 | 12.6 | 1160.4 |
| T182_CpaPUP3_62 | 14.5 | 1349.5 |
| T184_PraPUP3_79 | 3.3 | 234.2 |
| T186_ScaPUP3_84 | 10.6 | 962.2 |
| T188_AcoPUP3_15 | 3.0 | 197.8 |
| T189_PsoPUP3_23 | 8.3 | 729.7 |
| T191_MdoPUP3_39 | 9.5 | 849.2 |
| T192_CmiPUP3_47 | 17.2 | 1618.5 |
| T193_AanPUP3_55 | 15.5 | 1454.4 |
| T194_CchPUP3_63 | 2.1 | 110.0 |
| T195_JcuPUP3_71 | 15.1 | 1413.6 |
| T196_PtrPUP3_80 | 10.9 | 986.6 |
| Control 2 | 1.0 | — |

Note:
Control 1 is used as the control for T101_McoPUP3_1, T102_PsoPUP3_1, T103_PsoPUP3_2, T104_PsoPUP3_3 and T105_PsoPUP-L. Control 2 was used as control for the rest of the PUP transporters. This is was done to account for any slight variations that may arise from different runs of LC-MS analysis.

TABLE 14-2
Purine Uptake Permease transporters which have demonstrated
herein to provide especially large improvements in the demethylase-
mediated bioconversion of Oripavine to Nororipavine.

| Transporter Genes | Latin Name for Origin of Sourced Genes |
|---|---|
| T149_AcoPUP3_59 | Aquilegia coerulea |
| T168_FvePUP3_37 | Fragaria vesca subsp. vesca |
| T116_HanPUP3_56 | Helianthus annuus |
| T192_CmiPUP3_47 | Cinnamomum micranthum f. kanehirae |
| T109_GflPUP3_83 | Glaucium Flavum |
| T180_McoPUP3_46 | Macleaya cordata |
| T193_AanPUP3_55 | Artemisia annua |
| T165_AcoPUP3_13 | Aquilegia coerulea |
| T195_JcuPUP3_71 | Jatropha curcas |
| T143_CmiPUP3_11 | Cinnamomum micranthum f. kanehirae |

### Conclusions

Table 14-2 shows some of the PUP transporters that have been demonstrated herein for the first time to shown particularly high improvements in the demethylase-mediated bioconversion of oripavine to nororipavine. Amongst the transporters examined in this example, PUP transporters T149_AcoPUP3_59 from Aquilegia coerulea, T168_FvePUP3_37 from Fragaria vesca subsp. vesca, T116_HanPUP3_56 from Helianthus annuus, T192_CmiPUP3_47 from Cinnamomum micranthum f. kanehirae, T109_GflPUP3_83 from Glaucium flavum, T180_McoPUP3_46 from Macleaya cordata, T193_AanPUP3_55 from Artemisia annua, T165_AcoPUP3_13 from Aquilegia coerulea, T195_JcuPUP3_71 from Jatropha curcas and T143_CmiPUP3_11 from Cinnamomum micranthum f. kanehirae, exhibited improvements in the range of 1400-1662% more demethylase-mediated bioconversion of thebaine to northebaine in comparison to the control strain expressing demethylase but not expressing a heterologous transporter. Such improvements in yield are particularly remarkable and represent a significant step forward towards a sustainable, secure, and scalable biosynthetic means of producing these compounds.

### Example 15. Identification of Transporters Capable of Improving Bioconversion of Thebaine to Northebaine with Insect Demethylase from Helicoverpa armigera and Heliothis virescens

### Bioconversion

In this example, the impact of transporter proteins on bioconversion of thebaine to northebaine was studied by transforming yeast strain with a plasmid containing a demethylase comparable to the above examples that was capable of acting on reticuline derivatives such as thebaine and/or oripavine using the backbone plasmid p415TEF. A plasmid containing demethylase-CPR (pOD1184 from Example 7) was also expressed in combination with various candidate transporter proteins. Yeast strain construction and method of screening for transporters were as previously described in Example 7. Table 15-1 shows the result of percentage bioconversion from thebaine to northebaine with the expression of various transporters. The screening was performed at pH 7. Table 15-1 also presents the percentage improvement in the bioconversion when normalized for a control strain expressing demethylase but not expressing any heterologous transporter.

### Improvement of Bioconversion.

When compared to a control strain without a heterologous transporter, several strains engineered with various transporters exhibited at least 50% greater or improvement in bioconversion of the 500 μM thebaine fed in this assay. For strains expressing demethylase from Helicoverpa armigera, HaCYP6AE15v2, amongst the heterologous transporters examined, transporters T122_PsoPUP3_17, T149_AcoPUP3_59, T198_AcoT97_GA, T132_CmiPUP3_10, T152_GflPUP3_87, T144_PsoPUP3_19, T157_RchPUP_36 and T168_FvePUP3_37 exhibited improvements in bioconversion of thebaine to northebaine in the range of 54-73% in comparison to the control strain without a heterologous transporter (Table 15-1). Expression of some transporters, such as T122_PsoPUP3_17 from Papaver somniferum, T149_AcoPUP3_59 from Aquilegia coerulea, and T198_AcoT97_GA from Aquilegia coerulea, gave remarkable improvements in the demethylase-mediated bioconversion of thebaine to northebaine.

For strains expressing demethylase from Heliothis virescens, Hv_CYP_A0A2A4JAM9, amongst the heterologous transporters examined, transporters T193_AanPUP3_55, T198_AcoT97_GA, T122_PsoPUP3_17, T157_RchPUP_36, T182_CpaPUP3_62, and T109_GflPUP3_83 exhibited improvements in bioconversion of thebaine to northebaine in the range of 37-50% in comparison to the control strain without a heterologous transporter (Table 15-1). Expression of some transporters, such as T193_AanPUP3_55 from Artemisia annua and T198_AcoT97_GA from Aquilegia coerulea gave remarkable improvements in the demethylase-mediated bioconversion of thebaine to northebaine. In addition, as shown in Table 15-1, several transporters from Helicoverpa armigera and Heliothis virescens have also been sourced and tested. T201_HarPUP3_GA, T212_HarPUP3_GA, T213_HarPUP3_GA, T215_HarPUP3_GA and T216_HarPUP3_GA are from Helicoverpa armigera while T205_HviPUP3_GA is from Heliothis virescens. Only minor thebaine bioconversion of thebaine has been observed with these transporters. For expression with HaCYP6AE15v2, T215_HarPUP3_GA exhibited 6.1% more thebaine bioconversion than the control strain without a heterologous transporter. For expression with Hv_CYP_A0A2A4JAM9, T213_HarPUP3_GA exhibited 3.8% more thebaine bioconversion than the control strain without a heterologous transporter.

TABLE 15-1
Percentage demethylase-mediated bioconversion from Thebaine to Northebaine with the expression of various transporters and
percentage improvements in the bioconversion as compared to a control strains not expressing any heterologous transporters.

| | | Percentage Bioconversion of | | Percentage Improvement in Bioconversion of | |
|---|---|---|---|---|---|
| | | Thebaine to Northebaine, pH 7 (%) | | Thebaine to Northebaine, pH 7 (%) | |
| | SEQ | Demethylase: | Demethylase: | Demethylase: | Demethylase: |
| Transporter | ID NO: | HaCYP6AE15v2 | Hv_CYP_A0A2A4JAM9 | HaCYP6AE15v2 | Hv_CYP_A0A2A4JAM9 |
| T97_ScaT14_GA | 462 | 26.1 | 38.1 | 31.0 | 21.8 |
| T102_PsoPUP3_1 | 466 | 28.6 | 41.7 | 43.7 | 33.3 |
| T105_PsoPUP-L | 472 | 25.2 | 37.8 | 26.6 | 20.7 |
| T109_GflPUP3_83 | 474 | 25.6 | 43.0 | 28.7 | 37.3 |
| T116_HanPUP3_56 | 482 | 24.3 | 40.5 | 22.2 | 29.4 |
| T122_PsoPUP3_17 | 488 | 34.5 | 44.2 | 73.2 | 41.2 |
| T132_CmiPUP3_10 | 508 | 32.5 | 42.5 | 63.3 | 35.6 |
| T141_EcaPUP3_88 | 524 | 29.1 | 39.5 | 46.2 | 26.3 |
| T142_McoPUP3_4 | 526 | 28.8 | 42.6 | 44.5 | 36.2 |
| T143_CmiPUP3_11 | 528 | 21.6 | 27.3 | 8.6 | −13.0 |
| T144_PsoPUP3_19 | 530 | 31.0 | 40.0 | 55.6 | 27.8 |
| T149_AcoPUP3_59 | 538 | 33.4 | 32.5 | 67.6 | 3.7 |
| T152_GflPUP3_87 | 544 | 31.5 | 40.5 | 58.3 | 29.5 |
| T157_RchPUP_36 | 552 | 31.0 | 43.6 | 55.4 | 39.2 |
| T165_AcoPUP3_13 | 568 | 24.4 | 36.8 | 22.7 | 17.6 |
| T168_FvePUP3_37 | 572 | 30.8 | 39.9 | 54.7 | 27.5 |
| T175_PsoPUP3_6 | 586 | 23.3 | 36.9 | 17.0 | 17.8 |
| T180_McoPUP3_46 | 596 | 26.3 | 41.3 | 32.2 | 31.8 |
| T182_CpaPUP3_62 | 600 | 28.5 | 43.5 | 43.2 | 39.1 |
| T186_ScaPUP3_84 | 604 | 25.5 | 40.6 | 28.0 | 29.7 |
| T192_CmiPUP3_47 | 612 | 26.3 | 39.7 | 32.1 | 26.6 |
| T193_AanPUP3_55 | 614 | 30.2 | 47.2 | 51.5 | 50.6 |
| T195_JcuPUP3_71 | 618 | 23.5 | 35.1 | 18.2 | 12.2 |
| T197_AcoT97_GA | 622 | 19.7 | 33.1 | −1.3 | 5.7 |
| T198_AcoT97_GA | 624 | 33.2 | 44.7 | 66.5 | 42.7 |
| T199_NnuT97_GA | 626 | 19.9 | 32.3 | −0.2 | 3.0 |
| T200_T97_GA | 628 | 20.1 | 31.3 | 1.0 | −0.1 |
| T201_HarPUP3_GA | 630 | 19.5 | 32.5 | −1.9 | 3.7 |
| T202_PgoPUP3_GA | 632 | 17.7 | 31.1 | −10.9 | −0.5 |
| T204_RcoPUP3_GA | 636 | 22.6 | 35.3 | 13.7 | 12.6 |
| T205_HviPUP3_GA | 638 | 20.6 | 32.3 | 3.2 | 3.3 |
| T206_VviPUP3_3_GA | 640 | 20.8 | 39.6 | 4.6 | 26.5 |
| T207_MprPUP3_GA | 642 | 28.5 | 37.8 | 43.2 | 20.8 |
| T208_McoPUP3_GA | 644 | 27.7 | 37.6 | 38.9 | 19.9 |
| T209_RcoPUP3_GA | 646 | 24.3 | 39.5 | 22.0 | 26.1 |
| T210_NnuPUP3_GA | 648 | 25.3 | 38.9 | 27.0 | 24.3 |
| T212_HarPUP3_GA | 652 | 24.7 | 30.4 | 23.9 | −3.0 |
| T213_HarPUP3_GA | 654 | 20.1 | 35.1 | 0.9 | 12.2 |
| T215_HarPUP3_GA | 658 | 26.0 | 31.4 | 30.4 | 0.4 |
| T216_HarPUP3_GA | 660 | 20.1 | 31.6 | 1.1 | 1.0 |
| T217_AcoPUP3_GA | 662 | 19.4 | 28.0 | −2.8 | −10.6 |
| Control | | 19.9 | 31.3 | — | — |

Note:
Demethylase: HaCYP6AE15v2 represents demethylase from Helicoverpa armigera; Demethylase: Hv_CYP_A0A2A4JAM9 represents demethylase from Heliothis virescens. Control strain only contains a copy of demethylase, a copy of demethylase-CPR, HaCPR_E7E2N6 from Helicoverpa armigera, and an empty plasmid p416TEF. The demethylase-CPR, HaCPR_E7E2N6 is present in all strains.

### Conclusion

Table 15-1 shows some of the transporters that have been herein demonstrated to have shown very considerable improvements in the bioconversion from thebaine to northebaine by 2 different demethylases. In particular, the results of this example demonstrate that together with demethylase, HaCYP6AE15v2, expression of transporters T122_PsoPUP3_17 from Papaver somniferum, T149_AcoPUP3_59 from Aquilegia coerulea, T198_AcoT97_GA from Aquilegia coerulea, T132_CmiPUP3_10 from Cinnamomum micranthum f. kanehirae, T152_GflPUP3_87 from Glaucium Flavum, T144_PsoPUP3_19 from Papaver somniferum, T157_RchPUP_36 from Rosa chinensis and T168_FvePUP3_37 from Fragaria vesca subsp. vesca, each stimulated somewhere in the range of 54-73% more bioconversion of thebaine to northebaine. As for transporters expressing together with demethylase, Hv_CYP_A0A2A4JAM9, transporters T193_AanPUP3_55 from Artemisia annua and T198_AcoT97_GA from Aquilegia coerulea. The improvements in yield shown herein are both unexpected and highly valuable given the nature of the opioid-related compounds produced.

### Example 16. The Efficiency of Bioconversion from Thebaine to Northebaine is Demethylase and Transporter Dependent

### Combination of Demethylase and Transporter

Table 16-1 shows the top 5 transporters that demonstrate sufficient efficiency in thebaine to northebaine bioconversion when expressing together with demethylase, CYPDN43 (SEQ ID NO: 202) from Lichtheimia corymbifera. The best transporter/Demethylase combination is T152_GflPUP3_87/CYPDN43 which was capable of converting 8% of the 500 μM thebaine fed to northebaine. T152_GflPUP3_87 is a PUP transporter from Glaucium flavum. This is followed by the combination of T142_McoPUP3_4/CYPDN43 and T144_PsoPUP3_19/CYPDN43. T142_McoPUP3_4 and T144_PsoPUP3_19 are PUP transporters from Macleaya cordata and Papaver somniferum, respectively.

TABLE 16-1
Top 5 transporters ranking list when expressing with Lichtheimia
corymbifera demethylase, CYPDN43. The ranking is based
on percentage demethylase-mediated bioconversion from
Thebaine to Northebaine from Table 13-1 in Example 13.

| | | | Percentage |
|---|---|---|---|
| | | | Bioconversion |
| | | | of Thebaine |
| | | | to Northebaine |
| Rank of Top 5 Transporters | | Demethylase | (%) |
| 1 | T152_GflPUP3_87 | CYPDN43 | 8.0 |
| 2 | T142_McoPUP3_4 | CYPDN43 | 7.7 |
| 3 | T144_PsoPUP3_19 | CYPDN43 | 7.7 |
| 4 | T186_ScaPUP3_84 | CYPDN43 | 7.0 |
| 5 | T149_AcoPUP3_59 | CYPDN43 | 6.9 |

Table 16-2 shows the top 5 transporters that demonstrate remarkable efficiency in thebaine to northebaine bioconversion when expressing together with demethylase, HaCYP6AE15v2 from Helicoverpa armigera. The best transporter/Demethylase combination is T122_PsoPUP3_17/HaCYP6AE15v2 which was capable of converting as high as 34.5% of the 500 μM thebaine fed to northebaine. T122_PsoPUP3_17 is a PUP transporter from Papaver somniferum. This is followed by the combination of T149_AcoPUP3_59/HaCYP6AE15v2 and T198_AcoT97_GA/HaCYP6AE15v2. Both T149_AcoPUP3_59 and T198_AcoT97_GA are transporters from Aquilegia coerulea.

TABLE 16-2
Top 5 transporters ranking list when expressing with Helicoverpa
armigera demethylase, HaCYP6AE15v2. The ranking is based
on percentage demethylase-mediated bioconversion from
Thebaine to Northebaine from Table 15-1 in Example 15.

| | | | Percentage |
|---|---|---|---|
| | | | Bioconversion |
| | | | of Thebaine |
| | | | to Northebaine |
| Rank of Top 5 Transporters | | Demethylase | (%) |
| 1 | T122_PsoPUP3_17 | HaCYP6AE15v2 | 34.5 |
| 2 | T149_AcoPUP3_59 | HaCYP6AE15v2 | 33.4 |
| 3 | T198_AcoT97_GA | HaCYP6AE15v2 | 33.2 |
| 4 | T132_CmiPUP3_10 | HaCYP6AE15v2 | 32.5 |
| 5 | T152_GflPUP3_87 | HaCYP6AE15v2 | 31.5 |

Table 16-3 shows the top 5 transporters that demonstrate remarkable efficiency in thebaine to northebaine bioconversion when expressing together with demethylase, Hv_CYP_A0A2A4JAM9 from Heliothis virescens. The best transporter/demethylase combination is T193_AanPUP3_55/Hv_CYP_A0A2A4JAM9 which was capable of converting 47.2% of the 500 μM thebaine fed to northebaine. T193_AanPUP3_55 is a PUP transporter from Artemisia annua. This is followed by the combination of T198_AcoT97_GA/Hv_CYP_A0A2A4JAM9 and T122_PsoPUP3_17/Hv_CYP_A0A2A4JAM9. T198_AcoT97_GA and T122_PsoPUP3_17 are transporters from Aquilegia coerulea and Papaver somniferum, respectively.

TABLE 16-3
Top 5 transporters ranking list when expressing with Heliothis virescens
demethylase, Hv_CYP_A0A2A4JAM9. The ranking is based
on percentage demethylase-mediated bioconversion from Thebaine
to Northebaine from Table 15-1 in Example 15.

| | | | Percentage |
|---|---|---|---|
| | | | Bioconversion |
| | | | of Thebaine |
| | | | to Northebaine |
| Rank of Top 5 Transporters | | Demethylase | (%) |
| 1 | T193_AanPUP3_55 | Hv_CYP_A0A2A4JAM9 | 47.2 |
| 2 | T198_AcoT97_GA | Hv_CYP_A0A2A4JAM9 | 44.7 |
| 3 | T122_PsoPUP3_17 | Hv_CYP_A0A2A4JAM9 | 44.2 |
| 4 | T157_RchPUP_36 | Hv_CYP_A0A2A4JAM9 | 43.6 |
| 5 | T182_CpaPUP3_62 | Hv_CYP_A0A2A4JAM9 | 43.5 |

### Conclusion

Based on the data presented in Table 16-1, Table 16-2 and Table 16-3 as well as the previous data in Examples 10 and 11, it shows that the best combination of transporter and demethylase for bioconversion of thebaine varies depending on which demethylase, the transporter is co-expressing with. Based on the overall result, T122_PsoPUP3_17 from Papaver somniferum and HaCYP6AE15v2 from Helicoverpa armigera is the best combination of demethylase/transporter for thebaine to northebaine bioconversion. The efficiency of bioconversion for thebaine to northebaine is demethylase and transporter dependent.

### Example 17. pH Dependency of the Efficiency of Bioconversion from Thebaine to Northebaine

### Comparison of Efficiency of Thebaine Bioconversion in Different pH

Several yeast strains presented in Table 15-1 have been tested in growth medium at different pH in order to investigate if pH has any effect on thebaine bioconversion. The growth medium used in this experiment was DELFT minimal medium and the medium was buffered with 1M succinic acid to pH4.5 or buffered with 1M sodium hydroxide to pH7. The result in Table 17-1 shows that when the strains were grown at pH 4.5, the efficiency of thebaine bioconversion is generally lower than at pH7. At pH 4.5, the control strain without any expression of a heterologous transporter hardly able to convert any thebaine to northebaine, only 0.1% of thebaine was converted. However, the same strain was able to convert 19.8% of thebaine at pH 7. As shown in Table 17-1, when a heterologous transporter was expressed, the efficiency of thebaine bioconversion was improved. When PUP transporter, T122_PsoPUP3_17 was expressed together with Helicoverpa armigera demethylase, HaCYP6AE15v2, 39.7% of the 500 lpM thebaine fed was converted to northebaine. This means 19.9% more thebaine was converted to northebaine when transporter, T122_PsoPUP3_17 from Papaver somniferum was expressed. At pH4.5, the same strain only converted 22.2% of Thebaine to Northebaine.

TABLE 17-1
Percentage demethylase-mediated bioconversion from Thebaine
to Northebaine at different pH. The demethylase used in this
experiment is HaCYP6AE15v2 from Helicoverpa armigera.

| | | Percentage | Percentage |
|---|---|---|---|
| | | Bioconversion | Bioconversion |
| | SEQ | of Thebaine to | of Thebaine to |
| | ID | Northebaine, | Northebaine, |
| Transporter | NO: | pH 4.5 (%) | pH 7 (%) |
| T102_PsoPUP3_1 | 466 | 16.3 | 28.6 |
| T122_PsoPUP3_17 | 488 | 22.2 | 39.7 |
| T149_AcoPUP3_59 | 538 | 12.9 | 33.4 |
| T168_FvePUP3_37 | 568 | 7.7 | 30.8 |
| T169_ZjuPUP3_45 | 574 | 5.4 | 27.6 |
| T193_AanPUP3_55 | 614 | 7.0 | 30.2 |
| Control | — | 0.5 | 19.8 |

### Conclusion

The result presented in Table 17-1 demonstrates that pH is important for the bioconversion of thebaine to northebaine. Using a pH around 7 is more optimal for the bioconversion of thebaine than at lower pH. Similar pH test has also been performed with yeast strains expressing other demethylase and transporters, same conclusion has been reached. The pH appears to make a huge impact on transporting the substrate across membranes, so adjusting pH impacts the bioconversion capability of cells.

### Example 18. Identification of Uptake Transporters Capable of Improving Bioconversion of Oripavine to Nororipavine with Demethylase from Helicoverpa armigera and Heliothis virescens

### Bioconversion

The impact of transporter proteins on bioconversion of oripavine to nororipavine was studied by transforming yeast with a plasmid containing a comparable demethylase that was capable of acting on reticuline derivatives such as thebaine and/or oripavine using the backbone plasmid p415TEF. A plasmid containing demethylase-CPR (pOD1184 from Example 7) was also expressed in combination with various possible transporter proteins. Yeast strain construction and method of screening for transporters were as previously described in Example 7. Table 18-1 shows the result of percentage bioconversion from oripavine to nororipavine with the expression of various uptake transporters. Table 18-1 also presents the percentage improvement in the bioconversion when normalized for a control strain expressing demethylase but not expressing any heterologous transporter.

### Improvement of Bioconversion.

When compared to a control strain without a heterologous transporter, some strains engineered with various transporters exhibited more than 50% bioconversion of the 500 μM oripavine fed in this assay. For strains expressing demethylase from Helicoverpa armigera, HaCYP6AE15v2, amongst the heterologous transporters examined, transporters T165_AcoPUP3_13, T149_AcoPUP3_59, T193_AanPUP3_55, T168_FvePUP3_37 and T180_McoPUP3_46 exhibited improvements in bioconversion of oripavine to nororipavine in the range of 2125-2327% in comparison to the control strain without a heterologous transporter (Table 18-1). Expression of some transporters, such as T165_AcoPUP3_13 and T149_AcoPUP3_59 from Aquilegia coerulea, and T193_AanPUP3_55 from Artemisia annua, gave particularly remarkable improvements in the demethylase-mediated bioconversion of oripavine to nororipavine.

For strains expressing demethylase from Heliothis virescens, Hv_CYP_A0A2A4JAM9, amongst the heterologous transporters examined, transporters T193_AanPUP3_55, T180_McoPUP3_46, T149_AcoPUP3_59, T165_AcoPUP3_13 and T198_AcoT97_GA exhibited improvements in bioconversion of oripavine to nororipavine in the range of 3502-4033% in comparison to the control strain without a heterologous transporter (Table 18-1). Expression of some transporters, such as T193_AanPUP3_55 from Artemisia annua and T180_McoPUP3_46 from Macleaya cordata demonstrated particularly outstanding improvements in the demethylase-mediated bioconversion of oripavine to nororipavine.

In addition, in Table 18-1, for the first time, several transporters from Helicoverpa armigera and Heliothis virescens have also been tested in bioconversion of oripavine to nororipavine. T201_HarPUP3_GA, T212_HarPUP3_GA, T213_HarPUP3_GA, T215_HarPUP3_GA and T216_HarPUP3_GA transporters are from Helicoverpa armigera while T205_HviPUP3_GA transporter is from Heliothis virescens. Some of these Helicoverpa armigera and Heliothis virescens transporters exhibited great effect on bioconversion in cells of oripavine to nororipavine. For expression with HaCYP6AE15v2, T212_HarPUP3_GA and T215_HarPUP3_GA exhibited 1952.7% and 1280.9%, respectively, more bioconversion of oripavine to nororipavine than the control strain without a heterologous transporter. For expression with Hv_CYP_A0A2A4JAM9, T213_HarPUP3_GA exhibited 26.0% more oripavine bioconversion than the control strain without a heterologous transporter.

TABLE 18-1
Percentage demethylase-mediated bioconversion from Oripavine to Nororipavine with the expression of various transporters and
percentage improvements in the bioconversion as compared to a control strains not expressing any heterologous transporters.

| | | Percentage Bioconversion of Oripavine | | Percentage Improvement in Oripavine | |
|---|---|---|---|---|---|
| | | to Nororipavine, pH 4.5 (%) | | to Nororipavine bioconversion, pH 4.5 (%) | |
| | SEQ | Demethylase: | Demethylase: | Demethylase: | Demethylase: |
| Transporter | ID NO: | HaCYP6AE15v2 | Hv_CYP_A0A2A4JAM9 | HaCYP6AE15v2 | Hv_CYP_A0A2A4JAM9 |
| T97_ScaT14_GA | 462 | 25.3 | 35.4 | 1343.4 | 2561.3 |
| T102_PsoPUP3_1 | 466 | 27.5 | 36.3 | 1472.2 | 2630.4 |
| T105_PsoPUP-L | 472 | 10.4 | 15.5 | 491.8 | 1067.8 |
| T109_GflPUP3_83 | 474 | 27.6 | 30.1 | 1477.0 | 2163.3 |
| T116_HanPUP3_56 | 482 | 33.6 | 44.7 | 1817.6 | 3258.3 |
| T122_PsoPUP3_17 | 488 | 23.5 | 29.5 | 1243.2 | 2114.9 |
| T132_CmiPUP3_10 | 508 | 18.6 | 20.9 | 961.3 | 1469.1 |
| T141_EcaPUP3_88 | 524 | 24.4 | 28.1 | 1296.3 | 2013.6 |
| T142_McoPUP3_4 | 526 | 32.3 | 38.2 | 1742.7 | 2772.6 |
| T143_CmiPUP3_11 | 528 | 17.5 | 10.4 | 901.3 | 685.4 |
| T144_PsoPUP3_19 | 530 | 31.1 | 34.5 | 1674.0 | 2492.1 |
| T149_AcoPUP3_59 | 538 | 42.2 | 50.7 | 2308.0 | 3710.7 |
| T152_GflPUP3_87 | 544 | 21.9 | 20.2 | 1153.0 | 1419.8 |
| T157_RchPUP_36 | 552 | 22.0 | 30.7 | 1159.1 | 2209.3 |
| T165_AcoPUP3_13 | 568 | 42.5 | 42.4 | 2327.3 | 3089.5 |
| T168_FvePUP3_37 | 572 | 39.2 | 50.3 | 2139.6 | 3683.8 |
| T175_PsoPUP3_6 | 586 | 10.0 | 16.2 | 471.4 | 1114.2 |
| T180_McoPUP3_46 | 596 | 39.0 | 52.7 | 2125.8 | 3864.2 |
| T182_CpaPUP3_62 | 600 | 34.5 | 41.1 | 1870.7 | 2991.0 |
| T186_ScaPUP3_84 | 604 | 16.1 | 24.6 | 818.8 | 1747.2 |
| T192_CmiPUP3_47 | 612 | 30.9 | 40.3 | 1663.9 | 2930.2 |
| T193_AanPUP3_55 | 614 | 40.0 | 55.0 | 2182.9 | 4033.0 |
| T195_JcuPUP3_71 | 618 | 34.7 | 40.5 | 1884.9 | 2943.3 |
| T197_AcoT97_GA | 622 | 1.5 | 1.8 | −11.7 | 35.4 |
| T198_AcoT97_GA | 624 | 37.3 | 47.9 | 2033.5 | 3502.3 |
| T199_NnuT97_GA | 626 | 1.5 | 1.6 | −15.8 | 19.1 |
| T200_T97_GA | 628 | 1.3 | 1.4 | −23.0 | 5.0 |
| T201_HarPUP3_GA | 630 | 1.4 | 1.4 | −22.5 | 7.8 |
| T202_PgoPUP3_GA | 632 | 1.2 | 1.4 | −29.8 | 1.7 |
| T204_RcoPUP3_GA | 636 | 3.9 | 7.1 | 123.9 | 433.2 |
| T205_HviPUP3_GA | 638 | 1.3 | 1.3 | −25.3 | −0.2 |
| T206_VviPUP3_3_GA | 640 | 1.3 | 31.3 | −27.4 | 2253.5 |
| T207_MprPUP3_GA | 642 | 24.7 | 39.7 | 1312.6 | 2884.7 |
| T208_McoPUP3_GA | 644 | 33.3 | 2.8 | 1804.0 | 113.7 |
| T209_RcoPUP3_GA | 646 | 2.2 | 23.5 | 25.3 | 1663.4 |
| T210_NnuPUP3_GA | 648 | 17.0 | 44.6 | 873.0 | 3251.0 |
| T212_HarPUP3_GA | 652 | 35.9 | 1.4 | 1952.7 | 6.0 |
| T213_HarPUP3_GA | 654 | 1.3 | 27.3 | −27.5 | 1949.7 |
| T215_HarPUP3_GA | 658 | 24.2 | 1.3 | 1280.9 | 0.2 |
| T216_HarPUP3_GA | 660 | 1.3 | 1.3 | −26.7 | −1.9 |
| T217_AcoPUP3_GA | 662 | 1.2 | 2.3 | −29.7 | 69.6 |
| Control | | 1.8 | 1.3 | — | — |

Note:
Demethylase: HaCYP6AE15v2 represents demethylase from Helicoverpa armigera; Demethylase: Hv_CYP_A0A2A4JAM9 represents demethylase from Heliothis virescens. Control strain only contains a copy of demethylase, a copy of demethylase-CPR, HaCPR_E7E2N6 from Helicoverpa armigera, and an empty plasmid p416TEF. The demethylase-CPR, HaCPR_E7E2N6 is present in all strains.

### Conclusion

Table 18-1 shows various uptake transporters that have been demonstrated herein some for the first time to shown particularly high improvements in the demethylase-mediated bioconversion of oripavine to nororipavine. Amongst the transporters tested in this example, transporters T193_AanPUP3_55 from Artemisia annua, T180_McoPUP3_46 from Macleaya cordata, T149_AcoPUP3_59 from Aquilegia coerulea, T165_AcoPUP3_13 from Aquilegia coerulea, and T198_AcoT97_GA from Aquilegia coerulea, exhibited improvements in the range of 3502-4033% more demethylase-mediated bioconversion of oripavine to nororipavine in comparison to the control strain expressing demethylase but not expressing a heterologous transporter. In addition, several uptake transporters from Helicoverpa armigera such as T212_HarPUP3_GA, T213_HarPUP3_GA and T215_HarPUP3_GA have also exhibited excellent demethylase-mediated bioconversion of oripavine to nororipavine. Such improvements in yield are particularly remarkable and represent a significant step forward towards a sustainable, secure, and scalable biosynthetic means of producing these compounds.

### Example 19. The Efficiency of Bioconversion from Oripavine to Nororipavine is Demethylase Dependent Combination of Demethylase and Transporter

Table 19-1 shows the top 5 transporters that demonstrate sufficient efficiency in oripavine to nororipavine bioconversion when expressing together with demethylase, CYPDN43 from Lichtheimia corymbifera. The best transporter/demethylase combination is T168_FvePUP3_37/CYPDN43 which was capable of converting 17.6% of the 500 μM oripavine fed to nororipavine. T168_FvePUP3_37 is a PUP transporter from Fragaria vesca subsp. vesca. This is followed by the combination of T116_HanPUP3_56/CYPDN43 and T149_AcoPUP3_59/CYPDN43. T116_HanPUP3_56 and T149_AcoPUP3_59 are PUP transporters from Helianthus annuus and Aquilegia coerulea, respectively.

TABLE 19-1
Top 5 transporters ranking list when expressing with Lichtheimia
corymbifera demethylase, CYPDN43. The ranking is based
on percentage demethylase-mediated bioconversion from
Oripavine to Nororipavine from Table 14-1 in Example 14.

| | | | Percentage |
|---|---|---|---|
| | | | Bioconversion |
| | | | of Oripavine to |
| | | | Nororipavine |
| Rank of Top 5 Transporters | | Demethylase | (%) |
| 1 | T168_FvePUP3_37 | CYPDN43 | 17.6 |
| 2 | T116_HanPUP3_56 | CYPDN43 | 17.5 |
| 3 | T149_AcoPUP3_59 | CYPDN43 | 17.4 |
| 4 | T192_CmiPUP3_47 | CYPDN43 | 17.2 |
| 5 | T180_McoPUP3_46 | CYPDN43 | 16.8 |

Table 19-2 shows the top 5 transporters that demonstrate remarkable efficiency in oripavine to nororipavine bioconversion when expressing together with demethylase, HaCYP6AE15v2 from Helicoverpa armigera. The best transporter/demethylase combination is T165_AcoPUP3_13/HaCYP6AE15v2 which was capable of converting as high as 42.5% of the 500 μM oripavine fed to nororipavine. T165_AcoPUP3_13 is a PUP transporter from Aquilegia coerulea. This is followed by the combination of T149_AcoPUP3_59/HaCYP6AE15v2 and T193_AanPUP3_55/HaCYP6AE15v2. Transporters T149_AcoPUP3_59 from Aquilegia coerulea and T193_AanPUP3_55 from Artemisia annua.

TABLE 19-2
Top 5 transporters ranking list when expressing with Helicoverpa
armigera demethylase, HaCYP6AE15v2. The ranking is based
on percentage demethylase-mediated bioconversion from
Oripavine to Nororipavine from Table 18-1 in Example 18.

| | | | Percentage |
|---|---|---|---|
| | | | Bioconversion |
| | | | of Oripavine to |
| | | | Nororipavine |
| Rank of Top 5 Transporters | | Demethylase | (%) |
| 1 | T165_AcoPUP3_13 | HaCYP6AE15v2 | 42.5 |
| 2 | T149_AcoPUP3_59 | HaCYP6AE15v2 | 42.2 |
| 3 | T193_AanPUP3_55 | HaCYP6AE15v2 | 40.0 |
| 4 | T168_FvePUP3_37 | HaCYP6AE15v2 | 39.2 |
| 5 | T180_McoPUP3_46 | HaCYP6AE15v2 | 39.0 |

Table 19-3 shows the top 5 transporters that demonstrate remarkable efficiency in oripavine to nororipavine bioconversion when expressing together with demethylase, Hv_CYP_A0A2A4JAM9 from Heliothis virescens. The best transporter/demethylase combination is T193_AanPUP3_55/Hv_CYP_A0A2A4JAM9 which was capable of converting 55.0% of the 500 μM oripavine fed to northebaine. T193_AanPUP3_55 is a PUP transporter from Artemisia annua. This is followed by the combination of T180_McoPUP3_46/Hv_CYP_A0A2A4JAM9 and T149_AcoPUP3_59/Hv_CYP_A0A2A4JAM9. T180_McoPUP3_46 and T149_AcoPUP3_59 are transporters from Macleaya cordata and Aquilegia coerulea, respectively.

TABLE 19-3
Top 5 transporters ranking list when expressing with Heliothis virescens
demethylase, Hv_CYP_A0A2A4JAM9. The ranking is based
on percentage demethylase-mediated bioconversion from Oripavine
to Nororipavine from Table 18-1 in Example 18.

| | | | Percentage |
|---|---|---|---|
| | | | Bioconversion |
| | | | of Oripavine to |
| | | | Nororipavine |
| Rank of Top 5 Transporters | | Demethylase | (%) |
| 1 | T193_AanPUP3_55 | Hv_CYP_A0A2A4JAM9 | 55.0 |
| 2 | T180_McoPUP3_46 | Hv_CYP_A0A2A4JAM9 | 52.7 |
| 3 | T149_AcoPUP3_59 | Hv_CYP_A0A2A4JAM9 | 50.7 |
| 4 | T165_AcoPUP3_13 | Hv_CYP_A0A2A4JAM9 | 50.3 |
| 5 | T198_AcoT97_GA | Hv_CYP_A0A2A4JAM9 | 47.9 |

### Conclusion

Based on the data presented in Table 19-1, Table 19-2 and Table 19-3 as well as the previous data in Table 10-2, 12-1 and 14-1, it shows that the best combination of transporter and demethylase for bioconversion of oripavine varies depending on which demethylase, the transporter is co-expressing with. Based on the overall result, T193_AanPUP3_55 from Artemisia annua and Hv_CYP_A0A2A4JAM9 from Heliothis virescens are the best combination of demethylase/transporter for oripavine to nororipavine bioconversion. The efficiency of bioconversion for oripavine to nororipavine is demethylase and transporter dependent.

### Example 20. the Efficiency of Bioconversion from Oripavine to Nororipavine is pH Dependent

### Comparison of Efficiency of Oripavine Bioconversion in Different pH

Several yeast strains presented in Table 18-1 have been tested in growth medium at different pH in order to investigate if pH has any effect on bioconversion of oripavine. The growth medium used in this experiment was DELFT minimal medium and the medium was buffered with 1 M succinic acid to pH 4.5 or buffered with 1M sodium hydroxide to pH 7. The result in Table 20-1 shows that when the strains were grown at pH 4.5, the efficiency of oripavine bioconversion to nororipavine is generally higher than at pH 7. At pH 4.5, the control strain without any expression of a heterologous transporter hardly converts any oripavine to nororipavine. At pH 7, the same strain was able to convert merely 4.0% of oripavine. As shown in Table 20-1, when a heterologous transporter was expressed, the efficiency of oripavine bioconversion was significantly improved. When a PUP transporter, T168_FvePUP3_37 was expressed together with Helicoverpa armigera demethylase, HaCYP6AE15v2, 20.7% of the 500 μM oripavine fed was converted to nororipavine at pH 7. At a culture condition of pH 4.5, the same strain was able to convert 33.3% of oripavine to nororipavine. This means 12.6% more of oripavine was converted to nororipavine when the pH condition of the cell was lowered to pH 4.5.

TABLE 20-1
Percentage demethylase-mediated bioconversion from Oripavine
to Nororipavine at different pH. The demethylase used in this
experiment is HaCYP6AE15v2 from Helicoverpa armigera.

| | Percentage | Percentage |
|---|---|---|
| | Bioconversion | Bioconversion |
| | of Oripavine to | of Oripavine to |
| | Nororipavine, | Nororipavine, |
| Transporter | pH 4.5 (%) | pH 7 (%) |
| T102_PsoPUP3_1 | 22.4 | 0.5 |
| T122_PsoPUP3_17 | 19.9 | 19.8 |
| T149_AcoPUP3_59 | 32.9 | 20.3 |
| T168_FvePUP3_37 | 33.3 | 20.7 |
| T169_ZjuPUP3_45 | 23.5 | 15.6 |
| T193_AanPUP3_55 | 28.6 | 19.1 |
| Control | 0.0 | 4.0 |

### Conclusion

The result presented in Table 20-1 demonstrates that an optimal pH condition is extremely important for the bioconversion of oripavine to nororipavine. Screening at lower pH of 4.5 is more optimal for the bioconversion of oripavine than at higher pH. Similar pH test has also been performed with yeast strains expressing other demethylase and transporters, same conclusion has been reached.

### Example 21. Improvement of Bioconversion from Oripavine to Nororipavine with Multiple Genes Expression of Demethylase and Transporter

### Optimization of Bioconversion Efficiency of Oripavine by Ty Integration

Optimization of the bioconversion was performed by multiple genes overexpression. It is generally known that higher copy number of genes causes higher level of transcription and therefore more efficient production of (heterologous) proteins (Bitter B G A et al, 1987). Method of integration has been previously described in Example 7. In this example, several best demethylase/transporter combinations for oripavine have been expressed in the same yeast strain background. As shown in Table 21-1, multiple genes expression of HaCYP6AE15v2/T102_PsoPUP3-1 in sOD343 and HaCYP6AE15v2/T149_AcoPUP3-1 in sOD344 increase the oripavine bioconversion to 66.7% and 81.6%, respectively. Single copy gene expression of HaCYP6AE15v2/T149_AcoPUP3-1 only managed to convert 42.2% of the 500 μM oripavine fed to nororipavine (Table 19-2).

TABLE 21-1
Percentage demethylase-mediated bioconversion from Oripavine to Nororipavine with
multiple genes overexpression of demethylase and transporter by Ty integration.

| | | | Oripavine | Percentage Bioconversion |
|---|---|---|---|---|
| | Multiple gene expression by Ty | | fed | of Oripavine to |
| Strains | Demethylase | Transporter | (μM) | Nororipavine, pH 4.5 (%) |
| sOD343 | HaCYP6AE15v2 | T102_PsoPUP3-1 | 500 | 66.7 |
| sOD344 | HaCYP6AE15v2 | T149_AcoPUP3-1 | 500 | 81.6 |
| sOD398 | Hv_CYP_A0A2A4JAM9 | T180_McoPUP3_46 | 500 | 95.5 |
| sOD398 | Hv_CYP_A0A2A4JAM9 | T180_McoPUP3_46 | 1000 | 84.7 |

Previously in Table 19-3, one of the best single copy gene expression combination for oripavine bioconversion was Hv_CYP_A0A2A4JAM9/T180_McoPUP3_46. 52.7% of the 500 μM oripavine fed was converted to nororipavine. In Table 21-1, when 500 μM of oripavine was fed, multiple genes expression of Hv_CYP_A0A2A4JAM9/T180_McoPUP3_46 as shown by sOD398 increased the oripavine bioconversion from 52.7% to 95.5%. When 1000 μM of oripavine was fed, sOD398 was able to convert 84.7% of oripavine to nororipavine.

### Conclusion

The result presented in this example demonstrates that multiple genes expression greatly improves the efficiency of bioconversion from oripavine to nororipavine. Various source of demethylase and transporter have shown to exert the same improvement. The level of improvement is dependent on the demethylase/transporter combination.

### Example 22 Modification of Base Strain to Express DRS-DRR

The S. cerevisiae strain BY4741 was deleted for the gene ARI1 and engineered to overexpress the following genes: ARO4fbr (SEQ ID NO: 2), PpDODC (SEQ ID NO: 72), CYP76AD1_2mut (SEQ ID NO: 66), HDEL_CjNCS_V152 (SEQ ID NO: 77), Ps60MT_Q6WUC1 (SEQ ID NO: 80), Cj40MT (SEQ ID NO: 90), AtATR1 (SEQ ID NO: 115), EcNMCH (SEQ ID NO: 86), CjCNMT (SEQ ID NO: 83), PbSaIR (SEQ ID NO:121), PbSAS (SEQ ID NO: 117), PsSAT (SEQ ID NO: 124), PsCPR (SEQ ID NO: 113) and PsTHS1 (SEQ ID NO: 130). All genes were codon optimized for expression in S. cerevisiae except the genes that already originated from yeast. The promoters used for driving expression of these genes were pTDH3, pPDC1, pTEF1, pTEF2, pTPI1 and pPGK1. Expression cassettes with these genes and promoters were integrated into different yeast chromosomes using vectors as described by Mikkelsen et al (Metabolic Engineering Volume 14, Issue 2, March 2012, Pages 104-111. Michael Dalgaard Mikkelsen, Line Due Buron, Bo Salomonsen, Carl Erik Olsen, Bjarne Gram Hansen, Uffe Hasbro Mortensen, Barbara Ann Halkier. Microbial production of indolylglucosinolate through engineering of a multi-gene pathway in a versatile yeast expression platform).

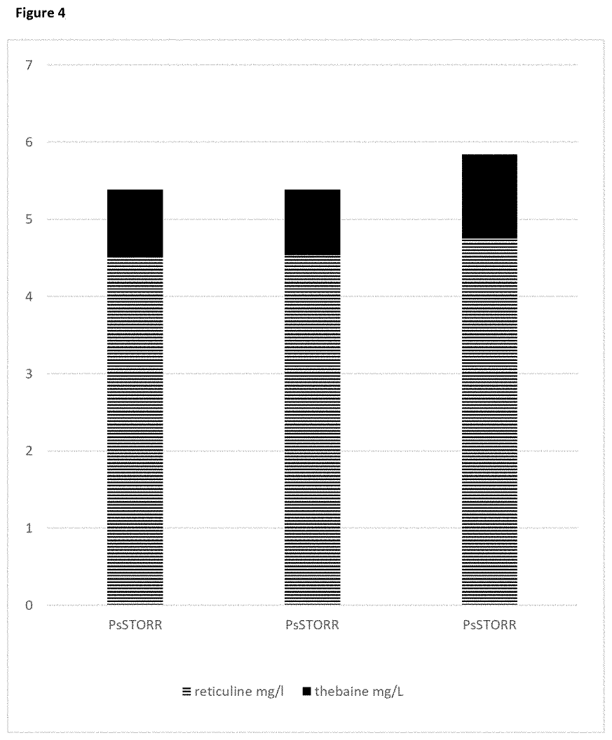

To complete the thebaine biosynthesis pathway in this yeast strain, different DRS-DRR enzymes were introduced for isomerization of S to R reticuline. The full length P. somniferum DRS-DRR enzyme (SEQ ID NO: 96), the P. somniferum DRS-DRR enzyme expressed as separate DRS (CYP82Y2—SEQ ID NO: 98) and DRR (PsAKR—SEQ ID NO: 108) enzymes, or the DRS CYP82Y2 enzyme co-expressed with an Imine reductase (StIRED SEQ ID NO: 94) was expressed from pTEF2 and pFBA1 promoters and integrated in chromosome site XI-5 as described by Mikkelsen et al (2012). As can be seen in FIG. 4, a strain expressing the full-length P. somniferum DRS-DRR showed accumulation of both S-reticulin and thebaine showing that even at this rather low production level the DRS-DRR enzyme is a bottleneck.

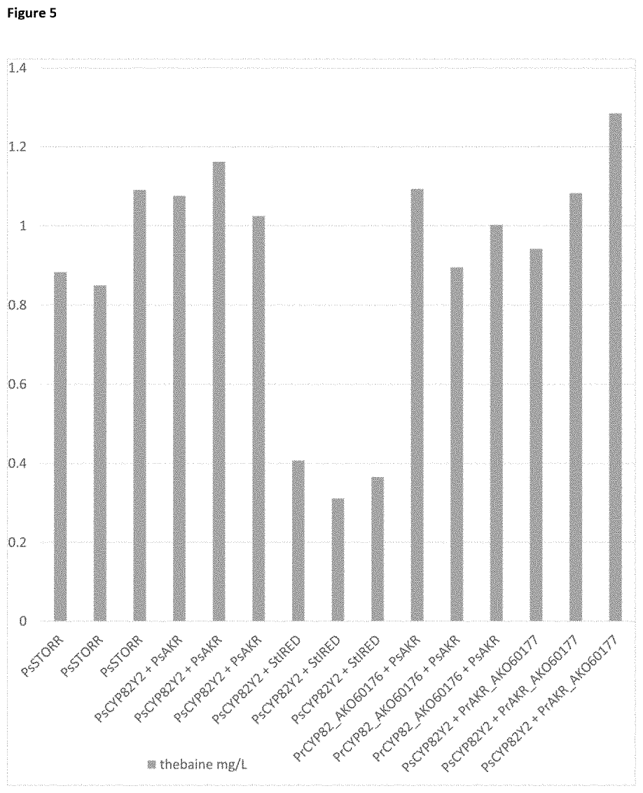

To improve the activity of the DRS-DRR enzyme, the two separate coding regions were expressed individually (the CYP82Y2 and PsAKR functional proteins were expressed as separate enzymes), and a series of variants were created. As can be seen in FIG. 5 expression of the full length PsDRS-DRR gene, the PsCYP82Y2 (SEQ ID NO: 99) and PsAKR genes separately, the PsCYP82Y2 and PrAKR (SEQ ID NO: 111) genes separately, the PrCYP82Y2-like gene (SEQ ID NO: 101) and PsAKR separately, as well as expression of the PsCYP82Y2 together with the StIRED enzyme all results in production of thebaine. As there is seemingly no accumulation of (R)-reticuline, salutaridine and salutaridinol, the level of thebaine production reflects the S-to-R-reticuline conversion efficiency. As can be seen in FIG. 5, the fused PsDRS-DRR and the individually expressed PsCYP82Y2 and PsAKR are equally effective in converting (S)-reticuline to (R)-reticuline as seen by an equal accumulation of thebaine. When the PsCYP82Y2 is co-expressed with the StIRED (SEQ ID NO: 94) (an Imine reductase from Streptomyces tsukubaensis) there is also thebaine production albeit at a lower level. This shows that the StIRED is quite effective in reducing 1,2-dehydroreticuline to R-reticuline, but at low production levels not as effective as the PsAKR (aldo-keto reductase). What can also be deduced from FIG. 5 is that the PrCYP82Y2-like enzyme and PrAKR enzyme homolog are as effective in converting (S)-reticuline to (R)-reticuline as the PsDRS-DRR enzymes.

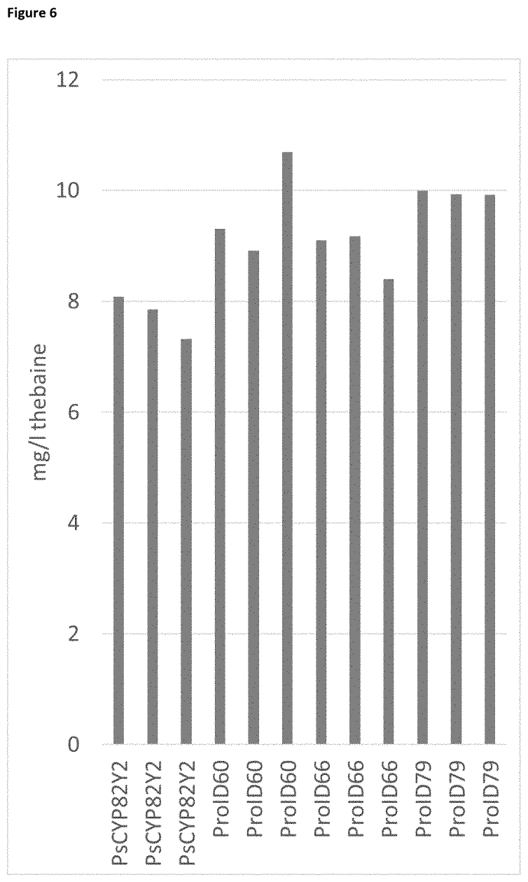

To improve the activity of the S-to-R Reticuline conversion, several PsCYP82Y2 variants were created and tested for activity. These PsCYP82Y2 variants were expressed together with the PsAKR in the strain background described above except with expression of the THS2 gene (SEQ ID NO: 132) instead of the THS1 gene. Thebaine production was then measured by LC-MS (FIG. 6). As shown in FIG. 6, three of these PsCYP82Y2 variants (called proID60 (SEQ ID NO: 102), proID66 (SEQ ID NO: 104) and proID79 (SEQ ID NO: 106)) significantly improve production of thebaine as compared to the native PsCYP82Y2 when co-expressed with the native PsAKR.

### Methods

Yeast transformants were grown as triplicates in 96 deep-well plates in 500 μL liquid Synthetic Complete media for 3 days at 30° C. with shaking at 250 rpm in a Kuhner Climo-Shaker ISF1-X. Culture samples for LC-MS were prepared by extraction as follows: 96% ethanol and culture sample were mixed 1:1 and incubated on a heating block at 80° C. for 10 min. After heating cells were pelleted in an Eppendorf tabletop centrifuge by centrifugation and the supernatant was then transferred to a new tube and diluted 1:20 in water.

LC-MS dopamine, norcoclaurine, reticuline, 1,2-dehydroreticuline, salutaridine, salutaridinol and thebaine targeted LC-MS analysis was performed to quantify opioid metabolites produced in the yeast transformants. Liquid chromatography was performed on an Agilent 1290 Infinity II UHPLC with a binary pump and multisampler (Agilent Technologies, Palo Alto, CA, USA). Separation was achieved on a Kinetex XB—C18 column (100×2.1 mm, 1.7 μm, 100 Å, Phenomenex, Torrance, CA, USA) using 0.1% (v/v) formic acid in H₂O and 0.1% (v/v) formic acid in acetonitrile as mobile phases A and B, respectively. Gradient conditions used were: 0.0-0.3 min 2% B; 0.3-4 min 2-30% B; 4-4.4 min 30-98% B; 4.4-4.9 min 98% B; 4.9-5 min 98-2% B; 5-6 min 2% B. The injection volume was 2 μL and the mobile phase flow rate was 400 μL/min. The column temperature was maintained at 30° C. The liquid chromatography system was coupled to an Ultivo-Triple Quadrupole mass spectrometer (Agilent Technologies, Palo Alto, CA, USA) equipped with an electrospray ion source (ESI) operated in positive and negative mode. The Capillary voltage was maintained at 3500V and the Nozzle voltage at 500V. Source gas temperature was set at 340° C. and source gas flow was set at 12 L/min. Source sheath gas temperature was set at 380° C. and source sheath gas flow set at 12 L/min. Nebulizing gas was set to 30 psi. Nitrogen was used as a dry gas, nebulizing gas and collision gas. Metabolites were detected in dMRM mode were the MRM transitions and mass spectrometer parameters (fragmentation voltage, collision energy, dwell time) were optimized for each metabolite (see Table 22-1). Standards of Dopamine, Reticuline, 1,2-dehydroreticuline, Salutaridine, Salutaridinol and Thebaine in concentration between 0.05-10 μM were analysed and used for quantification of the samples.

TABLE 22-1

| | Precursor | Transition 1 | | Transition 2 | Fragmentor | Retention |
|---|---|---|---|---|---|---|
| Metabolite | m/z [M + H]⁺ | m/z (CE) | | m/z (CE) | voltage | time |
| Dopamine | 154 | 137 | (5) | 91 (5) | 110 | 0.7 |
| Norcoclaurine | 272 | 255 | (5) | 107 (5) | 110 | 2.35 |
| Reticuline | 330 | 299 | (15) | 192 (15) | 110 | 3.16 |
| 1,2-dehydroreticuline | 328 | 313 | (15) | 284 (15) | 110 | 2.96 |
| Salutaridine | 328 | 58 | (15) | 237 (15) | 110 | 3.1 |
| Salutaridinol | 330 | 58 | (15) | 213 (15) | 110 | 2.49 |
| Thebaine | 312 | 58 | (10) | 266 (5) | 110 | 3.55 |

### Example 23 Testing Different THS Enzymes

7-O-acetylsalutaridinol can spontaneously be degraded to hydroxylated byproducts with m/z 330 (Chen, X et al Nature Chemical Biology (2018), A pathogenesis-related 10 protein catalysis the final step in thebaine biosynthesis). To avoid a loss of 7-O-acetylsalutaridinol through formation of this side product, we wanted to see if ProShuffle and ASA mutagenesis of THS2 would improve enzyme activity in a similar way as was done by us for the DRS-DRR enzyme.

S. cerevisiae strain BY4741 was deleted for the gene ARI1 and engineered to overexpress the following genes: ARO4fbr, PpDODC, CYP76AD1_2mut, HDEL_CjNCS_V152, Ps6OMT_Q6WUC1, Cj40MT, AtATR1, EcNMCH, CjCNMT, PbSaIR, PbSAS, PsSAT, PsCPR and PsCYP82Y2 and PsAKR enzymes expressed separately. The promoters used for driving expression of these genes were pTDH3, pPDC1, pTEF1, pTEF2, pTP11 and pPGK1. Expression cassettes with these genes and promoters were integrated into different yeast chromosomes using vectors as described by Mikkelsen et al. (2012). To complete the thebaine biosynthesis pathway in this yeast strain, it was transformed by p415 TEF plasmid (Mumberg et al, Gene 156 (1995), yeast vectors for the controlled expression of heterologous proteins in different genetic backgrounds) or with the p415 TEF plasmid having the Papaver somniferum THS2 or variants of THS2 inserted by restriction cloning in the SpeI and XhoI sites.

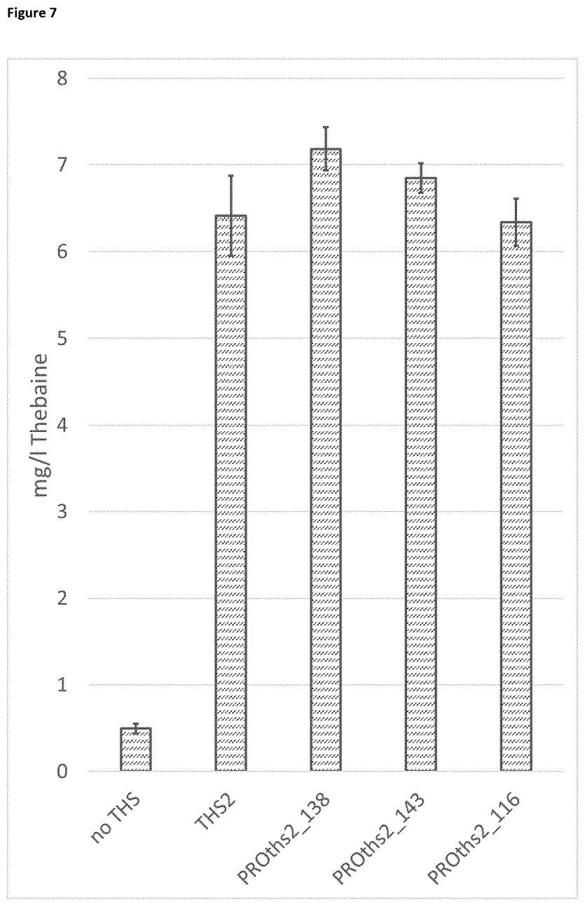

The mutant genes were created, and their expression products tested for activity. FIG. 7 shows the results from 3 mutants. While the variant called PROths2_116 (SEQ ID NO: 138) shows roughly the same thebaine production level as P. somniferum THS2, the two other variants (PROths2_138 and PROths2_143—SEQ ID NO: 134 and 136 respectively) show higher average thebaine production compared to the native enzyme. Especially PROths2_138 appears to produce a significant improvement compared to the native THS2.

### Methods

Yeast transformants were grown as triplicates in 96 deep-well plates in 500 ptL liquid Synthetic Complete media for 3 days at 30° C. with shaking at 250 rpm in a Kuhner Climo-Shaker ISF1-X. Culture samples for LC-MS were prepared by extraction as follows: 96% ethanol and culture sample were mixed 1:1 and incubated on a heating block at 80° C. for 10 min. After heating cells were pelleted in an Eppendorf tabletop centrifuge by centrifugation and the supernatant was then transferred to a new tube and diluted 1:20 in water.

Targeted LC-MS analysis Dopamine, Norcoclaurine, Reticuline, 1,2-dehydroreticuline, Salutaridine, Salutaridinol and Thebaine was performed as described in example 22.

### Example 24 Preparing a Yeast Strain Comprising Full Pathway from Glucose to Northebaine

A thebaine-producing S. cerevisiae strain called sOD310 was constructed from a BY4741 background wherein the ORF of the native genes PDR1, PDR3, PDR5, ARI1, ADH6, YPR1 and GRE2 genes was deleted. Overexpression of thebaine pathway genes in this strain was done by expression cassette integrations in Chromosome sites X-2, XI-5 and XVI-21 as described by Mikkelsen et al. (2012). In these cassettes, promoters pPGK1, pTEF1, pTDH3, pTEF2, pTPI1, pFBA1 and pPDC1 were driving the expression of thebaine pathway genes as well as genes encoding the S. cerevisiae TYR1 gene and feedback resistant versions of ARO7 and ARO4.

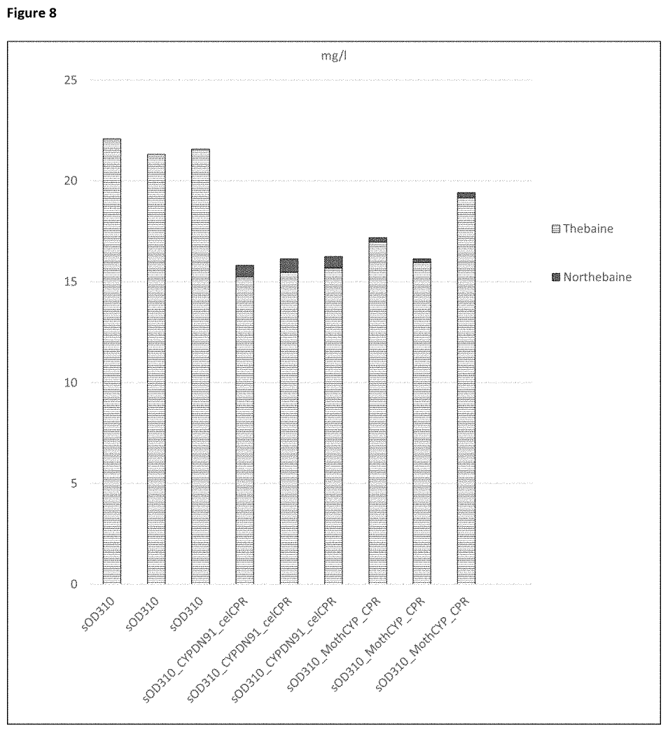

Pathway gene synthesis was done by Twist Bioscience. Genes expressed were: ARO4fbr, ARO7fbr (SEQ ID NO: 4), ScTYR1 (SEQ ID NO: 6), PpDODC, SoCYP76ADr9 (SEQ ID NO: 8), d19CjNCS (SEQ ID NO: 75), Ps60MT_Q6WUC1, AtATR1, EcNMCH, Cj40MT, CjCNMT, PsCPR (SEQ ID NO: 113), PsCYP82Y2, PsAKR, PbSAS1, PbSaIR, PsSAT and PsTHS2 (SEQ ID NO: 132). The d19CjNCS gene encodes an N-terminally truncated Coptis japonica Norcoclaurine Synthase. The truncation replaced the first 19 amino acids of the Cj NCS with a methionine thereby removing a putative signal peptide. In addition, this strain was engineered to further overexpress the d19CjNCS by multicopy integration using the Ty integration-based plasmid called pRIV40 (SEQ ID NO: 78) with insertion, in the AsiSI site, of a pPGK1 promoter operably linked to the d19CjNCS gene. A fragment released by restriction enzyme digest of this plasmid with BssHII was used for transformation and integration into the Ty sites of the yeast strain. Multicopy integration was assured by selection on SC leucine drop-out plates. Since the LEU2 gene used in this plasmid has a truncated promoter (LEU2dT), only transformants with multiple copies integrated are able to grow on SC leucine drop-out plates. The resulting strain was called sOD310 and produced thebaine when grown in small scale 96 deep-well plates to levels of 20-30 mg/l (see FIG. 8, 3 first bars from left).

Yeast transformants were grown as triplicates in 96 deep-well plates in 500 μL liquid Synthetic Complete media lacking leucine for 3 days at 30° C. with shaking at 250 rpm in a Kuhner Climo-Shaker ISF1-X. Culture samples for LC-MS were prepared by extraction as follows: 96% ethanol and culture sample were mixed 1:1 and incubated on a heating block at 80° C. for 10 min. After heating cells were pelleted in an Eppendorf tabletop centrifuge by centrifugation and the supernatant was then transferred to a new tube and diluted 1:20 in water.

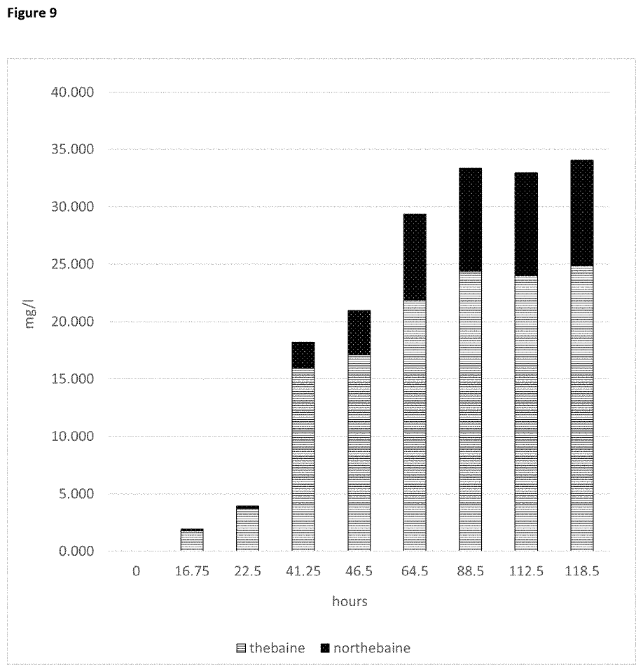

Fungal pair of demethylase gene CYPDN_91 (SEQ ID NO: 251) and CPR gene ceICPR (SEQ ID NO: 306) as well as insect pair of demethylase gene (HaCYP6AE15v2 (SEQ ID NO: 141) and CPR gene HaCPR_E7E2N6 (SEQ ID NO: 304) were then expressed in the thebaine producing strain sOD310 to test for their ability to N-demethylate thebaine to northebaine. These genes were expressed by use of the pPGK1 and pTEF1 promoters and again integration was done as described by Mikkelsen et al. (2012). As can be seen in FIG. 9 both demethylase-CPR pairs demethylated thebaine to northebaine. When grown in the fermenter the fraction of thebaine converted to northebaine by the strain expressing CYPDN_91 and ceICPR was much improved as shown in FIG. 9.

### Fermentation Process and Parameters

### Seed Train Preparation (Media, Conditions)

a) Seed-Train Medium:
The seed-train medium consisted of a mineral medium supplemented with yeast extract containing 2% glucose as the main source of carbon. Its composition was the following (g·L⁻¹): 7.5 yeast extract, 5.0 (NH₄)₂SO₄, 3.0 KH₂PO₄, 0.5 MgSO₄·7H₂O, 22 glucose monohydrate with the addition of 10 mL/L of trace metal stock solution (Hoek et al., 2000) and 12 ml/L of (Delft) vitamin stock solution (Hoek et al., 2000). The medium was sterilized at 121° C. for 20 min before use.

Day 1 Preparation of Pre-Seeding Cultures:
From a frozen glycerol stock a suitable number of cells was transferred into culture tubes containing about 5 ml of seed-train medium. Culture tubes were then incubated on an orbital shaker (180 rpm) at 30° C. for c.a. 24 h in order to reach a final OD₆₀₀ of about 3.00-4.00.

Day 2 Preparation of Seeding Cultures:
Seeding cultures were prepared in 250 mL Erlenmeyer flasks each containing 60 mL of seed-train medium. Each flask was inoculated with a suitable amount of yeast cells which were harvested at the end of the previous propagation step. Seeding cultures were initiated with a starting OD of about 0.05 and then incubated on an orbital shaker (180 rpm) at 30° C. for c.a. 30 h in order to reach a final OD₆₀₀ of about 5.00-6.00.

Day 3_Inoculation of 2-L Fermenter:
The batch-phase was started with a fix working volume consisting in 500 mL of fresh broth. The fermenter was inoculated by transferring into the vessel 50 mL of the seeding culture (with an initial OD of 0.5-0.6) after removal of an equal volume of batch medium.

b) Batch and Feed Medium Composition and Preparation Steps; pH Control Agents

Batch Medium:
The batch medium consisted of a mineral medium supplemented with yeast extract containing 1% glucose as the main source of carbon. Its composition is the following (g·L⁻¹): 7.5 yeast extract, 5.0 (NH₄)₂SO₄, 3.0 KH₂PO₄, 0.5 MgSO₄·7H₂O, 3.0 SB2020 (antifoam), 13 glucose monohydrate with the addition of 10 mL/L of trace metal stock solution (Hoek et al., 2000) and 12 ml/L of (Delft) vitamin stock solution (Hoek et al., 2000). The medium was sterilized at 121° C. for 20 min before use. The pH was stabilized around a set point value of 6.5 and then automatically controlled during the cultivation by adding 12.5% ammonium hydroxide with a peristaltic pump.

Fed-Batch Medium:
The fed-batch medium consisted of a minimal mineral medium containing 62% glucose as the only source of carbon. Its composition was the following (g·L⁻¹): 5.0 (NH₄)₂SO₄, 11.2 KH₂PO₄, 6.3 MgSO₄·7H₂O, 4.3 K₂SO₄, 0.347 Na₂SO₄, 1.5 SB2020 (antifoam), 682 glucose monohydrate with the addition of 14.4 mL/L of trace metal stock solution (Hoek et al., 2000) and 14.8 ml/L of (Delft) vitamin stock solution (Hoek et al., 2000). The medium was sterilized at 121° C. for 20 min before use.

c) Process Parameters for Batch & Fed-Batch Phases of Cultivation
The fermentation process was operated as a series of two stages carried out in the same vessel. During the first stage, that coincided with the first 8 hours of cultivation run, the yeast culture was grown batchwise in 0.5 L of batch medium: the temperature was set at 30° C. while the pH value was kept around a set point of 6.5. Fully aerobic conditions were ensured by flowing 1 vvm of air through the vessel; stirring was kept at a constant rate of 1100 rpm. At the end of the 8 hours of batch run the second fed-batch phase was initiated by starting the glucose feed. Process parameters for the fed-batch phase were again the same used during the previous batch phase (i.e., Temperature=30° C., pH=6.5, Aeration rate=1 vvm, Stirring rate=1100 rpm). In particular, the air flow was increased stepwise in order to compensate for the increase in volume and to maintain the aeration rate value at around 1 vvm during the course of fermentation.

d) Feeding Strategy (Dosage, Profile, Control-Trigger)
We employed a constant specific growth rate (p) strategy consisting of four consecutive exponential feeding phases where each one of them was occurring at a different specific growth rate value. The conditions for the four phases are summarized in the below table:

| | μᵢ [h⁻¹] | Xᵢ [g/L] | Vᵢ [L] | t [h] | Air flow [*] |
|---|---|---|---|---|---|
| Phase1 | 0.080 | 1.5 | 0.500 | (0) 8 | 620 |
| Phase2 | 0.040 | 54.32 | 0.618 | (48) 56 | 750 |
| Phase3 | 0.025 | 94.42 | 0.752 | (67) 75 | 950 |
| Phase4 | 0.012 | 162.50 | 0.947 | (90) 98 | 1300 |
| End | 0.012 | 200.61 | 1.407 | (143) 151 | 1400 |

[*] ccm/min

The actual growth rate value during the fed-batch cultivation was primary controlled by the feeding rate profile of the main limiting substrate (glucose). The actual volumetric feed rate F [L·h⁻¹, (mL·min⁻¹)] was calculated according to the following equation:

F=Fᵢ exp(μᵢt)
where
Fᵢ, initial volumetric feed rate [L·h⁻¹, (mL·min⁻¹)];
μᵢ, is specific feed rate for the constant specific growth rate phase [h⁻¹]

$${F}_{i} = {μ}_{i} \frac{{X}_{i} {V}_{i}}{{Y}_{X / S} {S}_{F}}$$

with:
Xᵢ, dry microbial mass concentration in the culture vessel at the start of the phase [g·L⁻¹]
Xᵢ, volume of the culture at the start of the phase [L]
S_{F}, glucose concentration in the feed=620 g·L⁻¹
Y_{x/s}, microbial mass yield on the limiting substrate (i.e., glucose)=0.45
The transition from each one of the four phases to the next one was based on a previously optimized time profile that was able to guaranty that the system did not incur into oxygen limitation during the course of the run of each exponential phase.
   P. V., Hoek, E. d., Hulster, J. P. v., Dijken, J. T., Pronk. Fermentative capacity in high-cell-density fed-batch cultures of baker's yeast, Biotechnol Bioeng 68: 517-523, 2000.

### Example 25 Preparing a Yeast Strain Comprising Full Pathway from Glucose to Oripavine

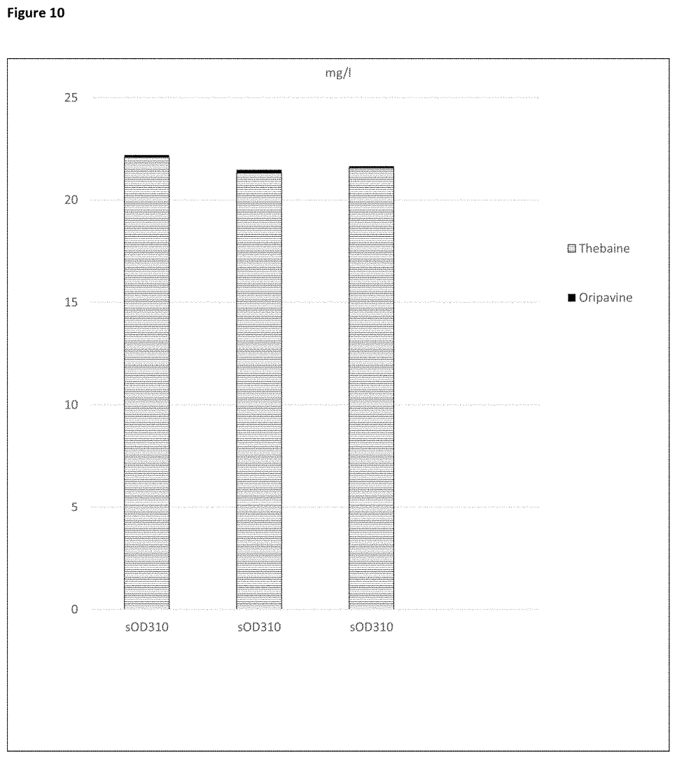

When constructing the thebaine production strains in example 24, an unexpected peak was observed in the LC-MS trace. Surprisingly, the thebaine production strain produced a small amount of oripavine as shown in FIG. 10. Since feeding of thebaine to growing yeast does not result in formation of oripavine (data not shown), it is unlikely that demethylation is the mechanism of this formation. Instead, the oripavine was likely formed by occasional omission of the 4′ methylation of (S)-3′Hydroxy-N-Methylcoclaurine to reticuline and the ability of the DRS-DRR enzyme to accept (S)-3′Hydroxy-N-Methylcoclaurine as a substrate. It is thought that SAS, SaIR, SAT and THS2 also accept their substrates missing this methyl group, ultimately leading to the formation of oripavine as the end product.

A sOD310 strains is prepared as described in example 24 omitting the Cj40MT. The resulting strain is grown in small scale 96 deep-well plates produces significant levels of oripavine (data not shown).

### Example 26. Preparation of Compound BnO—VI-Bn from Nororipavine (Step a)

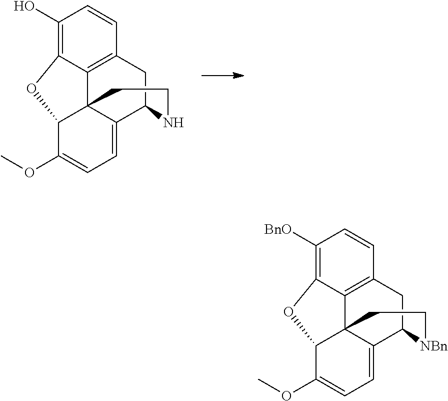 [SMILES: COC1=CC=C2[C@H]3Cc4ccc(O)c5c4[C@@]2(CCN3)[C@H]1O5 ; COC1=CC=C2[C@H]3Cc4ccc(OCc5ccccc5)c5c4[C@@]2(CCN3Cc2ccccc2)[C@H]1O5]

A 500 mL flask was charged with nororipavine (1.00 g, 3.5 mmol), iPrOH (20 mL), and water (10 mL). The suspension was stirred at room temperature and NaOH-pellets (0.42 g, 10.6 mmol, 3 equiv) were added. After 10 min a light brown solution was obtained and benzyl bromide (1.50 g, 8.8 mmol, 2.5 equiv) was added over a period of 1 min. A slight exotherm was observed and after 10 min a precipitate was formed. After 2 h to the mixture was added water (20 mL). The resulting suspension was cooled in ice-water for 1 h and then filtered. The solid was washed with water (2×10 mL) and dried under vacuum to afford Compound BnO-II-Bn (1.6 g, 96%). Analytical data were in agreement with the literature.

### Example 27. Solvent Screening Optimization for Preparation of Compound BnO-VII-Bn

It is known that Diels-Alder reactions often result in a mixture of two adducts. It was hypothesized that this may contribute to poor yields observed by the present inventors in the synthesis of Compound BnO-II-Bn. It was further hypothesized that solvent may be an effective variable to influence the relative proportions of diastereomers produced and can thereby be modified to favor the desired product. To test this hypothesis, a solvent screening experiment was carried out with the typical solvent (Toluene) and several test solvents. Notably, the test solvents were designed to be polar solvents, in contrast to toluene. The results are summarized in Table 27-1:

TABLE 27-1
Solvent Effects on Synthesis Yield

| | | | 2 h | 5 h | 20 h | 26 h | 74 h |
|---|---|---|---|---|---|---|---|
| | | | s.m. | s.m. | s.m. | s.m. | s.m. |
| | | MVK | major | major | major | major | major |
| Exp. | Solvent | equiv. | minor | minor | minor | minor | minor |
| A | iPrOH | 16 | 26.2 | 3.45 | 0.0 | | |
| | | | 59.6 | 82.0 | 186.4 | | |
| | | | 1.34 | 2.00 | 2.24 | | |
| | | | | | (isomer | | |
| | | | | | ratio: 38) | | |
| B | 33% | | 13.1 | 1.35 | 0.30 | 0.0 | |
| | water | 16 | 71.7 | 83.4 | 83.4 | 84.2 | |
| | 67% | | 1.02 | 1.36 | 1.45 | 1.40 | |
| | iPrOH | | | | | (isomer | |
| | | | | | | ratio: 60) | |
| C | 33% | | 86.2 | 50.8 | 4.00 | 1.65 | 0.0 |
| | water | 4 | 13.9 | 43.2 | 82.6 | 84.1 | 83.8 |
| | 67% | | 0.0 | 0.94 | 1.32 | 1.25 | 1.41 |
| | iPrOH | | | | | | (isomer |
| | | | | | | | ratio: 59) |
| D | iPrOAc | | 69.2 | 50.0 | 1.87 | 1.37 | 0.0 |
| | | 16 | 19.4 | 36.3 | 79.0 | 83.2 | 82.5 |
| | | | 0.81 | 1.65 | 3.76 | 3.65 | 3.76 |
| | | | | | | | (isomer |
| | | | | | | | ratio: 22) |
| ref. | Toluene | 20 | | | 0.0 | | |
| | | | | | 93.8 | | |
| | | | | | 4.4 | | |
| | | | | | (isomer | | |
| | | | | | ratio: 21) | | |

s.m.: starting material.

### Example 28. Preparation of Compound BnO—VII-Bn (Step b)

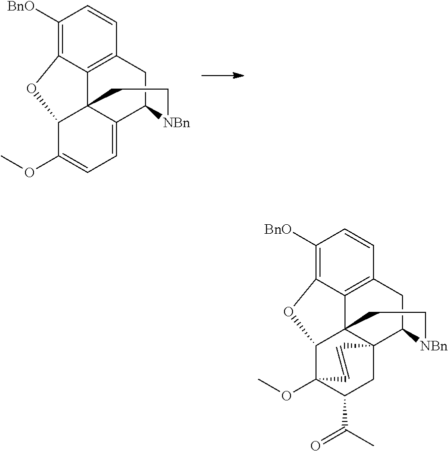 [SMILES: COC1=CC=C2[C@H]3Cc4ccc(OCc5ccccc5)c5c4[C@@]2(CCN3Cc2ccccc2)[C@H]1O5 ; CO[C@]12C=C[C@@]3(C[C@@H]1C(C)=O)[C@H]1Cc4ccc(OCc5ccccc5)c5c4[C@@]3(CCN1Cc1ccccc1)[C@H]2O5]

A solution of Compound BnO-II-Bn (1.54 g, 3.3 mmol) was suspended in 15 mL iPrOH and 5 mL of toluene and heated to 85° C. for 1 h. Next, methyl vinyl ketone (1.2 mL, 13.3 mmol) was added dropwise. After 20 h, the reaction mixture was cooled to room temperature and solvent removed under vacuum. The target material was purified by column chromatography (120 g SiO₂, elution with 0-20% EtOAc in heptane, R_{f} 0.3) to afford Compound BnO-II-Bn as a colorless solid (1.75 g, 93% yield). 7α-Acetyl-N,O-dibenzyl-6,14-endo(etheno)tetrahydro-nororipavine

Analytical data were in agreement with the literature.

### Example 29. Telescoped Preparation of Compound BnO—VII-Bn (Step b)

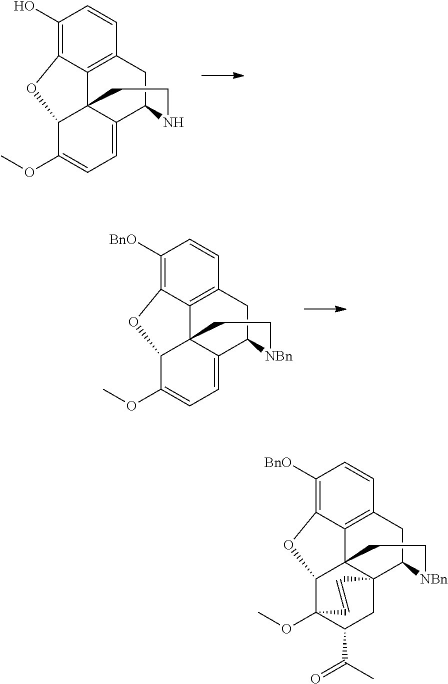 [SMILES: COC1=CC=C2[C@H]3Cc4ccc(O)c5c4[C@@]2(CCN3)[C@H]1O5 ; COC1=CC=C2[C@H]3Cc4ccc(OCc5ccccc5)c5c4[C@@]2(CCN3Cc2ccccc2)[C@H]1O5 ; CO[C@]12C=C[C@@]3(C[C@@H]1C(C)=O)[C@H]1Cc4ccc(OCc5ccccc5)c5c4[C@@]3(CCN1Cc1ccccc1)[C@H]2O5]

A 500 mL round bottom flask was charged with nororipavine (1.00 g, 3.5 mmol), iPrOH (15 mL), and water (6 mL). The suspension was stirred at room temperature and NaOH pellets (0.42 g, 10.6 mmol, 3 eq.) added. After 10 min a light brown solution was obtained and benzyl bromide (1.50 g, 8.8 mmol, 2.5 eq.) was added over a period of 1 min. A slight exotherm was observed and after 10 min a precipitate was formed. The mixture was stirred for 3 h and then the pH adjusted from basic to a pH of 7.3 by addition of a 10% acetic acid solution. The resulting suspension was heated to 85° C. for 1 h and methyl vinyl ketone (0.53 mL, 4 eq.) added dropwise under a partial argon atmosphere. After letting react for 20 h, the mixture was cooled to room temperature and solvent removed under vacuum. The target material was purified by column chromatography (120 g SiO₂, 33% EtOAc in petroleum ether, R_{f} 0.3). The title compound was obtained as a colorless solid (1.45 g, 76% yield). Analytical data were in agreement with the literature.

The above results show that, desirably, Step F can be prepared in the same solvent mixture as Step B. Further, Step F can be efficiently performed utilizing the crude reaction product of Step B without substantial intervening purification or solvent removal.

### Example 30. Preparation of Compound BnO-IIIA-Bn (Step c)

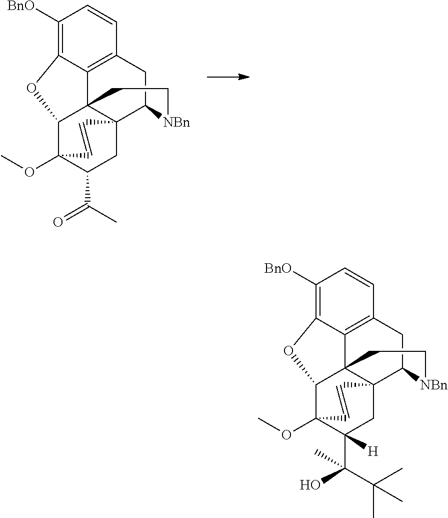 [SMILES: CO[C@]12C=C[C@@]3(C[C@@H]1C(C)=O)[C@H]1Cc4ccc(OCc5ccccc5)c5c4[C@@]3(CCN1Cc1ccccc1)[C@H]2O5 ; CO[C@]12C=C[C@@]3(C[C@@H]1[C@](C)(O)C(C)(C)C)[C@H]1Cc4ccc(OCc5ccccc5)c5c4[C@@]3(CCN1Cc1ccccc1)[C@H]2O5]

A 50 mL flask was charged with a solution of freshly prepared tert-butylmagnesium chloride (e.g., about 0.5 to 2 M, about 4-16 eq., preferably about 1.5 to 2M) in a mixture of THF and cyclohexane. To the flask was then added a solution of Compound BnO-II-Bn (0.5 g, 0.93 mmol) in dry toluene (8 mL). The reaction mixture was reacted overnight and then cooled in an ice-water bath and quenched by addition of 10% aqueous ammonium chloride (25 mL). The layers were separated and the aqueous layer was extracted with toluene (3×25 mL). The combined organic layers were washed with brine, dried with sodium sulfate, and concentrated. Purification by column chromatography (120 g SiO2, elution with 20% EtOAc in heptane) afforded Compound BnO-III-Bn as white solid (0.42 g, 83%). The spectral data were in agreement with literature data and of that reported in WO 2018/211331.

### Example 31. Preparation of Compound HO—IX—H (Step d)

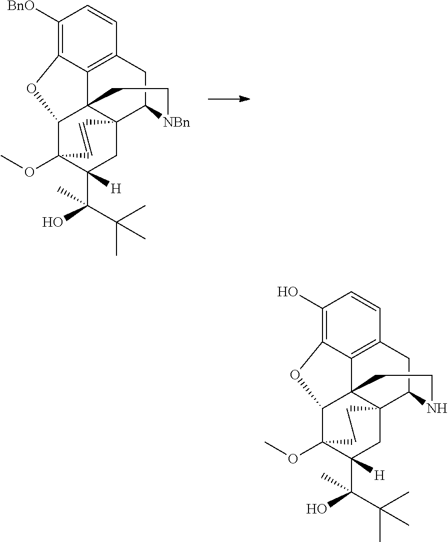 [SMILES: CO[C@]12C=C[C@@]3(C[C@@H]1[C@](C)(O)C(C)(C)C)[C@H]1Cc4ccc(OCc5ccccc5)c5c4[C@@]3(CCN1Cc1ccccc1)[C@H]2O5 ; CO[C@]12CC[C@@]3(C[C@@H]1[C@](C)(O)C(C)(C)C)[C@H]1Cc4ccc(O)c5c4[C@@]3(CCN1)[C@H]2O5]

A vigorously stirred mixture of Compound BnO-III-Bn (355 mg, 0.6 mmol), and Pd/C (10%, 30 mg) in iPrOH (10 mL), water (0.2 mL), and acetic acid (0.1 mL) was hydrogenated at 60° C. for 16 h under 1 atmosphere of hydrogen. IPC NMR showed that both benzyl groups were removed, and the double bond was only partly reduced. The catalyst was refreshed, and hydrogenation was continued at 80° C. for 60 h. ICP NMR showed no more double bond signals. The mixture was filtered over Celite. The filter was flushed with iPrOH and DCM. The filtrate was concentrated to give Compound HO—IV-H as acetate salt (300 mg, 100%).

Norbuprenorphine HPLC-purity 89% at 215 nm.

MS (ES-API pos) m/z 414.3 (M+H).

¹H NMR (300 MHz, CDCl₃) δ [ppm] 7.64 (br s, 2H), 6.76 (d, J=8.0 Hz, 1H), 6.49 (d, J=8.1 Hz, 1H), 5.80 (br s, 1H), 4.40 (s, 1H), 3.59 (d, J=6.4 Hz, 1H), 3.51 (s, 3H), 3.35-3.25 (m, 2H), 3.04 (t, J=13.5 Hz, 1H), 2.88 (dd, J=19.2, 6.4 Hz, 1H), 2.75 (t, J=13.5 Hz, 1H), 2.22-2.07 (m, 2H), 2.01 (s, 3H), 1.90-1.70 (m, 3H), 1.52 (dd, J=13.1, 9.0 Hz, 1H), 1.33 (s, 3H), 1.18 (m, 1H), 1.03 (s, 9H), 0.76 (t, J=12.3 Hz, 1H).

¹³C NMR (75 MHz, CDCl₃) δ [ppm] 145.91, 139.04, 129.99, 123.75, 120.29, 118.23, 95.53, 79.85, 79.62, 53.66, 52.69, 45.00, 42.97, 40.34, 34.40, 32.1, 31.8, 29.9, 29.1, 26.23, 22.9, 20.13, 17.8.

### Example 32. Preparation of Buprenorphine (Step Ei)

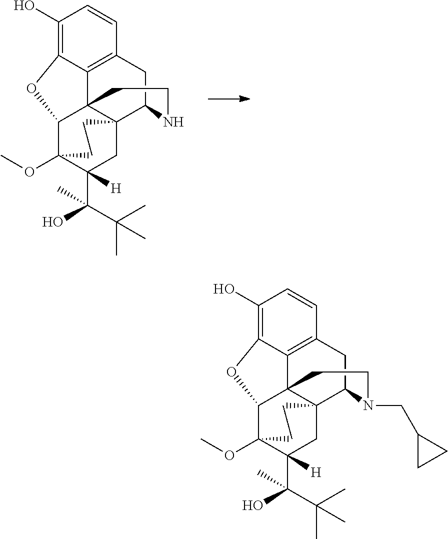 [SMILES: CO[C@]12CC[C@@]3(C[C@@H]1[C@](C)(O)C(C)(C)C)[C@H]1Cc4ccc(O)c5c4[C@@]3(CCN1)[C@H]2O5 ; CO[C@]12CC[C@@]3(C[C@@H]1[C@](C)(O)C(C)(C)C)[C@H]1Cc4ccc(O)c5c4[C@@]3(CCN1CC1CC1)[C@H]2O5]

A 50 mL flask was charged with Compound HO-1-H (210 mg, 0.44 mmol), cyclopropane carboxaldehyde (80 μL, 1 mmol), dichloro(p-cymene)ruthenium(II) dimer (10 mg, 0.016 mmol), triethylamine (0.42 mL, 3.1 mmol), and acetonitrile (5 mL). The mixture was stirred under nitrogen at room temperature and formic acid (0.24 mL, 6.2 mmol) was added dropwise. The resulting mixture was heated at 60° C. for 1 h. The mixture was cooled to room temperature and concentrated under vacuum. The residue was partitioned between toluene and 1 N aqueous NaOH. The aqueous layer was extracted twice with toluene. The combined organic layers were washed with brine, dried on sodium sulfate, and concentrated under vacuum to afford buprenorphine (160 mg, 78%). HPLC-purity 85.6% at 215 nm.

MS and NMR data were in agreement with those obtained in previous examples.

### Example 33. Production of Nororipavine from Oripavine by Heterologous Expression of Genes Encoding Demethylases and CPRs in Aspergillus nidulans

Cytochrome P450 demethylase genes Hv_CYP_A0A2A4JAM9_A110N+H242P+V224I (SEQ ID NO: 771/772) or HaCYP6AE15v2 (SEQ ID NO: 140/141) together with the HaCPR_E0A3A7 (SEQ ID NO: 292/293) and transporter T193_AanPUP3_55 (SEQ ID NO: 613/614) are tested in A. nidulans strain NID1 (argB2, pyrG89, veA1, nkuAA) (Nielsen et al 2008), in order to evaluate their demethylation capacity of oripavine to nororipavine (N-demethylation). All tested gene sequences are codon optimized for Saccharomyces cerevisiae expression using known standard methodology and ordered from GeneArt. Additionally, coding sequences optimized for expression in Aspergillus are also tested. The synthesized fragments are cloned using the Uracil-Specific Excision Reagent (USER) cloning system (Nour-Eldin et al., 2006) and introduced into a vector system designed for expression and genomic integration in A. nidulans integration site 1 (151) (Hansen et al. 2011). The vector used is pU1111-1, together with the gpdA promoter and trpC terminator as described by Hansen et al. 2011. Transformants are selected using the auxotrophic argB marker in the pU1111-1 plasmid. Correct genomic insertion of the expression cassettes are verified by PCR on fungal colonies, as described by Hansen et al. 2011. Five colonies from each transformation are inoculated in Minimal Medium (MM) containing uridine and uracil at pH 7 and 0.5 mM oripavine added from a stock solution as described in Example 3. The cultures are incubated at 37° C. with 130 rpm agitation for 84 hours.

Metabolites are extracted from 0.6 ml of culture broth with 0.5 ml of extraction buffer as described in Example 3 harvest, methanol is added to enhance extraction as needed. The supernatant is isolated and analysis is as described in Example 4. Production of nororipavine is achieved upon the heterologous expression of the N-demethylase genes above the levels detected in the vector control.

### Example 34. In Planta Production of Nororipavine by Heterologous Expression of Genes Encoding N-Demethylases

### Transient Expression of Gene Constructs in Nicotiana benthamiana

Synthetic DNA fragments, codon optimized for Saccharomyces cerevisiae expression and encoding the demethylase enzymes Hv_CYP_A0A2A4JAM9_A110N+H242P+V224I (SEQ ID NO: 771/772) or HaCYP6AE15v2 (SEQ ID NO: 140/141) together with the HaCPR_E0A3A7 (SEQ ID NO: 292/293) and transporter T193 (SEQ ID NO: 613/614) are PCR amplified using standard deoxyuracil(dU)-containing primers. All amplified fragments are cloned into a modified version of the pCAMBIA130035Su plasmid under the control of the doubled enhancer element from CaMV 35S promoter, by using Uracil-Specific Excision Reagent (USER) cloning technology (Nour-Eldin et al., 2006). The modified pCAMBIA130035Su plasmid is generated by PCR amplifying the pCAMBIA130035Su plasmid using a standard deoxyuracil(dU)-containing primer pair and the amplified plasmid backbone is then treated with DpnI (New England BioLabs). A synthetic DNA fragment encoding the OCS (Octapine Synthase) terminator from Agrobacterium tumefaciens (Genbank accession no. CP011249.1) is purchased from Integrated DNA Technologies and PCR amplified using a set of standard deoxyuracil(dU)-containing primers. The amplified OCS terminator is cloned in the DpnI-treated plasmid backbone with USER technology, yielding the modified pCAMBIA130035Su plasmid, pCAMBIA130035Su_MOD which is verified by DNA sequencing.

All plasmid-gene constructs along with a pCAMBIA130035Su_MOD plasmid containing the tomato p19 viral suppressor gene (Baulcombe and Molnar, 2004) are transformed into the Agrobacterium tumefaciens strain, AGL-1 and infiltrated into leaves of Nicotiana benthamiana plants as described in (Bach et al., 2014). After 4 days, agroinfiltrated leaves are re-infiltrated with 0.5 mM oripavine. Plants are thereafter left to grow for another 1 day in the green house.

Metabolites are extracted from discs (0=3 cm) of agroinfiltrated N. benthamiana leaves. Leaf discs, excised with a cork borer, are flash frozen in liquid nitrogen. 0.5 ml of extraction buffer (60% (v/v) methanol, 0.1% (v/v) formic acid), equilibrated to 50° C., are added to each frozen leaf disc followed by incubation for 1 hour at 50° C., agitating at 600 rpm. The supernatant is isolated and passed through a Multiscreen HTS HV 0.45 μm filter plate (Merck Milipore) before analysis by HPLC, as described in Example 4. Production of nororipavine is achieved upon the heterologous expression of the N-demethylase genes above the levels detected in the vector control.

### Example 35. Production of Nororipavine from Oripavine by Heterologous Expression of Genes Encoding Demethylases, CPR and Transporters in 22 Alternative Saccharomyces Strains

Twenty two different S. cerevisiae strains from the National Collection of Yeast Cultures (NCYC) were transformed with the following genes for integration into the genome: HaCPR_E0A3A7 (SEQ ID NO: 292/293), HaCYP6AE15v2 (SEQ ID NO: 140/141) and HaCYP6AE19 (SEQ ID NO: 142/143) from Helicoverpa armigera, T149_AcoPUP3_59 (SEQ ID NO: 537/538) from Aquilegia coerulea and T168_FvePUP3_37 (SEQ ID NO: 571/572) from Fragaria vesca subsp. vesca.

The strains were as follows using the references of the National Collection of Yeast Cultures (NCYC): NCYC 3582, NCYC 3585, NCYC 3586, NCYC 3588, NCYC 3590, NCYC 3591, NCYC 3592, NCYC 3594, NCYC 3595, NCYC 3596, NCYC 3598, NCYC 3599, NCYC 3600, NCYC 3601, NCYC 3602, NCYC 3603, NCYC 3604, NCYC 3605, NCYC 3606, NCYC 3607, NCYC 3610 and NCYC 35936.

The cells were grown as in Example 3 except for using YPD medium with 60 mg/L of phleomycin instead of SC-His-Leu-Ura for the pre-cultures. The samples were extracted and analyzed by HPLC as described in Example 4.

The 22 strains showed detectable nororipavine levels; fifteen of them showed lower production levels than a standard S. cerevisiae laboratory reference strain sOD157, one converted similar amounts of nororipavine as the reference strain, and six had higher production than strain sOD157. This experiment illustrates that the pathways exemplified in the previous examples are able to be transferred to numerous other Saccharomyces species successfully. One skilled in the art would know how to further optimize these strains for higher productivity and titer.

### Example 36. Improved Oripavine Bioconversion by Increasing Heme Cofactor within Cells

Three different strategies were tested to increase heme availability in the strain sOD465 (strain sOD398 as described in Example 21 with an extra copy of the cytochrome P450 Hv_CYP_A0A2A4JAM9 (SEQ ID NO: 152/153) from Heliothis virescens). The first approach consisted of boosting heme biosynthesis by overexpressing three rate-limiting enzymes from the heme pathway, HEM2, HEM3 and HEM12 (Hoffman et al., 2003 and Michener et al., 2012). Since an excess of free heme can be detrimental for the cells (Krishnamurthy et al., 2007), several combinations of the genes expressed under weak or strong promoters were analysed as shown in Table 36-1. Alternatively, deletion of the heme-down regulating gene HMX1 or addition of “heme” boosting agents such as hemin (Protchenko et al., 2003 and Krainer et al., 2015, respectively) have been also studied in the sOD465 strain background.

TABLE 36-1
Promoters used to achieve different expression
levels of heme biosynthesis pathway genes.

| | Weak | Strong |
|---|---|---|
| GENE | promoter | promoter |
| HEM2 | pPYK1 | pTEF1 |
| HEM3 | pSED1 | pTDH3 |
| HEM12 | pKEX2 | pPGK1 |

The cells were grown as in Example 3 except for the oripavine stock solution which was 50 mM in DELFT medium at pH 4.5. Samples were extracted and analysed by HPLC as described in Example 4.

TABLE 36-2
Impact of different overexpressions of HEM biosynthesis genes in oripavine bioconversion
to nororipavine and improvement in the bioconversion compared with a reference strain
without any extra copies of the tested HEM genes. Cells were fed with 4 mM oripavine
in DELFT media pH 4.5 and grown at 30° C. with shaking at 250 rpm for 72 h. The
standard deviation values refer from 3 to 6 different biological replicates.

| | Weak promoters | | | Strong promoters | | |
|---|---|---|---|---|---|---|
| | % | | % increase vs | % | | % increase vs |
| GENES | Nororipavine | STD | reference strain | Nororipavine | STD | reference strain |
| HEM2 + | 38.71 | 3.24 | 31.76 | 22.18 | 1.54 | −24.51 |
| HEM3 + HEM12 | | | | | | |
| HEM2 + HEM12 | 31.37 | 4.44 | 6.79 | 28.68 | 4.09 | −2.38 |
| HEM2 + HEM3 | 34.58 | 4.16 | 17.70 | 24.49 | 3.73 | −16.64 |
| HEM3 + HEM12 | 31.20 | 2.69 | 6.19 | | | |
| HEM2 | 33.42 | 3.74 | 13.76 | 33.25 | 2.16 | 13.18 |
| HEM12 | 26.16 | 4.24 | −10.97 | 32.42 | 2.96 | 10.33 |
| HEM3 | 28.80 | 2.95 | −1.98 | 28.53 | 3.66 | −2.89 |
| — | 29.38 | 2.48 | 0.00 | 29.38 | 2.48 | 0.00 |

TABLE 36-3
Effect of alternative strategies to increase heme pool within the
cells on the oripavine to nororipavine bioconversion and their
improvement as compared to a reference strain. Cells were fed
with 4 mM oripavine in DELFT media pH 4.5 and grown at 30°
C. with shaking at 250 rpm for 72 h. The standard deviation (STD)
values were calculated from 3 to 6 different biological replicates.

| | % | | % increase vs |
|---|---|---|---|
| Strategy | Nororipavine | STD | reference strain |
| Deletion of HMX1 | 34.34 | 2.02 | 28.89 |
| 10 μM Hemin supplementation | 39.08 | 3.92 | 46.68 |
| — | 26.64 | 3.25 | 0.00 |

All strategies increased the nororipavine production showing that heme in the strain is a limiting factor in production of demethylated nor-benzylisoquinoline alkaloids such as nororipavine and northebaine and that modifications to the cell increasing the heme levels will benefit production of demethylated nor-benzylisoquinoline alkaloids.

Modest overexpression of most of the tested HEM gene combinations improved the bioconversion of oripavine to nororipavine, with the highest increase achieved when all three genes are overexpressed simultaneously (31.76% more nororipavine production than the control strain). At the same time, significant reduction of oripavine to nororipavine bioconversion was observed when genes overexpressed under strong promoters apart from HEM2 and HEM12. Only the single HEM2 overexpression enhanced nororipavine production independently of the promoter strength under the conditions tested. The results suggest the need for appropriate levels of heme production in order to see a positive impact on the bioconversion of oripavine to nororipavine.

### Example 37. Enhanced Nororipavine Production by Overexpressing Different P450 Helper Genes

Several genes were overexpressed in the strains sOD398 (previously described in the examples 21) or sOD435 built with the same genetic constructs as described for sOD438 in Example 47) for cytochrome P450 activity optimization. The different selected genes and their biological roles were the follows: DAP1, which encodes a heme-binding protein involved in the regulation the function of cytochrome P450 (Hughes et al., 2007); HAC1, a transcription factor that modulates the unfolded protein response (Kawahara T, et al., 1997); and several genes involved in protein processing as well as heat shock response (Yu et al., 2017). The cells were grown as in Example 36 and the samples were extracted and analysed by HPLC as described in Example 4.

TABLE 37-1
Impact of different P450 helper genes in oripavine bioconversion
to nororipavine and improvement in the bioconversion compared
with a reference. Cells were fed with 4 mM oripavine in DELFT
media pH 4.5 and grown at 30° C. with shaking at 250 rpm for 72 h.

| | | % | % increase vs |
|---|---|---|---|
| GENE | STRAIN | NORORIPAVINE | reference strain |
| KAR2 | sOD398 | 31.70 | 27.50 |
| HSP82 | sOD398 | 34.38 | 38.28 |
| CNE1 | sOD398 | 39.61 | 59.34 |
| SSA1 | sOD398 | 42.18 | 69.68 |
| CPR6 | sOD398 | 37.83 | 52.19 |
| FES1 | sOD398 | 39.43 | 58.60 |
| HSP104 | sOD398 | 38.60 | 55.26 |
| STI1 | sOD398 | 41.38 | 66.44 |
| | sOD398 (ref strain) | 24.86 | — |
| DAP1 | sOD435 | 57.84 | 7.32 |
| HAC1 | sOD435 | 58.83 | 9.14 |
| | sOD435 (ref strain) | 53.90 | — |

All tested genes enhanced oripavine to nororipavine bioconversion when overexpressed in the tester strains, indicating significant potential in refining cytochrome P450 biological function by improving different processes within the hosts.

### Example 38. Influence of NADPH Boost in Oripavine to Nororipavine Bioconversion

To study the effect of improved cytosolic NADPH generation on nororipavine production, ZWF1 (SEQ ID NO: 765) and GND1 (SEQ ID NO: 767) genes from the pentose phosphate pathway (Stincone et al., 2015) were overexpressed in the strain sOD344 (previously described in the example 21). The cells were grown as in Example 3 and the samples were extracted and analysed by HPLC as described in Example 4.

TABLE 38-1
Impact of increasing cytosolic NADPH content in oripavine
bioconversion to nororipavine and improvement in the
bioconversion compared with a reference. Cells were
fed with 1 mM oripavine in DELFT media pH 4.5 and grown
at 30° C. with shaking at 250 rpm for 72 h.

| | % | % increase vs |
|---|---|---|
| GENE | NORORIPAVINE | reference strain |
| ZWF1 | 65.7 | 5.1 |
| ZWF1 + GND1 | 76.3 | 22 |
| — | 62.5 | |

Under the conditions tested there was a significant positive effect of co-expressing ZWF1 and GND1 on bioconversion of oripavine to nororipavine as compared to the tester strain (22% more bioconversion), demonstrating this strategy's potential in improving cytochrome P450 function within the host cells.

### Example 39. Formaldehyde Detoxification Consequences on Nororipavine Production

SFA1 (SEQ ID NO: 769) was overexpressed in the strain sOD344 (previously described in the example 21) to analyse the effect of its biological role on detoxifying formaldehyde (Wehner E P et al., 1993), a toxic by-product released during cytochrome P450 N-demethylation reaction (Kalász H et al., 1998), in oripavine to nororipavine bioconversion. The cells were grown as in Example 3 and the samples were extracted and analysed by HPLC as described in Example 4.

TABLE 39-1
Formaldehyde detoxification effect in nororipavine production
and improvement in the bioconversion compared with a reference.
Cells were fed with 1 mM oripavine in DELFT media pH 4.5 and
grown at 30° C. with shaking at 250 rpm for 72 h.

| | % | % increase vs |
|---|---|---|
| GENE | NORORIPAVINE | reference strain |
| SFA1 | 71.0 | 13.6 |
| — | 62.5 | |

The overexpression of SFA1 improved oripavine to nororipavine bioconversion by 13.6% more than the tester strain, expecting even higher impact on the bioconversion when analyse strains in the fermentor since larger amounts of formaldehyde should be released during the fermentation process.

### Example 40. Unused

### Example 41—Identification of Enzyme Variants of HaCYP6AE15v2 and Hv_CYP_A0A2A4JAM9 for the Demethylation of Thebaine and Oripavine

Various cytochrome P450 enzymes which are able to demethylate thebaine to northebaine and oripavine to nororipavine were identified. The cytochrome P450s HaCYP6AE15v2 from Helicoverpa armigera and Hv_CYP_A0A2A4JAM9 from Heliothis virescens have demonstrated the highest thebaine and oripavine demethylation activities. New variants of HaCYP6AE15v2 and Hv_CYP_A0A2A4JAM9 were engineered, in order to improve its activities towards the demethylation of thebaine and/or oripavine.

Models of HaCYP6AE15v2 and Hv_CYP_A0A2A4JAM9 were constructed and thebaine was docked into the best working model in a reactive conformation and possible mutations across the protein structure were analyzed using proprietary scoring methodologies. Although mutations are likely to be compatible with each other, they were each tested individually first. A shortlist of single mutations that were expected to be tolerated and/or to enhance the protein activity, was generated.

Mutant versions of HaCYP6AE15v2 and Hv_CYP_A0A2A4JAM9 were tested with a cytochrome P450 reductase (CPR) from Helicoverpa armigera (SEQ ID NO 293) and a permease from Papaver somniferum (SEQ ID NO 466). For the demethylation of thebaine to northebaine, mutants were tested in Delft media pH 7.0 with the addition of 500 μM of thebaine. For the demethylation of oripavine to nororipavine, mutants were tested in Delft media pH 4.5 with the addition of 500 μM of oripavine.

For HaCYP6AE15v2, a set of 43 mutations have been tested individually for thebaine and oripavine demethylation. Mutation A316G gives 90% more northebaine compared with wild-type (see Table 41-1). Mutations A316G and D392E give 6.9% and 26% more oripavine compared with wild-type, respectively (see Table 41-2). For the 43 mutations of HaCYP6AE15v2 tested, no significant changes were observed for northebaine oxaziridine, nororipavine oxaziridine, thebaine N-oxide and oripavine N-oxide compounds.

For Hv_CYP_A0A2A4JAM9 a set of 58 mutations have been tested individually for thebaine and oripavine demethylation. Mutation Q393E gives 8% more northebaine compared to the wild-type (see Table 41-3). Mutation A110S gives 12% more nororipavine compared to the wild-type (see Table 41-5). For the 58 mutations of Hv_CYP_A0A2A4JAM9 tested, no significant changes were observed for northebaine oxaziridine, nororipavine oxaziridine, thebaine N-oxide and oripavine N-oxide compounds.

Following the 12% increase in oripavine demethylation by A110S mutation in Hv_CYP_A0A2A4JAM9, other variants of this active mutant were also tested. Mutant variants A110T, A110N and A110V were individually tested and also combined with proximal neutrals mutants previously identified on single mutation test experiment. Proximal neutrals mutants R112K, H242P and V224I are likely to influence A110 binding position. Taking this into consideration the active mutants were tested combinatorially with proximal mutants. Mutations 110N+H242P+V224I gives 8% more nororipavine compared to the wild-type (see Table 41-6). For the 32 mutant combinations of Hv_CYP_A0A2A4JAM9 tested, no significant changes were observed for northebaine oxaziridine, nororipavine oxaziridine, thebaine N-oxide and oripavine N-oxide compounds.

TABLE 41-1
Bioconversion of thebaine to northebaine in strains expressing
single mutations of HaCYP6AE15v2 cytochrome P450 enzyme from
Helicoverpa armigera, grown in DELFT minimal medium
at pH 7.0 with 500 μM of thebaine. The values represent
the comparison of activity between the mutants compared
to the wild-type version of HaCYP6AE15v2 in percentage
of conversion of thebaine to northebaine.

| HaCYP6AE15v2 mutation | Northebaine (%) |
|---|---|
| Wild-type | 0.00 |
| A20S | 6.93 |
| K31N | 0.72 |
| F44Y | 3.19 |
| F53Y | −7.82 |
| I54L | 3.26 |
| V105I | −2.89 |
| F114V | −21.02 |
| S115T | 4.49 |
| A120F | −20.09 |
| A178R | 0.07 |
| V188A | −6.11 |
| D192E | 12.42 |
| V210L | 8.10 |
| I211F | −93.43 |
| Q213D | 4.51 |
| Q213S | 3.86 |
| N215S | 14.82 |
| Q240F | −28.08 |
| M252L | −1.32 |
| E293D | 10.25 |
| L313V | −80.20 |
| A316G | 90.08 |
| Y319F | −0.56 |
| D349E | 4.45 |
| N354H | 19.64 |
| D355N | 8.12 |
| N374D | −23.75 |
| A384G | −7.03 |
| I385V | −9.35 |
| I385N | −4.43 |
| P386L | −46.89 |
| D392E | 23.54 |
| I408V | 8.96 |
| Q416H | 17.53 |
| S418N | 12.95 |
| D451E | 0.17 |
| V455I | −0.81 |
| I459M | 32.77 |
| R463K | 26.00 |
| S495T | −1.037 |
| V496F | 18.18 |
| V497L | 19.34 |
| T498S | −5.43 |
| Empty plasmid control | −94.44 |
| Media control | −100.00 |

TABLE 41-2
Bioconversion of oripavine to nororipavine in strains expressing
single mutations of HaCYP6AE15v2 cytochrome P450 enzyme from
Helicoverpa armigera, grown in DELFT minimal medium
at pH 4.5 with 500 μM of oripavine. The values represent
the comparison of activity between the mutants compared
to the wild-type version of HaCYP6AE15v2 in percentage
of conversion of oripavine to nororipavine.

| HaCYP6AE15v2 mutation | Nororipavine (%) |
|---|---|
| Wild-type | 0.00 |
| A20S | 7.51 |
| K31N | 1.94 |
| F44Y | −0.90 |
| F53Y | 0.53 |
| I54L | −4.81 |
| V105I | −12.71 |
| F114V | −29.05 |
| S115T | 5.74 |
| A120F | −28.31 |
| A178R | 5.81 |
| V188A | 1.38 |
| D192E | 12.34 |
| V210L | 0.59 |
| I211F | −91.21 |
| Q213D | −9.12 |
| Q213S | −19.09 |
| N215S | −2.38 |
| Q240F | −67.28 |
| M252L | −9.66 |
| E293D | 16.912 |
| L313V | −69.00 |
| A316G | 6.91 |
| Y319F | −14.44 |
| D349E | −3.84 |
| N354H | −2.91 |
| D355N | −0.51 |
| N374D | −21.80 |
| A384G | −6.52 |
| I385V | −13.44 |
| I385N | −4.36 |
| P386L | −47.56 |
| D392E | 26.13 |
| I408V | 13.63 |
| Q416H | 2.24 |
| S418N | −1.32 |
| D451E | 7.12 |
| V455I | −11.85 |
| I459M | 6.35 |
| R463K | −2.09 |
| S495T | −9.22 |
| V496F | −34.92 |
| V497L | −29.38 |
| T498S | −10.84 |
| Empty plasmid control | −93.81 |
| Media control | −100 |

TABLE 41-3
Bioconversion of thebaine to northebaine in strains expressing
single mutations of Hv_CYP_A0A2A4JAM9 cytochrome
P450 enzyme from Heliothis virescens, grown in DELFT
minimal medium at pH 7.0 with 500 μM of thebaine. The values
represent the comparison of activity between the mutants
compared to the wild-type version of Hv_CYP_A0A2A4JAM9
in percentage of conversion of thebaine to northebaine.

| Hv_CYP_A0A2A4JAM9 mutation | Northebaine (%) |
|---|---|
| WT | 0.00 |
| K23R | −19.61 |
| H104R | −89.83 |
| K117Q | −26.25 |
| I192V | −22.88 |
| I241F | 1.85 |
| T317A | −11.73 |
| L386V | −19.15 |
| V501P | −6.03 |
| V36L | 4.58 |
| I106V | −19.50 |
| A121F | −23.52 |
| A211S | −0.24 |
| H242P | 5.49 |
| H337N | 6.28 |
| L388T | −17.46 |
| N508K | −1.91 |
| F45Y | −40.05 |
| E108D | 5.66 |
| S122T | −1.79 |
| F212I | −23.34 |
| E246D | −74.65 |
| D350E | 2.45 |
| Q393E | 8.40 |
| I55L | −46.88 |
| F109Y | −0.83 |
| K126R | 6.42 |
| N216S | −4.30 |
| M252L | 5.24 |
| Q353R | 6.37 |
| F410H | −15.76 |
| V71Y | −2.40 |
| A110S | −1.55 |
| T139S | −39.26 |
| S220G | −9.71 |
| I256V | −49.85 |
| E358K | 4.44 |
| R443K | −11.02 |
| A77G | −6.89 |
| R112K | −0.28 |
| L160M | −2.08 |
| Q222K | −54.28 |
| D261N | 2.05 |
| F369Y | 1.32 |
| D452E | −6.46 |
| M83I | −17.64 |
| L115F | 4.21 |
| V171I | −15.67 |
| V224 | −4.40 |
| K280N | −4.83 |
| M380L | 3.19 |
| I497F | −66.75 |
| F100Y | −6.36 |
| G116T | −38.07 |
| G184C | 32.74 |
| Y229W | −12.85 |
| I291M | 1.27 |
| A383V | −27.60 |
| I498V | −18.68 |
| Plasmid control | −94.90 |
| Media control | −98.00 |

TABLE 41-4
Bioconversion of thebaine to northebaine in strains expressing
multiple mutations of Hv_CYP_A0A2A4JAM9 cytochrome
P450 enzyme from Heliothis virescens, grown in DELFT
minimal medium at pH 7.0 with 500 μM of thebaine. The values
represent the comparison of activity between the mutants
compared to the wild-type version of Hv_CYP_A0A2A4JAM9
in percentage of conversion of thebaine to northebaine.

| Hv_CYP_A0A2A4JAM9 mutations | Northebaine (%) |
|---|---|
| WT | 0.00 |
| A110S | −2.86 |
| A110T | −7.52 |
| A110N | −3.25 |
| A110V | −11.76 |
| A110S + R112K | −2.30 |
| A110S + H242P | 0.76 |
| A110S + 224I | −8.28 |
| A110S + R112K + H242P | 0.64 |
| A110S + R112K + V224I | −6.13 |
| A110S + H242P + V224I | 1.53 |
| A110S + R112K + H242P + V224I | −1.60 |
| A110T + R112K | −7.22 |
| A110T + H242P | −4.15 |
| A110T + V224I | −11.28 |
| A110T + R112K + H242P | −3.96 |
| A110T + R112K + V224I | −11.96 |
| A110T + H242P + V224I | −7.11 |
| A110T + R112K + H242P + V224I | −2.37 |
| A110N + R112K | −3.93 |
| A110N + H242P | 1.20 |
| A110N + V224I | −6.94 |
| A110N + R112K + H242P | 0.40 |
| A110N + R112K + V224I | −8.48 |
| A110N + H242P + V224I | −0.97 |
| A110N + R112K + H242P + V224I | −2.86 |
| A110V + R112K | −8.79 |
| A110V + H242P | −7.34 |
| A110V + V224I | −16.57 |
| A110V + R112K + H242P | −5.03 |
| A110V + R112K + V224I | −13.85 |
| A110V + H242P + V224I | −10.68 |
| A110V + R112K + H242P + V224I | −9.13 |
| Plasmid control | −95.54 |
| Media control | −100 |

TABLE 41-5
Bioconversion of oripavine to nororipavine in strains expressing
single mutations of Hv_CYP_A0A2A4JAM9 cytochrome
P450 enzyme from Heliothis virescens, grown in DELFT
minimal medium at pH 4.5 with 500 μM of oripavine. The values
represent the comparison of activity between the mutants
compared to the wild-type version of Hv_CYP_A0A2A4JAM9
in percentage of conversion of oripavine to nororipavine.

| Hv_CYP_A0A2A4JAM9 mutation | Nororipavine (%) |
|---|---|
| WT | 0 |
| K23R | −19.30 |
| H104R | 96.75 |
| K117Q | −23.24 |
| I192V | −15.78 |
| I241F | −16.21 |
| T317A | 0.80 |
| L386V | −11.28 |
| V501P | −16.76 |
| V36L | −5.43 |
| I106V | −19.51 |
| A121F | −69.57 |
| A211S | 0.21 |
| H242P | 3.67 |
| H337N | −15.67 |
| L388T | −44.02 |
| N508K | −1.95 |
| F45Y | −38.13 |
| E108D | −10.98 |
| S122T | −17.98 |
| F212I | −16.82 |
| E246D | −63.56 |
| D350E | −2.85 |
| Q393E | 1.15 |
| I55L | −46.24 |
| F109Y | −13.22 |
| K126R | 0.37 |
| N216S | −13.48 |
| M252L | 0.04 |
| Q353R | 0.77 |
| F410H | −17.15 |
| V71Y | −1.57 |
| A110S | 11.96 |
| T139S | −20.17 |
| S220G | −16.55 |
| I256V | −44.65 |
| E358K | 7.07 |
| R443K | −23.66 |
| A77G | −8.93 |
| R112K | −4.13 |
| L160M | 0.89 |
| Q222K | −58.56 |
| D261N | 0.26 |
| F369Y | −4.61 |
| D452E | 0.59 |
| M83I | −9.67 |
| L115F | −13.58 |
| V171I | −14.32 |
| V224 | −0.83 |
| K280N | −2.14 |
| M380L | −9.77 |
| I497F | −81.37 |
| F100Y | −0.03 |
| G116T | −20.80 |
| G184C | −14.01 |
| Y229W | −13.51 |
| I291M | 3.92 |
| A383V | 1.52 |
| I498V | −8.40 |
| Plasmid control | −97.10 |
| Media control | −100.00 |

TABLE 41-6
Bioconversion of oripavine to nororipavine in strains expressing
multiple mutations of Hv_CYP_A0A2A4JAM9 cytochrome
P450 enzyme from Heliothis virescens, grown in DELFT
minimal medium at pH 4.5 with 500 μM of oripavine. The values
represent the comparison of activity between the mutants
compared to the wild-type version of Hv_CYP_A0A2A4JAM9
in percentage of conversion of oripavine to nororipavine.

| Hv_CYP_A0A2A4JAM9 mutations | Nororipavine (%) |
|---|---|
| WT | 0.00 |
| A110S | 1.02 |
| A110T | −1.71 |
| A110N | 3.85 |
| A110V | −2.40 |
| A110S + R112K | 2.71 |
| A110S + H242P | 5.34 |
| A110S + 224I | 1.31 |
| A110S + R112K + H242P | 5.36 |
| A110S + R112K + V224I | 0.83 |
| A110S + H242P + V224I | 4.53 |
| A110S + R112K + H242P + V224I | 5.40 |
| A110T + R112K | 0.04 |
| A110T + H242P | 3.01 |
| A110T + V224I | −2.35 |
| A110T + R112K + H242P | 3.27 |
| A110T + R112K + V224I | −1.92 |
| A110T + H242P + V224I | 1.83 |
| A110T + R112K + H242P + V224I | 3.51 |
| A110N + R112K | 3.68 |
| A110N + H242P | 7.52 |
| A110N + V224I | 2.61 |
| A110N + R112K + H242P | 6.95 |
| A110N + R112K + V224I | 2.38 |
| A110N + H242P + V224I | 7.94 |
| A110N + R112K + H242P + V224I | 6.02 |
| A110V + R112K | −1.33 |
| A110V + H242P | 1.01 |
| A110V + V224I | −4.61 |
| A110V + R112K + H242P | 1.92 |
| A110V + R112K + V224I | −3.78 |
| A110V + H242P + V224I | 0.65 |
| A110V + R112K + H242P + V224I | 1.03 |
| Plasmid control | −97.75 |
| Media control | −100.00 |

### Example 42—Functional Expression of HaCYP6AE15v2 and Hv_CYP_A0A2A4JAM9 N-Terminal Variants in S. cerevisiae

Additional enzymes mutations were designed and tested for better activity, e.g., from expression/stability/yield improvements in the host cell. Mutants were created by replacing the native N-terminal membrane-spanning domain of cytochrome P450 enzymes HaCYP6AE15v2 and Hv_CYP_A0A2A4JAM9 with the N-terminal membrane spanning domain from the plant cytochrome P450 Eschscholzia californica N-methylcoclaurine 3′-hydroxylase or Eschscholzia californica cheilanthifoline synthase.

The demethylases H. armigera HaCYP6AE15v2 and H. virescens Hv_CYP_A0A2A4JAM9 are insect cytochrome P450s, which are usually membrane-bound enzymes and localize to the microsomes in yeast. Analysis of the sequence of H. armigera HaCYP6AE15v2 using SignalP 4.1 {http://www.cbs.dtu.dk/services/SignalP) allowed identification of a possible HaCYP6AE15v2 N-terminal α-helix of 21 amino acids for membrane localization. The HaCYP6AE15v2 protein was truncated between amino acids 2 and 21 and the truncated version was ordered from Twist Bioscience cloned into vector p415 (Mumberg, Müller and Funk 1995). The same analysis was then performed on H. virescens Hv_CYP_A0A2A4JAM9 cytochrome P450, and fusion proteins were generated in silico with the modified N-terminal α-helices and truncated Hv_CYP_A0A2A4JAM9.

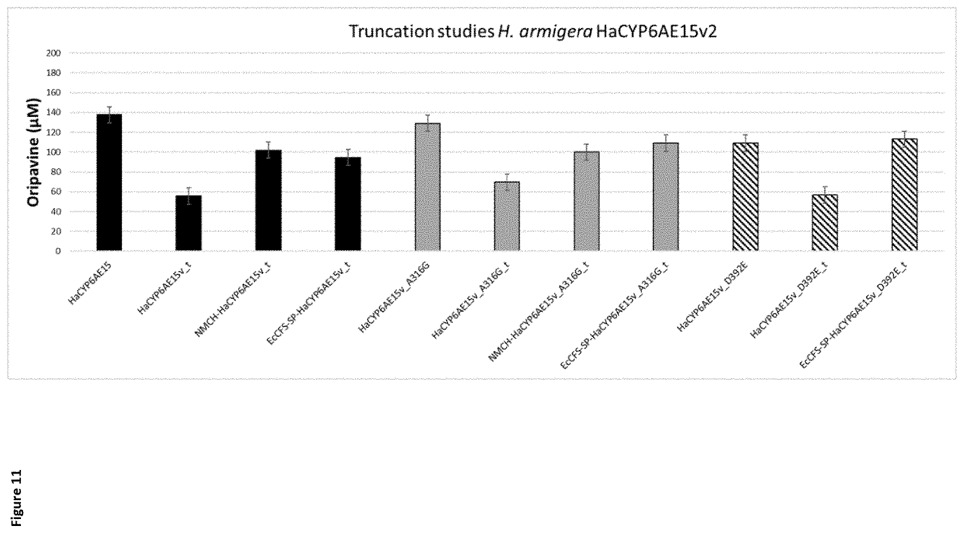

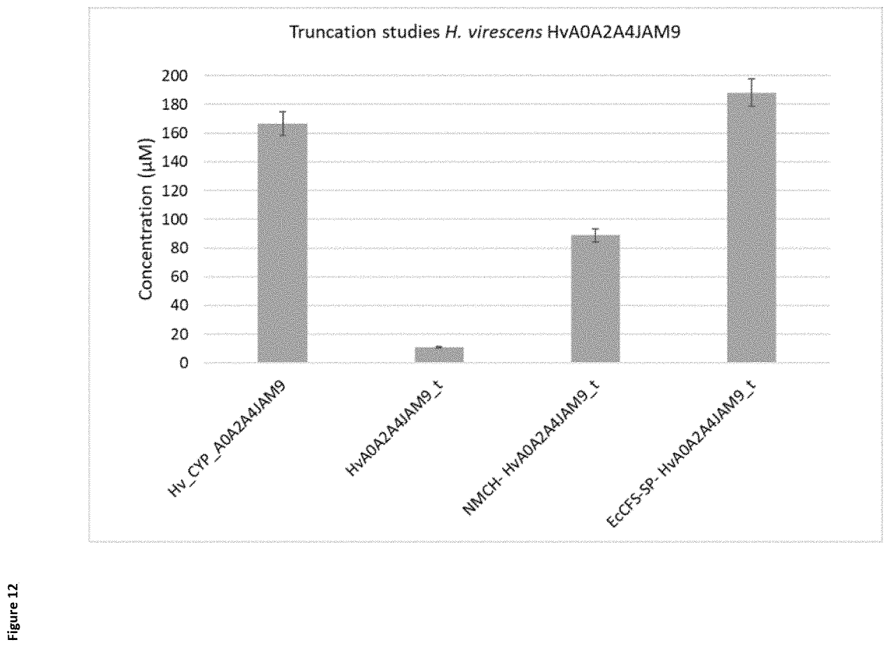

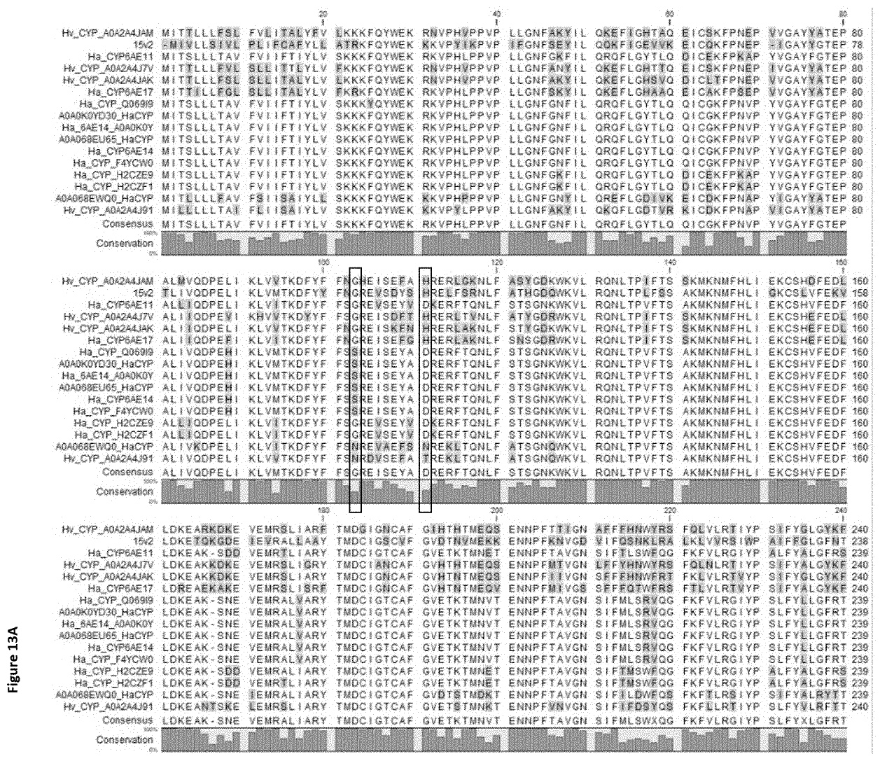

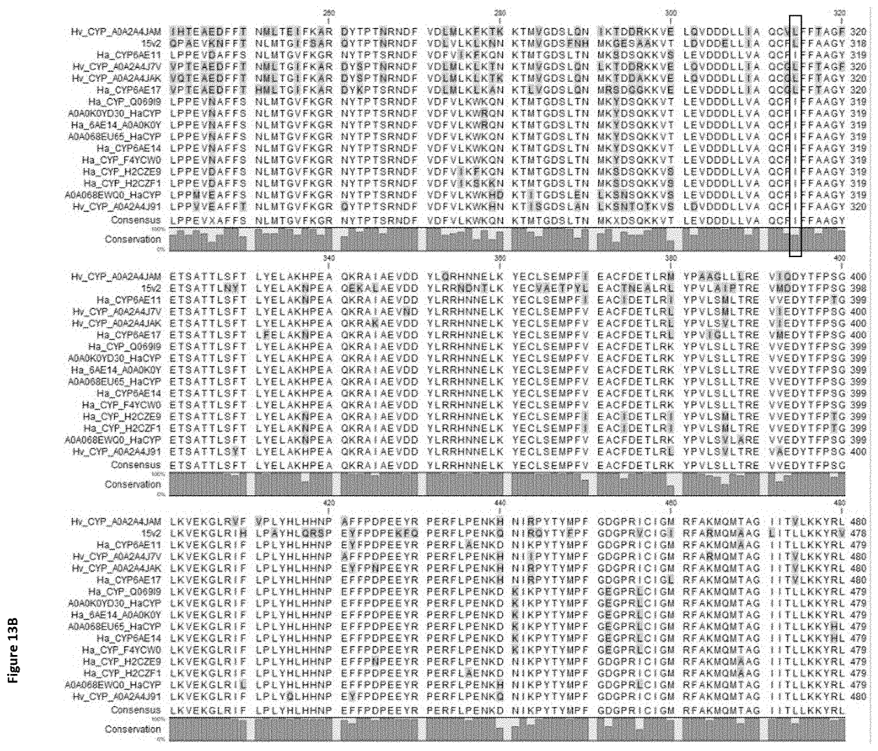

The variants of HaCYP6AE15v2 and Hv_CYP_A0A2A4JAM9 were all tested with a cytochrome P450 reductase (CPR) from H. armigera (SEQ ID NO 293) and a permease from Papaver somniferum (SEQ ID NO 466). For the demethylation of thebaine to northebaine, mutants were tested in Delft media pH 7.0 with the addition of 500 μM of thebaine. For the demethylation of oripavine to nororipavine, mutants were tested in Delft media pH 4.5 with the addition of 500 μM of oripavine (see FIG. 11 and FIG. 12).

For HaCYP6AE15v2, all variants tested showed activity in the demethylation of oripavine to nororipavine, but the wild-type HaCYP6AE15v2 showed the highest activity. In all variants the introduction of one of the N-terminal membrane-spanning domains tested, had a positive effected in the H. armigera compared with the truncated version.

For Hv_CYP_A0A2A4JAM9, the truncated version of the demethylase showed very low activity. The introduction of an N-terminal membrane-spanning domain had a positive effect in Hv_CYP_A0A2A4JAM9 activity. EcCFS—SP-Hv_CYP_A0A2A4JAM9_t variant had a 13% increase of nororipavine production compared with wild-type, showing that this is a very effective strategy to improve the activity of demethylases for the production of nororipavine.

FIG. 11 shows the activity of N-terminal variants of HaCYP6AE15v2 expressed in S. cerevisiae and its bioconversion of oripavine to nororipavine in strains expressing N-terminal variants and N-terminal variants combined with single mutations of HaCYP6AE15v2 cytochrome P450 enzyme, grown in DELFT minimal medium at pH 4.5 with 500 μM of oripavine. HaCYP6AE15v2 was truncated between amino acids 2 and 21 to generate truncated HaCYP6AE15v2_t. In FIG. 11 HaCYP6AE15v2 is also referred to as HaCYP6AE15v or HaCYP6AE15.

NMCH-HaCYP6AE15v2_t is a fusion protein of the N-terminal domain of EcNMCH and truncated HvCYP6AE15v2, EcCFS—SP-HaCYP6AE15v2_t is a fusion protein of the N-terminal domain of EcCFS and truncated HvCYP6AE15v2, NMCH-HaCYP6AE15v2_A316G_t is a fusion protein of the N-terminal domain of EcNMCH and truncated HvCYP6AE15v2_A316G, EcCFS—SP-HaCYP6AE15v2_A316G_t is a fusion protein of the N-terminal domain of EcCFS and truncated HvCYP6AE15v2_A316G, NMCH-HaCYP6AE15v2_D392E_t is a fusion protein of the N-terminal domain of EcNMCH and truncated HvCYP6AE15v2_D392E, and EcCFS—SP-HaCYP6AE15v2_D392E_t is a fusion protein of the N-terminal domain of EcCFS and truncated HvCYP6AE15v2_D392E.

FIG. 12 show the activity of N-terminal variants of Hv_CYP_A0A2A4JAM9 expressed in S. cerevisiae and its bioconversion of oripavine to nororipavine in strains expressing N-terminal of Hv_CYP_A0A2A4JAM9 cytochrome P450 enzyme, grown in DELFT minimal medium at pH 4.5 with 500 μM of oripavine. Hv_CYP_A0A2A4JAM9 was truncated between amino acids 2 and 21 to generate truncated Hv_CYP_A0A2A4JAM9_t. In FIG. 12 Hv_CYP_A0A2A4JAM9 is also referred to as Hv_A0A2A4JAM9 or HvA0A2A4JAM9.

NMCH-Hv_CYP_A0A2A4JAM9_t is a fusion protein of the N-terminal domain of EcNMCH and truncated Hv_CYP_A0A2A4JAM9, EcCFS—SP-Hv_CYP_A0A2A4JAM9_t is a fusion protein of the N-terminal domain of EcCFS and truncated Hv_CYP_A0A2A4JAM9.

### Example 43—Pattern Analysis of Enzyme Variants of HaCYP6AE15v2 and Hv_CYP_A0A2A4JAM9 for the Demethylation of Thebaine and Oripavine

Various datasets were generated based on identity thresholds of the two most active insect cytochrome P450's for the demethylation of thebaine and oripavine—Hv_CYP_A0A2A4JAM9 and HaCYP5AE15v2. A multiple alignment of sequences was performed with Clustal Omega program (EMBL-EBI) using parameters which for analytical purposes deviated from the methods described in the definitions. Clustal parameters were as follows:
MAX HMM ITERATIONS=−1
MAX_GUIDE_TREE_ITERATION=−1
NUMBER_COMBINED_ITERATIONS=0
MBED_LIKE_CLUSTERING_GUIDE_TREE=TRUE

Based on this alignment, different groups of sequences were extracted according to % identity to Hv_CYP_A0A2A4JAM9 and HaCYP5AE15v2, separately. The sequences were grouped according to a defined % identity homology dataset (>70%, >60% and >50% homology). Sequences were then assigned as active or inactive based on the data shown in Example 5. The composition overview for the cytochrome P450 sequences compared to Hv_CYP_A0A2A4JAM9 is shown in Table 43-1 and to HaCYP5AE15v2 in Table 43-2.

TABLE 43-1
Composition of dataset based on alignment and
identity to Hv_CYP_A0A2A4JAM9.

| | Total | | |
|---|---|---|---|
| Data Set | Number | Active | Inactive |
| >70% ID to | 16 | 6 (does not include | 10 |
| Hv_CYP_A0A2A4JAM9 | | HaCYP5AE15v2) | |
| >60% ID to | 23 | 8 (includes | 15 |
| Hv_CYP_A0A2A4JAM9 | | HaCYP5AE15v2) | |
| >50% ID to | 64 | 27 | 37 |
| Hv_CYP_A0A2A4JAM9 | | | |

TABLE 43-2
Composition of dataset based on alignment
and identity to HaCYP5AE15v2.

| | Total | | |
|---|---|---|---|
| DataSet | number | Active | Inactive |
| >70% ID to | 5 | 2 (does not include | 3 |
| HaCYP5AE15v2 | | Hv_CYP_A0A2A4JAM9) | |
| >60% ID to | 33 | 17 (includes | 16 |
| HaCYP5AE15v2 | | Hv_CYP_A0A2A4JAM9) | |
| >50% ID to | 70 | 28 | 42 |
| HaCYP5AE15v2 | | | |

In total 129 cytochrome P450's were tested for the demethylation of thebaine to northebaine and oripavine to nororipavine. From 129 demethylases tested, 33 were classified as active and 96 were classified inactive. An inactive demethylase enzyme is defined as having a demethylation activity of thebaine or oripavine below the detection level. Generally, enzymes that are able to demethylate thebaine are the same enzymes which are able to demethylate oripavine (i.e. these are not mutually exclusive sets, despite variance in degrees of demethylation).

Structure models were created for Hv_CYP_A0A2A4JAM9 and HaCYP5AE15v2 sequences and identified binding site locations for thebaine. Sequence dependence was screened at single amino acid positions. In addition, structural models of all proteins in the whole dataset were generated, and active structures were modelled with thebaine to identify potential binding site residues positions. Models were aligned to establish variance in coordinates of sequences and to determine the sequence dependence on activity within some of the datasets from Tables 43-1 and 43-2. For Hv_CYP_A0A2A4JAM9, an analysis of single residue positions was performed based on the datasets >70% ID to Hv_CYP_A0A2A4JAM9 and >60% ID to Hv_CYP_A0A2A4JAM9 from Table 43-1. Single amino acid positions which most effectively separate active and inactive sequences were screened with banded % identity to Hv_CYP_A0A2A4JAM9 (see Table 43-3). The column dataset denotes which dataset the results relate to. The residues in bold correspond to active site residues, according to modelling predictions. The remaining columns on Table 43-3 show key statistics for individual single amino acid results. The resulting sub-alignment from >70% ID to Hv_CYP_A0A2A4JAM9 data set used to generate the data described in Table 43-3 is represented in FIG. 13.

In the analysis of single residue screening of Hv_CYP_A0A2A4JAM9 based on >70% ID data set, thirteen single residues were identified (Table 43-3). From the thirteen single residues identified, three single residues are classified as active site residues—G103, H111 and L314 (FIG. 13). In the analysis of single residue screening of Hv_CYP_A0A2A4JAM9 based on >60% ID data set, seven single residues were identified (Table 43-3).

TABLE 43-3
Single residue screening relative to Hv_CYP_AOA2A4JAM9. Residues in bold are predicted
to be in the binding site from modelling. The standard statistical calculations of PPV, NPV,
sensitivity and specificity are calculated according to the following formula:
PPV = TP/(TP + FP); NPV = TN/(TN + FN); Sensitivity = TP/(TP + FN);
Specificity = TN/(FP + TN). Where TN = True Negative, FN = False Negative,
TP = True positive, FP = False Positive.

| | Single | | | | | | | |
|---|---|---|---|---|---|---|---|---|
| DataSet | AA‡ | Actives | | Inactives | PPV | NPV | Sensitivity | Specificity |
| >70% ID to | G103 | 7/7 | | 2/10 | 0.78 | 1 | 1 | 0.8 |
| Hv_CYP_A0A2A4JAM9 + | H111 | 6/7* | | 0/10 | 1 | 0.91 | 0.86 | 1 |
| HaCYP5AE15v2 | K167 | 6/7* | | 0/10 | 1 | 0.91 | 0.86 | 1 |
| | E198 | 6/7* | | 0/10 | 1 | 0.91 | 0.86 | 1 |
| | R219 | 6/7* | | 0/10 | 1 | 0.91 | 0.86 | 1 |
| | L223 | 6/7* | | 0/10 | 1 | 0.91 | 0.86 | 1 |
| | I256 | 6/7* | | 0/10 | 1 | 0.91 | 0.86 | 1 |
| | A259 | 6/7* | | 0/10 | 1 | 0.91 | 0.86 | 1 |
| | L273 | 6/7* | | 0/10 | 1 | 0.91 | 0.86 | 1 |
| | V284 | 6/7* | | 0/10 | 1 | 0.91 | 0.86 | 1 |
| | I309 | 6/7* | | 0/10 | 1 | 0.91 | 0.86 | 1 |
| | L314 | 6/7* | | 0/10 | 1 | 0.91 | 0.86 | 1 |
| | Q517 | 6/7* | | 0/10 | 1 | 0.91 | 0.86 | 1 |
| >60% ID to | L223 | 6/8 | | 0/16 | 1 | 0.89 | 0.75 | 1 |
| Hv_CYP_A0A2A4JAM9 | L160 | 5/8 | (miss 15v2) | 0/16 | 1 | 0.84 | 0.63 | 1 |
| (inc HaCYP5AE15v2) | L160 | 7/8 | (inc 15v2) | 3/16 | 0.7 | 0.93 | 0.88 | 0.81 |
| | L or V | | | | | | | |
| | N216 | 5/8 | (inc 15v2) | 0/16 | 1 | 0.84 | 0.63 | 1 |
| | A259 | 5/8 | (inc 15v2) | 0/16 | 1 | 0.84 | 0.63 | 1 |
| | V284 | 5/8 | (inc 15v2) | 0/16 | 1 | 0.84 | 0.63 | 1 |
| | R443 | 5/8 | (inc 15v2) | 0/16 | 1 | 0.84 | 0.63 | 1 |

‡symbolize the position in HaCYP5AE15v2.
*symbolize the active missed is HaCYP6AE11 2.57% northebaine conversion, other actives are: Hv_CYP_A0A2A4JAM9 44.33%; HaCYP5AE15v2 30.74%; Hv_CYP_A0A2A4J7V4 10.24%; Hv_CYP_A0A2A4JAK3 22.62%; Ha_CYPAE17 6.31%.

In the table the asterisk denotes HaCYP6AE11 in every case, the one “missed active,” which exhibited a very low enzymatic activity as compared to the other proteins included in the table. The analysis has very good predictability of what constitutes a good demethylase enzyme for the reactions considered, in regards to the conservation at specific residues and relative homology to the identified best enzymes. It is noteworthy, that some of these conserved sites that are highly predictive are also expected to be near the active site of the enzyme.

In addition it is expected that single residue conservative changes to the sites listed in Table 43-3 will also be active enzymes for the reactions studied. For example, enzymes tested with single point mutations in Hv_CYP_A0A2A4JAM9 shown in Table 43-4 below still retained some demethylase activity, although not as good as some of the preferred residues listed in Table 43-3. In the table % Northebaine corresponds to the percentage of conversion of thebaine to northebaine compare to wild-type and % Nororipavine corresponds to the percentage of conversion of oripavine to nororipavine compare to wild-type.

TABLE 43-4

| Mutation Hv_CYP_A0A2A4JAM9 | % Northebaine | % Nororipavine |
|---|---|---|
| I256V | −49.848 | −44.649 |
| L160M | −2.08 | 0.889 |
| N216S | −4.297 | −13.482 |
| R443K | −11.022 | −23.661 |

FIG. 13 shows sequence alignment of data set >70% ID to Hv_CYP_A0A2A4JAM9 including HaCYP6AE15v2. The amino acids shaded in grey, represents the different residues compared with the consensus sequence. The residues in the black box correspond to the active site residues, according to modeling predictions. In this alignment the most active sequences Hv_CYP_A0A2A4JAM9 and HaCYP6AE15v2 are provided as the top sequences in the alignment for reference. This multiple sequence alignment was performed locally with Clustal Omega program and alignment visualization with CLC workbench 8.0. In FIG. 13 Hv_CYP_A0A2A4JAM9 is also be referred to as Hv_CYP_A0A2A4JAM, while HaCYP6AE15v2 is referred to as 15v2. Additionally in FIG. 13 underscore symbols are sometimes inserted in protein names eg (Ha_CYP6AE11 which is equivalent to HaCYP6AE11).

### Example 44. Identification of Equilibrative Nucleoside Transporters from Insects Capable of Improving Bioconversion of Oripavine to Nororipavine with Insect Demethylase from Helicoverpa armigera and Heliothis virescens

### Bioconversion

In this example, the impact of insect transporter proteins on bioconversion of oripavine to nororipavine was studied by transforming yeast strain with a plasmid containing a demethylase comparable to the above examples that was capable of acting on reticuline derivatives such as oripavine using the backbone plasmid p415TEF. A plasmid containing demethylase-CPR (pOD1184 from Example 7) was also expressed in combination with various candidate transporter proteins. Yeast strain construction and method of screening for transporters were as previously described in Example 7. Table 43-1 shows the result of percentage bioconversion from thebaine to northebaine with the expression of various transporters. The screening was performed at pH 4.5. Table 43-1 also presents the percentage improvement in the bioconversion when normalized for a control strain expressing demethylase but not expressing any heterologous transporter.

### Improvement of Bioconversion.

When compared to a control strain without a heterologous transporter, several strains engineered with various insect transporters exhibited moderate to high percentage bioconversion of the 500 μM oripavine fed in this assay. For strains expressing demethylase from Helicoverpa armigera, HaCYP6AE15v2, amongst the heterologous insect transporters examined, transporters T218_HviENT3_GA, T220_CsuENT3_GA, T221_BmoENT3_GA, and T227_AcuENT3_GA exhibited improvements in bioconversion of oripavine to nororipavine in the range of 413-1252% in comparison to the control strain without a heterologous transporter (Table 43-1). Expression of some insect transporters, such as T218_HviENT3_GA from Heliothis virescens and T220_CsuENT3_GA from Chilo suppressalis gave particularly remarkable improvements in the demethylase-mediated bioconversion of oripavine to nororipavine.

For strains expressing demethylase from Heliothis virescens, Hv_CYP_A0A2A4JAM9, amongst the heterologous insect transporters examined, again transporters T218_HviENT3_GA, T220_CsuENT3_GA, T221_BmoENT3_GA, and T227_AcuENT3_GA exhibited improvements in bioconversion of oripavine to nororipavine in the range of 443-1675% in comparison to the control strain without a heterologous transporter (Table 43-1). Expression of some transporters, such as T218_HviENT3_GA from Heliothis virescens and T220_CsuENT3_GA from Chilo suppressalis demonstrated particularly outstanding improvements in the demethylase-mediated bioconversion of oripavine to nororipavine.

In Table 44-1, for the first time, transporters from Heliothis virescens, Chilo suppressalis, Bombyx mori and Anopheles culicifacies (Malaria vector) have also been tested and shown to be capable in bioconversion of oripavine to nororipavine. T221_BmoENT3_GA from Bombyx mori and T227_AcuENT3_GA from Anopheles culicifacies demonstrate low activity of bioconversion, 6-11% with HaCYP6AE15v2 and Hv_CYP_A0A2A4JAM9. In contrast, with both insect demethylases, T218_HviENT3_GA from Heliothis virescens and T220_CsuENT3_GA from Chilo suppressalis exhibited great effect on bioconversion of oripavine with improvement as high as 1675% in comparison to the control strain without a heterologous transporter.

TABLE 44-1
Percentage demethylase-mediated bioconversion from Oripavine to Nororipavine with the expression of various transporters and
percentage improvements in the bioconversion as compared to a control strains not expressing any heterologous transporters.

| | | Percentage Bioconversion of Oripavine | | Percentage Improvement in Bioconversion | |
|---|---|---|---|---|---|
| | | to Nororipavine, pH 4.5 (%) | | of Oripavine to Nororipavine, pH 4.5 (%) | |
| | SEQ | Demethylase: | Demethylase: | Demethylase: | Demethylase: |
| Insect Transporter | ID NO: | HaCYP6AE15v2 | Hv_CYP_A0A2A4JAM9 | HaCYP6AE15v2 | Hv_CYP_A0A2A4JAM9 |
| T218_HviENT3_GA | 795 | 21.2 | 28.8 | 1030.0 | 1437.3 |
| T220_CsuENT3_GA | 797 | 25.4 | 33.3 | 1252.6 | 1675.7 |
| T221_BmoENT3_GA | 799 | 6.6 | 10.2 | 252.0 | 443.6 |
| T227_AcuENT3_GA | 801 | 9.6 | 11.9 | 413.7 | 535.1 |
| Control | | 1.9 | 1.9 | — | — |

Note:
Demethylase: HaCYP6AE15v2 represents demethylase from Helicoverpa armigera; Demethylase: Hv_CYP_A0A2A4JAM9 represents demethylase from Heliothis virescens. Control strain only contains a copy of demethylase, a copy of demethylase-CPR, HaCPR_E0A3A7 from Helicoverpa armigera, and an empty plasmid p416TEF. The demethylase-CPR, HaCPR_E0A3A7 is present in all strains.

### Insect Equilibrative Nucleoside Transporters (ENTs) for Bioconversion of Oripavine

All insect transporters tested in Example 18 and 44 are summarized in Table 44-2. The collection contains transporters from Helicoverpa armigera, Heliothis virescens, Chilo suppressalis, Bombyx mori and Anopheles culicifacies. These insects belong to the families of Noctuidae, Crambidae, Bombycidae and Culicidae. According to NCBI, T212_HarPUP3_GA and T215_HarPUP3_GA are categorized as Equilibrative Nucleoside Transporter 1. T213_HarPUP3_GA is categorized as Equilibrative Nucleoside Transporter 3. T218_HviENT3_GA, T220_CsuENT3_GA, T221_BmoENT3_GA, and T227_AcuENT3_GA were found based on BLAST against T213_HarPUP3_GA in Uniprot. These 4 insect transporters belong to the SLC29A/ENT transporter (TC 2.A.57) family. All insect transporters found contain nucleoside transmembrane transporter activity, which according to Uniprot, “enables the transfer of a nucleoside, a nucleobase linked to either beta-D-ribofuranose (ribonucleoside) or 2-deoxy-beta-D-ribofuranose, (a deoxyribonucleotide) from one side of a membrane to the other.”

TABLE 44-2
Equilibrative nucleoside transporters (ENTs) from insects that are
capable of improving bioconversion of oripavine to nororipavine.

| | SEQ | Gene origin | | |
|---|---|---|---|---|
| Transporter | ID NO: | (Latin name) | Family | Source |
| T212_HarPUP3_GA | 652 | Helicoverpa | Noctuidae | NCBI: PZC80691.1. Equilibrative nucleoside |
| | | armigera | | transporter 1 |
| T213_HarPUP3_GA | 654 | Helicoverpa | Noctuidae | NCBI: PZC85076.1. Equilibrative nucleoside |
| | | armigera | | transporter 3 |
| T215_HarPUP3_GA | 658 | Helicoverpa | Noctuidae | NCBI: XP_021186538.1. Equilibrative |
| | | armigera | | nucleoside transporter 1 isoform X2 |
| T218_HviENT3_GA | 795 | Heliothis | Noctuidae | Uniprot: A0A2A4JNN3. SLC29A/ENT |
| | | virescens | | transporter (TC 2.A.57) family. |
| T220_CsuENT3_GA | 797 | Chilo | Crambidae | Uniprot: A0A437BH16. SLC29A/ENT |
| | | suppressalis | | transporter (TC 2.A.57) family. |
| T221_BmoENT3_GA | 799 | Bombyx mori | Bombycidae | Uniprot: H9J6Q8. SLC29A/ENT |
| | | | | transporter (TC 2.A.57) family. |
| T227_AcuENT3_GA | 801 | Anopheles | Culicidae | Uniprot: A0A182MLN2. Uncharacterized |
| | | culicifacies | | protein. SLC29A/ENT transporter |
| | | | | (TC 2.A.57) family |

### Conclusion

In Table 44-1, insect transporters T218_HviENT3_GA, T220_CsuENT3_GA, T221_BmoENT3_GA, and T227_AcuENT3_GA have been demonstrated herein some for the first time to shown particularly high improvements in the demethylase-mediated bioconversion of oripavine to nororipavine. Beside that, in Example 18, several insect uptake transporters from Helicoverpa armigera such as T212_HarPUP3_GA, T213_HarPUP3_GA and T215_HarPUP3_GA had been shown to also exhibited excellent demethylase-mediated bioconversion of oripavine to nororipavine. In this Example, Equilibrative Nucleoside Transporters including those belong to SLC29A/ENT transporter (TC 2.A.57) family (https://www.uniprot.org) have been shown to be capable of demethylase-mediated bioconversion of oripavine to nororipavine in an efficient manner. Such improvements in yield are particularly remarkable and represent a significant step forward towards a sustainable, secure, and scalable biosynthetic means of producing these compounds.

### Example 45. Screening of Transporters with Mutated Insect Demethylases from Heliothis virescens Improves Bioconversion of Thebaine to Northebaine

### Bioconversion

In this example, the impact of transporter proteins on bioconversion of thebaine to northebaine was studied by transforming yeast strain with a plasmid containing a demethylase comparable to the above examples that was capable of acting on reticuline derivatives such as thebaine using the backbone plasmid p415TEF. A plasmid containing demethylase-CPR (pOD1184 from Example 7) was also expressed in combination with various candidate transporter proteins. Yeast strain construction and method of screening for transporters were as previously described in Example 7. Table 45-1 shows the result of percentage bioconversion from thebaine to northebaine with the expression of various transporters. The screening was performed at pH 7. Table 45-1 also presents the percentage improvement in the bioconversion when normalized for a control strain expressing demethylase but not expressing any heterologous transporter.

Improvement of bioconversion with mutated versions of Hv_CYP_A0A2A4JAM9.

In this Example, various transporters, some for the first time, were screened together with three mutated versions of Hv_CYP_A0A2A4JAM9 from Heliothis virescens, Hv_CYP_A0A2A4JAM9_A110S, Hv_CYP_A0A2A4JAM9_A110N+H242P and Hv_CYP_A0A2A4JAM9_A110N+H242P+V224I. The ranking of best transporters does not vary in a very significant way amongst these mutated Hv_CYP_A0A2A4JAM9. T198_AcoT97_GA and T149_AcoPUP3_59 have been shown to be the best transporters paring with these mutated demethylases. For certain transporters such as T198_AcoT97_GA, the increment in number of mutations in Hv_CYP_A0A2A4JAM9 increases the percentage bioconversion of thebaine to northebaine. For other transporters such as T193_AanPUP3_55, double mutations in Hv_CYP_A0A2A4JAM9_A110N+H242P is preferred for best bioconversion of thebaine. The result also shows additional insect Equilibrative Nucleoside Transporters, T218_HviENT3_GA and T238_HviENT3_GA from Heliothis virescens, T220_CsuENT3_GA and T234_CsuENT3_GA from Chilo suppressalis and T237_PxuENT3_GA from Papilio Xuthus that can mediate bioconversion of thebaine to northebaine with the mutants of Hv_CYP_A0A2A4JAM9.

TABLE 45-1
Percentage demethylase-mediated bioconversion from Thebaine to Northebaine with the expression of various transporters together
with mutated versions of Hv_CYP_A0A2A4JAM9 as compared to a control strains not expressing any heterologous transporters.

| | SEQ | Percentage Bioconversion of thebaine to northebaine at pH 7 (%) | | |
|---|---|---|---|---|
| | ID | | Hv_CYP_A0A2A4JAM9_A110N + | Hv_CYP_A0A2A4JAM9_A110N + |
| Transporter | NO: | Hv_CYP_A0A2A4JAM9_A110S | H242P | H242P + V224I |
| T102_PsoPUP3_1 | 466 | 27.5 | 32.1 | 31.7 |
| T116_HanPUP3_56 | 482 | 19.6 | 23.6 | 22.8 |
| T149_AcoPUP3_59 | 538 | NA | 40.4 | 38.2 |
| T165_AcoPUP3_13 | 568 | 30.1 | NA | NA |
| T168_FvePUP3_37 | 572 | 23.4 | 26.1 | 26.4 |
| T180_McoPUP3_46 | 596 | 20.3 | 20.6 | 21.5 |
| T182_CpaPUP3_62 | 600 | 28.4 | 28.6 | 34.9 |
| T192_CmiPUP3_47 | 612 | 21.4 | 24.2 | 22.7 |
| T193_AanPUP3_55 | 614 | 24.5 | 29.8 | 25.3 |
| T195_JcuPUP3_71 | 618 | 16.1 | 17.4 | 19.2 |
| T198_AcoT97_GA | 624 | 38.3 | 43.4 | 44.9 |
| T210_NnuPUP3_GA | 648 | 29.0 | 31.5 | 31.7 |
| T212_HarPUP3_GA | 652 | NA | 11.2 | 11.1 |
| T213_HarPUP3_GA | 654 | 26.4 | 31.8 | 27.8 |
| T215_HviENT3_GA | 658 | NA | 10.5 | 10.6 |
| T218_HviENT3_GA | 795 | 24.4 | 28.5 | 27.7 |
| T220_CsuENT3_GA | 797 | 25.8 | 28.2 | 28.5 |
| T234_CsuENT3_GA | 803 | NA | 12.9 | 12.0 |
| T237_PxuENT3_GA | 805 | NA | 11.5 | 11.1 |
| T238_HviENT3_GA | 807 | NA | 12.5 | 10.7 |
| T243_EguPUP3_GA | 815 | NA | 19.1 | 9.0 |
| T244_CcaPUP3_GA | 817 | NA | 18.7 | 14.7 |
| T253_AanPUP3_GA | 823 | NA | 20.4 | 18.0 |
| T254_CcaPUP3_GA | 825 | NA | 19.5 | 17.9 |
| Control | | 11.3 | 10.9 | 11.8 |

Note:
Demethylase: Hv_CYP_A0A2A4JAM9_A110S represents demethylase from Heliothis virescens with single mutation at amino acid residue 110. Hv_CYP_A0A2A4JAM9_A110N + H242P represents demethylase from Heliothis virescens with double mutations at amino acid residues 110 and 242. Hv_CYP_A0A2A4JAM9_A110N + H242P + V224I represents demethylase from Heliothis virescens with triple mutations at amino acid residues 110, 242 and 224. Control strain only contains a copy of demethylase, a copy of demethylase-CPR, HaCPR_E0A3A7 from Helicoverpa armigera, and an empty plasmid p416TEF. The demethylase-CPR, HaCPR_E0A3A7 is present in all strains.

### Insect Equilibrative Nucleoside Transporters for Bioconversion of Thebaine

All insect transporters tested with significant transporter activity in this example are summarized in Table 45-2. The list contains transporters from Helicoverpa armigera, Heliothis virescens, Chilo suppressalis. These insects belong to the families of Noctuidae and Crombidae. In example 44, T212_HarPUP3_GA, T213_HarPUP3_GA, T215_HarPUP3_GA, T218_HviENT3_GA, T220_CsuENT3_GA, T221_BmoENT3_GA, and T227_AcuENT3_GA has previously been described to have nucleoside transmembrane transporter activity for bioconversion of oripavine. In this example, only T213_HarPUP3_GA, T218_HviENT3_GA and T220_CsuENT3_GA have transporter activity for bioconversion of thebaine. This shows indicates that some transporters are substrate specific while other transporters may be promiscuous.

TABLE 45-2
Equilibrative nucleoside transporters from insects that are capable
of improving bioconversion of thebaine to northebaine.

| | SEQ | Gene origin | | |
|---|---|---|---|---|
| Transporter | ID NO: | (Latin name) | Family | Source |
| T213_HarPUP3_GA | 654 | Helicoverpa | Noctuidae | NCBI: PZC85076.1. Equilibrative nucleoside |
| | | armigera | | transporter 3 |
| T218_HviENT3_GA | 795 | Heliothis | Noctuidae | Uniprot: A0A2A4JNN3. SLC29A/ENT |
| | | virescens | | transporter (TC 2.A.57) family. |
| T220_CsuENT3_GA | 797 | Chilo | Crambidae | Uniprot: A0A437BH16. SLC29A/ENT |
| | | suppressalis | | transporter (TC 2.A.57) family. |

### Conclusion

Table 45-1 shows some of the transporters that have been herein demonstrated to have shown very considerable improvements in the bioconversion from thebaine to northebaine by 3 different mutated demethylases Hv_CYP_A0A2A4JAM9. In particular, the result of this example demonstrates that together with 1 of the 3 mutated demethylases, expression of transporter T198_AcoT97_GA from Aquilegia coerulea stimulated somewhere in the range of 238-298% more in bioconversion of thebaine to northebaine, when compared to a control strain without transporter. Several insect equilibrative nucleoside transporters have also been identified in this example. The great yield shown herein are highly valuable given the nature of the opioid-related compounds produced.

### Example 46. Screening of Transporters with Mutated Insect Demethylases from Heliothis virescens Improves Bioconversion of Oripavine to Nororipavine

### Bioconversion

In this example, the impact of transporter proteins on bioconversion of oripavine to nororipavine was studied by transforming yeast strain with a plasmid containing a demethylase comparable to the above examples that was capable of acting on reticuline derivatives such as oripavine using the backbone plasmid p415TEF. A plasmid containing demethylase-CPR (pOD1184 from Example 7) was also expressed in combination with various candidate transporter proteins. Yeast strain construction and method of screening for transporters were as previously described in Example 7. Table 46-1 shows the result of percentage bioconversion from oripavine to nororipavine with the expression of various transporters. The screening was performed at pH 4.5. Table 46-1 also presents the percentage improvement in the bioconversion when normalized for a control strain expressing demethylase but not expressing any heterologous transporter.

Improvement of bioconversion with mutated versions of Hv_CYP_A0A2A4JAM9.

In this Example, various transporters, some for the first time, were screened together with three mutated versions of Hv_CYP_A0A2A4JAM9 from Heliothis virescens, Hv_CYP_A0A2A4JAM9_A110S, Hv_CYP_A0A2A4JAM9_A110N+H242P and Hv_CYP_A0A2A4JAM9_A110N+H242P+V224I. As shown in Table 46-1, the ranking of best transporters does not vary in a very significant way amongst these mutated Hv_CYP_A0A2A4JAM9. T193_AanPUP3_55 have been shown to be the best transporters paring with single mutant, Hv_CYP_A0A2A4JAM9_A110S and double mutant, Hv_CYP_A0A2A4JAM9_A110N+H242P, but percentage of bioconversion for oripavine slightly decreased with the triple mutant Hv_CYP_A0A2A4JAM9_A110N+H242P+V224I. From 58.1% and 57.5%, respectively to 52.5% in bioconversion from oripavine to nororipavine. For certain transporters such as T149_AcoPUP3_59 and T168_FvePUP3_37, the increment in number of mutations in Hv_CYP_A0A2A4JAM9 increases the percentage bioconversion of oripavine to nororipavine. For other transporters such as T253_AanPUP3_GA from Artemisia annua, double mutations in Hv_CYP_A0A2A4JAM9_A110N+H242P is preferred for best bioconversion of oripavine. The result also shows additional insect Equilibrative Nucleoside Transporters, T218_HviENT3_GA Heliothis virescens and T220_CsuENT3_GA from Chilo suppressalis that can mediate bioconversion of oripavine to nororipavine with the mutants of Hv_CYP_A0A2A4JAM9. T220_CsuENT3_GA, together with Hv_CYP_A0A2A4JAM9_A110S-mediated bioconversion, as much as 42.5% of the 500 μM oripavine fed was converted to nororipavine.

TABLE 46-1
Percentage demethylase-mediated bioconversion from oripavine to nororipavine with the expression of various transporters together
with mutated versions of Hv_CYP_A0A2A4JAM9 as compared to a control strains not expressing any heterologous transporters.

| | SEQ | Percentage bioconversion of oripavine at pH 4.5 (%) | | |
|---|---|---|---|---|
| | ID | | Hv_CYP_A0A2A4JAM9_A110N + | Hv_CYP_A0A2A4JAM9_A110N + |
| Transporter | NO: | Hv_CYP_A0A2A4JAM9_A110S | H242P | H242P + V224I |
| T102_PsoPUP3_1 | 466 | 40.1 | 38.6 | 38.4 |
| T116_HanPUP3_56 | 482 | 46.8 | 48.8 | 47.5 |
| T149_AcoPUP3_59 | 538 | NA | 49.6 | 54.3 |
| T165_AcoPUP3_13 | 568 | 43.0 | NA | NA |
| T168_FvePUP3_37 | 572 | 52.1 | 54.5 | 55.6 |
| T180_McoPUP3_46 | 596 | 56.0 | 52.7 | 53.0 |
| T182_CpaPUP3_62 | 600 | 54.8 | 47.1 | 48.4 |
| T192_CmiPUP3_47 | 612 | NA | 47.8 | 47.4 |
| T193_AanPUP3_55 | 614 | 58.1 | 57.5 | 52.5 |
| T195_JcuPUP3_71 | 618 | 51.6 | 45.1 | 45.3 |
| T198_AcoT97_GA | 624 | 50.4 | 49.1 | 49.5 |
| T210_NnuPUP3_GA | 648 | 53.1 | 46.3 | 48.7 |
| T213_HarPUP3_GA | 654 | 33.8 | 32.3 | 31.4 |
| T218_HviENT3_GA | 795 | NA | 26.9 | 26.0 |
| T220_CsuENT3_GA | 797 | 42.5 | 34.2 | 38.1 |
| T239_CmePUP3_GA | 809 | 30.5 | NA | NA |
| T240_PpePUP3_GA | 811 | 6.2 | NA | NA |
| T242_AchPUP3_GA | 813 | 19.0 | NA | NA |
| T243_EguPUP3_GA | 815 | 29.4 | 41.5 | 30.6 |
| T244_CcaPUP3_GA | 817 | 49.3 | 49.2 | 44.1 |
| T245_CcaPUP3_GA | 819 | 28.7 | NA | NA |
| T248_McoPUP3_GA | 821 | 14.9 | NA | NA |
| T253_AanPUP3_GA | 823 | 35.2 | 42.3 | 38.7 |
| T254_CcaPUP3_GA | 825 | 26.7 | 32.1 | 39.1 |
| Control | | 1.7 | 1.7 | 1.7 |

Note:
Demethylase: Hv_CYP_A0A2A4JAM9_A110S represents demethylase from Heliothis virescens with single mutation at amino acid residue 110. Hv_CYP_A0A2A4JAM9_A110N + H242P represents demethylase from Heliothis virescens with double mutations at amino acid residues 110 and 242. Hv_CYP_A0A2A4JAM9_A110N + H242P + V224I represents demethylase from Heliothis virescens with triple mutations at amino acid residues 110, 242 and 224. Control strain contains only a copy of demethylase, a copy of demethylase-CPR, HaCPR_E0A3A7 from Helicoverpa armigera, and an empty plasmid p416TEF. The demethylase-CPR, HaCPR_E0A3A7 is present in all strains.

### Conclusion

Table 46-1 shows some of the transporters that have been herein demonstrated to have shown very considerable improvements in the bioconversion from oripavine to nororipavine by 3 different mutated demethylases Hv_CYP_A0A2A4JAM9. In particular, the result of this example demonstrates that together with 1 out of the 3 mutated demethylases, expression of transporter T193_AanPUP3_55 from Artemisia annua stimulated somewhere in the range of 2987-3331% more in bioconversion of oripavine to nororipavine, when compared to a control strain without transporter. The significant yield shown herein are highly valuable given the nature of the opioid-related compounds produced.

### Example 47. Improvement of Bioconversion from Oripavine to Nororipavine with Multiple Ty Integration

### Optimization of Bioconversion Efficiency of Oripavine with Additional Ty Integration

Example 21 showed single round of multiple gene expression of demethylase and transporter by Ty integration improved bioconversion of oripavine substantially. In this example, as shown in Table 47-1, two rounds of Ty integration of demethylase, Hv_CYP_A0A2A4JAM9 and transporter, T193_AanPUP3_55 resulted in strain sOD438. When fed with 3000 μM of oripavine, sOD398 which was constructed by single Ty integration managed to convert 67.9% of oripavine to nororipavine. As for sOD438, with the same amount of oripavine fed, 88.3% of the 3000 μM of oripavine was converted to nororipavine. This clearly demonstrates that additional round of Ty integration greatly improved the bioconversion of oripavine to nororipavine. When fed with 5000 μM of oripavine, bioconversion of oripavine was 43.7% and 61.9%, respectively for sOD398 and sOD438. The percentage conversion decreased when more oripavine was fed which was due to substrate inhibition.

TABLE 47-1
Percentage demethylase-mediated bioconversion from oripavine to nororipavine
with multiple genes overexpression of demethylase and transporter
by single Ty integration versus double Ty integration.

| | | | Oripavine | Percentage Bioconversion |
|---|---|---|---|---|
| | Ty integrated genes | | fed | of Oripavine to |
| Strains | Ty1: P450/Transporter | Ty2: P450/Transporter | (μM) | Nororipavine (%) |
| sOD398 | Hv_CYP_A0A2A4JAM9/ | — | 3000 | 67.9 |
| | T180_McoPUP3_46 | | | |
| sOD438 | Hv_CYP_A0A2A4JAM9/ | Hv_CYP_A0A2A4JAM9/ | 3000 | 88.3 |
| | T193_AanPUP3_55 | T193_AanPUP3_55 | | |
| sOD398 | Hv_CYP_A0A2A4JAM9/ | — | 5000 | 43.7 |
| | T180_McoPUP3_46 | | | |
| sOD438 | Hv_CYP_A0A2A4JAM9/ | Hv_CYP_A0A2A4JAM9/ | 5000 | 61.9 |
| | T193_AanPUP3_55 | T193_AanPUP3_55 | | |

### Conclusion

The result presented in this example demonstrates that several rounds of multiple genes expression can greatly improves the efficiency of bioconversion from oripavine to nororipavine. Various source of demethylase and transporter have shown to exert the same improvement. The level of improvement is dependent on the demethylase/transporter combination and can be affected by substrate inhibition.

### REFERENCES

Gietz R D, Schiestl R H, Mortensen U H et al. Quick and easy yeast transformation using the LiAc/SS carrier DNA/PEG method. Nat Protoc 2007; 2:35-7.
Mumberg D, Müller R, Funk M.; Yeast Vectors for the Controlled Expression of Heterologous Proteins in Different Genetic Backgrounds; Gene; 156; 1995.
M E Pyne, K Kevvai, P S Grewal, L Narcross, B Choi, L Bourgeois, J E Dueber, & V J J Martin; A yeast platform for high-level synthesis of natural and unnatural tetrahydroisoquinoline alkaloids; BioRxiv preprint doi: https://doi.org/10.1101/863506, posted Dec. 5, 2019.
Tomas Hudlicky; “Recent advances in process development for opiate-derived pharmaceutical agents”; Can. J. Chem. 93: 492-501 (2015)
Galanie et al. (“Complete biosynthesis of opioids in yeast”; Science. 2015 Sep. 4; 349(6252): 1095-1100)
Jørgensen et al. (“Origin and evolution of transporter substrate specificity within the NPF family”; eLife 2017; 6:e19466. DOI: 10.7554/eLife.19466)
Jørgensen et al. (“A Functional EXXEK Motif is Essential for Proton Coupling and Active Glu-cosinolate Transport by NPF2.11”; Plant Cell Physiol. 56(12): 2340-2350 (2015))
Sipos et al. (“First Synthesis and Utilization of Oripavidine—a concise and Efficient Route to Important Morphinans and Apomorphines”; Helvetica Chimica Acta. Vol. 92: 1359-1365 (2009))
Dastmalchi et al. (“Purine permease-type benzylisquinoline alkaloid transporters in opium poppy”; Plant Physiology Preview. DOI:10.1104/pp. 19.00565 (2019))
Maury J, et al. EasyCloneMulti: A Set of Vectors for Simultaneous and Multiple Genomic Integrations in Saccharomyces cerevisiae. PLoS One 11(3):e0150394 (2016)
Bitter B G A, Egan K M, Koski R A, Jones M O, Elliott S G, Giffin J C. Expression and secretion vectors for yeast. Methods Enzymol. 153: 516-44. pmid:2828848 (1987)
Mikkelsen et al.; Microbial production of indolylglucosinolate through engineering of a multi-gene pathway in a versatile yeast expression platform; Metabolic Engineering; Volume 14; Issue 2; Pages 104-111; March 2012.
Nour-Eldin H. H., Hansen B. G., Norholm M. H., Jensen J. K., Halkier B. A. (2006); Advancing uracil-excision based cloning towards an ideal technique for cloning PCR fragments; Nucleic Acids Res; 34:e122.
Hansen B. G. et al (2011); Versatile enzyme expression and characterization system for Aspergillus nidulans, with the Penicillium brevicompactum polyketide synthase gene from the mycophenolic acid gene cluster as a test case; App. and Environmental Microbiology; 77(9):3044-3051.
Nielsen, J. B., M. L. Nielsen, and U. H. Mortensen; (2008); Transient disruption of nonhomologous end-joining facilitates targeted genome manipulation in the filamentous fungus Aspergillus nidulans; Fungal Genet. Biol.; 45:165-170.
Hoffman, M., et al.; 2003; Identification of rate-limiting steps in yeast heme biosynthesis; Biochemical and biophysical research communications; 310; 1247-1253.
Krainer, F. W., et al.; 2015; Optimizing cofactor availability for the production of recombinant heme peroxidase in Pichia pastoris; Microbial cell factories; 14, 4.
Krishnamurthy P, Xie T, Schuetz J D.; 2007; The role of transporters in cellular heme and porphyrin homeostasis; Pharmacol Ther.; 114, 345-358.
Michener, J. K., et al.; 2012; Identification and treatment of heme depletion attributed to overexpression of a lineage of evolved P450 monooxygenases. Proceedings of the National Academy of Sciences. 109; 19504-19509.
Protchenko, O., and C. C. Philpott; 2003; Regulation of intracellular heme levels by HMX1, a homologue of heme oxygenase, in Saccharomyces cerevisiae; J. Biol. Chem.; 278:36582-36587.
Hughes A L, et al.; 2007; Dap1/PGRMC1 binds and regulates cytochrome P450 enzymes; Cell Metabolism; February 5(2):143-149.
Kawahara T, et al.; 1997; Endoplasmic reticulum stress-induced mRNA splicing permits synthesis of transcription factor Hac1p/Ern4p that activates the unfolded protein response; Molecular Biology Cell; 8(10):1845-1862
Yu X W, et al.; 2017; Identification of novel factors enhancing recombinant protein production in multicopy Komagataella phaffii based on transcriptomic analysis of overexpression effects; Sci. Rep. November; 24; 7(1):16249
Stincone A, et al.; 2015; The return of metabolism: biochemistry and physiology of the pentose phosphate pathway; Biol Rev Camb Philos Soc.; August; 90(3):927-963.
Kalász H, et al.; 1998; Formaldehyde generation by N-demethylation; Acta Biol Hung.; 49(2-4):339-44.
Wehner E P, et al.; (1993); Molecular structure and genetic regulation of SFA, a gene responsible for resistance to formaldehyde in Saccharomyces cerevisiae, and characterization of its protein product; Mol. Gen. Genet.; 237(3):351-358.
Rebba C. Boswell-Casteel and Franklin A. Hays; Equilibrative Nucleoside Transporters—A Review; Nucleosides Nucleotides Nucleic Acids; 2017 Jan. 2; 36(1): 7-30.
Sonnhammer et al., Nucl. Acids Res., 26:320-322 (1998).
Sonnhammer et al., Proteins, 28:405-420 (1997).
Bateman et al., Nucl. Acids Res., 27:260-262 (1999).
Osmani et al., 2009, Phytochemistry 70: 325-347
Tolkachev, O. N., Shemeryankin, B. V. & Pronina, N. V. Isolation and purification of alkaloids. Chem Nat Compd 19, 387-400 (1983).
Janicot J L, Caude M, Rosset R. Separation of opium alkaloids by carbon dioxide sub- and supercritical fluid chromatography with packed columns. Application to the quantitative analysis of poppy straw extracts. J Chromatogr. 1988 Mar. 25; 437(2):351-64.
A. Barbier, UNODC Bulletin on Narcotics-1950 Issue 3
V. S. Ramanathan and P. Chandra, Bulletin on Narcotics-1980, Issue 2.
Hosztafi, S., Advances in Bioscience and Biotechnology 2014 Volume 5, pp 704-717.
Frank E-Hamerslag “Technology and Chemistry of Alkaloids” D Van Nostrand Company Inc Publishers 1950
John D, Thorton, Science and practice of liquid-liquid extraction Oxford:Clarendon Press, 1992—Chapter by Michael S. Verrall
Walter R. Heumann, Bulletin on Narcotics—1957 Issue 2-006—UNODC The manufacture of Alkaloids

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

1. A genetically modified host cell comprising a pathway having enhanced production of one or more benzylisoquinoline alkaloids wherein the cell expresses of one or more heterologous insect genes encoding one or more insect demethylases capable of converting thebaine into northebaine, thebaine into oripavine, thebaine into nororipavine and/or oripavine into nororipavine.

2. The cell of claim 1, wherein the insect demethylases have a product:by-product molar ratio of at least 2.0, such as at least 2.25, such as at least 2.5, such as at least 2.75, such as at least 3.0, such as at least 3.25, such as at least 3.5, such as at least 3.75, such as at least 4.0, such as at least 4.5, such as at least 5.0, such as at least 10.0 and wherein when the product is northebaine then the by-product is thebaine N-oxide and/or northebaine oxaziridine and when the product is nororipavine then the by-product is oripavine N-oxide and/or nororipavine oxaziridine.

3. The cell of claim 1, wherein the insect demethylases are of family CYP6, optionally of a genus selected from Helicoverpa, Heliothis and Spodoptera, optionally of a species selected from Helicoverpa annigera, Heliothis virescens and Spodoptera exigua.

4. The cell of claim 1, wherein the insect demethylase comprises a polypeptide selected from the group consisting of:
a) a demethylase which is at least 70%, optionally 80% or 90% identical to the insect demethylase comprised in any one of SEQ ID NO: 140, 142, 144, 146, 148, 150, 152, 154, 156, 158, 160, 162, 164, 166, 168, 170, 172, 174, 176, 178, 180, 182, 184, 186, 188, 190, 192, 194, 196, 827, 829, 831, 833, 835, 837, 839, 841, 843, 845, 847, 849, 851, 853, 855, 857, 859, 861, 863, 865, 867 and 869;
b) a demethylase encoded by a polynucleotide which is at least 70% identical to the polynucleotide comprised in any one of SEQ ID NO: 141, 143, 145, 147, 149, 151, 153, 155, 157, 159, 161, 163, 165, 167, 169, 171, 173, 175, 177, 179, 181, 183, 185, 187, 189, 191, 193, 195, 197, 828, 830, 832, 834, 836, 838, 840, 842, 844, 846, 848, 850, 852, 854, 856, 858, 860, 862, 864, 866, 868 and 870 or genomic DNA thereof; and
c) a functional variant of the insect demethylase of (a) or (b) capable of converting thebaine into northebaine, thebaine into oripavine, thebaine into nororipavine and/or oripavine into nororipavine.

5. The cell of claim 1, wherein the demethylases are artificial mutants comprising one or more mutations in a signal sequence, optionally wherein the signal sequence of the demethylases has been wholly or partially been replaced by a signal sequence from another enzyme.

6. The cell of claim 5, wherein the demethylases are artificial mutants having least 70%, optionally 80% or 90% identity to the demethylase comprised in SEQ ID NO: 152 and comprises one or more mutations corresponding to A110X, H242X, and/or V224X, such as A110N, H242P and/or V224I.

7. The cell of claim 5, wherein the demethylases are artificial mutants having at least 70%, optionally 80% or 90% identity to the demethylase comprised in SEQ ID NO: 140 and comprises one or more mutations corresponding to A316X and/or D392X, such as A316G and/or D392E.

8. The cell of claim 1, wherein the demethylase comprises one or more conserved amino acids corresponding to positions G103, H111, K167, E198, R219, L223, I256, A259, L273, V284, I309, L314, Q517, L160, N216, R443 of SEQ ID NO: 152 or conservative substitutions thereof.

9. The cell of claim 8, wherein the demethylase comprises a polypeptide which is at least 60% identical to the insect demethylase comprised in SEQ ID NO: 152.

10. The cell of claim 8, wherein the selected one or more conserved amino acid is/are in or near the active site of the demethylase, optionally corresponding to positions G103, H111 and L314 of SEQ ID NO: 152 or conservative substitutions thereof.

11. The cell of claim 1, further comprising a demethylase-CPR capable of reducing and/or regenerating the demethylase enzyme.

12. The cell of claim 11, wherein the demethylase-CPR is heterologous to the cell.

13. The cell of claim 11, wherein the demethylase-CPR is derived from an insect.

14. The cell of claim 13, wherein the insect demethylase-CPR is from an insect of a genus selected from Helicoverpa, Heliothis and Spodoptera, optionally of a species selected from Helicoverpa armigera, Heliothis virescens and Spodoptera exigua.

15. The cell of claim 13, wherein the demethylase-CPR comprises a polypeptide selected from the group consisting of:
a) a polypeptide which is at least 70% identical to the demethylase-CPR comprised in SEQ ID NO: 292, 294, 296, 298, 300 or 302;
b) a polypeptide encoded by a polynucleotide which is at least 70% identical to the polynucleotide comprised in SEQ ID NO: 293, 295, 297, 299, 301, 303 or 304 or genomic DNA thereof; and
c) a functional variant of the demethylase-CPR of (a) or (b) capable of reducing/regenerating the demethylase.

16. The cell of claim 1, wherein the cell comprises one or more features selected from:
a) expression of one or more heterologous genes encoding a tyrosine hydroxylase (TH) converting L-tyrosine into L-dopa, wherein the TH has at least 70% identity to the TH comprised in 7, 9, 11, 13, 15, 17, 19, 21, 23, 25, 27, 29, 31, 33, 35, 37, 39, 41, 43, 45, 47, 49, 51, 53, 55, 57, 59, 61, 63 or 65;
b) reduction or elimination of activity of one or more dehydrogenases native to the host cell comprised in SEQ ID NO: 663, 665, 667, 669, 671, 673, 675, 677, 679, 681, 683, 685, 687, 689, 691, 693, 695, 697, 699, 701, 703 or 705;
c) reduction or elimination of activity of one or more reductases native to the host cell comprised in SEQ ID NO: 707, 709, 711, 713, 715, 717, 719, 721, 723, 725, 727, 729 or 731;
d) expression of one or more heterologous genes encoding a norcoclaurine synthase (NCS) converting Dopamine and 4-HPAA into (S)-norcoclaurine, wherein the NCS has at least 70% identity to the NCS comprised in SEQ ID NO: 73 OR 76;
e) expression of one or more heterologous genes encoding
i) a fused 1,2-dehydroreticuline synthase-1,2-dehydroreticuline reductase (DRS-DRR) converting (S)-Reticuline into (R)-reticuline, wherein
ia) the DRS-DDR has at least 70% identity to the DRS-DRR comprised in SEQ ID NO: 92, 94, 96;
or
ib) the DRS moiety has at least 70%, identity to the DRS comprised in SEQ ID NO: 98, 100, 102, 104 or 106; and the DRR moiety has at least 70% identity to the DRR comprised in SEQ ID NO: 108 or 110; or
ii) a DRS having at least 70% identity to the DRS comprised in SEQ ID NO: 98, 100, 102, 104 or 106; and a DRR having at least 70% identity to the DRR comprised in SEQ ID NO: 108 or 110;
iii) a fused 1,2-dehydroreticuline synthase-1,2-dehydroreticuline reductase (DRS-DRR) converting (S)-Reticuline into (R)-reticuline selected from DRS-DDR's having at least 70% identity to the DRS-DRR comprised in SEQ ID NO: 92, 94, 96; and/or
iv) a 1,2-dehydroreticuline synthase (DRS) selected from DRSs having at least 70% identity to the DRS comprised in SEQ ID NO: 98, 100, 102, 104 or 106; and a 1,2-dehydroreticuline reductases (DDR) selected from DDR's having at least 70% identity to the DRR comprised in SEQ ID NO: 108 or 110;
f) expression of one or more heterologous genes encoding a thebaine synthase (THS) converting 7-O-acetylsalutaridinol or 7-O-acetylsalutaridinol acetate into thebaine, wherein the THS has at least 70% identity to the THS comprised in SEQ ID NO: 126, 127, 128, 129, 131, 133, 134, 136 or 138; and
g) expression of one or more heterologous genes encoding a transporter protein capable of increasing uptake or export in the host cell of a reticuline derivative selected from transporter proteins having at least 70% identity to the transporter protein comprised in SEQ ID NO: 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 733, 735, 795, 797, 799, 801, 803, 805, 807, 809, 811, 813, 815, 817, 819, 821, 823 or 825.

17. The cell of claim 1, further expressing one or more genes encoding polypeptides selected from:
a) a 3-deoxy-D-arabino-2-heptulosonic acid 7-phosphate synthase (DAHP synthase) converting PEP and E4P into DAHP;
b) a 5-enolpyruvylshikimate-3-phosphate (EPSP) synthase (aro1) converting 3-phosphoshikimate and PEP into EPSP;
c) an aro1 polypeptide converting DHAP and PEP into EPSP;
d) a chorismate synthase converting EPSP into Chorismate;
e) a chorismate mutase converting Chorismate into prephenate;
f) a prephenate dehydrogenase (Tyr1) converting prephenate into 4-HPP;
g) an aromatic aminotransferase converting 4-HPP into L-Tyrosine;
h) a TH-CPR capable of reducing TH;
i) a L-dopa decarboxylase (DODC) converting L-dopa into dopamine;
j) a Tyrosine decarboxylase (TYDC) converting L-dopa into dopamine;
k) a hydroxyphenylpyruvate decarboxylase (HPPDC) converting 4-HPP into 4-HPPA;
l) a monoamine oxidase converting dopamine into 3,4-DHPAA;
m) a 6-O-methyltransferase (6-OMT) converting (S)-norcoclaurine into (S)-Coclaurine and/or norlaudanosoline into (S)-3′-Hydroxy-coclaurine;
n) a Coclaurine-N-methyltransferase (CNMT) converting (S)-Coclaurine into (S)—N-Methylcoclaurine and/or (S)-3′-hydroxycoclaurine into (S)-3′-hydroxy-N-methyl-coclaurine;
o) a N-methylcoclaurine hydroxylase (NMCH) converting (S)-Coclaurine into (S)-3′-hydroxycoclaurine and/or (S)—N-Methylcoclaurine into (S)-3′-Hydroxy-N-Methylcoclaurine;
p) a 3′-hydroxy-N-methyl-(S)-coclaurine 4′-O-methyltransferase (4′-OMT) converting (S)-3′-Hydroxy-N-Methylcoclaurine into (S)-Reticuline;
q) a DRS-CPR capable of reducing DRS-DRR;
r) a salutaridine synthase (SAS) converting (R)-reticuline into Salutaridine;
s) a salutaridine reductase (SAR) converting Salutaridine to Salutaridinol; and
t) a salutaridinol 7-O-acetyltransferase (SAT) converting Salutaridinol into 7-O-acetylsalutaridinol.

18. The cell of claim 17, wherein the corresponding:
a) DAHP synthase has at least 70% identity to the DAHP synthase comprised in SEQ ID NO: 1
b) chorismate mutase has at least 70% identity to the chorismate synthase comprised in SEQ ID NO: 3;
c) TH-CPR has at least 70% identity to the TH-CPR comprised in SEQ ID NO: 67;
d) DODC has at least 70% identity to the DODC comprised in SEQ ID NO: 69 or 71;
e) 6-OMT has at least 70% identity to the 6-OMT comprised in SEQ ID NO: 79 or 81;
f) CNMT has at least 70% identity to the CNMT comprised in SEQ ID NO: 82 or 84;
g) NMCH has at least 70% identity to the NMCH comprised in SEQ ID NO: 85 OR 87;
h) 4′-OMT has at least 70% identity to the 4′-OMT comprised in SEQ ID NO: 89 or 91;
i) demethylase-CPR has at least 70% identity to the demethylase-CPR comprised in SEQ ID NO: 112 or 114;
j) SAS has at least 70% identity to the SAS comprised in SEQ ID NO: 116 or 118;
k) SAR has at least 70% identity to the SAR comprised in SEQ ID NO: 120 or 122;
l) SAT has at least 70% identity to the SAT comprised in SEQ ID NO: 123 or 125; and
m) ODM has at least 70% identity to the ODM comprised in SEQ ID NO: 218, 220, 222, 224, 226, 228, 236, 240, 250, 252, 254 and 268.

19. The cell of claim 1, wherein the cell is further modified to increase cytosolic levels of heme, optionally by
a) overexpressing and/or co-expressing one or more rate-limiting proteins in the heme pathway, such as HEM 2, HEM3 and/or HEM12 optionally by increasing the number of copies of the genes integrated in the host cell and/or by linking the genes to a combination of stronger and weaker promoters, such as promoters selected from pPYK1, pSEDh, pKEX2, pTEF1, pTDH3 and pPGK1, where pTEF1, pTDH3 and pPGK1; and/or
b) disrupting, deleting and/or attenuating any heme-down regulating genes, such as HMX1.

20. The cell of claim 1, wherein the cell is further modified by overexpressing and/or co-expressing P450 helper genes, optionally selected from DAP1, HAC1, KAR2, HSP82, CNE1, SSA1, CPR6, FES1, HSP104 and STI1.

21. The cell of claim 1, wherein the cell is further modified by overexpressing and/or co-expressing one or more genes in the pentose metabolic pathway, optionally selected from ZWF1 and GND1.

22. The cell of claim 1, wherein the cell is further modified by overexpressing and/or co-expressing one or more genes encoding factors lowering and/or detoxifying cytosolic formaldehyde, optionally selected from SFA1.

23. The cell of claim 1, wherein the cell is eukaryote selected from the group consisting of mammalian, insect, plant, or fungal cells.

24. The cell of claim 23, wherein the cell is a plant cell of the genus Physcomitrella or Papaver or Nicotiana.

25. The cell of claim 24, wherein the cell is a plant cell of the species Papaver soniferum or Nicotiana benthamiana.

26. The cell of claim 23, wherein the cell is a fungal cell selected from the phylas consisting of Ascomycota, Basidiomycota, Neocallimastigomycota, Glomeromycota, Blastocladiomycota, Chytridiomycota, Zygomycota, Oomycota and Microsporidia.

27. The cell of claim 26, wherein the fungal cell is a yeast selected from the group consisting of ascosporogenous yeast (Endomycetales), basidiosporogenous yeast, and Fungi Imperfecti yeast (Blastomycetes).

28. The cell of claim 27, wherein the yeast cell is selected from the genera consisting of Saccharomyces, Kluyveromyces, Candida, Pichia, Debaryomyces, Hansenula, Yarrowia, Zygosaccharomyces, and Schizosaccharomyces.

29. The cell of claim 28, wherein the yeast cell is selected from the species consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica.

30. The cell of claim 26, wherein the fungal cell is a filamentous fungus.

31. The cell of claim 30, wherein the filamentous fungal cell is selected from the phylas consisting of Ascomycota, Eumycota and Oomycota.

32. The cell of claim 31, wherein the filamentous fungal cell is selected from the genera consisting of Acremonium, Aspergillus, Aureobasidium, Bjerkandera, Ceriporiopsis, Chrysosporium, Coprinus, Corio/us, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma

33. The cell of claim 32, wherein the filamentous fungal cell is selected from the species consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvernispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatun, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.

34. A polynucleotide construct comprising a polynucleotide sequence encoding a heterologous enzymes or transporter protein of any preceding claim operably linked to one or more control sequences.

35. The polynucleotide construct of claim 34, wherein the control sequence is heterologous to the polynucleotide.

36. The polynucleotide construct of claim 35, wherein the construct is an expression vector.

37. The cell of claim 1 comprising the polynucleotide construct of claim 34.

38. A cell culture, comprising the cell of any preceding claim and a growth medium.

39. A method for producing a benzylisoquinoline alkaloid comprising
a) culturing the cell culture of claim 38 at conditions allowing the cell to produce the benzylisoquinoline alkaloid; and
b) optionally recovering and/or isolating the benzylisoquinoline alkaloid.

40. The method of claim 39, wherein one or more steps of producing the benzylisoquinoline alkaloid is performed in vitro.

41. The method of claim 39, comprising converting thebaine to northebaine in the cell, wherein the conversion is performed at a pH from 6 to 8, such as from 6.5 to 7.5, such as about 7.0.

42. The method of claim 39, comprising converting oripavine to nororipavine in the cell, wherein the conversion is performed at a pH from 3.5 to 5.5, such as from 3.0 to 5.0, such as about 4.5.

43. The method of claim 39, comprising feeding the cell culture with one or more exogenous benzylisoquinoline alkaloid precursors.

44. The method of claim 43, wherein the exogenous benzylisoquinoline alkaloid precursor is thebaine and/or oripavine.

45. The method of claim 39, wherein the benzylisoquinoline alkaloid is of the general formula R1-V-H (V):
(V)
or a salt thereof.

46. The method of claim 46, wherein the benzylisoquinoline alkaloid is a nororipavine, HO—V—H (VI), of the general formula:
(VI)
or a salt thereof.

47. The method of claim 45, further comprising chemically or biologically modifying the benzylisoquinoline alkaloid.

48. The method of claim 47, wherein the modified benzylisoquinoline alkaloid is selected from one or more of buprenorphine, naltrexone, naloxone and nalbuphine.

49. The method of claim 47, wherein the benzylisoquinoline alkaloid to be modified is one or more of thebaine, northebaine, oripavine or nororipavine and the method further comprises subjecting the benzylisoquinoline alkaloid in sequence to a bis-benzylation step, a Diels-Alder step and a Grignard step converting the benzylisoquinoline alkaloid into buprenorphine.

50. The method of claim 49, wherein the benzylisoquinoline alkaloid to be modified is HO—VI-H (VI).

51. The method of claim 50, further comprising:
a) in a first solvent system S-1 comprising a polar protic solvent, reacting the compound HO—VI-H (VI), with benzyl halide, benzyl sulfonate, or activated benzyl alcohol to provide a compound BnO—VI-Bn (VII) of the general formula:
(VII);
b) in a second solvent system S-2 comprising a polar protic solvent, reacting compound BnO—VI-Bn (VII) with methyl vinyl ketone to provide a compound BnO—VII-Bn (VIII) of the general formula:
(VIII);
c) in a third solvent system S-3 comprising a nonpolar solvent, reacting compound BnO—VII-Bn (VIII) with a tert-butylmagnesium compound to provide a compound BnO-VIIIA-Bn (LX) of the general formula:
(IX);
d) reacting Compound BnO-VIIIA-Bn (IX) with H2 in the presence of a hydrogenation catalyst to provide a compound HO—IX—H (X) of the general formula:
(X);
e) reacting Compound HO—IX—H (X) with
i. cyclopropane carboxaldehyde followed by a hydride source; or:
ii. cyclopropanecarboxylic acid halide followed by a reducing agent; or
iii. cyclopropylmethyl halide or activated cyclopropane methanol;
to provide buprenorphine.

52. The method of claim 51, wherein S-1 comprises at least one protic solvent having a dielectric constant of at least about 12, or at least about 14, or at least about 16.

53. The method of claim 52, wherein S-1 comprises at least about 50 vol. %, or at least about 75 vol. %, or at least about 90 vol. % of the at least one protic solvent having a dielectric constant of at least about 12 (e.g. at least 14, or at least 16).

54. The method of claim 51, wherein S-1 comprises at least one protic solvent having a polarity index of at least about 3, or at least about 3.5, or at least about 4.

55. The method of claim 54, wherein S-1 comprises at least about 50 vol. %, or at least about 75 vol. %, or at least about 90 vol. % of the at least one protic solvent having a polarity index of at least about 3, e.g., at least 3.5, or at least 4.

56. The method of claim 51, wherein S-2 comprises at least one protic solvent having a dielectric constant of at least about 12, or at least about 14, or at least about 16.

57. The method of claim 56, wherein S-2 comprises at least about 50 vol. %, or at least about 75 vol. %, or at least about 90 vol. % of the at least one protic solvent having a dielectric constant of at least about 12, e.g. at least 14, or at least 16.

58. The method of claim 51, wherein S-2 comprises at least one protic solvent having a polarity index of at least about 3, or at least about 3.5, or at least about 4.

59. The method of claim 58, wherein S-2 comprises at least about 50 vol. %, or at least about 75 vol. %, or at least about 90 vol. % of the at least one protic solvent having a polarity index of at least about 3, e.g. at least 3.5, or at least 4.

60. The method of claim 51, wherein S-1 comprises isopropanol and optionally water.

61. The method of claim 51, wherein S-2 comprises isopropanol and optionally water.

62. The method of claim 60, wherein S-1 and/or S-2 comprises about 50-100 vol. % isopropanol and 0 to about 50 vol. % water.

63. The method of claim 51, wherein step 51.b) is conducted in the presence of oxygen.

64. The method of claim 51, wherein the methyl vinyl ketone of step 51.b) is added to a crude reaction product of step 51.a), the crude reaction product comprising solvent S-1 and compound BnO—II-Bn (VII).

65. The method of claim 51, wherein S-3 comprises at least one nonpolar solvent having a dielectric constant of at most about 6, or at most about 5, or at most about 4.

66. The method of claim 65, wherein S-3 comprises at least about 50 vol. %, or at least about 75 vol. %, or at least about 90 vol. % of the at least one nonpolar solvent having a dielectric constant of at most 6, e.g. at most 5, or at most 4.

67. The method of claim 51, wherein S-3 comprises at least one nonpolar solvent having a polarity index of less than 3, or less than 2, or less than 1.

68. The method of claim 67, wherein S-3 comprises at least about 50 vol. %, or at least about 75 vol. %, or at least about 90 vol. % of the at least one nonpolar solvent having a polarity index of less than 3, e.g. less than 2, or less than 1.

69. The method of claim 51, wherein S-3 comprises less than about 10 vol. %, or less than about 5 vol. %, or less than about 2 vol. %, or less than about 1 vol. % of a total amount of solvents having a dielectric constant of greater than 6.

70. The method of claim 51, wherein S-3 comprises less than 10 vol. %, or less than 5 vol. %, or less than 2 vol. %, or less than 1 vol. % of total amount of solvents having a polarity index of 3 or greater.

71. The method of claim 51, wherein S-3 comprises 30-90 vol. % of one or more alkanes and/or cycloalkanes.

72. The method of claim 71, wherein the one or more alkanes and/or cycloalkanes comprises, e.g. is cyclohexane.

73. The method of claim 51, wherein S-3 comprises 10-50 vol. % toluene, 30-90 vol. % cyclohexane, and up to 30 vol. % tetrahydrofuran.

74. The method of claim 51, wherein the tert-butylmagnesium compound comprises one or both of a tert-butylmagnesium halide and di-tert-butylmagnesium.

75. The method of claim 51, wherein the tert-butylmagnesium compound comprises a tert-butylmagnesium halide and di-tert-butylmagnesium.

76. A fermentation composition comprising the cell culture of claim 38 and the benzylisoquinoline alkaloid comprised therein.

77. The fermentation composition of claim 76, wherein at least 10%, 25%, 50%, such as at least 75%, such as at least 95%, such as at least 99% of the cells are lysed.

78. The fermentation composition of claim 76, wherein at least 10%, 25%, 50%, such as at least 75%, such as at least 95%, such as at least 99% of solid cellular material has separated from the liquid.

79. The fermentation composition of claim 76, further comprising one or more compounds selected from trace metals, vitamins, salts, yeast nitrogen base, carbon source, YNB, and/or amino acids of the fermentation: wherein the concentration of the benzylisoquinoline alkaloid is at least 1 mg/kg composition.

80. A composition comprising the fermentation composition of claim 76 and one or more carriers, agents, additives and/or excipients.

81. A pharmaceutical composition comprising the fermentation composition of claim 76 and one or more pharmaceutical grade excipient, additives and/or adjuvants.

82. The pharmaceutical composition of claim 81, wherein the pharmaceutical preparation is in form of a powder, tablet or a capsule.

83. The pharmaceutical composition of claim 81, wherein the pharmaceutical preparation is in form of a pharmaceutical solution, suspension, lotion or ointment.

84. The pharmaceutical composition of claim 81 for use as a medicament for prevention, treatment and/or relief of a disease in a mammal.

85. The pharmaceutical composition of claim 84 for use in the prevention, treatment and/or relief of pain, infections, tussive conditions, parasitic conditions, cytotoxic conditions, opiate poisoning conditions and/or cancerous conditions in a mammal.

86. A method for preparing the pharmaceutical composition of claim 81 comprising mixing the fermentation composition of claim 76 with one or more pharmaceutical grade excipient, additives and/or adjuvants.

87. A method for preventing, treating and/or relieving a disease comprising administering a therapeutically effective amount of the pharmaceutical composition of claim 81 to a mammal.

88. The method of claim 87, wherein the disease is pain, infections, tussive conditions, parasitic conditions, cytotoxic conditions, opiate poisoning conditions and/or cancerous conditions.

89. A mutant insect demethylase comprising one or more mutations in the signal sequence of the naturally occurring insect demethylase.

90. The mutant demethylase of claim 89, wherein the signal sequence of the demethylase has been wholly or partially been replaced by a signal sequence from another enzyme.

91. The mutant demethylase of claim 89, wherein the demethylase has least 70%, such as at least 80%, such as at least 90%, such as at least 95%, such as at least 96%, such as at least 97%, such as at least 98%, such as at least 99%, such as 100% identity to the demethylase comprised in SEQ ID NO: 845, 847, 851, 853, 857, 859, 863, 865, 867 or 869.

92. A mutant insect demethylase having least 70%, such as at least 80%, such as at least 90%, such as at least 95%, such as at least 96%, such as at least 97%, such as at least 98%, such as at least 99%, such as 100% identity to the demethylase comprised in SEQ ID NO: 152 and comprising one or more mutations corresponding to A110X, H242X, and/or V224X, optionally A110N, H242P and/or V224I.

93. A mutant insect demethylase having at least 70%, such as at least 80%, such as at least 90%, such as at least 95%, such as at least 96%, such as at least 97%, such as at least 98%, such as at least 99%, such as 100% identity to the demethylase comprised in SEQ ID NO: 140 and comprising one or more mutations corresponding to A316X and/or D392X, optionally A316G and/or D392E.
